(19) United States
(12) Patent Application Publication
(10) Pub. No.: US 2024/0117345 A1
(43) Pub. Date: Apr. 11, 2024

(54) COMPOSITIONS AND METHODS FOR TREATING TRANSTHYRETIN AMYLOIDOSIS

(71) Applicant: Beam Therapeutics Inc., Cambridge, MA (US)

(72) Inventors: Michael PACKER, Cambridge, MA (US); Lo-I CHENG, Cambridge, MA (US); Tanggis BOHNUUD, Cambridge, MA (US)

(73) Assignee: Beam Therapeutics Inc., Cambridge, MA (US)

(21) Appl. No.: 18/507,980

(22) Filed: Nov. 13, 2023

Related U.S. Application Data
(63) Continuation of application No. PCT/US2022/029278, filed on May 13, 2022.
(60) Provisional application No. 63/189,060, filed on May 14, 2021.

(51) Int. Cl. C12N 15/11; A61K 48/00; C12N 9/22; C12N 9/78

(52) U.S. Cl. CPC C12N 15/111; A61K 48/005; C12N 9/22; C12N 9/78; C12N 15/11; C12Y 305/04004; C12Y 305/04005; C12N 2310/20; C12N 2320/34; C12N 2800/80

(57) ABSTRACT

The invention features compositions and methods for editing a transthyretin polynucleotide sequence to treat amyloidosis.

### CROSS-REFERENCE TO RELATED APPLICATIONS

This application is a continuation under 35 U.S.C. § 111(a) of PCT International Patent Application No. PCT/US2022/029278, filed May 13, 2022, designating the United States and published in English, which claims priority to and the benefit of U.S. Provisional Application No. 63/189,060, filed May 14, 2021, the entire contents of each of which are incorporated by reference herein.

### SEQUENCE LISTING

This present application contains a Sequence Listing which has been submitted electronically in XML format and is herein incorporated by reference in its entirety. The Sequence Listing XML file, created on Nov. 13, 2023, is named 180802-055003US_SL.xml and is 1,694,928 bytes in size.

### BACKGROUND OF THE INVENTION

Amyloidosis is a condition characterized by the buildup of abnormal deposits of amyloid protein in the body's organs and tissues. These protein deposits can occur in the peripheral nervous system, which is made up of nerves connecting the brain and spinal cord to muscles and sensory cells that detect sensations such as touch, pain, heat, and sound. Protein deposits in these nerves can result in a loss of sensation in the extremities (peripheral neuropathy). The autonomic nervous system, which controls involuntary body functions such as blood pressure, heart rate, and digestion, can also be affected by amyloidosis. In some cases, the brain and spinal cord (central nervous system) are affected. Mutations in the transthyretin (TTR) gene can cause transthyretin amyloidosis. Furthermore, patients expressing wild-type TTR may also develop amyloidosis. Liver transplant remains the gold standard for treating transthyretin amyloidosis.

Thus, there remains a need for compositions and methods for editing transthyretin polynucleotide sequences. These methods can be used for the treatment of amyloidosis.

### SUMMARY OF THE INVENTION

As described below, the present invention features compositions and methods for editing a transthyretin polynucleotide sequence to treat transthyretin amyloidosis.

In one aspect, the invention of the disclosure features a method for editing a transthyretin (TTR) polynucleotide sequence. The method involves: contacting the polynucleotide sequence with a guide RNA and a base editor containing a polynucleotide programmable DNA binding polypeptide and a deaminase. The guide RNA targets the base editor to effect an alteration of a nucleobase of the TTR polynucleotide sequence.

In another aspect, the invention of the disclosure features a method for editing a transthyretin (TTR) polynucleotide sequence. The method involves: contacting the polynucleotide sequence with a guide RNA and a fusion protein containing a polynucleotide programmable DNA binding domain and an adenosine deaminase domain. The adenosine deaminase domain contains an arginine (R) or a threonine (T) at amino acid position 147 of the following amino acid sequence, and the adenosine deaminase domain has at least about 85% sequence identity to the following amino acid sequence: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 4; TadA*7.10). The guide RNA targets the fusion protein to effect an alteration of a nucleobase of the TTR polynucleotide sequence.

In another aspect, the invention of the disclosure features a method for editing a transthyretin (TTR) polynucleotide sequence. The method involves: contacting the polynucleotide sequence with a guide RNA and a fusion protein containing a polynucleotide programmable DNA binding domain and a cytidine deaminase domain. The cytidine deaminase domain contains an amino acid sequence with at least about 85% sequence identity to the amino acid sequence: MSSETGPVAVDPTLRRRIEPHEFEVFFDPRELRKETCLLYEINWGGRHSIWRHTSQNTNKHVEV NFIEKFTTERYFCPNTRCSITWFLSWSPCGECSRAITEFLSRYPHVTLFIYIARLYHHADPRNR QGLRDLISSGVTIQIMTEQESGYCWRNFVNYSPSNEAHWPRYPHLLWVRLYVLELYCIILGLPPC LNILRRKQPQLTFFTIALQSCHYQRLPPHILWATGLK (SEQ ID NO: 15; BE4 cytidine deaminase domain). The guide RNA targets the fusion protein to effect an alteration of a nucleobase of the TTR polynucleotide sequence.

In another aspect, the invention of the disclosure features a method for editing a transthyretin (TTR) polynucleotide sequence. The method involves: contacting the polynucleotide sequence with a guide RNA and a Cas12b endonuclease, where the guide RNA targets the endonuclease to effect a double-stranded break of the TTR polynucleotide sequence.

In another aspect, the invention of the disclosure features a method for treating amyloidosis in a subject. The method involves administering to the subject a guide RNA and a fusion protein containing a polynucleotide programmable DNA binding domain and an adenosine deaminase domain. The adenosine deaminase domain contains an arginine (R) or a threonine (T) at amino acid position 147 of the following amino acid sequence, and the adenosine deaminase domain has at least about 85% sequence identity to the following amino acid sequence MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 4; TadA*7.10). The guide RNA targets the fusion protein to effect an alteration of a nucleobase of the TTR polynucleotide sequence.

In another aspect, the invention of the disclosure features a method for treating amyloidosis in a subject. The method involves administering to the subject a guide RNA and a fusion protein containing a polynucleotide programmable DNA binding domain and a cytidine deaminase domain. The cytidine deaminase domain contains an amino acid sequence with at least about 85% sequence identity to the amino acid sequence: MSSETGPVAVDPTLRRRIEPHEFEVFFDPRELRKETCLLYEINWGGRHSIWRHTSQNTNKHVEV NFIEKFTTERYFCPNTRCSITWFLSWSPCGECSRAITEFLSRYPHVTLFIYIARLYHHADPRNR QGLRDLISSGVTIQIMTEQESGYCWRNFVNYSPSNEAHWPRYPHLWVRLYVLELYCIILGLPPC LNILRRKQPQLTFFTIALQSCHYQRLPPHILWATGLK (SEQ ID NO: 15). The guide RNA targets the fusion protein to effect an alteration of a nucleobase of the TTR polynucleotide sequence.

In another aspect, the invention of the disclosure features a method for treating amyloidosis in a subject. The method involves administering to the subject a guide RNA and a polynucleotide encoding a base editor containing a polynucleotide programmable DNA binding polypeptide and a deaminase. The guide RNA targets the base editor to effect an alteration of a nucleobase of the TTR polynucleotide sequence.

In another aspect, the invention of the disclosure features a method for editing a transthyretin (TTR) polynucleotide sequence in a subject. The method involves administering to a subject a guide RNA and a Cas12b endonuclease. The guide RNA targets the endonuclease to effect a double-stranded break of the TTR polynucleotide sequence.

In another aspect, the invention of the disclosure features a composition containing one or more polynucleotides encoding a fusion protein and a guide RNA. The guide RNA contains a nucleic acid sequence that is complementary to a transthyretin (TTR) polynucleotide. The fusion protein contains a polynucleotide programmable DNA binding domain and a deaminase domain.

In another aspect, the invention of the disclosure features a composition containing one or more polynucleotides encoding an endonuclease and a guide RNA. The guide RNA contains a nucleic acid sequence that is complementary to a transthyretin (TTR) polynucleotide. The endonuclease contains the amino acid sequence:

bhCas12b v4MAPKKKRKVGIHGVPAAATRSFILKIEPNEEVKKGLWKTHEVLNHGIAYYMNILKLIRQEAI YEHHEQDPKNPKKVSKAEIQAELWDFVLKMQKCNSFTHEVDKDEVFNILRELYEELVPSSVEKK GEANQLSNKFLYPLVDPNSQSGKGTASSGRKPRWYNLKIAGDPSWEEEKKKWEEDKKKDPLAKI LGKLAEYGLIPLFIPYTDSNEPIVKEIKWMEKSRNQSVRRLDKDMFIQALERFLSWESWNLKVK EEYEKVEKEYKTLEERIKEDIQALKALEQYEKERQEQLLRDTLNTNEYRLSKRGLRGWREIIQK WLKMDENEPSEKYLEVFKDYQRKHPREAGDYSVYEFLSKKENHFIWRNHPEYPYLYATFCEIDK KKKDAKQQATFTLADPINHPLWVRFEERSGSNLNKYRILTEQLHTEKLKKKLTVQLDRLIYPTE SGGWEEKGKVDIVLLPSRQFYNQIFLDIEEKGKHAFTYKDESIKFPLKGTLGGARVQFDRDHLR RYPHKVESGNVGRIYFNMTVNIEPTESPVSKSLKIHRDDFPKVVNFKPKELTEWIKDSKGKKLK SGIESLEIGLRVMSIDLGQRQAAAASIFEVVDQKPDIEGKLFFPIKGTELYAVHRASFNIKLPG ETLVKSREVLRKAREDNLKLMNQKLNFLRNVLHFQQFEDITEREKRVTKWISRQENSDVPLVYQ DELIQIRELMYKPYKDWVAFLKQLHKRLEVEIGKEVKHWRKSLSDGRKGLYGISLKNIDEIDRT RKFLLRWSLRPTEPGEVRRLEPGQRFAIDQLNHLNALKEDRLKKMANTIIMHALGYCYDVRKKK WQAKNPACQIILFEDLSNYNPYGERSRFENSRLMKWSRREIPRQVALQGEIYGLQVGEVGAQFS SRFHAKTGSPGIRCRVVTKEKLQDNRFFKNLQREGRLTLDKIAVLKEGDLYPDKGGEKFISLSK DRKCVTTHADINAAQNLQKRFWTRTHGFYKVYCKAYQVDGQTVYIPESKDQKQKIIEEFGEGYF ILKDGVYEWVNAGKLKIKKGSSKQSSSELVDSDILKDSFDLASELKGEKLMLYRDPSGNVFPSD KWMAAGVFFGKLERILISKLTNQYSISTIEDDSSKQSMSGGSKRTADGSEFESPKKKRKVE (SEQ ID NO: 450). The guide RNA targets the endonuclease to effect a double-stranded break of the TTR polynucleotide sequence.

In another aspect, the invention of the disclosure features a pharmaceutical composition for the treatment of transthyretin (TTR) amyloidosis. The pharmaceutical composition contains: an endonuclease, or a nucleic acid encoding the endonuclease, and a guide RNA (gRNA) containing a nucleic acid sequence complementary to an transthyretin (TTR) polynucleotide in a pharmaceutically acceptable excipient. The endonuclease contains the amino acid sequence:

bhCas12b
(SEQ ID NO: 450)
v4MAPKKKRKVGIHGVPAAATRSFILKIEPNEEVKKGLWKTHEVLNHGIAYYMNILKLIRQEAI
YEHHEQDPKNPKKVSKAEIQAELWDFVLKMQKCNSFTHEVDKDEVENILRELYEELVPSSVEKK
GEANQLSNKFLYPLVDPNSQSGKGTASSGRKPRWYNLKIAGDPSWEEEKKKWEEDKKKDPLAKI
LGKLAEYGLIPLFIPYTDSNEPIVKEIKWMEKSRNQSVRRLDKDMFIQALERFLSWESWNLKVK
EEYEKVEKEYKTLEERIKEDIQALKALEQYEKERQEQLLRDTLNTNEYRLSKRGLRGWREIIQK
WLKMDENEPSEKYLEVFKDYQRKHPREAGDYSVYEFLSKKENHFIWRNHPEYPYLYATFCEIDK
KKKDAKQQATFTLADPINHPLWVRFEERSGSNLNKYRILTEQLHTEKLKKKLTVQLDRLIYPTE
SGGWEEKGKVDIVLLPSRQFYNQIFLDIEEKGKHAFTYKDESIKFPLKGTLGGARVQFDRDHLR
RYPHKVESGNVGRIYFNMTVNIEPTESPVSKSLKIHRDDFPKVVNFKPKELTEWIKDSKGKKLK
SGIESLEIGLRVMSIDLGQRQAAAASIFEVVDQKPDIEGKLFFPIKGTELYAVHRASENIKLPG
ETLVKSREVLRKAREDNLKLMNQKLNFLRNVLHFQQFEDITEREKRVTKWISRQENSDVPLVYQ
DELIQIRELMYKPYKDWVAFLKQLHKRLEVEIGKEVKHWRKSLSDGRKGLYGISLKNIDEIDRT
RKFLLRWSLRPTEPGEVRRLEPGQRFAIDQLNHLNALKEDRLKKMANTIIMHALGYCYDVRKKK
WQAKNPACQIILFEDLSNYNPYGERSRFENSRLMKWSRREIPRQVALQGEIYGLQVGEVGAQFS
SRFHAKTGSPGIRCRVVTKEKLQDNRFFKNLQREGRLTLDKIAVLKEGDLYPDKGGEKFISLSK
DRKCVTTHADINAAQNLQKRFWTRTHGFYKVYCKAYQVDGQTVYIPESKDQKQKIIEEFGEGYF
ILKDGVYEWVNAGKLKIKKGSSKQSSSELVDSDILKDSFDLASELKGEKLMLYRDPSGNVEPSD
KWMAAGVFFGKLERILISKLTNQYSISTIEDDSSKQSMSGGSKRTADGSEFESPKKKRKVE,

where the guide RNA targets the endonuclease to effect a double-stranded break of the TTR polynucleotide sequence.

In another aspect, the invention of the disclosure features a pharmaceutical composition for the treatment of transthyretin (TTR) amyloidosis. The pharmaceutical composition contains the composition of any of the above aspects, or embodiments thereof, and a pharmaceutically acceptable excipient.

In another aspect, the invention of the disclosure features a method of treating transthyretin (TTR) amyloidosis. The method involves administering to a subject in need thereof the pharmaceutical composition of any of the above aspects, or embodiments thereof.

In another aspect, the invention of the disclosure features use of the composition of any of the above aspects, or embodiments thereof, in the treatment of transthyretin (TTR) amyloidosis in a subject.

In another aspect, the invention of the disclosure features a method for treating amyloidosis in a subject. The method involves systemically administering to the subject a guide RNA and a fusion protein containing a polynucleotide programmable DNA binding domain and a deaminase domain. The guide RNA targets the base editor to effect an alteration of a nucleobase of the TTR polynucleotide sequence present in a liver cell of the subject.

In any of the above aspects, or embodiments thereof, the deaminase is an adenosine deaminase or a cytidine deaminase.

In any of the above aspects, or embodiments thereof, the editing introduces an alteration that corrects a mutation in a TTR polynucleotide. In any of the above aspects, or embodiments thereof, the editing introduces an alteration that reduces or eliminates expression of a TTR polypeptide. In any of the above aspects, or embodiments thereof, the editing introduces an alteration that reduces or eliminates expression of a TTR polypeptide by at least about 50% relative to a reference. In any of the above aspects, or embodiments thereof, the alteration is in a splice acceptor, splice donor, intronic sequence, exonic sequence, enhancer, or promoter.

In any of the above aspects, or embodiments thereof, the base editor contains a deaminase in complex with the polynucleotide programmable DNA binding polypeptide and the guide RNA, or the base editor is a fusion protein containing the polynucleotide programmable DNA binding polypeptide and the deaminase.

In any of the above aspects, or embodiments thereof, the alteration is in a promoter. In any of the above aspects, or embodiments thereof, the alteration is in a region of the TTR promoter corresponding to nucleotide positions +1 to −225 of the TTR promoter, where position +1 corresponds to A of the start codon (ATG) of the TTR polynucleotide sequence. In any of the above aspects, or embodiments thereof, the alteration is in a region of the TTR promoter corresponding to nucleotide positions +1 to −198 of the TTR promoter, where position +1 corresponds to A of the start codon (ATG) of the TTR polynucleotide sequence. In any of the above aspects, or embodiments thereof, the alteration is in a region of the TTR promoter corresponding to nucleotide positions +1 to −177 of the TTR promoter, where position +1 corresponds to A of the start codon (ATG) of the TTR polynucleotide sequence. In any of the above aspects, or embodiments thereof, the alteration is in a region of the TTR promoter corresponding to nucleotide positions −106 to −176 of the TTR promoter, where position +1 corresponds to A of the start codon (ATG) of the TTR polynucleotide sequence. In any of the above aspects, or embodiments thereof, the alteration is in a TATA box or ATG start codon.

In any of the above aspects, or embodiments thereof, alteration of the nucleobase disrupts gene splicing.

In any of the above aspects, or embodiments thereof, the TTR polynucleotide sequence encodes a mature TTR polypeptide containing a pathogenic alteration selected from one or more of T60A, V30M, V30A, V30G, V30L, V122I, and V122A. In any of the above aspects, or embodiments thereof, the pathogenic alteration is V122I.

In any of the above aspects, or embodiments thereof, the adenosine deaminase converts a target A•T to G•C in the TTR polynucleotide sequence. In any of the above aspects, or embodiments thereof, the cytidine deaminase converts a target C•G to T•A in the TTR polynucleotide sequence.

In any of the above aspects, or embodiments thereof, the altered nucleobase is 4A of the nucleotide sequence TATAGGAAAACCAGTGAGTC (SEQ ID NO: 425; TSBT×2602/gRNA1598 target site sequence corresponding to sgRNA_361); 6A of the nucleotide sequence TACTCACCTCTGCATGCTCA (SEQ ID NO: 426; TSBT×2603/gRNA1599 target site sequence corresponding to sgRNA_362); 5A of the nucleotide sequence ACTCACCTCTGCATGCTCAT (SEQ ID NO: 427; TSBT×2604/gRNA1606 target site sequence corresponding to sgRNA_363); 7A of the nucleotide sequence ATACTCACCTCTGCATGCTCA (SEQ ID NO: 429; TSBT×2606 target site sequence corresponding to sgRNA_365); 6A of the nucleotide sequence TTGGCAGGATGGCTTCTCATCG (SEQ ID NO: 431; TSBT×2608/gRNA-#19 target site corresponding to sgRNA_367); 9A of the sequence TTGGCAGGATGGCTTCTCATCG (SEQ ID NO: 431; TSBT×2608/gRNA-#19 target site corresponding to sgRNA_367); 5A of the sequence GGCTATCGTCACCAATCCCA (SEQ ID NO: 439; corresponding to sgRNA 375); or 4A of the sequence GCTATCGTCACCAATCCCAA (SEQ ID NO: 440; corresponding to sgRNA_376). In any of the above aspects, or embodiments thereof, the altered nucleobase is 7C of the nucleotide sequence TACT CACCTCTGCATGCTCA (SEQ ID NO: 426; TSBT×2603/gRNA1599 target site corresponding to sgRNA_362); 6C of the nucleotide sequence ACTCACCTCTGCATGCTCAT (SEQ ID NO: 427; TSBT×2604/gRNA1606 target site corresponding to sgRNA_363); 7C of the nucleotide sequence TACCACCTATGAGAGAAGAC (SEQ ID NO: 428; TSBT×2605 target site corresponding to sgRNA_364); 8C of the nucleotide sequence ATACTCACCTCTGCATGCTCA (SEQ ID NO: 429; TSBT×2606 target site corresponding to sgRNA_365); or 11C of the nucleotide sequence ACTGGTTTTCCTATAAGGTGT (SEQ ID NO: 430; TSBT×2607 target site corresponding to sgRNA_366).

In any of the above aspects, or embodiments thereof, the polynucleotide programmable DNA binding domain contains a Cas polypeptide. In any of the above aspects, or embodiments thereof, the polynucleotide programmable DNA binding domain contains a Cas9 or a Cas12 polypeptide or a fragment thereof. In embodiments, the Cas9 polypeptide contains a Streptococcus pyogenes Cas9 (SpCas9), Staphylococcus aureus Cas9 (SaCas9), Streptococcus thermophilus 1 Cas9 (St1Cas9), or Streptococcus canis Cas9 (ScCas9). In embodiments, the Cas 12 polypeptide contains a Cas12a, Cas12b, Cas12c, Cas12d, Cas12e, Cas12g, Cas12h, or Cas12i. In embodiments, the Cas12 polypeptide contains a sequence with at least about 85% amino acid sequence identity to Bacillus hisashii Cas12b, Bacillus thermoamylovorans Cas12b, Bacillus sp. V3-13 Cas12b, or Alicyclobacillus acidiphilus Cas12b.

In any of the above aspects, or embodiments thereof, the polynucleotide programmable DNA binding domain contains a Cas9 polypeptide with a protospacer-adjacent motif (PAM) specificity for a nucleic acid sequence selected from 5′-NGG-3′, 5′-NAG-3′, 5′-NGA-3′, 5′-NAA-3′, 5′-NNAGGA-3′, 5′-NNGRRT-3′, or 5′-NNACCA-3′. In any of the above aspects, or embodiments thereof, the polynucleotide programmable DNA binding domain contains a Cas9 polypeptide with specificity for an altered protospacer-adjacent motif (PAM). In embodiments, the nucleic acid sequence of the altered PAM is selected from 5′-NNNRRT-3′, 5′-NGA-3′, 5′-NGCG-3′, 5′-NGN-3′, 5′-NGCN-3′, 5′-NGTN-3′, and 5′-NAA-3′.

In any of the above aspects, or embodiments thereof, the polynucleotide programmable DNA binding domain is a nuclease inactive or nickase variant. In embodiments, the nuclease inactivated variant is a Cas9 (dCas9) containing the amino acid substitution D10A or a substitution at a corresponding amino acid position. In embodiments, the nuclease inactivated variant is a bhCas12b containing the amino acid substitutions D952A, S893R, K846R, and E837G, or substitutions at corresponding amino acid positions.

In any of the above aspects, or embodiments thereof, the adenosine deaminase domain is capable of deaminating adenine in deoxyribonucleic acid (DNA). In any of the above aspects, or embodiments thereof, the cytidine deaminase domain is capable of deaminating cytidine in deoxyribonucleic acid (DNA). In embodiments, the adenosine deaminase is a TadA deaminase.

In embodiments, the TadA deaminase is TadA*7.10, TadA*8.1, TadA*8.2, TadA*8.8, TadA*8.9, TadA*8.10, TadA*8.11, TadA*8.12, TadA*8.13, TadA*8.15, TadA*8.16, TadA*8.19, TadA*8.20, TadA*8.21, or TadA*8.24. In embodiments, the TadA deaminase is TadA*7.10. TadA*8.8, or TadA*8.13.

In any of the above aspects, or embodiments thereof, the base editor contains a fusion protein containing the deaminase flanked by an N-terminal fragment and a C-terminal fragment of the programmable DNA binding polypeptide, where the DNA binding polypeptide is a Cas9 polypeptide. In any of the above aspects, or embodiments thereof, the deaminase is inserted between amino acid positions 1029-1030 or 1247-1248 of a sequence with at least about 70%, 80%, 85%, 90%, 95%, or 100% sequence identity to the following amino acid sequence: spCas9

(SEQ ID NO: 201)
MDKKYSIGLDIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNE
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNEDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDELDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTE
KEDIQKAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRV
ILADANLDKVLSAYNKHRDKPIREQAENIIHLFTLTNLGAPAAFKYEDTTIDRKRYTSTKEVLD
ATLIHQSITGLYETRIDLSQLGGD.

In any of the above aspects, or embodiments thereof, the cytidine deaminase is an APOBEC or a variant thereof. In any of the above aspects, or embodiments thereof, the cytidine deaminase contains the amino acid sequence: MSSETGPVAVDPTLRRRIEPHEFEVFFDPRELRKETCLLYEINWGGRHSIWRHTSQNTNKHVEV NFIEKFTTERYFCPNTRCSITWFLSWSPCGECSRAITEFLSRYPHVTLFIYIARLYHHADPRNR QGLRDLISSGVTIQIMTEQESGYCWRNFVNYSPSNEAHWPRYPHLWVRLYVLELYCIILGLPPC LNILRRKQPQLTFFTIALQSCHYQRLPPHILWATGLK (SEQ ID NO: 15; BE4 cytidine deaminase domain), or a version of the amino acid sequence omitting the first methionine (M).

In any of the above aspects, or embodiments thereof, the base editor further contains one or more uracil glycosylase inhibitors (UGIs).

In any of the above aspects, or embodiments thereof, the base editor further contains one or more nuclear localization signals (NLS). In embodiments, the NLS is a bipartite NLS.

In any of the above aspects, or embodiments thereof, the guide RNA contains a CRISPR RNA (crRNA) and a trans-encoded small RNA (tracrRNA). The crRNA contains a nucleic acid sequence complementary to the TTR polynucleotide sequence.

In any of the above aspects, or embodiments thereof, the base editor is in complex or forms a complex with a single guide RNA (sgRNA) containing a nucleic acid sequence complementary to the TTR polynucleotide sequence.

In any of the above aspects, or embodiments thereof, the method further involves altering two or more nucleobases. In any of the above aspects, or embodiments thereof, the method further involves contacting the polynucleotide sequence with two or more distinct guide RNAs that target the TTR polynucleotide sequence.

In any of the above aspects, or embodiments thereof, the guide RNA(s) contains a nucleotide sequence selected from one or more of those sequences listed in Table 1, Table 2A, or Table 2B; or any of the aforementioned sequences where 1, 2, 3, 4, or 5 nucleotides is deleted from the 5′ and/or 3′ terminus of the nucleotide sequence.

In any of the above aspects, or embodiments thereof, the guide RNA(s) contains a nucleotide sequence selected from one or more of:

(SEQ ID NO: 408; sgRNA_361/gRNA1598)
5′-UAUAGGAAAACCAGUGAGUC-3′;
(SEQ ID NO: 409; sgRNA_362/gRNA1599)
5′-UACUCACCUCUGCAUGCUCA-3′;
(SEQ ID NO: 410; sgRNA_363/gRNA1606)
5′-ACUCACCUCUGCAUGCUCAU-3′;
(SEQ ID NO: 412; sgRNA_365)
5′-AUACUCACCUCUGCAUGCUCA-3′;
(SEQ ID NO: 414; sgRNA_367/gRNA-#19)
5′-UUGGCAGGAUGGCUUCUCAUCG-3′;
(SEQ ID NO: 422; sgRNA_375)
5′-GGCUAUCGUCACCAAUCCCA-3′;
(SEQ ID NO: 423; sgRNA_376)
5′-GCUAUCGUCACCAAUCCCAA-3′;
(SEQ ID NO: 561; gRNA1604)
5′-ACACCUUAUAGGAAAACCAG-3′;
(SEQ ID NO: 554; gRNA1597)
5′-CUCUCAUAGGUGGUAUUCAC-3′;
(SEQ ID NO: 557; gRNA1600)
5′-GCAACUUACCCAGAGGCAAA-3′;
(SEQ ID NO: 551; gRNA1594)
5′-CAACUUACCCAGAGGCAAAU-3′;
(SEQ ID NO: 558; gRNA1601)
5′-UCUGUAUACUCACCUCUGCA-3′;
(SEQ ID NO: 462; gRNA1756)
5′-CAAAUAUGAACCUUGUCUAG-3′;
(SEQ ID NO: 470; gRNA1764)
5′-GAACCUUGUCUAGAGAGAUU-3′;
(SEQ ID NO: 492; gRNA1786)
5′-UGAGUAUAAAAGCCCCAGGC-3′;
and
(SEQ ID NO: 478; gRNA1772)
5′-GCCAUCCUGCCAAGAAUGAG-3′;

or any of the aforementioned sequences where 1, 2, 3, 4, or 5 nucleotides is deleted from the 5′ and/or 3′ terminus of the nucleotide sequence.

In any of the above aspects, or embodiments thereof, the guide RNA(s) contains a nucleotide sequence selected from one or more of:

(SEQ ID NO: 409; sgRNA_362/gRNA1599)
5′-UACUCACCUCUGCAUGCUCA-3′,
(SEQ ID NO: 410; sgRNA_363/gRNA1606)
5′-ACUCACCUCUGCAUGCUCAU-3′,
(SEQ ID NO: 411; sgRNA_364)
5′-UACCACCUAUGAGAGAAGAC-3′,
(SEQ ID NO: 412; sgRNA_365)
5′-AUACUCACCUCUGCAUGCUCA-3′,
(SEQ ID NO: 413; sgRNA_366)
5′-ACUGGUUUUCCUAUAAGGUGU-3′,
(SEQ ID NO: 551; gRNA1594)
5′-CAACUUACCCAGAGGCAAAU-3′,
and
(SEQ ID NO: 496; gRNA1790)
5′-UGUUGACUAAGUCAAUAAUC-3′;

or any of the aforementioned sequences where 1, 2, 3, 4, or 5 nucleotides is deleted from the 5′ and/or 3′ terminus of the nucleotide sequence.

In any of the above aspects, or embodiments thereof, the guide RNA contains a nucleotide sequence, selected from one or more of:

(SEQ ID NO: 415; sgRNA_368)
5′-UCCUAUAAGGUGUGAAAGUCUG-3′,
(SEQ ID NO: 416; sgRNA_369)
5′-UGAGCCCAUGCAGCUCUCCAGA-3′,
(SEQ ID NO: 417; sgRNA_370)
5′-CUCCUCAGUUGUGAGCCCAUGC-3′,
(SEQ ID NO: 418; sgRNA_371)
5′-GUAGAAGGGAUAUACAAAGUGG-3′,
(SEQ ID NO: 419; sgRNA_372)
5′-CCACUUUGUAUAUCCCUUCUAC-3′,
(SEQ ID NO: 420; sgRNA_373)
5′-GGUGUCUAUUUCCACUUUGUAU-3′,
and
(SEQ ID NO: 421; sgRNA_374)
5′-CAUGAGCAUGCAGAGGUGAGUA-3′;

or any of the aforementioned sequences where 1, 2, 3, 4, or 5 nucleotides is deleted from the 5′ and/or 3′ terminus of the nucleotide sequence.

In any of the above aspects, or embodiments thereof, the guide RNA(s) contains 2-5 contiguous 2′-O-methylated nucleobases at the 3′ end and at the 5′ end. In any of the above aspects, or embodiments thereof, the guide RNA(s) contains 2-5 contiguous nucleobases at the 3′ end and at the 5′ end that contain phosphorothioate internucleotide linkages.

In any of the above aspects, or embodiments thereof, the Cas12b polypeptide is a bhCAS12b polypeptide. In any of the above aspects, or embodiments thereof, the bhCAS12b polypeptide contains the amino acid sequence:

bhCas12b
(SEQ ID NO: 450)
v4MAPKKKRKVGIHGVPAAATRSFILKIEPNEEVKKGLWKTHEVLNHGIAYYMNILKLIRQEAI
YEHHEQDPKNPKKVSKAEIQAELWDFVLKMQKCNSFTHEVDKDEVENILRELYEELVPSSVEKK
GEANQLSNKFLYPLVDPNSQSGKGTASSGRKPRWYNIKIAGDPSWEEEKKKWEEDKKKDPLAKI
LGKLAEYGLIPLFIPYTDSNEPIVKEIKWMEKSRNQSVRRLDKDMFIQALERFLSWESWNLKVK
EEYEKVEKEYKTLEERIKEDIQALKALEQYEKERQEQLLRDTLNTNEYRLSKRGLRGWREIIQK
WLKMDENEPSEKYLEVFKDYQRKHPREAGDYSVYEFLSKKENHFIWRNHPEYPYLYATFCEIDK
KKKDAKQQATFTLADPINHPLWVRFEERSGSNLNKYRILTEQLHTEKLKKKLTVQLDRLIYPTE
SGGWEEKGKVDIVLLPSRQFYNQIFLDIEEKGKHAFTYKDESIKFPLKGTLGGARVQFDRDHLR
RYPHKVESGNVGRIYFNMTVNIEPTESPVSKSLKIHRDDFPKVVNFKPKELTEWIKDSKGKKLK
SGIESLEIGLRVMSIDLGQRQAAAASIFEVVDQKPDIEGKLFFPIKGTELYAVHRASENIKLPG
ETLVKSREVLRKAREDNLKLMNQKLNFLRNVLHFQQFEDITEREKRVTKWISRQENSDVPLVYQ
DELIQIRELMYKPYKDWVAFLKQLHKRLEVEIGKEVKHWRKSLSDGRKGLYGISLKNIDEIDRT
RKFLLRWSLRPTEPGEVRRLEPGQRFAIDQLNHLNALKEDRLKKMANTIIMHALGYCYDVRKKK
WQAKNPACQIILFEDLSNYNPYGERSRFENSRLMKWSRREIPRQVALQGEIYGLQVGEVGAQFS
SRFHAKTGSPGIRCRVVTKEKLQDNRFFKNLQREGRLTLDKIAVLKEGDLYPDKGGEKFISLSK
DRKCVTTHADINAAQNLQKRFWTRTHGFYKVYCKAYQVDGQTVYIPESKDQKQKIIEEFGEGYF
ILKDGVYEWVNAGKLKIKKGSSKQSSSELVDSDILKDSFDLASELKGEKLMLYRDPSGNVFPSD
KWMAAGVFFGKLERILISKLTNQYSISTIEDDSSKQSMSGGSKRTADGSEFESPKKKRKVE.

In any of the above aspects, or embodiments thereof, the contacting is in a mammalian cell. In any of the above aspects, or embodiments thereof, the cell is a primate cell. In embodiments, primate cell is a human cell or a Macaca fascicularis cell. In any of the above aspects, or embodiments thereof, the cell is a liver cell. In embodiments, the liver cell is a primate liver cell in vivo. In embodiments, the primate cell is a human cell or a Macaca fascicularis cell.

In any of the above aspects, or embodiments thereof, repair of the double-stranded break by the cell results in the introduction of an indel mutation in the TTR polynucleotide sequence.

In any of the above aspects, or embodiments thereof, the method further involves contacting the polynucleotide sequence with two or more distinct guide RNAs that target the TTR polynucleotide sequence.

In any of the above aspects, or embodiments thereof, the deaminase is in complex with the polynucleotide programmable DNA binding polypeptide and the guide RNA. In any of the above aspects, or embodiments thereof, the base editor is a fusion protein containing the polynucleotide programmable DNA binding polypeptide and the deaminase.

In any of the above aspects, or embodiments thereof, the alteration of the nucleobase replaces a pathogenic alteration with a non-pathogenic alteration or a wild-type amino acid.

In any of the above aspects, or embodiments thereof, the subject is a primate. In embodiments, the primate is a human. In any of the above aspects, or embodiments thereof, the subject is a mammal. In embodiments, the primate is a human or Macaca fascicularis.

In any of the above aspects, or embodiments thereof, the polynucleotide sequence is in a hepatocyte. In embodiments, the hepatocyte is a primary hepatocyte. In embodiments, the hepatocyte is a primary cyno hepatocyte.

In any of the above aspects, or embodiments thereof, the adenosine deaminase domain contains an arginine (R) or a threonine (T) at amino acid position 147 of the following amino acid sequence, and the adenosine deaminase domain has at least about 85% sequence identity to the following amino acid sequence: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 4; TadA*7.10). The guide RNA targets the fusion protein to effect an alteration of a nucleobase of a TTR polynucleotide sequence.

In any of the above aspects, or embodiments thereof, the cytidine deaminase domain contains an amino acid sequence with at least about 85% sequence identity to the amino acid sequence: MSSETGPVAVDPTLRRRIEPHEFEVFFDPRELRKETCLLYEINWGGRHSIWRHTSQNTNKHVEV NFIEKFTTERYFCPNTRCSITWFLSWSPCGECSRAITEFLSRYPHVTLFIYIARLYHHADPRNR QGLRDLISSGVTIQIMTEQESGYCWRNFVNYSPSNEAHWPRYPHLWVRLYVLELYCIILGLPPC LNILRRKQPQLTFFTIALQSCHYQRLPPHILWATGLK (SEQ ID NO: 15), where the guide RNA targets the fusion protein to effect an alteration of a nucleobase of a TTR polynucleotide sequence.

In any of the above aspects, or embodiments thereof, the base editor does not contain a uracil glycosylase inhibitor (UGI).

In any of the above aspects, or embodiments thereof, the fusion protein:
(i) contains an amino acid sequence that is at least 80%, 85%, 90%, 95%, 96%, 97%, 98%, 99%, or 100% identical to:

ABE8.8
(SEQ ID NO: 442)
MSEVEFSHEYWMRHALTLAKRARDEREVPVGAVLVLNNRVIGEGWNRAIGLHDPTAHAEIMALR
QGGLVMQNYRLIDATLYVTFEPCVMCAGAMIHSRIGRVVFGVRNAKTGAAGSLMDVLHHPGMNH
RVEITEGILADECAALLCRFFRMPRRVENAQKKAQSSTDSGGSSGGSSGSETPGTSESATPESS
GGSSGGSDKKYSIGLAIGTNSVGWAVITDEYKVPSKKFKVLGNTDRHSIKKNLIGALLEDSGET
AEATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKERGHFLIEGDLNPDNSDVDKLFI
QLVQTYNQLFEENPINASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAILLSDILRVNTEITK
APLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPELKDNREKIEK
ILTFRIPYYVGPLARGNSRFAWMTRKSEETITPWNFEEVVDKGASAQSFIERMTNEDKNLPNEK
VLPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDELDNEENEDILEDIVLTLTLFEDREMIEE
RLKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMNTKYDENDKLIREVK
VITLKSKLVSDFRKDFQFYKVREINNYHHAHDAYLNAVVGTALIKKYPKLESEFVYGDYKVYDV
RKMIAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAAFKYFDTTIDRKRYTS
TKEVLDATLIHQSITGLYETRIDLSQLGGDEGADKRTADGSEFESPKKKRKV;

(ii) contains an amino acid sequence that is at least 80%, 85%, 90%, 95%, 96%, 97%, 98%, 99%, or 100% identical to:

BE4
(SEQ ID NO: 443)
MSSETGPVAVDPTLRRRIEPHEFEVFFDPRELRKETCLLYEINWGGRHSIWRHTSQNTNKHVEV
NFIEKFTTERYFCPNTRCSITWELSWSPCGECSRAITEFLSRYPHVTLFIYIARLYHHADPRNR
QGLRDLISSGVTIQIMTEQESGYCWRNFVNYSPSNEAHWPRYPHLWVRLYVLELYCIILGLPPC
LNILRRKQPQLTFFTIALQSCHYQRLPPHILWATGLKSGGSSGGSSGSETPGTSESATPESSGG
SSGGSDKKYSIGLAIGTNSVGWAVITDEYKVPSKKFKVLGNTDRHSIKKNLIGALLEDSGETAE
ATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKERGHFLIEGDLNPDNSDVDKLFIQL
VQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKAP
LSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNEDKNLPNEKVL
PKHSLLYEYFTVYNELTKVKYVTEGMRKPAFLSGEQKKAIVDLLFKTNRKVTVKQLKEDYFKKI
ECFDSVEISGVEDRENASLGTYHDLLKIIKDKDELDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVR
KVLSMPQVNIVKKTEVQTGGESKESILPKRNSDKLIARKKDWDPKKYGGFDSPTVAYSVLVVAK
VEKGKSKKLKSVKELLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQHKHYLDEIIEQISEF
SKRVILADANLDKVLSAYNKHRDKPIREQAENIIHLFTLTNLGAPAAFKYEDTTIDRKRYTSTK
EVLDATLIHQSITGLYETRIDLSQLGGDSGGSGGSGGSTNLSDIIEKETGKQLVIQESILMLPE
EVEEVIGNKPESDILVHTAYDESTDENVMLLTSDAPEYKPWALVIQDSNGENKIKMLSGGSGGS
GGSTNLSDIIEKETGKQLVIQESILMLPEEVEEVIGNKPESDILVHTAYDESTDENVMLLTSDA
PEYKPWALVIQDSNGENKIKMLSGGSKRTADGSEFESPKKKRKVE;

(iii) contains an amino acid sequence that is at least 80%, 85%, 90%, 95%, 96%, 97%, 98%, 99%, or 100% identical to:

ABE8.8-VRQR
(SEQ ID NO: 444)
MSEVEFSHEYWMRHALTLAKRARDEREVPVGAVLVLNNRVIGEGWNRAIGLHDPTAHAEIMALR
QGGLVMQNYRLIDATLYVTFEPCVMCAGAMIHSRIGRVVFGVRNAKTGAAGSLMDVLHHPGMNH
RVEITEGILADECAALLCRFFRMPRRVENAQKKAQSSTDSGGSSGGSSGSETPGTSESATPESS
GGSSGGSDKKYSIGLAIGTNSVGWAVITDEYKVPSKKFKVLGNTDRHSIKKNLIGALLEDSGET
AEATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAILLSDILRVNTEITK
APLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMNTKYDENDKLIREVK
VITLKSKLVSDFRKDFQFYKVREINNYHHAHDAYLNAVVGTALIKKYPKLESEFVYGDYKVYDV
RKMIAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFVSPTVAYSVLVV
AKVEKGKSKKLKSVKELLGITIMERSSFEKNPIDFLEAKGYKEVKKDLIIKLPKYSLFELENGR
KRMLASARELQKGNELALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAAFKYEDTTIDRKQYRS
TKEVLDATLIHQSITGLYETRIDLSQLGGDEGADKRTADGSEFESPKKKRKV;

(iv) contains an amino acid sequence that is at least 80%, 85%, 90%, 95%, 96%, 97%, 98%, 99%, or 100% identical to:

BE4-VRQR
(SEQ ID NO: 445)
MSSETGPVAVDPTLRRRIEPHEFEVFFDPRELRKETCLLYEINWGGRHSIWRHTSQNTNKHVEV
NFIEKFTTERYFCPNTRCSITWFLSWSPCGECSRAITEFLSRYPHVTLFIYIARLYHHADPRNR
QGLRDLISSGVTIQIMTEQESGYCWRNFVNYSPSNEAHWPRYPHLWVRLYVLELYCIILGLPPC
LNILRRKQPQLTFFTIALQSCHYQRLPPHILWATGLKSGGSSGGSSGSETPGTSESATPESSGG
SSGGSDKKYSIGLAIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKAP
LSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNEDKNLPNEKVL
PKHSLLYEYFTVYNELTKVKYVTEGMRKPAFLSGEQKKAIVDLLFKTNRKVTVKQLKEDYFKKI
ECFDSVEISGVEDRENASLGTYHDLLKIIKDKDELDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGFVSPTVAYSVLVVAK
VEKGKSKKLKSVKELLGITIMERSSFEKNPIDFLEAKGYKEVKKDLIIKLPKYSLFELENGRKR
MLASARELQKGNELALPSKYVNFLYLASHYEKLKGSPEDNEQKQLFVEQHKHYLDEIIEQISEF
SKRVILADANLDKVLSAYNKHRDKPIREQAENIIHLFTLTNLGAPAAFKYEDTTIDRKQYRSTK
EVLDATLIHQSITGLYETRIDLSQLGGDSGGSGGSGGSTNLSDIIEKETGKQLVIQESILMLPE
EVEEVIGNKPESDILVHTAYDESTDENVMLLTSDAPEYKPWALVIQDSNGENKIKMLSGGSGGS
GGSTNLSDIIEKETGKQLVIQESILMLPEEVEEVIGNKPESDILVHTAYDESTDENVMLLTSDA
PEYKPWALVIQDSNGENKIKMLSGGSKRTADGSEFESPKKKRKVE;

(v) contains an amino acid sequence that is at least 80%, 85%, 90%, 95%, 96%, 97%, 98%, 99%, or 100% identical to:

saABE8.8
(SEQ ID NO: 446)
MSEVEFSHEYWMRHALTLAKRARDEREVPVGAVLVLNNRVIGEGWNRAIGLHDPTAHAEIMALR
QGGLVMQNYRLIDATLYVTFEPCVMCAGAMIHSRIGRVVFGVRNAKTGAAGSLMDVLHHPGMNH
RVEITEGILADECAALLCRFERMPRRVENAQKKAQSSTDSGGSSGGSSGSETPGTSESATPESS
GGSSGGSKRNYILGLAIGITSVGYGIIDYETRDVIDAGVRLFKEANVENNEGRRSKRGARRLKR
RRRHRIQRVKKLLFDYNLLTDHSELSGINPYEARVKGLSQKLSEEEFSAALLHLAKRRGVHNVN
EVEEDTGNELSTKEQISRNSKALEEKYVAELQLERLKKDGEVRGSINRFKTSDYVKEAKQLLKV
QKAYHQLDQSFIDTYIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIAKEILVNEEDIKGYR
VTSTGKPEFTNLKVYHDIKDITARKEIIENAELLDQIAKILTIYQSSEDIQEELTNLNSELTQE
EIEQISNLKGYTGTHNLSLKAINLILDELWHTNDNQIAIFNRLKLVPKKVDLSQQKEIPTTLVD
DFILSPVVKRSFIQSIKVINAIIKKYGLPNDIIIELAREKNSKDAQKMINEMQKRNRQTNERIE
EIIRTTGKENAKYLIEKIKLHDMQEGKCLYSLEAIPLEDLLNNPFNYEVDHIIPRSVSEDNSEN
NKVLVKQEENSKKGNRTPFQYLSSSDSKISYETFKKHILNLAKGKGRISKTKKEYLLEERDINR
FSVQKDFINRNLVDTRYATRGLMNLLRSYFRVNNLDVKVKSINGGFTSFLRRKWKFKKERNKGY
KHHAEDALIIANADFIFKEWKKLDKAKKVMENQMFEEKQAESMPEIETEQEYKEIFITPHQIKH
IKDFKDYKYSHRVDKKPNRELINDTLYSTRKDDKGNTLIVNNLNGLYDKDNDKLKKLINKSPEK
LLMYHHDPQTYQKLKLIMEQYGDEKNPLYKYYEETGNYLTKYSKKDNGPVIKKIKYYGNKLNAH
LDITDDYPNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPRIIK
TIASKTQSIKKYSTDILGNLYEVKSKKHPQIIKKGEGADKRTADGSEFESPKKKRKV;

(vi) contains an amino acid sequence that is at least 80%, 85%, 90%, 95%, 96%, 97%, 98%, 99%, or 100% identical to:

saBE4
(SEQ ID NO: 447)
MSSETGPVAVDPTLRRRIEPHEFEVFFDPRELRKETCLLYEINWGGRHSIWRHTSQNTNKHVEV
NFIEKFTTERYFCPNTRCSITWFLSWSPCGECSRAITEFLSRYPHVTLFIYIARLYHHADPRNR
QGLRDLISSGVTIQIMTEQESGYCWRNFVNYSPSNEAHWPRYPHLWVRLYVLELYCIILGLPPC
LNILRRKQPQLTFFTIALQSCHYQRLPPHILWATGLKSGGSSGGSSGSETPGTSESATPESSGG
SSGGSGKRNYILGLAIGITSVGYGIIDYETRDVIDAGVRLFKEANVENNEGRRSKRGARRLKRR
RRHRIQRVKKLLFDYNLLTDHSELSGINPYEARVKGLSQKLSEEEFSAALLHLAKRRGVHNVNE
VEEDTGNELSTKEQISRNSKALEEKYVAELQLERLKKDGEVRGSINRFKTSDYVKEAKQLLKVQ
KAYHQLDQSFIDTYIDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAKEILVNEEDIKGYRV
TSTGKPEFTNLKVYHDIKDITARKEIIENAELLDQIAKILTIYQSSEDIQEELTNLNSELTQEE
IEQISNLKGYTGTHNLSLKAINLILDELWHTNDNQIAIFNRLKLVPKKVDLSQQKEIPTTLVDD
FILSPVVKRSFIQSIKVINAIIKKYGLPNDIIIELAREKNSKDAQKMINEMQKRNRQTNERIEE
IIRTTGKENAKYLIEKIKLHDMQEGKCLYSLEAIPLEDLLNNPFNYEVDHIIPRSVSEDNSENN
KVLVKQEENSKKGNRTPFQYLSSSDSKISYETFKKHILNLAKGKGRISKTKKEYLLEERDINRF
SVQKDFINRNLVDTRYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRELINDTLYSTRKDDKGNTLIVNNLNGLYDKDNDKLKKLINKSPEKL
LMYHHDPQTYQKLKLIMEQYGDEKNPLYKYYEETGNYLTKYSKKDNGPVIKKIKYYGNKLNAHL
DITDDYPNSRNKVVKLSLKPYRFDVYLDNGVYKFVTVKNLDVIKKENYYEVNSKCYEEAKKLKK
ISNQAEFIASFYNNDLIKINGELYRVIGVNNDLLNRIEVNMIDITYREYLENMNDKRPPRIIKT
IASKTQSIKKYSTDILGNLYEVKSKKHPQIIKKGGSPKKKRKVSSDYKDHDGDYKDHDIDYKDD
DDKSGGSGGSGGSTNLSDIIEKETGKQLVIQESILMLPEEVEEVIGNKPESDILVHTAYDESTD
ENVMLLTSDAPEYKPWALVIQDSNGENKIKMLSGGSGGSGGSTNLSDIIEKETGKQLVIQESIL
MLPEEVEEVIGNKPESDILVHTAYDESTDENVMLLTSDAPEYKPWALVIQDSNGENKIKMLSGG
SKRTADGSEFESPKKKRKVE;

(vii) contains an amino acid sequence that is at least 80%, 85%, 90%, 95%, 96%, 97%, 98%, 99%, or 100% identical to:

saBE4-KKH
(SEQ ID NO: 448)
MSSETGPVAVDPTLRRRIEPHEFEVFFDPRELRKETCLLYEINWGGRHSIWRHTSQNTNKHVEV
NFIEKFTTERYFCPNTRCSITWFLSWSPCGECSRAITEFLSRYPHVTLFIYIARLYHHADPRNR
QGLRDLISSGVTIQIMTEQESGYCWRNFVNYSPSNEAHWPRYPHLWVRLYVLELYCIILGLPPC
LNILRRKQPQLTFFTIALQSCHYQRLPPHILWATGLKSGGSSGGSSGSETPGTSESATPESSGG
SSGGSGKRNYILGLAIGITSVGYGIIDYETRDVIDAGVRLFKEANVENNEGRRSKRGARRLKRR
RRHRIQRVKKLLFDYNLLTDHSELSGINPYEARVKGLSQKLSEEEFSAALLHLAKRRGVHNVNE
VEEDTGNELSTKEQISRNSKALEEKYVAELQLERLKKDGEVRGSINRFKTSDYVKEAKQLLKVQ
KAYHQLDQSFIDTYIDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAKEILVNEEDIKGYRV
TSTGKPEFTNLKVYHDIKDITARKEIIENAELLDQIAKILTIYQSSEDIQEELTNLNSELTQEE
IEQISNLKGYTGTHNLSLKAINLILDELWHTNDNQIAIFNRLKLVPKKVDLSQQKEIPTTLVDD
FILSPVVKRSFIQSIKVINAIIKKYGLPNDIIIELAREKNSKDAQKMINEMQKRNRQTNERIEE
IIRTTGKENAKYLIEKIKLHDMQEGKCLYSLEAIPLEDLLNNPENYEVDHIIPRSVSEDNSENN
KVLVKQEENSKKGNRTPFQYLSSSDSKISYETFKKHILNLAKGKGRISKTKKEYLLEERDINRF
SVQKDFINRNLVDTRYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRKLINDTLYSTRKDDKGNTLIVNNLNGLYDKDNDKLKKLINKSPEKL
LMYHHDPQTYQKLKLIMEQYGDEKNPLYKYYEETGNYLTKYSKKDNGPVIKKIKYYGNKLNAHL
DITDDYPNSRNKVVKLSLKPYRFDVYLDNGVYKFVTVKNLDVIKKENYYEVNSKCYEEAKKLKK
ISNQAEFIASFYKNDLIKINGELYRVIGVNNDLLNRIEVNMIDITYREYLENMNDKRPPHIIKT
IASKTQSIKKYSTDILGNLYEVKSKKHPQIIKKGGSPKKKRKVSSDYKDHDGDYKDHDIDYKDD
DDKSGGSGGSGGSTNLSDIIEKETGKQLVIQESILMLPEEVEEVIGNKPESDILVHTAYDESTD
ENVMLLTSDAPEYKPWALVIQDSNGENKIKMLSGGSGGSGGSTNLSDIIEKETGKQLVIQESIL
MLPEEVEEVIGNKPESDILVHTAYDESTDENVMLLTSDAPEYKPWALVIQDSNGENKIKMLSGG
SKRTADGSEFESPKKKRKVE;

or
(viii) contains an amino acid sequence that is at least 80%, 85%, 90%, 95%, 96%, 97%, 98%, 99%, or 100% identical to:

ABE-bhCAS12b
(SEQ ID NO: 449)
MSEVEFSHEYWMRHALTLAKRARDEREVPVGAVLVLNNRVIGEGWNRAIGLHDPTAHAEIMALR
QGGLVMQNYRLYDATLYVTFEPCVMCAGAMIHSRIGRVVFGVRNAKTGAAGSLMDVLHHPGMNH
RVEITEGILADECAALLCRFFRMPRRVENAQKKAQSSTDGSSGSETPGTSESATPESSGAPKKK
RKVGIHGVPAAATRSFILKIEPNEEVKKGLWKTHEVLNHGIAYYMNILKLIRQEAIYEHHEQDP
KNPKKVSKAEIQAELWDFVLKMQKCNSFTHEVDKDEVENILRELYEELVPSSVEKKGEANQLSN
KFLYPLVDPNSQSGKGTASSGRKPRWYNLKIAGDPSWEEEKKKWEEDKKKDPLAKILGKLAEYG
LIPLFIPYTDSNEPIVKEIKWMEKSRNQSVRRLDKDMFIQALERFLSWESWNLKVKEEYEKVEK
EYKTLEERIKEDIQALKALEQYEKERQEQLLRDTLNTNEYRLSKRGLRGWREIIQKWLKMDENE
PSEKYLEVEKDYQRKHPREAGDYSVYEFLSKKENHFIWRNHPEYPYLYATFCEIDKKKKDAKQQ
ATFTLADPINHPLWVRFEERSGSNLNKYRILTEQLHTEKLKKKLTVQLDRLIYPTESGGWEEKG
KVDIVLLPSRQFYNQIFLDIEEKGKHAFTYKDESIKFPLKGTLGGARVQFDRDHLRRYPHKVES
GNVGRIYENMTVNIEPTESPVSKSLKIHRDDFPKVVNFKPKELTEWIKDSKGKKLKSGIESLEI
GLRVMSIALGQRQAAAASIFEVVDQKPDIEGKLFFPIKGTELYAVHRASFNIKLPGETLVKSRE
VLRKAREDNLKLMNQKLNFLRNVLHFQQFEDITEREKRVTKWISRQENSDVPLVYQDELIQIRE
LMYKPYKDWVAFLKQLHKRLEVEIGKEVKHWRKSLSDGRKGLYGISLKNIDEIDRTRKELLRWS
LRPTEPGEVRRLEPGQRFAIDQLNHLNALKEDRLKKMANTIIMHALGYCYDVRKKKWQAKNPAC
QIILFEDLSNYNPYKERSRFENSRLMKWSRREIPRQVALQGEIYGLQVGEVGAQFSSRFHAKTG
SPGIRCRVVTKEKLQDNRFFKNLQREGRLTLDKIAVLKEGDLYPDKGGEKFISLSKDRKCVTTH
ADINAAQNLQKRFWTRTHGFYKVYCKAYQVDGQTVYIPESKDQKQKIIEEFGEGYFILKDGVYE
WVNAGKLKIKKGSSKQSSSELVDSDILKDSFDLASELKGEKLMLYRDPSGNVEPSDKWMAAGVE
FGKLERILISKLTNQYSISTIEDDSSKQSMKRPAATKKAGQAKKKK.

In any of the above aspects, or embodiments thereof, the guide RNA(s) contains 15, 16, 17, 18, 19, 20, 21, 22, 23, 24, 25, 26, 27, 28, 29, or 30 contiguous nucleotides that are perfectly complementary to the TTR polynucleotide. In any of the above aspects, or embodiments thereof, the guide RNA contains a nucleic acid sequence containing 15, 16, 17, 18, 19, 20, 21, 22, 23, 24, 25, 26, 27, 28, 29, 30, 31, 32, 33, 34, 35, 36, 37, 38, 39, or 40 contiguous nucleotides that are complementary to the TTR polynucleotide sequence.

In any of the above aspects, or embodiments thereof, the composition or pharmaceutical composition further contains a lipid or lipid nanoparticle. In embodiments, the lipid is a cationic lipid. In any of the above aspects, or embodiments thereof, the guide RNA contains a nucleic acid sequence contains at least 10 contiguous nucleotides that are complementary to the TTR polynucleotide sequence.

In any of the above aspects, or embodiments thereof, the one or more polynucleotides encoding the fusion protein contains mRNA.

In any of the above aspects, or embodiments thereof, the composition or pharmaceutical composition further contains a pharmaceutically acceptable excipient. In any of the above aspects, or embodiments thereof, the gRNA and the base editor are formulated together or separately.

In any of the above aspects, or embodiments thereof, the polynucleotide is present in a vector suitable for expression in a mammalian cell. In embodiments, the vector is a viral vector. In embodiments, the viral vector is a retroviral vector, adenoviral vector, lentiviral vector, herpesvirus vector, or adeno-associated viral vector (AAV).

In any of the above aspects, or embodiments thereof, the alteration reduces or eliminates expression of a wild-type or mutant TTR polypeptide.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “transthyretin (TTR) polypeptide” is meant a polypeptide or fragment thereof having at least about 95% amino acid sequence identity to an amino acid sequence provided at NCBI Reference Sequence No. NP_000362.1, or a fragment thereof that binds an anti-TTR antibody. In some embodiments, a TTR polypeptide or fragment thereof has holo-retinol-binding protein (RBP) and/or thyroxine (T4) transport activity. Typically, amino acid locations for mutations to the TTR polypeptide are numbered with reference to the mature TTR polypeptide (i.e., the TTR polypeptide without a signal sequence). In embodiments, TTR is capable of forming a tetramer. An exemplary TTR polypeptide sequence follows (the signal peptide sequence is in bold; therefore, the mature TTR polypeptide corresponds to amino acids 21 to 147 of the following sequence):

(SEQ ID NO: 1)
MASHRLLLLCLAGLVFVSEAGPTGTGESKCPLMVKVLDAVRGSPAINVA
VHVFRKAADDTWEPFASGKTSESGELHGLTTEEEFVEGIYKVEIDTKSY
WKALGISPFHEHAEVVFTANDSGPRRYTIAALLSPYSYSTTAVVTNPK
E.

By “transthyretin (TTR) polynucleotide” is meant a nucleic acid molecule that encodes a TTR, as well as the introns, exons, 3′ untranslated regions, 5′ untranslated regions, and regulatory sequences associated with its expression, or fragments thereof. In embodiments, the regulatory sequence is a promoter region. In embodiments, a TTR polynucleotide is the genomic sequence, cDNA, mRNA, or gene associated with and/or required for TTR expression. An exemplary TTR polynucleotide sequence (corresponding to Consensus Coding Sequence (CCDS) No. 11899.1) is provided below. Further exemplary TTR polynucleotide sequences include Gene Ensembl ID: ENSG00000118271 and Transcript Ensembl ID: ENST00000237014.8.

(SEQ ID NO: 2)
ATGGCTTCTCATCGTCTGCTCCTCCTCTGCCTTGCTGGACTGGTATTTG
TGTCTGAGGCTGGCCCTACGGGCACCGGTGAATCCAAGTGTCCTCTGAT
GGTCAAAGTTCTAGATGCTGTCCGAGGCAGTCCTGCCATCAATGTGGCC
GTGCATGTGTTCAGAAAGGCTGCTGATGACACCTGGGAGCCATTTGCCT
CTGGGAAAACCAGTGAGTCTGGAGAGCTGCATGGGCTCACAACTGAGGA
GGAATTTGTAGAAGGGATATACAAAGTGGAAATAGACACCAAATCTTAC
TGGAAGGCACTTGGCATCTCCCCATTCCATGAGCATGCAGAGGTGGTAT
TCACAGCCAACGACTCCGGCCCCCGCCGCTACACCATTGCCGCCCTGCT
GAGCCCCTACTCCTATTCCACCACGGCTGTCGTCACCAATCCCAAGGAA
TGA.

A further exemplary TTR polynucleotide sequence is provided at NCBI Reference Sequence No. NG_009490.1 and follows (where exons encoding the TTR polypeptide are in bold, introns are in italics, and exemplary promoter regions are indicated by the combined underlined and bold-underlined text (promoter positions −1 to −177) and by the bold-underlined text (promoter positions −106 to −176); further exemplary promoter regions are shown in FIGS. 9A, 9B, 12A, and 12B):

(SEQ ID NO: 3)
TTATGTGTTTATTCAACAATGGCGGAGGAGAGGCATGCCAGATAAGGCAGACACGG
GCATTCCAAACACAAGAAAGGTATGTGCTGCAGAGAAGTCAGATAACTTTCCTAGG
CTCTCCTGCAGTCCGGATGAAATACTCTCAAAAAATTAGCCCGGGCCCTTTGCTCCA
ATTTTTCGCTTACCTAGCAACCATCTAACTATTAATTAAATTGGTATTATGGTTTTAA
CATGAATCTTTTATGATTTGCTTACCATTAATCAAACCCCCGAGGCTTATTCACCTCA
AGGGGAGCTGACAAAGTTGAATTATTCAACCTGCAAAGATCCAGGGCCCCCAAATA
CTGTCATTTCCACTCTCCCCTAACCCCCACCATGAGGCCCAGTCTCAGCACTCGGCC
AGCCTATGCCCAACTCGGGGTAATCAGCTTAGACATATTAATATTAGTGGGCATTTC
AGTATCAACAGATCACTGTCTAGCAGCTGACAGGCACCCTCAGAAAATAAACCAAG
AAGAAAGGGTTTATCTATAATATCAAAATTTTTCATAGATAAACCTGCCCATTATAA
GGAAGAGGGCAGAAGAACCCTAAACTAAGAGCCAGGCAACTTGTTCATTAATCACA
GCATATTCCATAGAAGGAGGAGAAATGTTGTCATCAAATTCATCTTTTTCACCTCAA
TTAAACATCTATGCTACAGCTCCACAGTCAGATTGAGAGGAAAAACAGTACGTAGC
TAAGAAAAGACATAGACTTGTAACTGAAATGCTTCACTGGTGCTCCTTTTGTTTTAA
GGCATTGGATCTTCATAGCTACTGATCGTGCCCAAGCACACAGTATCTGCAGCAACC
ACTTAGGCCTCCAGGAATGTGGTGACCATTGACCCTAATTCATTCCCCTTCATGGAT
CCTATGTAACCATCCTCCAAAAAGAGCTTTCGCAAACTCAAATAAACACAGGAAAG
GAAGACCTTCTTATCTTTGAGAGTATATGTTTAGCCCTATAACCCTCTCTTATCATAA
ATTGCTTCTTAGGCAAGAAACACTGGATTTTTCTTGTATTTGTCATTGCCATTGGTTC
CATACAAGCATTCATTTAACAAATAATACATTCCATCTCCGTTTTTTGCTTTTTCCTT
CATGCTTCGGCCTTGGTTTTCTCTCACCTAAAACACTACAAGCTTCTTCTCCCAGAGC
TCTCACTTTGACTCCAGACTACCTACATTTAATCTTTAATTCTCTACCAAAATTTTCT
CTTATCTTTATATCTTTTTAATTTTAATTTTATTTTTTGTGGATACATAGTAGGTGTAT
ATATTTATGGGGTACATGCAATGTTTTAATACAGGCATGCAATGTGAAATAAGGACA
TCATGGAGAATGGGGTATCCATCTCCTCAAGCATTTATCTTTTCAGTTACAAACAAT
CCAATTACACTCTTTATTTTAAAATATACAATTATTAATTATAGTCACTCTGTTGTGC
TATCAAATAGTAGATCTTATTCTTTCTATTTTTTGTACCCTCTCGTCTTTTTATATTAA
AAAATAATCTTTATCTCTGTAAGCTTCATCAGTGATTTTCCAATGAAATTTAGGATCT
TCTCTATACCCTGAATTGCCTTACTTTCTCCCCACTTCCTTGTCTTATTCAAATGCAG
ATTTCATTAATGATTTCCAGATCAATGATAGTTCAGAAAGCAAGCAAGTCAAAGTGA
CCAAGGGCATGGCCTGAAAACTGTTCTAAGAGAGGAATTTACAGAACAACTATTAA
ATGATGTCAATAGGATTGTATTAGTCCGTTTTCATACGGCTATAAAGAACTGCCTGA
GACTGGGTAATTTATAAAGGAAAGAGGTTTAATTGACTCACAGTTCAGCACAACTG
GGCAGGCCTCAGGAAACTTACAATCATGGTAGAAGGTGAAGGGGAAGCAAAGCAC
CTTCCTCACAAGGCGTCAGGAAGAAGTGCCAAGCAAAGGGGGAAAAGCCCCTTGTA
AAACTACCAGAACCTGTGAGAACTCAATCACTATCACAAGAACAGCATGAGGGAAC
CGCCCCTCGTGATTCAATTACCTCCACCTGGTCTCTCCCTTGACACATGGGGATTATG
GGTGTTACAATTCAAGATGAGATTTGGGTGGGGACACAAAGCCTAACCATATCAAG
GATCAAGTGGTGGGTTGAAACTAACAGGATGAGATATATCAGATACAAACACAGGG
TCCCATATTTGGGTTAAAATTCATAAATGATCAAAGCACAGGATGACAGATAATATA
GGTCATTTTAGATTATTGTGGCCAACAGATCACAGTGGGTAGTGTTATGACGAAGGG
AGGGTCACAGTTACTACAGTTACAGATGGATTCTGGGTACAACATTTGCACTAAAGT
GCCTTTGCCAAGGGAGGCAACAGTCTCGACATCCTGTGGCCTGATCTACTTCAGGGA
CTGTGTCTTGTTCAGAGCATCACATTTGAAGAGAACTTTGACCAAGGGGAATATGCC
AGAAAAGGAAGTTCGGGATGCTGAGGATCTTAGGAACTATGTCTAAACAAGATTCA
TTCACAGAAGTGGGAATGTCTATTTGGCAAAAAGAAAATACTACTTACATGGCTGTT
GGAAGACCAGCAATCACAAACTCAGTTTTTCAAAAGGCTGGGCAGAAACACAGATG
AAAGAAACAGGCCATGTTTAAGAAAAGATAAAAGCTCACGCATGATATGCCACTAG
AGAATCACCTAGCCTCAGTGTTGGCGGGGAGGCCTGGGGAGTCTTGATGTCTGAGA
GTGACATTCTGATGATCACTGTCATGTGTAAATGTTGGCCTAAAGCTGCCAATATTT
TTGATTTAAGAGAAGCAAGAAATGCAAATTTTTATGCAGCATGTCTCAATTTTTAAT
TTTGGCAACTATTACAAAATGTTTAAAGAGACTCTGTGCAGCCCAAATATAACATAT
CTATGGGCTGATGGCAGCCCAGCGTTGCCAGTTCACAGGGTCTACAAGAGATGATTC
TTAGTTTCAACAGGGTGCAGTGCTGAAACGCGTGCACAGTAGATTTTGCTTCGGTTA
TGAAAGAACTTCCAAATATTTATGATTCATAGCCAGAGAAAAGGCTCTCTATCCAGG
TTCTGAACAATAGGAAATCATCAAGAGGATATTGGATGACAATATATGAAAGATGT
TATTTGAGAAAGGATTCTCTCCTGAGGCATAGATGTTGAACCAAATTCTATTAGTTA
TGCTTTTACAGCAAGATAGTGGTTTACAGCTTACAAAAGGCTTGTACATCCTCTCAT
ATTAAAAGTTATTAGAACAGTCCTTTGAAGTAGAAAAGTAGGCATTTCTATTTTACA
AACGAGTTGGCCGAGTATCTGAGATAGTAGATAACTCATAGAAGGTCATCCGGGAA
ACGGGGCAGCAGAACTGGGATCGAATGACTCTGGTCATCCAACTCCAAATGCAAAA
GTCTTTCTGCTGCTGCTTCCTAGTTAAACTCTAAGGGTCTAAGACTCCATTCCTAGTT
ATGGTCTCAACTACATTTGCTCATTGCTGTGAGGGGTCAACCCACCTCCCGGAGTCC
TCTCCTGCACATTCTCATGTTCCTGAAAGGCTTTTCTGTCCCTTCCACTACTCCCTGT
AAGCTCCTGTGCTTCACAATTTCTTGTTGAATTTTTTCTAATCTGACTCTATCAGTTAT
GGGAATGTTCCCTCAATTCTTAGTGCTCCAAACCGGACTTGCTCTTGGCTTGTATTTG
TCCAAAATATTTGTCTTCTCTATGTTTTCTACATGTTTGTCTTATAAGGACAAAAACC
TGCCTTAGTTTATCCATGAACAAAGCCACGCATGCTAGTGGACACACACACACATGC
GCGTGCGCGCGCACACACACACACACACACATACACACAGAGACTTTGTATGTGAG
TAATGAATCATCAAATCATCATAATTTCTGGACTTGTATTAATAAGTCGGCCAGGAG
GAAAAGAATCTGCTGTCAATCATGGCTTCTGGTTCTCACAGTCATCTCTACTTTCTTC
CAGCAAGTTTGGTTCTGTCAAAAACCAGCTGTCAGCCTTGTTCCTGCATGCCCAATG
CAGAAGAGTCAGTAAAGAAGATTTGGTTCTCTGTATTTCAGGGGCATCAATGCCAG
GTTGAAATATGCCATTCTGGCCCAGCTCAGTGGCTCACACGTGTAATCCCAGCACTT
TGGAAGGCCAAAGCGGGTGGATTGCTTGAGCTCAGGAGTTCGAGACCAGCCTGGGC
AAGAGGCTGAGGTGGGAGGATGACCTGAGCCCGGGAGGTCAAGGCTGCAGCGAGC
TGTGATCGTGCCACTGCACTCGAGCCAGGGCGTTGGAGTGAGACCCTGTCAAAAAA
AAAAAAAAAAAGGAAGGAAAAAAGGAAGGAAGGAAGGGAGGGAGGGAAGATGCC
ATTCTTAGATTGAAGTGGACTTTATCTGGGCAGAACACACACACACATACACACATG
CACACACACATTGTGGAGAAATTGCTGACTAAGCAAAGCTTCCAAATGACTTAGTTT
GGCTAAAATGTAGGCTTTTAAAAATGTGAGCACTGCCAAGGGTTTTTCCTTGTTGAC
CCATGGATCCATCAAGTGCAAACATTTTCTAATGCACTATATTTAAGCCTGTGCAGC
TAGATGTCATTCAACATGAAATACATTATTACAACTTGCATCTGTCTAAAATCTTGC
ATCTAAAATGAGAGACAAAAAATCTATAAAAATGGAAAACATGCATAGAAATATGT
GAGGGAGGAAAAAATTACCCCCAAGAATGTTAGTGCACGCAGTCACACAGGGAGA
AGACTATTTTTGTTTTGTTTTGATTGTTTTGTTTTGTTTTGGTTGTTTTGTTTTGGTGAC
CTAACTGGTCAAATGACCTATTAAGAATATTTCATAGAACGAATGTTCCGATGCTCT
AATCTCTCTAGACAAGGTTCATATTTGTATGGGTTACTTATTCTCTCTTTGTTGA
CTAAGTCAATAATCAGAATCAGCAGGTTTGCAGTCAGATTGGCAGGGATAAGCAG
CCTAGCTCAGGAGAAGTGAGTATAAAAGCCCCAGGCTGGGAGCAGCCATCACAGAA
GTCCACTCATTCTTGGCAGGATGGCTTCTCATCGTCTGCTCCTCCTCTGCCTTGC
TGGACTGGTATTTGTGTCTGAGGCTGGCCCTACGGTGAGTGTTTCTGTGACATCCC
ATTCCTACATTTAAGATTCACGCTAAATGAAGTAGAAGTGACTCCTTCCAGCTTTGCCAACC
AGCTTTTATTACTAGGGCAAGGGTACCCAGCATCTATTTTTAATATAATTAATTCAAACTTCA
AAAAGAATGAAGTTCCACTGAGCTTACTGAGCTGGGACTTGAACTCTGAGCATTCTACCTC
ATTGCTTTGGTGCATTAGGTTTGTAATATCTGGTACCTCTGTTTCCTCAGATAGATGATAGA
AATAAAGATATGATATTAAGGAAGCTGTTAATACTGAATTTTCAGAAAAGTATCCCTCCATAA
AATGTATTTGGGGGACAAACTGCAGGAGATTATATTCTGGCCCTATAGTTATTCAAAACGTA
TTTATTGATTAATCTTTAAAAGGCTTAGTGAACAATATTCTAGTCAGATATCTAATTCTTAAAT
CCTCTAGAAGAATTAACTAATACTATAAAATGGGTCTGGATGTAGTTCTGACATTATTTTATA
ACAACTGGTAAGAGGGAGTGACTATAGCAACAACTAAAATGATCTCAGGAAAACCTGTTTG
GCCCTATGTATGGTACATTACATCTTTTCAGTAATTCCACTCAAATGGAGACTTTTAACAAA
GCAACTGTTCTCAGGGGACCTATTTTCTCCCTTAAAATTCATTATACACATCCCTGGTTGAT
AGCAGTGTGTCTGGAGGCAGAAACCATTCTTGCTTTGGAAACAATTACGTCTGTGTTATAC
TGAGTAGGGAAGCTCATTAATTGTCGACACTTACGTTCCTGATAATGGGATCAGTGTGTAA
TTCTTGTTTCGCTCCAGATTTCTAATACCACAAAGAATAAATCCTTTCACTCTGATCAATTTT
GTTAACTTCTCACGTGTCTTCTCTACACCCAGGGCACCGGTGAATCCAAGTGTCCTCT
GATGGTCAAAGTTCTAGATGCTGTCCGAGGCAGTCCTGCCATCAATGTGGCCG
TGCATGTGTTCAGAAAGGCTGCTGATGACACCTGGGAGCCATTTGCCTCTGGGT
AAGTTGCCAAAGAACCCTCCCACAGGACTTGGTTTTATCTTCCCGTTTGCCCCTCACTTGG
TAGAGAGAGGCTCACATCATCTGCTAAAGAATTTACAAGTAGATTGAAAAACGTAGGCAGA
GGTCAAGTATGCCCTCTGAAGGATGCCCTCTTTTTGTTTTGCTTAGCTAGGAAGTGACCAG
GAACCTGAGCATCATTTAGGGGCAGACAGTAGAGAAAAGAAGGAATCAGAACTCCTCTCC
TCTAGCTGTGGTTTGCAACCCTTTTGGGTCACAGAACACTTTATGTAGGTGATGAAAAGTA
AACATTCTATGCCCAGAAAAAATGCACAGATACACACACATACAAAATCATATATGTGATTT
TAGGAGTTTCACAGATTCCCTGGTGTCCCTGGGTAACACCAAAGCTAAGTGTCCTTGTCTT
AGAATTTTAGGAAAAGGTATAATGTGTATTAACCCATTAACAAAAGGAAAGGAATTCAGAAA
TATTATTAACCAGGCATCTGTCTGTAGTTAATATGGATCACCCAAAACCCAAGGCTTTTGCC
TAATGAACACTTTGGGGCACCTACTGTGTGCAAGGCTGGGGGCTGTCAAGCTCAGTTAAA
AAAAAAAAGATAGAAGAGATGGATCCATGAGGCAAAGTACAGCCCCAGGCTAATCCCACG
ATCACCCGACTTCATGTCCAAGAGTGGCTTCTCACCTTCATTAGCCAGTTCACAATTTTCAT
GGAGTTTTTCTACCTGCACTAGCAAAAACTTCAAGGAAAATACATATTAATAAATCTAAGCA
AAGTGACCAGAAGACAGAGCAATCAGGAGACCCTTTGCATCCAGCAGAAGAGGAACTGCT
AAGTATTTACATCTCCACAGAGAAGAATTTCTGTTGGGTTTTAATTGAACCCCAAGAACCAC
ATGATTCTTCAACCATTATTGGGAAGATCATTTTCTTAGGTCTGGTTTTAACTGGCTTTTTAT
TTGGGAATTCATTTATGTTTATATAAAATGCCAAGCATAACATGAAAAGTGGTTACAGGACT
ATTCTAAGGGAGAGACAGAATGGACACCAAAAATATTCCAATGTTCTTGTGAATCTTTTCCT
TGCACCAGGACAAAAAAAAAAAGAAGTGAAAAGAAGAAAGGAGGAGGGGCATAATCAGAG
TCAGTAAAGACAACTGCTATTTTTATCTATCGTAGCTGTTGCAGTCAAATGGGAAGCAATTT
CCAACATTCAACTATGGAGCTGGTACTTACATGGAAATAGAAGTTGCCTAGTGTTTGTTGCT
GGCAAAGAGTTATCAGAGAGGTTAAATATATAAAAGGGAAAAGAGTCAGATACAGGTTCTT
CTTCCTACTTTAGGTTTTCCACTGTGTGTGCAAATGATACTCCCTGGTGGTGTGCAGATGC
CTCAAAGCTATCCTCACACCACAAGGGAGAGGAGCGAGATCCTGCTGTCCTGGAGAAGTG
CAGAGTTAGAACAGCTGTGGCCACTTGCATCCAATCATCAATCTTGAATCACAGGGACTCT
TTCTTAAGTAAACATTATACCTGGCCGGGCACGGTGGCTCACGCCTGTAATCCCAGCACTT
TGGGATGCCAAAGTGGGCATATCATCTGAGGTCAGGAGTTCAAGACCAGCCTGGCCAACA
TGGCAAAACTCCGTCTTTATGAAAAATACAAAAATTAGCCAGGCATGGTGGCAGGCGCCTG
TAATCCCAGCTAATTGGGAGGCTGAGGCTGGAGAATCCCTTGAATCTAGGAGGCAGAGGT
TGCAGTGAGCTGAGATCGTGCCATTGCACTCCAGCCTGGGTGACAAGAGTAAAACTCTGT
CTCAAAAAAAAAAAATTATACCTACATTCTCTTCTTATCAGAGAAAAAAATCTACAGTGAGCT
TTTCAAAAAGTTTTTACAAACTTTTTGCCATTTAATTTCAGTTAGGAGTTTTCCCTACTTCTGA
CTTAGTTGAGGGGAAATGTTCATAACATGTTTATAACATGTTTATGTGTGTTAGTTGGTGGG
GGTGTATTACTTTGCCATGCCATTTGTTTCCTCCATGCGTAACTTAATCCAGACTTTCACAC
CTTATAGGAAAACCAGTGAGTCTGGAGAGCTGCATGGGCTCACAACTGAGGAGG
AATTTGTAGAAGGGATATACAAAGTGGAAATAGACACCAAATCTTACTGGAAG
GCACTTGGCATCTCCCCATTCCATGAGCATGCAGAGGTGAGTATACAGACCTTCGA
GGGTTGTTTTGGTTTTGGTTTTTGCTTTTGGCATTCCAGGAAATGCACAGTTTTACTCAGTG
TACCACAGAAATGTCCTAAGGAAGGTGATGAATGACCAAAGGTTCCCTTTCCTATTATACAA
GAAAAAATTCACAACACTCTGAGAAGCAAATTTCTTTTTGACTTTGATGAAAATCCACTTAGT
AACATGACTTGAACTTACATGAAACTACTCATAGTCTATTCATTCCACTTTATATGAATATTG
ATGTATCTGCTGTTGAAATAATAGTTTATGAGGCAGCCCTCCAGACCCCACGTAGAGTGTA
TGTAACAAGAGATGCACCATTTTATTTCTCGAAAACCCGTAACATTCTTCATTCCAAAACAC
ATCTGGCTTCTCGGAGGTCTGGACAAGTGATTCTTGGCAACACATACCTATAGAGACAATA
AAATCAAAGTAATAATGGCAACACAATAGATAACATTTACCAAGCATACACCATGTGGCAGA
CACAATTATAAGTGTTTTCCATATTTAACCTACTTAATCCTCAGGAATAAGCCACTGAGGTC
AGTCCTATTATTATCCCCATCTTATAGATGAAGAAAATGAGGCACCAGGAAGTCAAATAACT
TGTCAAAGGTCACAAGACTAGGAAATACACAAGTAGAAATGTTTACAATTAAGGCCCAGGC
TGGGTTTGCCCTCAGTTCTGCTATGCCTCGCATTATGCCCCAGGAAACTTTTTCCCTTGTG
AAAGCCAAGCTTAAAAAAAGAAAAGCCACATTTGTAACGTGCTCTGTTCCCCTGCCTATGG
TGAGGATCTTCAAACAGTTATACATGGACCCAGTCCCCCTGCCTTCTCCTTAATTTCTTAAG
TCATTTGAAACAGATGGCTGTCATGGAAATAGAATCCAGACATGTTGGTCAGAGTTAAAGA
TCAACTAATTCCATCAAAAATAGCTCGGCATGAAAGGGAACTATTCTCTGGCTTAGTCATG
GATGAGACTTTCAATTGCTATAAAGTGGTTCCTTTATTAGACAATGTTACCAGGGAAACAAC
AGGGGTTTGTTTGACTTCTGGGGCCCACAAGTCAACAAGAGAGCCCCATCTACCAAGGAG
CATGTCCCTGACTACCCCTCAGCCAGCAGCAAGACATGGACCCCAGTCAGGGCAGGAGC
AGGGTTTCGGCGGCGCCCAGCACAAGACATTGCCCCTAGAGTCTCAGCCCCTACCCTCG
AGTAATAGATCTGCCTACCTGAGACTGTTGTTTGCCCAAGAGCTGGGTCTCAGCCTGATG
GGAACCATATAAAAAGGTTCACTGACATACTGCCCACATGTTGTTCTCTTTCATTAGATCTT
AGCTTCCTTGTCTGCTCTTCATTCTTGCAGTATTCATTCAACAAACATTAAAAAAAAAAAAAA
GCATTCTATGTGTGGAACACTCTGCTAGATGCTGTGGATTTAGAAATGAAAATACATCCCG
ACCCTTGGAATGGAAGGGAAAGGACTGAAGTAAGACAGATTAAGCAGGACCGTCAGCCCA
GCTTGAAGCCCAGATAAATACGGAGAACAAGAGAGAGCGAGTAGTGAGAGATGAGTCCCA
ATGCCTCACTTTGGTGACGGGTGCGTGGTGGGCTTCATGCAGCTTCTTCTGATAAATGCCT
CCTTCAGAACTGGTCAACTCTACCTTGGCCAGTGACCCAGGTGGTCATAGTAGATTTACCA
AGGGAAAATGGAAACTTTTATTAGGAGCTCTTAGGCCTCTTCACTTCATGGATTTTTTTTTC
CTTTTTTTTTGAGATGGAGTTTTGCCCTGTCACCCAGGCTGGAATGCAGTGGTGCAATCTC
AGCTCACTGCAACCTCCGCCTCCCAGGTTCAAGCAATTCTCCTGCCTCAGCCTCCCGAGT
AGCTGGGACTACAGGTGTGCGCCACCACACCAGGCTAATTTTTGTATTTTTTGTAAAGACA
GGTTTTCACCACGTTGGCCAGGCTGGTCTGAACTCCAGACCTCAGGTGATTCACCTGTCT
CAGCCTCCCAAAGTGCTGGGATTACAGGTGTGAGCCACCGTGCCCGGCTACTTCATGGAT
TTTTGATTACAGATTATGCCTCTTACAATTTTTAAGAAGAATCAAGTGGGCTGAAGGTCAAT
GTCACCATAAGACAAAAGACATTTTTATTAGTTGATTCTAGGGAATTGGCCTTAAGGGGAG
CCCTTTCTTCCTAAGAGATTCTTAGGTGATTCTCACTTCCTCTTGCCCCAGTATTATTTTTGT
TTTTGGTATGGCTCACTCAGATCCTTTTTTCCTCCTATCCCTAAGTAATCCGGGTTTCTTTTT
CCCATATTTAGAACAAAATGTATTTATGCAGAGTGTGTCCAAACCTCAACCCAAGGCCTGTA
TACAAAATAAATCAAATTAAACACATCTTTACTGTCTTCTACCTCTTTCCTGACCTCAATATAT
CCCAACTTGCCTCACTCTGAGAACCAAGGCTGTCCCAGCACCTGAGTCGCAGATATTCTA
CTGATTTGACAGAACTGTGTGACTATCTGGAACAGCATTTTGATCCACAATTTGCCCAGTTA
CAAAGCTTAAATGAGCTCTAGTGCATGCATATATATTTCAAAATTCCACCATGATCTTCCAC
ACTCTGTATTGTAAATAGAGCCCTGTAATGCTTTTACTTCGTATTTCATTGCTTGTTATACAT
AAAAATATACTTTTCTTCTTCATGTTAGAAAATGCAAAGAATAGGAGGGTGGGGGAATCTCT
GGGCTTGGAGACAGGAGACTTGCCTTCCTACTATGGTTCCATCAGAATGTAGACTGGGAC
AATACAATAATTCAAGTCTGGTTTGCTCATCTGTAAATTGGGAAGAATGTTTCCAGCTCCAG
AATGCTAAATCTCTAAGTCTGTGGTTGGCAGCCACTATTGCAGCAGCTCTTCAATGACTCA
ATGCAGTTTTGCATTCTCCCTACCTTTTTTTTCTAAAACCAATAAAATAGATACAGCCTTTAG
GCTTTCTGGGATTTCCCTTAGTCAAGCTAGGGTCATCCTGACTTTCGGCGTGAATTTGCAA
AACAAGACCTGACTCTGTACTCCTGCTCTAAGGACTGTGCATGGTTCCAAAGGCTTAGCTT
GCCAGCATATTTGAGCTTTTTCCTTCTGTTCAAACTGTTCCAAAATATAAAAGAATAAAATTA
ATTAAGTTGGCACTGGACTTCCGGTGGTCAGTCATGTGTGTCATCTGTCACGTTTTTCGGG
CTCTGGTGGAAATGGATCTGTCTGTCTTCTCTCATAGGTGGTATTCACAGCCAACGAC
TCCGGCCCCCGCCGCTACACCATTGCCGCCCTGCTGAGCCCCTACTCCTATTCC
ACCACGGCTGTCGTCACCAATCCCAAGGAATGAGGGACTTCTCCTCCAGTGGACC
TGAAGGACGAGGGATGGGATTTCATGTAACCAAGAGTATTCCATTTTTACTAAAGCA
GTGTTTTCACCTCATATGCTATGTTAGAAGTCCAGGCAGAGACAATAAAACATTCCT
GTGAAAGGCACTTTTCATTCCACTTTAACTTGATTTTTTAAATTCCCTTATTGTCCCTT
CCAAAAAAAAGAGAATCAAAATTTTACAAAGAATCAAAGGAATTCTAGAAAGTATC
TGGGCAGAACGCTAGGAGAGATCCAAATTTCCATTGTCTTGCAAGCAAAGCACGTA
TTAAATATGATCTGCAGCCATTAAAAAGACACATTCTGTAAATGAGAGAGCCTTATT
TTCCTGTAACCTTCAGCAAATAGCAAAAGACACATTCCAAGGGCCCACTTCTTTACT
GTGGGCATTTCTTTTTTTTTCTTTTTTTCTTTTTTCCTTTTTTGAGACAAAGTCTCACTC
TGTTGCCCAGGCTAGAATGCAGTGGTGTAATCTCAGCTCACTGCAACCTCTGCTTCC
TGGGTTCAAGCGATTCTCCTGCCTCAGCCTCCCAAGTAACTGGGATTACAGGCGCAT
GCCACCACGCCTAGCTCATTTTTGTATTTTTAGTAGAGATGGGATTTTGCCATGTTGG
CTAGGCTGGTCTACGAACTCCTGACCTCAGGTGATCCACCTGCCTCAGCCTCCCAAA
GTGCTGGGATTACAGGCATGAGCCACTACACCCGGCCCCTACTCTGGGCATTTCTTT
GATTAAAGAGAAGGGGAGCTCCAACAAGATACACCTGCAGCAACTCAGGCCGTCTG
ATCAGTTCAGGCCAGATCTACACTGCAACCAGCCAGGTCAGGGGAAAACCAAAGAA
CCCCACACACCCAATTTACTTAGGCTGATCCAAAATCCATGTATGGAGAACTCACAT
GCACCAGGCACTATTTTAGGTGAACTGAATATAAAGAATAGGACCCAGTACCTGCA
TTTACTTAAAGAACTCACAATCTTTTGAGAACATAACTGTTTCATCATGGTTTGGCA
GGAGGCTATGGTACAAGGCACAGCAAGGGTAAGAAGGAGGAAGAAACCAACACCC
TACAGAAATCAGGGAATGACTCTGAATAGGTGTCACTTAATCTGAGTGTTGGTAATT
TGTCAGATAGACAAGGGAAAAGGTATTCTAGGTAGAGAGAATACAGTTTGCAAGGC
CCAGCCAAGTGAAACAATTTGATAAGTTGAGAGAGCAGACGACGATTCAGAATGTT
GAAGGGCAAAGGTATTGAGGTGGGATGGGTTATGCTGCTATCACAAATAACCCCAA
ATCTCGGGGGCTTAACAAAGTAAAAGTTTAGTCTCAGTTGTGCCAGGTCCAATGTAG
AACTCTTTGCTCTAGAGACTCTTTAGGGTGGCTTTCCTTCTAATGGTGACTGTTTGAG
ACAGTTTGATTTAGTCTTGTGGCTTCAAGGTCACTCTGGTGATATTTAGCCAGCAGA
CTGAGGGAACATAGTATGGTATTAGACCCCTCTGTGCTGAAGTGTCACACATGAGTC
CCATTGACTTCTCACTGGCCAGAGCTAGTTACATGCCCCCATCTAGATGTGCTGAGA
AATGTGGCCCCTGGCTGGGAGCCATTTCCCAGAACAACTAACTCTATGCTCTGGAAG
AGGAGCACTAATCTGAGTTGGCCAACAACCATCTCTACCACAGTAGGGTTGGGACT
GGTGGGGCATGAGGCTGGAGTGAAGGTTGGTTTTATCTGCCACGCGTTACAGCTGTG
AATTTGTCTTGAAAGCAACATGGGTCCATTGAAGGGAACCTTGACATCAGTCATGTG
GCTGGGACAAGAATAGTTACCACTTGCCCGTAATCTCCAACCAGGATTCTCCAGGAG
AACCTGAGTTAGACACATGGCTTAGGCCTAAACCTACCTGAGTGGTCTTTCTATTTT
CCTCCAAATTCAAATCTCAAATCTTGCTACCCTCTAACTGGCTATGTTGAGAGAGGA
AAAAACTTGAAGAGAATGCAGTGTAGCTTTGGAGTTTTTCACATGCACTTTTCCCAA
GATACATAGCAAAATCAATGTCTCCAATTCTATTAATGTTGTTAGCAAGTCCTTGTTC
CATGCATATTGGTTAATCCATAGCAAATTGCCATTTTTATAGACTAAATGGTCAAAT
ATTGGCAATTTCATAAGGTTCAGTCTATTACCTACCATGATTGTATTGGTCACTAACC
TGCCTATTTTTAGAATGCTACATATTCATTTGGCTGTTGTTAAATAGCTATGGATTTT
TATAATCAAAACAGGTTGAAAATATGAATCAGTTTAAAACCACATACA.

In the above TTR polynucleotide sequence provided at NCBI Reference Sequence No. NG_009490.1, exons encoding the TTR polypeptide correspond to the union of nucleotides 5137 . . . 5205, 6130 . . . 6260, 8354 . . . 8489, and 11802 . . . 11909, and the intervening sequences correspond to intron sequences. The union of nucleotides 5137 . . . 5205, 6130 . . . 6260, 8354 . . . 8489, and 11802 . . . 11909 corresponds to Consensus Coding Sequence (CCDS) No. 11899.1.

By “transthyretin amyloidosis” is meant a disease associated with a buildup of amyloid deposits comprising transthyretin in a tissue of a subject. The tissue can be organ tissue. The organ can be the liver.

By “amyloidosis” is meant a disease associated with buildup of amyloid in a tissue of a subject. The tissue can be organ tissue. The organ can be the liver.

By “adenine” or “9H-Purin-6-amine” is meant a purine nucleobase with the molecular formula C₅H₅N₅, having the structure

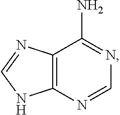

and corresponding to CAS No. 73-24-5.

By “adenosine” or “4-Amino-1-[(2R,3R,4S,5R)-3,4-dihy droxy-5-(hydroxymethyl)oxolan-2-yl]pyrimidin-2(1H)-one” is meant an adenine molecule attached to a ribose sugar via a glycosidic bond, having the structure

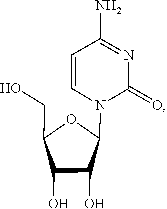

and corresponding to CAS No. 65-46-3. Its molecular formula is C₁₀H₁₃N₅O₄. The terms “adenine” and “adenosine” are used interchangeably throughout this document.

By “adenosine deaminase” or “adenine deaminase” is meant a polypeptide or fragment thereof capable of catalyzing the hydrolytic deamination of adenine or adenosine. The terms “adenine deaminase” and “adenosine deaminase” are used interchangeably throughout the application. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g. engineered adenosine deaminases, evolved adenosine deaminases) provided herein may be from any organism, such as a bacterium. In some embodiments, the adenosine deaminase is an adenosine deaminase variant with one or more alterations and is capable of deaminating both adenine and cytosine in a target polynucleotide (e.g., DNA). In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA.

By “adenosine deaminase activity” is meant catalyzing the deamination of adenine or adenosine to guanine in a polynucleotide. In some embodiments, an adenosine deaminase variant as provided herein maintains adenosine deaminase activity (e.g., at least about 30%, 40%, 50%, 60%, 70%, 80%, 90% or more of the activity of a reference adenosine deaminase (e.g., TadA*8.20 or TadA*8.19)).

By “Adenosine Base Editor 8.8 (ABE8.8) polypeptide” or “ABE8.8” is meant a base editor comprising an adenosine deaminase.

By “Adenosine Base Editor (ABE) polynucleotide” is meant a polynucleotide encoding an ABE.

By “Adenosine Base Editor 8 (ABE8.8)” or “ABE8.8” is meant a base editor as defined herein comprising an adenosine deaminase variant comprising the alterations Y123H, Y147R, and Q154R relative to the following reference sequence: MSEVEFSHEYWMRHALTLAKRARDEREVPVGAVLVLNNRVIGEGWNRAIGLHDPTAH AEIMALRQGGLVMQNYRLIDATLYVTFEPCVMCAGAMIHSRIGRVVFGVRNAKTGAAG SLMDVLHYPGMNHRVEITEGILADECAALLCYFFRMPRQVFNAQKKAQSSTD (SEQ ID NO: 4; TadA*7.10), or a corresponding position in another adenosine deaminase. In some embodiments, ABE8.8 comprises further alterations (e.g., 1, 2, 3, 4, 5, 6, 7, 8, 9, 10, 11, 13, 14, or 15 alterations) relative to the reference sequence, or a corresponding position in another adenosine deaminase.

By “Adenosine Base Editor 8.8 (ABE8.8) polynucleotide” is meant a polynucleotide encoding an ABE8.8 polypeptide.

By “Adenosine Base Editor 8.13 (ABE8.13) polypeptide” or “ABE8.13” is meant a base editor as defined herein comprising an adenosine deaminase variant comprising the alterations I76Y, Y123H, Y147R, and Q154R relative to the following reference sequence: MSEVEFSHEYWMRHALTLAKRARDEREVPVGAVLVLNNRVIGEGWNRAIGLHDPTAH AEIMALRQGGLVMQNYRLIDATLYVTFEPCVMCAGAMIHSRIGRVVFGVRNAKTGAAG SLMDVLHYPGMNHRVEITEGILADECAALLCYFFRMPRQVFNAQKKAQSSTD (SEQ ID NO: 4; TadA*7.10). In some embodiments, ABE8.13 comprises further alterations (e.g., 1, 2, 3, 4, 5, 6, 7, 8, 9, 10, 11, 13, 14, or 15 alterations) relative to the reference sequence.

By “Adenosine Base Editor 8.13 (ABE8.13) polynucleotide” is meant a polynucleotide encoding an ABE8.13 polypeptide.

“Administering” is referred to herein as providing one or more compositions described herein to a patient or a subject.

By “agent” is meant any small molecule chemical compound, antibody, nucleic acid molecule, or polypeptide, or fragments thereof.

By “alteration” is meant a change (increase or decrease) in the level, structure, or activity of an analyte, gene or polypeptide as detected by standard art known methods such as those described herein. As used herein, an alteration includes a 10% change in expression levels, a 25% change, a 40% change, and a 50% or greater change in expression levels. In some embodiments, an alteration includes an insertion, deletion, or substitution of a nucleobase or amino acid.

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1) in conjunction with a guide polynucleotide (e.g., guide RNA (gRNA)). Representative nucleic acid and protein sequences of base editors are provided in the Sequence Listing as SEQ ID NOs: 5-14.

By “Base Editor 4 polypeptide” or “BE4” is meant a base editor as defined herein comprising a cytidine deaminase variant comprising a sequence with at least about 85% sequence identity to the following reference sequence:

(SEQ ID NO: 15
MSSETGPVAVDPTLRRRIEPHEFEVFFDPRELRKETCLLYEINWGGRHS
IWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQ
ESGYCWRNFVNYSPSNEAHWPRYPHLWVRLYVLELYCIILGLPPCLNIL
RRKQPQLTFFTIALQSCHYQRLPPHILWATGLK;

BE4 cytidine deaminase domain). In some embodiments, BE4 comprises further alterations (e.g., 1, 2, 3, 4, 5, 6, 7, 8, 9, 10, 11, 13, 14, or 15 alterations) relative to the reference sequence.

By “Base Editor 4 polynucleotide” or “BE4 polynucleotide” is meant a polynucleotide encoding a BE4 polypeptide.

By “base editing activity” is meant acting to chemically alter a base within a polynucleotide. In one embodiment, a first base is converted to a second base. In one embodiment, the base editing activity is cytidine deaminase activity, e.g., converting target C•G to T•A. In another embodiment, the base editing activity is adenosine or adenine deaminase activity, e.g., converting A•T to G•C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine base editor (CBE).

By “base editing activity” is meant acting to chemically alter a base within a polynucleotide. In one embodiment, a first base is converted to a second base. In one embodiment, the base editing activity is cytidine deaminase activity, e.g., converting target C•G to T•A. In another embodiment, the base editing activity is adenosine deaminase activity, e.g., converting A•T to G•C.

By “bhCas12b v4 polypeptide” or “bhCas12b v4” is meant an endonuclease variant comprising a sequence with at least about 85% sequence identity to the following reference sequence and having endonuclease activity: MAPKKKRKVGIHGVPAAATRSFILKIEPNEEVKKGLWKTHEVLNHGIAYYMNILKLIRQ EAIYEHHEQDPKNPKKVSKAEIQAELWDFVLKMQKCNSFTHEVDKDEVFNILRELYEEL VPSSVEKKGEANQLSNKFLYPLVDPNSQSGKGTASSGRKPRWYNLKIAGDPSWEEEKK KWEEDKKKDPLAKILGKLAEYGLIPLFIPYTDSNEPIVKEIKWMEKSRNQSVRRLDKDM FIQALERFLSWESWNLKVKEEYEKVEKEYKTLEERIKEDIQALKALEQYEKERQEQLLR DTLNTNEYRLSKRGLRGWREIIQKWLKMDENEPSEKYLEVFKDYQRKHPREAGDYSVY EFLSKKENHFIWRNHPEYPYLYATFCEIDKKKKDAKQQATFTLADPINHPLWVRFEERS GSNLNKYRILTEQLHTEKLKKKLTVQLDRLIYPTESGGWEEKGKVDIVLLPSRQFYNQIF LDIEEKGKHAFTYKDESIKFPLKGTLGGARVQFDRDHLRRYPHKVESGNVGRIYFNMTV NIEPTESPVSKSLKIHRDDFPKVVNFKPKELTEWIKDSKGKKLKSGIESLEIGLRVMSIDL GQRQAAAASIFEVVDQKPDIEGKLFFPIKGTELYAVHRASFNIKLPGETLVKSREVLRKA REDNLKLMNQKLNFLRNVLHFQQFEDITEREKRVTKWISRQENSDVPLVYQDELIQIREL MYKPYKDWVAFLKQLHKRLEVEIGKEVKHWRKSLSDGRKGLYGISLKNIDEIDRTRKF LLRWSLRPTEPGEVRRLEPGQRFAIDQLNHLNALKEDRLKKMANTIIMHALGYCYDVR KKKWQAKNPACQIILFEDLSNYNPYGERSRFENSRLMKWSRREIPRQVALQGEIYGLQV GEVGAQFSSRFHAKTGSPGIRCRVVTKEKLQDNRFFKNLQREGRLTLDKIAVLKEGDLY PDKGGEKFISLSKDRKCVTTHADINAAQNLQKRFWTRTHGFYKVYCKAYQVDGQTVYI PESKDQKQKIIEEFGEGYFILKDGVYEWVNAGKLKIKKGSSKQSSSELVDSDILKDSFDL ASELKGEKLMLYRDPSGNVFPSDKWMAAGVFFGKLERILISKLTNQYSISTIEDDSSKQS MSGGSKRTADGSEFESPKKKRKVE (SEQ ID NO: 450). In some embodiments, bhCAS12b v4 comprises further alterations (e.g., 1, 2, 3, 4, 5, 6, 7, 8, 9, 10, 11, 13, 14, or 15 alterations) relative to the reference sequence.

By “bhCas12b v4 polynucleotide” is meant a polynucleotide encoding a bhCas12b v4.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following:

| | |
|---|---|
| Glutamine | CAG → TAG Stop codon |
| | CAA → TAA |
| Arginine | CGA → TGA |
| Tryptophan | TGG → TGA |
| | TGG → TAG |
| | TGG → TAA |

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and π-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds. Throughout the present disclosure, wherever an embodiment of a base editor is contemplated as containing a fusion protein, complexes comprising one or more domains of the base editor, or fragments thereof, are also contemplated.

By “cytidine” is meant a cytosine molecule attached to a ribose sugar via a glycosidic bond, having the structure

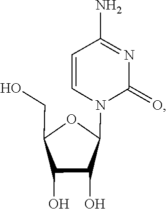

and corresponding to CAS No. 65-46-3. Its molecular formula is C₉H₁₃N₃O₅. The terms “cytosine” and “cytidine” are used interchangeably throughout this document.

By “cytidine deaminase” is meant a polypeptide or fragment thereof capable of catalyzing a deamination reaction that converts an amino group of cytidine to a carbonyl group. In one embodiment, the cytidine deaminase converts cytosine to uracil or 5-methylcytosine to thymine. The terms “cytidine deaminase” and “cytosine deaminase” are used interchangeably throughout the application. PmCDA1 (SEQ ID NO: 17-18), which is derived from Petromyzon marinus (Petromyzon marinus cytosine deaminase 1, “PmCDA1”), AID (Activation-induced cytidine deaminase; AICDA) (Exemplary AID polypeptide sequences are provided in the Sequence Listing as SEQ ID NOs: 19-25), which is derived from a mammal (e.g., human, swine, bovine, horse, monkey etc.), and APOBEC are exemplary cytidine deaminases (Exemplary APOBEC polypeptide sequences are provided in the Sequence Listing as SEQ ID NOs: 15 and 26-65. Further exemplary cytidine deaminase (CDA) sequences are provided in the Sequence Listing as SEQ ID NOs: 66-70. Additional exemplary cytidine deaminase sequences, including APOBEC polypeptide sequences, are provided in the Sequence Listing as SEQ ID NOs: 71-193.

By “cytosine” or “4-Aminopyrimidin-2(1H)-one” is meant a purine nucleobase with the molecular formula C₄H₅N₃O, having the structure

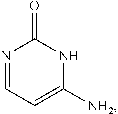

and corresponding to CAS No. 71-30-7.

By “cytosine deaminase activity” is meant catalyzing the deamination of cytosine in a polynucleotide, thereby converting an amino group to a carbonyl group. In one embodiment, a polypeptide having cytosine deaminase activity converts cytosine to uracil (i.e., C to U) or 5-methylcytosine to thymine (i.e., 5 mC to T). In some embodiments, an adenosine deaminase variant as provided herein has an increased cytosine deaminase activity (e.g., at least 10-fold, 20-fold, 30-fold, 40-fold, 50-fold, 60-fold, 70-fold, 80-fold, 90-fold, 100-fold or more) relative to a reference adenosine deaminase (e.g., TadA*8.20 or TadA*8.19).

The term “deaminase” or “deaminase domain,” as used herein, refers to a protein or enzyme that catalyzes a deamination reaction.

“Detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. Exemplary diseases include diseases amenable to treatment using the methods and/or compositions of the present disclosure include as non-limiting examples amyloidosis, cardiomyopathy, familial amyloid polyneuropathy (FAP), familial amyloid cardiomyopathy (FAC), familial transthyretin amyloidosis (FTA), senile systemic amyloidosis (SSA), transthyretin amyloidosis, and the like. The disease can be any disease associated with a mutation to a transthyretin (TTR) polynucleotide sequence.

By “effective amount” is meant the amount of an agent or active compound, e.g., a base editor as described herein, that is required to ameliorate the symptoms of a disease relative to an untreated patient or an individual without disease, i.e., a healthy individual, or is the amount of the agent or active compound sufficient to elicit a desired biological response. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the invention sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue or organ, but only to alter the pathogenic gene in about 1%, 5%, 10%, 25%, 50%, 75% or more of the cells present in a subject, tissue or organ. In one embodiment, an effective amount is sufficient to ameliorate one or more symptoms of a disease.

The term “exonuclease” refers to a protein or polypeptide capable of digesting a nucleic acid molecule from a free ends The nucleic acid can be DNA or RNA.

The term “endonuclease” refers to a protein or polypeptide capable of catalyzing internal regions in a nucleic acid molecule. The nucleic acid molecule can be DNA or RNA.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “guide RNA” or “gRNA” is meant a polynucleotide or polynucleotide complex which is specific for a target sequence and can form a complex with a polynucleotide programmable nucleotide binding domain protein (e.g., Cas12b, Cas9 or Cpf1). In an embodiment, the guide polynucleotide is a guide RNA (gRNA). gRNAs can exist as a complex of two or more RNAs, or as a single RNA molecule.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increases” is meant a positive alteration of at least 10%, 25%, 50%, 75%, or 100%.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In one embodiment, the term “linker” refers to a covalent linker (e.g., covalent bond) or a non-covalent linker.

By “marker” is meant any protein or polynucleotide having an alteration in expression, level, structure or activity that is associated with a disease or disorder. In an embodiment, the marker is an accumulation of amyloid protein. In an embodiment, the marker is an alteration (e.g., mutation) in the sequence of a in transthyretin polypeptide and/or a transthyretin polynucleotide.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (2′—e.g., fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. In some embodiments, anNLS comprises the amino acid sequence

(SEQ ID NO: 194)
KRTADGSEFESPKKKRKV,
(SEQ ID NO: 195)
KRPAATKKAGQAKKKK,
(SEQ ID NO: 196)
KKTELQTTNAENKTKKL,
(SEQ ID NO: 197)
KRGINDRNEWRGENGRKTR,
(SEQ ID NO: 198)
RKSGKIAAIVVKRPRK,
(SEQ ID NO: 199)
PKKKRKV,
or
(SEQ ID NO: 200)
MDSLLMNRRKFLYQFKNVRWAKGRRETYLC.

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U) —are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 201-234 and 383.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

By “subject” is meant a mammal, including, but not limited to, a human or non-human mammal, such as a bovine, equine, canine, ovine, rodent, or feline. In an embodiment, a “patient” or “subject” as used herein refers to a mammalian subject or individual diagnosed with, at risk of having or developing, or suspected of having or developing a disease or a disorder. In some embodiments, the term “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease casing mutation”, “disease causing variant”, “deleterious mutation”, or “predisposing mutation” refers to a genetic alteration or mutation that is associated with a disease or disorder that increases an individual's susceptibility or predisposition to a certain disease or disorder. In some embodiments, the pathogenic mutation comprises at least one wild-type amino acid substituted by at least one pathogenic amino acid in a protein encoded by a gene. In some embodiments, the pathogenic mutation is in a terminating region (e.g., stop codon). In some embodiments, the pathogenic mutation is in a non-coding region (e.g., intron, promoter, etc.)

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In one embodiment, the reference is a wild-type or healthy cell. In other embodiments and without limitation, a reference is an untreated cell that is not subjected to a test condition, or is subjected to placebo or normal saline, medium, buffer, and/or a control vector that does not harbor a polynucleotide of interest. The reference can be a cell or subject with a pathogenic mutation in a transthyretin (TTR) polynucleotide sequence and/or a transthyretin (TTR) polypeptide sequence. A reference can be a subject or cell with an amyloidosis (e.g., a transthyretin amyloidosis) or a subject or cell without an amyloidosis.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” are used with one or more RNA(s) that is not a target for cleavage. In some embodiments, an RNA-programmable nuclease, when in a complex with an RNA, may be referred to as a nuclease-RNA complex (alternatively, as a nuclease_RNA complex). Typically, the bound RNA(s) is referred to as a guide RNA (gRNA). In some embodiments, the RNA-programmable nuclease is the (CRISPR-associated system) Cas9 endonuclease, for example, Cas9 (Csn1) from Streptococcus pyogenes (e.g., SEQ ID NO: 201), Cas9 from Neisseria meningitidis (NmeCas9; SEQ ID NO: 212), Nme2Cas9 (SEQ ID NO: 213), or derivatives thereof (e.g. a sequence with at least about 85% sequence identity to a Cas9, such as Nme2Cas9 or spCas9).

The term “single nucleotide polymorphism (SNP)” is a variation in a single nucleotide that occurs at a specific position in the genome, where each variation is present to some appreciable degree within a population (e.g., >1%).

By “specifically binds” is meant a nucleic acid molecule, polypeptide, polypeptide/polynucleotide complex, compound, or molecule that recognizes and binds a polypeptide and/or nucleic acid molecule of the invention, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least 60%, 80%, 85%, 90%, 95% or even 99% identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. COBALT is used, for example, with the following parameters:
a) alignment parameters: Gap penalties-11,-1 and End-Gap penalties-5,-1,
b) CDD Parameters: Use RPS BLAST on; Blast E-value 0.003; Find Conserved columns and Recompute on, and
c) Query Clustering Parameters: Use query clusters on; Word Size 4; Max cluster distance 0.8; Alphabet Regular.
   EMBOSS Needle is used, for example, with the following parameters:
a) Matrix: BLOSUM62;
b) GAP OPEN: 10;
c) GAP EXTEND: 0.5;
d) OUTPUT FORMAT: pair;
e) END GAP PENALTY: false;
f) END GAP OPEN: 10; and
g) END GAP EXTEND: 0.5.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 370° C., and most preferably of at least about 420° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 300° C. in 750 mM NaCl, 75 mM trisodium citrate, and 10% SDS. In a more preferred embodiment, hybridization will occur at 370° C. in 500 mM NaCl, 50 mM trisodium citrate, 1% SDS, 35% formamide, and 100 μg/ml denatured salmon sperm DNA (ssDNA). In a most preferred embodiment, hybridization will occur at 420° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In an embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In a more preferred embodiment, wash steps will occur at 680° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “split” is meant divided into two or more fragments.

A “split Cas9 protein” or “split Cas9” refers to a Cas9 protein that is provided as an N-terminal fragment and a C-terminal fragment encoded by two separate nucleotide sequences. The polypeptides corresponding to the N-terminal portion and the C-terminal portion of the Cas9 protein may be spliced to form a “reconstituted” Cas9 protein.

The term “target site” refers to a sequence within a nucleic acid molecule that ismodified. In embodiments, the modification is deamination of a base. The deaminase can be a cytidine or an adenine deaminase. The fusion protein or base editing complex comprising a deaminase may comprise a dCas9-adenosine deaminase fusion protein, a Cas12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a compositions as described herein.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. Including an inhibitor of uracil DNA glycosylase (UGI) in the base editor prevents base excision repair which changes the U back to a C. An exemplary UGI comprises an amino acid sequence as follows: >splP14739IUNGI_BPPB2 Uracil-DNA glycosylase inhibitor

(SEQ ID NO: 235)
MTNLSDIIEKETGKQLVIQESILMLPEEVEEVIGNKPESDILVHTAYDES
TDENVMLLTSDAPEYKPWALVIQDSNGENKIKML.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, e.g., within 5-fold, within 2-fold of a value. Where particular values are described in the application and claims, unless otherwise stated, the term “about” means within an acceptable error range for the particular value should be assumed.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIGS. 1A-1C are plots showing base editing efficiency for base editor systems comprising the indicated base editors in combination with the indicated guide RNAs targeting a transthyretin (TTR) polynucleotide. FIG. 1A is a plot of A>G base editing efficiencies at a conserved splice site motif using the indicated base editors and guides. FIG. 1B is a plot of C>T base editing efficiencies in a splice site motif using the indicated base editors and guides. FIG. 1C is a plot of indel editing efficiencies.

FIG. 2 is a plot showing editing efficiency for a bhCas12b endonuclease used in combination with the indicated guide RNAs targeting a transthyretin (TTR) polynucleotide.

FIG. 3 provides a bar graph showing human TTR protein concentrations measured by ELISA in PXB-cell hepatocytes prior to transfection. Each condition was run in triplicate, as represented by each dot in the assay. Bar graphs illustrate the mean TTR protein concentrations and error bars indicate the standard deviation.

FIG. 4 provides a combined bar graph and plot showing editing rates in PXB-cell hepatocytes at the targeted site assessed at 13 days post-transfection by NGS (squares, right axis), and human TTR protein concentrations assessed 7 days post-transfection by ELISA (bars, left axis). Each condition was run in triplicate, as represented by each dot. In FIG. 4, the dotted line indicates the average human TTR concentration in cells edited using the base editing system ABE8.8_sgRNA_088. The starred sample (Cas9_gRNA991*) indicates that maximum indel rate within the protospacer region was measured, rather than rate of target base-editing.

FIG. 5 provides a combined bar graph and plot showing Editing rates in PXB-cell hepatocytes at the targeted site assessed at 13 days post-transfection by NGS (squares, right axis), and human TTR protein concentrations assessed 13 days post-transfection by ELISA (bars, left axis). Each condition was run in triplicate, as represented by each dot. In FIG. 5. The dotted line indicates the average human TTR concentration in cells edited using the base editing system ABE8.8_sgRNA_088. Starred sample indicates that maximum indel rate within the protospacer region was measured, rather than rate of target base-editing.

FIG. 6 provides a bar graph showing cyno TTR protein concentrations measured by ELISA in primary cyno hepatocyte co-culture supernatants prior to transfection. Each condition was run in triplicate, as represented by each dot in the assay. The bars illustrate the mean TTR protein concentrations and error bars indicate the standard deviation.

FIG. 7 provides a combined bar graph and plot showing editing rates in primary cyno hepatocyte co-cultures at the targeted site assessed at 13 days post-transfection by NGS (squares, right axis), and cyno TTR protein concentrations assessed 7 days post-transfection by ELISA (bars, left axis). Each condition was run in triplicate, as represented by each dot in the graph. The dotted line indicates the average cyno TTR concentration in cells edited using a base editing system including ABE8.8_sgRNA_088.

FIG. 8 provides a combined bar graph and plot showing editing rates in primary cyno hepatocyte co-cultures at the targeted site assessed at 13 days post-transfection by NGS (squares, right axis), and cyno TTR protein concentrations assessed 13 days post-transfection by ELISA (bars, left axis). Each condition was run in triplicate, as represented by each dot in the graph. The dotted line indicates the average cyno TTR concentration in cells edited using the base editing system ABE8.8_sgRNA_088.

FIGS. 9A and 9B present schematics showing the TTR promoter sequence aligned to gRNAs designed for a screen. In FIG. 9A, The gRNAs are shown above or below the sequence shown in the figure depending on their strand orientation. In each of FIGS. 9A and 9B, the gRNA protospacer sequence plus PAM sequence is shown in each annotation. The nucleotide sequence shown in FIGS. 9A and 9B is provided in the sequence listing as SEQ ID NO: 547 and the amino acid sequence shown in FIG. 9 is provided in the sequence listing as SEQ ID NO: 548.

FIG. 10 provides a bar graph showing next-generation sequencing (NGS) data from three replicates of HepG2 cells transfected with mRNA encoding the indicated editor (indicated above the bars) and gRNA encoding the indicated gRNA (indicated along the x-axis). Dots represent individual data points for each edit type (i.e., indel, max. A-to-G, max. C-to-T) shown. Max A-to-G or max. C-to-T reflects the highest editing frequency for any A or C base within the gRNA protospacer. Three replicates were performed on the same day.

FIG. 11 provides a bar graph showing TTR knockdown data. Individual data points for 2 replicates of TTR expression data are plotted. Three technical replicates for each data point for the RT-qPCR were performed and the mean is plotted for 2 biological data points. All data are from transfections were performed on the same day. RT-qPCR analysis was performed relative to untreated controls in the same RT-qPCR plate as the test well. ACTB was used as an internal control for each sample. Untreated cells had a different TTR:ACTB ratio than transfected cells, which led to artificially reduced relative TTR expression (0.30-0.42) in cells transfected with negative control catalytically dead Cas9 editor or gRNA that would not affect TTR expression.

FIGS. 12A and 12B provide a schematics showing the location of promoter tiling gRNAs effective in a TTR RT-qPCR knockdown assay. All gRNAs that demonstrated comparable or improved TTR knockdown as compared with a nuclease approach are shown. Five highly effective gRNAs, as measured by TTR RT-qPCR, were gRNA1756 ABE, gRNA1764 ABE, gRNA1790 CBE, gRNA1786 ABE, and gRNA1772 ABE. A few gRNAs that lowered TTR transcript levels overlapped with putative functional elements including a putative TATA box (transcription initiation site) and a start codon (translation initiation site) as indicated in FIGS. 12A and 12B. In FIGS. 12A and 12B, * indicates the gRNA was highly effective when paired with either an ABE or CBE; ** indicates editing frequency was <50% for this gRNA, not intending to be bound by theory, this could indicate that the gRNA was acting though a mechanism distinct from or in addition to base editing; and *** indicates both that the gRNA was highly effective when paired with either an ABE or CBE and that editing frequency was <50% for this gRNA. In FIG. 12B, five potent gRNA's, as measured by TTR RT-qPCR, are shown in white (gRNA1756 ABE, gRNA1764 ABE, gRNA1790 CBE, gRNA1786 ABE, and gRNA1772 ABE). The nucleotide sequence shown in FIG. 12A is provided in the sequence listing as SEQ ID NO: 549 and the amino acid sequence shown in FIG. 12A is provided in the sequence listing as SEQ ID NO: 550. The nucleotide sequence shown in FIG. 12B corresponds to SEQ ID NO: 1160.

FIG. 13 provides a bar graph showing editing rates at the targeted sites assessed at 72 hours post-transfection by NGS. Each experimental condition was run in triplicate and is displayed as an average with standard error of the mean. Total splice site disruption without unintended in-gene edits is shown as the left bar of each pair of bars, and unintended edits are shown as the right bar of each pair of bars. The total editing by the gRNA991 spCas9 control is displayed as the left bar for the “gRNA991+spCas9” sample.

### DETAILED DESCRIPTION OF THE INVENTION

The invention features compositions and methods for editing a transthyretin polynucleotide sequence to treat transthyretin amyloidosis.

The invention is based, at least in part, on the discovery that editing can be used to disrupt expression of a transthyretin polypeptide or to edit a pathogenic mutation in a transthyretin polypeptide. In one particular embodiment, the invention provides guide RNA sequences that are effective for use in conjunction with a base editing system for editing a transthyretin (TTR) gene sequence to disrupt splicing or correct a pathogenic mutation. In another embodiment, the invention provides guide RNA sequences that target a Cas12b nuclease to edit a TTR gene sequence, thereby disrupting TTR polypeptide expression.

Accordingly, the invention provides guide RNA sequences suitable for use with ABE and/or BE4 for transthyretin (TTR) gene splice site disruption and guide RNA sequences suitable for use with bhCas12b nucleases for disruption of the transthyretin (TTR) gene. In embodiments, the compositions and methods of the present invention can be used for editing a TTR gene in a hepatocyte. The methods provided herein can include reducing or eliminating expression of TTR in a hepatocyte cell to treat an amyloidosis.

### Amyloidosis

Amyloidosis is a disorder that involved extracellular deposition of amyloid in an organ or tissue (e.g., the liver). Amyloidosis can occur when mutant transthyretin polypeptides aggregate (e.g., as fibrils). An amyloidosis caused by a mutation to the transthyretin gene can be referred to as a “transthyretin amyloidosis”. Some forms of transthyretin amyloidosis are not associated with a mutation to the transthyretin gene. Non-limiting examples of mutations to the mature transthyretin (TTR) protein that can lead to amyloidosis include the alterations T60A, V30M, V30A, V30G, V30L, V122I, V122A, and V122(−). One method for treatment of transthyretin amyloidosis includes disrupting expression or activity of transthyretin in a cell of a subject, optionally a hepatocyte cell. Accordingly, provided herein are methods for reducing or eliminating expression of transthyretin in a cell. The transthyretin in the cell can be a pathogenic variant. Expression of transthyretin in a cell can be disrupted by disrupting splicing of a transthyretin transcript.

### Transthyretin Amyloidosis

Transthyretin amyloidosis is a progressive condition characterized by the buildup of protein deposits in organs and/or tissues. These protein deposits can occur in the peripheral nervous system, which is made up of nerves connecting the brain and spinal cord to muscles and sensory cells that detect sensations such as touch, pain, heat, and sound. Protein deposits in these nerves result in a loss of sensation in the extremities (peripheral neuropathy). The autonomic nervous system, which controls involuntary body functions such as blood pressure, heart rate, and digestion, may also be affected by amyloidosis. In some cases, the brain and spinal cord (i.e., central nervous system) are affected. Other areas of amyloidosis include the heart, kidneys, eyes, liver, and gastrointestinal tract. The age at which symptoms begin to develop can be between the ages of 20 and 70.

There are three major forms of transthyretin amyloidosis, which are distinguished by their symptoms and the body systems they effect: neuropathic, leptomeningeal, and cardiac.

The neuropathic form of transthyretin amyloidosis primarily affects the peripheral and autonomic nervous systems, resulting in peripheral neuropathy and difficulty controlling bodily functions. Impairments in bodily functions can include sexual impotence, diarrhea, constipation, problems with urination, and a sharp drop in blood pressure upon standing (orthostatic hypotension). Some people experience heart and kidney problems as well. Various eye problems may occur, such as cloudiness of the clear gel that fills the eyeball (vitreous opacity), dry eyes, increased pressure in the eyes (glaucoma), or pupils with an irregular or “scallope”d appearance. Some people with this form of transthyretin amyloidosis develop carpal tunnel syndrome, which can involve numbness, tingling, and weakness in the hands and fingers.

The leptomeningeal form of transthyretin amyloidosis primarily affects the central nervous system. In people with this form, amyloidosis occurs in the leptomeninges, which are two thin layers of tissue that cover the brain and spinal cord. A buildup of protein in this tissue can cause stroke and bleeding in the brain, an accumulation of fluid in the brain (hydrocephalus), difficulty coordinating movements (ataxia), muscle stiffness and weakness (spastic paralysis), seizures, and loss of intellectual function (dementia). Eye problems similar to those in the neuropathic form may also occur. When people with leptomeningeal transthyretin amyloidosis have associated eye problems, they are said to have the oculoleptomeningeal form.

The cardiac form of transthyretin amyloidosis affects the heart. People with cardiac amyloidosis may have an abnormal heartbeat (arrhythmia), an enlarged heart (cardiomegaly), or orthostatic hypertension. These abnormalities can lead to progressive heart failure and death. Occasionally, people with the cardiac form of transthyretin amyloidosis have mild peripheral neuropathy.

Mutations in the transthyretin (TTR) gene cause transthyretin amyloidosis. Transthyretin transports vitamin A (retinol) and a hormone called thyroxine throughout the body. Not being bound by theory, to transport retinol and thyroxine, transthyretin must form a tetramer. Transthyretin is produced primarily in the liver (i.e., in hepatic cells). A small amount of transthyretin (TTR) is produced in an area of the brain called the choroid plexus and in the retina.

TTR gene mutations can alter the structure of transthyretin, impairing its ability to bind to other transthyretin proteins. The TTR gene mutation can be autosomal dominant.

### Splice Sites

Gene splice sites and splice site motifs are well known in the art and it is within the skill of a practitioner to identify splice sites in sequence (see, e.g., Sheth, et al., “Comprehensive splice-site analysis using comparative genomics”, Nucleic Acids Research, 34:3955-3967 (2006); Dogan, et al., “AplicePort—an interactive splice-site analysis tool”, Nucleic Acids Research, 35:W285-W291 (2007); and Zuallaert, et al., “SpliceRover: interpretable convolutional neural networks for improved splice site prediction”, Bioinformatics, 34:4180-4188 (2018)).

### Editing of Target Genes

To edit the transthyretin (TTR) gene, a cell (e.g., a hepatocyte) is contacted with a guide RNA and a nucleobase editor polypeptide comprising a nucleic acid programmable DNA binding protein (napDNAbp) and a cytidine deaminase or adenosine deaminase to edit a base of a gene sequence. Editing of the base can result in disruption of a splice site (e.g, through alteration of a splice-site motif nucleobase). Editing of the base can result in replacement of a pathogenic variant amino acid with a non-pathogenic variant amino acid. As a non-limiting example, editing of the base can result in replacing a T60A, V30M, V30A, V30G, V30L, V122I, V122A, or a V122(−) alteration in the mature transthyretin (TTR) polypeptide with a non-pathogenic variant or the wild-type valine residue. The cytidine deaminase can be BE4 (e.g., saBE4). The adenosine deaminase can be ABE (e.g., saABE.8.8). In some embodiments, multiple target sites are edited simultaneously. In some embodiments, the TTR gene is edited by contacting a cell with a nuclease and a guide RNA to introduce an indel into a gene sequence. The indel can be associated with a reduction or elimination of expression of the gene. The nuclease can be Cas12b (e.g., bhCas12b). The cells can be edited in vivo or ex vivo. The guide RNA can be a single guide or a dual guide. In some embodiments, cells to be edited are contacted with at least one nucleic acid, wherein at least one nucleic acid encodes a guide RNA, or two or more guide RNAs, and a nucleobase editor polypeptide comprising a nucleic acid programmable DNA binding protein (napDNAbp) and a deaminase, e.g., an adenosine or a cytidine deaminase. In some embodiments, the gRNA comprises nucleotide analogs. These nucleotide analogs can inhibit degradation of the gRNA by cellular processes. Exemplary single guide RNA (sgRNA) sequences are provided in Table 1 and exemplary spacer sequences and target sequences are provided in Tables 2A, 2B, and 2C.

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science.1231143). In some cases, a 5′ terminal “G” is added to a guide polynucleotide that is to be expressed under the control of a promoter, but is optionally not added to the guide polynucleotide if or when the guide polynucleotide is not expressed under the control of a promoter.

Exemplary guide RNAs, spacer sequences, and target sequences are provided in the following Tables 1, 2A, 2B, and 2C.

In embodiments, a guide RNA comprises a sequence complementary to a promoter region of a TTR polynucleotide sequence. In embodiments, the promoter region spans from positions +10, +5, +1, −1, −2, −3, −4, −5, −6, −7, −8, −9, −10, −15, −20, −25, −30, −35, −40, −45, −50, −55, −60, −65, −70, −75, −80, −85, −90, −95, −100, −105, −110, −115, −120, −125, −130, −135, −140, −145, −150, −155, −160, −165, −170, −175, −180, −185, −190, −195, −200, −250, or −300 to position +5, +1, −1, −2, −3, −4, −5, −6, −7, −8, −9, −10, −15, −20, −25, −30, −35, −40, −45, −50, −55, −60, −65, −70, −75, −80, −85, −90, −95, −100, −105, −110, −115, −120, −125, −130, −135, −140, −145, −150, −155, −160, −165, −170, −175, −180, −185, −190, −195, −200, −250, −300, or −400, where position +1 corresponds to the first A of the start codon (ATG) of the TTR polynucleotide sequence.

TABLE 1
Guide RNAs for editing transthyretin (TTR) splice sites and/or introducing indels
into the TTR gene (e.g., using bhCas12b)

| | | SEQ ID |
|---|---|---|
| sgRNA ID | Sequence | NO |
| sgRNA_361 | mUsmAsmUsAGGAAAACCAGTGAGTCGUUUUAGAGCUAGAAAUA | 394 |
| | GCAAGUUAAAAUAAGGCUAGUCCGUUAUCAACUUGAAAAAGUGG | |
| | CACCGAGUCGGUGCmUsmUsmUsU | |
| sgRNA_362 | mUsmAsmCsUCACCUCUGCAUGCUCAGUUUUAGAGCUAGAAAUA | 395 |
| | GCAAGUUAAAAUAAGGCUAGUCCGUUAUCAACUUGAAAAAGUGG | |
| | CACCGAGUCGGUGCmUsmUsmUsU | |
| sgRNA_363 | mAsmCsmUsCACCUCUGCAUGCUCAUGUUUUAGAGCUAGAAAUA | 396 |
| | GCAAGUUAAAAUAAGGCUAGUCCGUUAUCAACUUGAAAAAGUGG | |
| | CACCGAGUCGGUGCmUsmUsmUsU | |
| sgRNA_364 | mUsmAsmCsCACCUAUGAGAGAAGACGUUUUAGAGCUAGAAAUA | 397 |
| | GCAAGUUAAAAUAAGGCUAGUCCGUUAUCAACUUGAAAAAGUGG | |
| | CACCGAGUCGGUGCmUsmUsmUsU | |
| sgRNA_365 | mAsmUsmAsCUCACCUCUGCAUGCUCAGUUUUAGUACUCUGUAA | 398 |
| | UGAAAAUUACAGAAUCUACUAAAACAAGGCAAAAUGCCGUGUUU | |
| | AUCUCGUCAACUUGUUGGCGAGAmUsmUsmUsU | |
| sgRNA_366 | mAsmCsmUsGGUUUUCCUAUAAGGUGUGUUUUAGUACUCUGUAA | 399 |
| | UGAAAAUUACAGAAUCUACUAAAACAAGGCAAAAUGCCGUGUUU | |
| | AUCUCGUCAACUUGUUGGCGAGAmUsmUsmUsU | |
| sgRNA_367 | mGsmUsmUsCUGUCUUUUGGUCAGGACAACCGUCUAGCUAUAAG | 400 |
| | UGCUGCAGGGUGUGAGAAACUCCUAUUGCUGGACGAUGUCUCUU | |
| | ACGAGGCAUUAGCACUUGGCAGGAUGGCUUCUCmAsmUsmCsG | |
| sgRNA_368 | mGsmUsmUsCUGUCUUUUGGUCAGGACAACCGUCUAGCUAUAAG | 401 |
| | UGCUGCAGGGUGUGAGAAACUCCUAUUGCUGGACGAUGUCUCUU | |
| | ACGAGGCAUUAGCACUCCUAUAAGGUGUGAAAGmUsmCsmUsG | |
| sgRNA_369 | mGsmUsmUsCUGUCUUUUGGUCAGGACAACCGUCUAGCUAUAAG | 402 |
| | UGCUGCAGGGUGUGAGAAACUCCUAUUGCUGGACGAUGUCUCUU | |
| | ACGAGGCAUUAGCACUGAGCCCAUGCAGCUCUCmCsmAsmGsA | |
| sgRNA_370 | mGsmUsmUsCUGUCUUUUGGUCAGGACAACCGUCUAGCUAUAAG | 403 |
| | UGCUGCAGGGUGUGAGAAACUCCUAUUGCUGGACGAUGUCUCUU | |
| | ACGAGGCAUUAGCACCUCCUCAGUUGUGAGCCCmAsmUsmGsC | |
| sgRNA_371 | mGsmUsmUsCUGUCUUUUGGUCAGGACAACCGUCUAGCUAUAAG | 404 |
| | UGCUGCAGGGUGUGAGAAACUCCUAUUGCUGGACGAUGUCUCUU | |
| | ACGAGGCAUUAGCACGUAGAAGGGAUAUACAAAmGsmUsmGsG | |
| sgRNA_372 | mGsmUsmUsCUGUCUUUUGGUCAGGACAACCGUCUAGCUAUAAG | 405 |
| | UGCUGCAGGGUGUGAGAAACUCCUAUUGCUGGACGAUGUCUCUU | |
| | ACGAGGCAUUAGCACCCACUUUGUAUAUCCCUUmCsmUsmAsC | |
| sgRNA_373 | mGsmUsmUsCUGUCUUUUGGUCAGGACAACCGUCUAGCUAUAAG | 406 |
| | UGCUGCAGGGUGUGAGAAACUCCUAUUGCUGGACGAUGUCUCUU | |
| | ACGAGGCAUUAGCACGGUGUCUAUUUCCACUUUmGsmUsmAsU | |
| sgRNA_374 | mGsmUsmUsCUGUCUUUUGGUCAGGACAACCGUCUAGCUAUAAG | 407 |
| | UGCUGCAGGGUGUGAGAAACUCCUAUUGCUGGACGAUGUCUCUU | |
| | ACGAGGCAUUAGCACCAUGAGCAUGCAGAGGUGmAsmGsmUsA | |
| gRNA1594 | mCsmAsmAsCUUACCCAGAGGCAAAUGUUUUAGAGCUAGAAAUA | 597 |
| | GCAAGUUAAAAUAAGGCUAGUCCGUUAUCAACUUGAAAAAGUGG | |
| | CACCGAGUCGGUGCUsmUsmUsmU | |
| gRNA1595 | mAsmAsmUsGGCUCCCAGGUGUCAUCGUUUUAGAGCUAGAAAUA | 598 |
| | GCAAGUUAAAAUAAGGCUAGUCCGUUAUCAACUUGAAAAAGUGG | |
| | CACCGAGUCGGUGCUsmUsmUsmU | |
| gRNA1596 | mGsmGsmCsUCCCAGGUGUCAUCAGCGUUUUAGAGCUAGAAAUA | 599 |
| | GCAAGUUAAAAUAAGGCUAGUCCGUUAUCAACUUGAAAAAGUGG | |
| | CACCGAGUCGGUGCUsmUsmUsmU | |
| gRNA1597 | mCsmUsmCsUCAUAGGUGGUAUUCACGUUUUAGAGCUAGAAAUA | 600 |
| | GCAAGUUAAAAUAAGGCUAGUCCGUUAUCAACUUGAAAAAGUGG | |
| | CACCGAGUCGGUGCUsmUsmUsmU | |
| gRNA1598 | mUsmAsmUsAGGAAAACCAGUGAGUCGUUUUAGAGCUAGAAAUA | 601 |
| | GCAAGUUAAAAUAAGGCUAGUCCGUUAUCAACUUGAAAAAGUGG | |
| | CACCGAGUCGGUGCUsmUsmUsmU | |
| gRNA1599 | mUsmAsmCsUCACCUCUGCAUGCUCAGUUUUAGAGCUAGAAAUA | 602 |
| | GCAAGUUAAAAUAAGGCUAGUCCGUUAUCAACUUGAAAAAGUGG | |
| | CACCGAGUCGGUGCUsmUsmUsmU | |
| gRNA1600 | mGsmCsmAsACUUACCCAGAGGCAAAGUUUUAGAGCUAGAAAUA | 603 |
| | GCAAGUUAAAAUAAGGCUAGUCCGUUAUCAACUUGAAAAAGUGG | |
| | CACCGAGUCGGUGCUsmUsmUsmU | |
| gRNA1601 | mUsmCsmUsGUAUACUCACCUCUGCAGUUUUAGAGCUAGAAAUA | 604 |
| | GCAAGUUAAAAUAAGGCUAGUCCGUUAUCAACUUGAAAAAGUGG | |
| | CACCGAGUCGGUGCUsmUsmUsmU | |
| gRNA1602 | mGsmAsmAsACACUCACCGUAGGGCCGUUUUAGAGCUAGAAAUA | 605 |
| | GCAAGUUAAAAUAAGGCUAGUCCGUUAUCAACUUGAAAAAGUGG | |
| | CACCGAGUCGGUGCUsmUsmUsmU | |
| gRNA1603 | mCsmUsmCsUACACCCAGGGCACCGGGUUUUAGAGCUAGAAAUA | 606 |
| | GCAAGUUAAAAUAAGGCUAGUCCGUUAUCAACUUGAAAAAGUGG | |
| | CACCGAGUCGGUGCUsmUsmUsmU | |
| gRNA1604 | mAsmCsmAsCCUUAUAGGAAAACCAGGUUUUAGAGCUAGAAAUA | 607 |
| | GCAAGUUAAAAUAAGGCUAGUCCGUUAUCAACUUGAAAAAGUGG | |
| | CACCGAGUCGGUGCUsmUsmUsmU | |
| gRNA1605 | mAsmUsmAsGGAAAACCAGUGAGUCUGUUUUAGAGCUAGAAAUA | 608 |
| | GCAAGUUAAAAUAAGGCUAGUCCGUUAUCAACUUGAAAAAGUGG | |
| | CACCGAGUCGGUGCUsmUsmUsmU | |
| gRNA1606 | mAsmCsmUsCACCUCUGCAUGCUCAUGUUUUAGAGCUAGAAAUA | 609 |
| | GCAAGUUAAAAUAAGGCUAGUCCGUUAUCAACUUGAAAAAGUGG | |
| | CACCGAGUCGGUGCUsmUsmUsmU | |
| gRNA1607 | mCsmUsmCsACCGUAGGGCCAGCCUCGUUUUAGAGCUAGAAAUA | 610 |
| | GCAAGUUAAAAUAAGGCUAGUCCGUUAUCAACUUGAAAAAGUGG | |
| | CACCGAGUCGGUGCUsmUsmUsmU | |
| gRNA1746 | mAsmAsmCsCUGCUGAUUCUGAUUAUGUUUUAGAGCUAGAAAUAG | 753 |
| | CAAGUUAAAAUAAGGCUAGUCCGUUAUCAACUUGAAAAAGUGGC | |
| | ACCGAGUCGGUGCUsmUsmUsmU | |
| gRNA1747 | mAsmAsmGsAGAGAAUAAGUAACCCAUGUUUUAGUACUCUGUAAU | 754 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1748 | mAsmAsmGsCAGCCUAGCUCAGGAGAAGUUUUAGUACUCUGUAAU | 755 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1749 | mAsmAsmGsUCCACUCAUUCUUGGCAGUUUUAGAGCUAGAAAUAG | 756 |
| | CAAGUUAAAAUAAGGCUAGUCCGUUAUCAACUUGAAAAAGUGGC | |
| | ACCGAGUCGGUGCUsmUsmUsmU | |
| gRNA1750 | mAsmCsmGsAUGAGAAGCCAUCCUGCCGUUUUAGUACUCUGUAAU | 757 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1751 | mAsmGsmAsCAAGGUUCAUAUUUGUAGUUUUAGAGCUAGAAAUAG | 758 |
| | CAAGUUAAAAUAAGGCUAGUCCGUUAUCAACUUGAAAAAGUGGC | |
| | ACCGAGUCGGUGCUsmUsmUsmU | |
| gRNA1752 | mAsmGsmGsCUGGGAGCAGCCAUCACGUUUUAGAGCUAGAAAUAG | 759 |
| | CAAGUUAAAAUAAGGCUAGUCCGUUAUCAACUUGAAAAAGUGGC | |
| | ACCGAGUCGGUGCUsmUsmUsmU | |
| gRNA1753 | mAsmUsmAsAGUAACCCAUACAAAUAGUUUUAGAGCUAGAAAUAG | 760 |
| | CAAGUUAAAAUAAGGCUAGUCCGUUAUCAACUUGAAAAAGUGGC | |
| | ACCGAGUCGGUGCUsmUsmUsmU | |
| gRNA1754 | mAsmUsmAsCUCACUUCUCCUGAGCUGUUUUAGAGCUAGAAAUAG | 761 |
| | CAAGUUAAAAUAAGGCUAGUCCGUUAUCAACUUGAAAAAGUGGC | |
| | ACCGAGUCGGUGCUsmUsmUsmU | |
| gRNA1755 | mAsmUsmUsAUUGACUUAGUCAACAAGUUUUAGAGCUAGAAAUAG | 762 |
| | CAAGUUAAAAUAAGGCUAGUCCGUUAUCAACUUGAAAAAGUGGC | |
| | ACCGAGUCGGUGCUsmUsmUsmU | |
| gRNA1756 | mCsmAsmAsAUAUGAACCUUGUCUAGGUUUUAGAGCUAGAAAUAG | 763 |
| | CAAGUUAAAAUAAGGCUAGUCCGUUAUCAACUUGAAAAAGUGGC | |
| | ACCGAGUCGGUGCUsmUsmUsmU | |
| gRNA1757 | mCsmAsmGsAAGUCCACUCAUUCUUGGGUUUUAGUACUCUGUAAU | 764 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1758 | mCsmAsmGsGCUGGGAGCAGCCAUCACGUUUUAGUACUCUGUAAU | 765 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1759 | mCsmCsmAsUCCUGCCAAGAAUGAGUGUUUUAGAGCUAGAAAUAG | 766 |
| | CAAGUUAAAAUAAGGCUAGUCCGUUAUCAACUUGAAAAAGUGGC | |
| | ACCGAGUCGGUGCUsmUsmUsmU | |
| gRNA1760 | mCsmCsmUsGCUGAUUCUGAUUAUUGAGUUUUAGUACUCUGUAAU | 767 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1761 | mCsmGsmAsUGCUCUAAUCUCUCUAGAGUUUUAGUACUCUGUAAU | 768 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1762 | mCsmUsmAsAGUCAAUAAUCAGAAUCAGUUUUAGUACUCUGUAAU | 769 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1763 | mCsmUsmAsGACAAGGUUCAUAUUUGUGUUUUAGUACUCUGUAAU | 770 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1764 | mGsmAsmAsCCUUGUCUAGAGAGAUUGUUUUAGAGCUAGAAAUAG | 771 |
| | CAAGUUAAAAUAAGGCUAGUCCGUUAUCAACUUGAAAAAGUGGC | |
| | ACCGAGUCGGUGCUsmUsmUsmU | |
| gRNA1765 | mGsmAsmAsGUCCACUCAUUCUUGGCGUUUUAGAGCUAGAAAUAG | 772 |
| | CAAGUUAAAAUAAGGCUAGUCCGUUAUCAACUUGAAAAAGUGGC | |
| | ACCGAGUCGGUGCUsmUsmUsmU | |
| gRNA1766 | mGsmAsmAsUCAGCAGGUUUGCAGUCGUUUUAGAGCUAGAAAUAG | 773 |
| | CAAGUUAAAAUAAGGCUAGUCCGUUAUCAACUUGAAAAAGUGGC | |
| | ACCGAGUCGGUGCUsmUsmUsmU | |
| gRNA1767 | mGsmAsmAsUGAGUGGACUUCUGUGAGUUUUAGAGCUAGAAAUAG | 774 |
| | CAAGUUAAAAUAAGGCUAGUCCGUUAUCAACUUGAAAAAGUGGC | |
| | ACCGAGUCGGUGCUsmUsmUsmU | |
| gRNA1768 | mGsmAsmCsUGCAAACCUGCUGAUUCGUUUUAGAGCUAGAAAUAG | 775 |
| | CAAGUUAAAAUAAGGCUAGUCCGUUAUCAACUUGAAAAAGUGGC | |
| | ACCGAGUCGGUGCUsmUsmUsmU | |
| gRNA1769 | mGsmAsmCsUUAGUCAACAAAGAGAGAGUUUUAGUACUCUGUAAU | 776 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1770 | mGsmAsmUsAAGCAGCCUAGCUCAGGGUUUUAGAGCUAGAAAUAG | 777 |
| | CAAGUUAAAAUAAGGCUAGUCCGUUAUCAACUUGAAAAAGUGGC | |
| | ACCGAGUCGGUGCUsmUsmUsmU | |
| gRNA1771 | mGsmAsmUsGAGAAGCCAUCCUGCCAGUUUUAGAGCUAGAAAUAG | 778 |
| | CAAGUUAAAAUAAGGCUAGUCCGUUAUCAACUUGAAAAAGUGGC | |
| | ACCGAGUCGGUGCUsmUsmUsmU | |
| gRNA1772 | mGsmCsmCsAUCCUGCCAAGAAUGAGGUUUUAGAGCUAGAAAUAG | 779 |
| | CAAGUUAAAAUAAGGCUAGUCCGUUAUCAACUUGAAAAAGUGGC | |
| | ACCGAGUCGGUGCUsmUsmUsmU | |
| gRNA1773 | mGsmCsmUsUUUAUACUCACUUCUCCGUUUUAGAGCUAGAAAUAG | 780 |
| | CAAGUUAAAAUAAGGCUAGUCCGUUAUCAACUUGAAAAAGUGGC | |
| | ACCGAGUCGGUGCUsmUsmUsmU | |
| gRNA1774 | mGsmGsmAsUAAGCAGCCUAGCUCAGGGUUUUAGUACUCUGUAAU | 781 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1775 | mGsmUsmCsUAGAGAGAUUAGAGCAUGUUUUAGAGCUAGAAAUAG | 782 |
| | CAAGUUAAAAUAAGGCUAGUCCGUUAUCAACUUGAAAAAGUGGC | |
| | ACCGAGUCGGUGCUsmUsmUsmU | |
| gRNA1776 | mGsmUsmGsAUGGCUGCUCCCAGCCUGUUUUAGAGCUAGAAAUAG | 783 |
| | CAAGUUAAAAUAAGGCUAGUCCGUUAUCAACUUGAAAAAGUGGC | |
| | ACCGAGUCGGUGCUsmUsmUsmU | |
| gRNA1777 | mUsmAsmCsUUAUUCUCUCUUUGUUGAGUUUUAGUACUCUGUAAU | 784 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1778 | mUsmAsmUsUCUCUCUUUGUUGACUAAGUUUUAGUACUCUGUAAU | 785 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1779 | mUsmAsmUsUGACUUAGUCAACAAAGGUUUUAGAGCUAGAAAUAG | 786 |
| | CAAGUUAAAAUAAGGCUAGUCCGUUAUCAACUUGAAAAAGUGGC | |
| | ACCGAGUCGGUGCUsmUsmUsmU | |
| gRNA1780 | mUsmAsmUsUGACUUAGUCAACAAAGAGUUUUAGUACUCUGUAAU | 787 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1781 | mUsmCsmAsGAAUCAGCAGGUUUGCAGGUUUUAGUACUCUGUAAU | 788 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1782 | mUsmCsmCsACUCAUUCUUGGCAGGAGUUUUAGAGCUAGAAAUAG | 789 |
| | CAAGUUAAAAUAAGGCUAGUCCGUUAUCAACUUGAAAAAGUGGC | |
| | ACCGAGUCGGUGCUsmUsmUsmU | |
| gRNA1783 | mUsmCsmUsCUCUUUGUUGACUAAGUCGUUUUAGUACUCUGUAAU | 790 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1784 | mUsmGsmAsGAAGCCAUCCUGCCAAGAGUUUUAGUACUCUGUAAU | 791 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1785 | mUsmGsmAsGCUAGGCUGCUUAUCCCUGUUUUAGUACUCUGUAAU | 792 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1786 | mUsmGsmAsGUAUAAAAGCCCCAGGCGUUUUAGAGCUAGAAAUAG | 793 |
| | CAAGUUAAAAUAAGGCUAGUCCGUUAUCAACUUGAAAAAGUGGC | |
| | ACCGAGUCGGUGCUsmUsmUsmU | |
| gRNA1787 | mUsmGsmAsUGGCUGCUCCCAGCCUGGUUUUAGAGCUAGAAAUAG | 794 |
| | CAAGUUAAAAUAAGGCUAGUCCGUUAUCAACUUGAAAAAGUGGC | |
| | ACCGAGUCGGUGCUsmUsmUsmU | |
| gRNA1788 | mUsmGsmCsCAAGAAUGAGUGGACUUCGUUUUAGUACUCUGUAAU | 795 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1789 | mUsmGsmCsCAAUCUGACUGCAAACCUGUUUUAGUACUCUGUAAU | 796 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA1790 | mUsmGsmUsUGACUAAGUCAAUAAUCGUUUUAGAGCUAGAAAUAG | 797 |
| | CAAGUUAAAAUAAGGCUAGUCCGUUAUCAACUUGAAAAAGUGGC | |
| | ACCGAGUCGGUGCUsmUsmUsmU | |
| gRNA1791 | mUsmUsmGsACUUAGUCAACAAAGAGGUUUUAGAGCUAGAAAUAG | 798 |
| | CAAGUUAAAAUAAGGCUAGUCCGUUAUCAACUUGAAAAAGUGGC | |
| | ACCGAGUCGGUGCUsmUsmUsmU | |
| gRNA1792 | mUsmUsmUsGUUGACUAAGUCAAUAAUGUUUUAGUACUCUGUAAU | 799 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA-#1 | mAsmAsmAsAGCCCCAGGCUGGGAGCGUUUUAGAGCUAGAAAUAG | 800 |
| | CAAGUUAAAAUAAGGCUAGUCCGUUAUCAACUUGAAAAAGUGGC | |
| | ACCGAGUCGGUGCUsmUsmUsmU | |
| gRNA-#2 | mAsmAsmGsUGAGUAUAAAAGCCCCAGUUUUAGAGCUAGAAAUAG | 801 |
| | CAAGUUAAAAUAAGGCUAGUCCGUUAUCAACUUGAAAAAGUGGC | |
| | ACCGAGUCGGUGCUsmUsmUsmU | |
| gRNA-#3 | mAsmAsmUsAAUCAGAAUCAGCAGGUUGUUUUAGUACUCUGUAAU | 802 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA-#4 | mAsmAsmUsAUGAACCUUGUCUAGAGGUUUUAGAGCUAGAAAUAG | 803 |
| | CAAGUUAAAAUAAGGCUAGUCCGUUAUCAACUUGAAAAAGUGGC | |
| | ACCGAGUCGGUGCUsmUsmUsmU | |
| gRNA-#5 | mAsmAsmUsGAGUGGACUUCUGUGAUGUUUUAGAGCUAGAAAUAG | 804 |
| | CAAGUUAAAAUAAGGCUAGUCCGUUAUCAACUUGAAAAAGUGGC | |
| | ACCGAGUCGGUGCUsmUsmUsmU | |
| gRNA-#6 | mAsmCsmAsAAUAUGAACCUUGUCUAGGUUUUAGUACUCUGUAAU | 805 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA-#7 | mAsmCsmAsGAAGUCCACUCAUUCUUGUUUUAGAGCUAGAAAUAG | 806 |
| | CAAGUUAAAAUAAGGCUAGUCCGUUAUCAACUUGAAAAAGUGGC | |
| | ACCGAGUCGGUGCUsmUsmUsmU | |
| gRNA-#8 | mAsmCsmCsUUGUCUAGAGAGAUUAGGUUUUAGAGCUAGAAAUAG | 807 |
| | CAAGUUAAAAUAAGGCUAGUCCGUUAUCAACUUGAAAAAGUGGC | |
| | ACCGAGUCGGUGCUsmUsmUsmU | |
| gRNA-#9 | mAsmGsmAsAGCCAUCCUGCCAAGAAGUUUUAGAGCUAGAAAUAG | 808 |
| | CAAGUUAAAAUAAGGCUAGUCCGUUAUCAACUUGAAAAAGUGGC | |
| | ACCGAGUCGGUGCUsmUsmUsmU | |
| gRNA-#10 | mAsmGsmCsAGGUUUGCAGUCAGAUUGUUUUAGAGCUAGAAAUAG | 809 |
| | CAAGUUAAAAUAAGGCUAGUCCGUUAUCAACUUGAAAAAGUGGC | |
| | ACCGAGUCGGUGCUsmUsmUsmU | |
| gRNA-#11 | mAsmGsmGsGAUAAGCAGCCUAGCUCGUUUUAGAGCUAGAAAUAG | 810 |
| | CAAGUUAAAAUAAGGCUAGUCCGUUAUCAACUUGAAAAAGUGGC | |
| | ACCGAGUCGGUGCUsmUsmUsmU | |
| gRNA-#12 | mAsmGsmGsUUUGCAGUCAGAUUGGCGUUUUAGAGCUAGAAAUAG | 811 |
| | CAAGUUAAAAUAAGGCUAGUCCGUUAUCAACUUGAAAAAGUGGC | |
| | ACCGAGUCGGUGCUsmUsmUsmU | |
| gRNA-#13 | mAsmGsmUsAUAAAAGCCCCAGGCUGGUUUUAGAGCUAGAAAUAG | 812 |
| | CAAGUUAAAAUAAGGCUAGUCCGUUAUCAACUUGAAAAAGUGGC | |
| | ACCGAGUCGGUGCUsmUsmUsmU | |
| gRNA-#14 | mAsmGsmUsCAAUAAUCAGAAUCAGCGUUUUAGAGCUAGAAAUAG | 813 |
| | CAAGUUAAAAUAAGGCUAGUCCGUUAUCAACUUGAAAAAGUGGC | |
| | ACCGAGUCGGUGCUsmUsmUsmU | |
| gRNA-#15 | mAsmUsmAsAUCAGAAUCAGCAGGUUGUUUUAGAGCUAGAAAUAG | 814 |
| | CAAGUUAAAAUAAGGCUAGUCCGUUAUCAACUUGAAAAAGUGGC | |
| | ACCGAGUCGGUGCUsmUsmUsmU | |
| gRNA-#16 | mGsmUsmUsCUGUCUUUUGGUCAGGACAACCGUCUAGCUAUAAGU | 815 |
| | GCUGCAGGGUGUGAGAAACUCCUAUUGCUGGACGAUGUCUCUUA | |
| | CGAGGCAUUAGCACUUGACUUAGUCAACAAAGAsmGsmAsmG | |
| gRNA-#17 | mGsmUsmUsCUGUCUUUUGGUCAGGACAACCGUCUAGCUAUAAGU | 816 |
| | GCUGCAGGGUGUGAGAAACUCCUAUUGCUGGACGAUGUCUCUUA | |
| | CGAGGCAUUAGCACUCUCUUUGUUGACUAAGUCsmAsmAsmU | |
| gRNA-#18 | mGsmUsmUsCUGUCUUUUGGUCAGGACAACCGUCUAGCUAUAAGU | 817 |
| | GCUGCAGGGUGUGAGAAACUCCUAUUGCUGGACGAUGUCUCUUA | |
| | CGAGGCAUUAGCACUGAUUAUUGACUUAGUCAAsmCsmAsmA | |
| gRNA-#19 | mGsmUsmUsCUGUCUUUUGGUCAGGACAACCGUCUAGCUAUAAGU | 818 |
| | GCUGCAGGGUGUGAGAAACUCCUAUUGCUGGACGAUGUCUCUUA | |
| | CGAGGCAUUAGCACUUGGCAGGAUGGCUUCUCAsmUsmCsmG | |
| gRNA-#20 | mGsmUsmUsCUGUCUUUUGGUCAGGACAACCGUCUAGCUAUAAGU | 819 |
| | GCUGCAGGGUGUGAGAAACUCCUAUUGCUGGACGAUGUCUCUUA | |
| | CGAGGCAUUAGCACACUUAGUCAACAAAGAGAGsmAsmAsmU | |
| gRNA-#21 | mGsmUsmUsCUGUCUUUUGGUCAGGACAACCGUCUAGCUAUAAGU | 820 |
| | GCUGCAGGGUGUGAGAAACUCCUAUUGCUGGACGAUGUCUCUUA | |
| | CGAGGCAUUAGCACGCAGGGAUAAGCAGCCUAGsmCsmUsmC | |
| gRNA-#22 | mGsmUsmUsCUGUCUUUUGGUCAGGACAACCGUCUAGCUAUAAGU | 821 |
| | GCUGCAGGGUGUGAGAAACUCCUAUUGCUGGACGAUGUCUCUUA | |
| | CGAGGCAUUAGCACGUAUGGGUUACUUAUUCUCsmUsmCsmU | |
| gRNA-#23 | mCsmAsmAsGAAUGAGUGGACUUCUGGUUUUAGAGCUAGAAAUAG | 822 |
| | CAAGUUAAAAUAAGGCUAGUCCGUUAUCAACUUGAAAAAGUGGC | |
| | ACCGAGUCGGUGCUsmUsmUsmU | |
| gRNA-#24 | mCsmAsmAsUCUGACUGCAAACCUGCGUUUUAGAGCUAGAAAUAG | 823 |
| | CAAGUUAAAAUAAGGCUAGUCCGUUAUCAACUUGAAAAAGUGGC | |
| | ACCGAGUCGGUGCUsmUsmUsmU | |
| gRNA-#25 | mCsmAsmCsAGAAGUCCACUCAUUCUGUUUUAGAGCUAGAAAUAG | 824 |
| | CAAGUUAAAAUAAGGCUAGUCCGUUAUCAACUUGAAAAAGUGGC | |
| | ACCGAGUCGGUGCUsmUsmUsmU | |
| gRNA-#26 | mCsmAsmGsACGAUGAGAAGCCAUCCGUUUUAGAGCUAGAAAUAG | 825 |
| | CAAGUUAAAAUAAGGCUAGUCCGUUAUCAACUUGAAAAAGUGGC | |
| | ACCGAGUCGGUGCUsmUsmUsmU | |
| gRNA-#27 | mCsmAsmGsCAGGUUUGCAGUCAGAUGUUUUAGAGCUAGAAAUAG | 826 |
| | CAAGUUAAAAUAAGGCUAGUCCGUUAUCAACUUGAAAAAGUGGC | |
| | ACCGAGUCGGUGCUsmUsmUsmU | |
| gRNA-#28 | mCsmAsmGsGAUGGCUUCUCAUCGUCGUUUUAGAGCUAGAAAUAG | 827 |
| | CAAGUUAAAAUAAGGCUAGUCCGUUAUCAACUUGAAAAAGUGGC | |
| | ACCGAGUCGGUGCUsmUsmUsmU | |
| gRNA-#29 | mCsmAsmGsGUUUGCAGUCAGAUUGGCGUUUUAGUACUCUGUAAU | 828 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA-#30 | mCsmAsmGsUCAGAUUGGCAGGGAUAGUUUUAGAGCUAGAAAUAG | 829 |
| | CAAGUUAAAAUAAGGCUAGUCCGUUAUCAACUUGAAAAAGUGGC | |
| | ACCGAGUCGGUGCUsmUsmUsmU | |
| gRNA-#31 | mCsmCsmAsCUCAUUCUUGGCAGGAUGUUUUAGAGCUAGAAAUAG | 830 |
| | CAAGUUAAAAUAAGGCUAGUCCGUUAUCAACUUGAAAAAGUGGC | |
| | ACCGAGUCGGUGCUsmUsmUsmU | |
| gRNA-#32 | mCsmUsmAsAGUCAAUAAUCAGAAUCGUUUUAGAGCUAGAAAUAG | 831 |
| | CAAGUUAAAAUAAGGCUAGUCCGUUAUCAACUUGAAAAAGUGGC | |
| | ACCGAGUCGGUGCUsmUsmUsmU | |
| gRNA-#33 | mGsmUsmUsCUGUCUUUUGGUCAGGACAACCGUCUAGCUAUAAGU | 832 |
| | GCUGCAGGGUGUGAGAAACUCCUAUUGCUGGACGAUGUCUCUUA | |
| | CGAGGCAUUAGCACGUCAACAAAGAGAGAAUAAsmGsmUsmA | |
| gRNA-#34 | mCsmUsmUsAUCCCUGCCAAUCUGACGUUUUAGAGCUAGAAAUAG | 833 |
| | CAAGUUAAAAUAAGGCUAGUCCGUUAUCAACUUGAAAAAGUGGC | |
| | ACCGAGUCGGUGCUsmUsmUsmU | |
| gRNA-#35 | mGsmUsmUsCUGUCUUUUGGUCAGGACAACCGUCUAGCUAUAAGU | 834 |
| | GCUGCAGGGUGUGAGAAACUCCUAUUGCUGGACGAUGUCUCUUA | |
| | CGAGGCAUUAGCACUCCCUGCCAAUCUGACUGCsmAsmAsmA | |
| gRNA-#36 | mGsmUsmUsCUGUCUUUUGGUCAGGACAACCGUCUAGCUAUAAGU | 835 |
| | GCUGCAGGGUGUGAGAAACUCCUAUUGCUGGACGAUGUCUCUUA | |
| | CGAGGCAUUAGCACUUCUCUCUUUGUUGACUAAsmGsmUsmC | |
| gRNA-#37 | mGsmUsmUsCUGUCUUUUGGUCAGGACAACCGUCUAGCUAUAAGU | 836 |
| | GCUGCAGGGUGUGAGAAACUCCUAUUGCUGGACGAUGUCUCUUA | |
| | CGAGGCAUUAGCACUCCUGAGCUAGGCUGCUUAsmUsmCsmC | |
| gRNA-#38 | mCsmUsmUsCUGUGAUGGCUGCUCCCGUUUUAGAGCUAGAAAUAG | 837 |
| | CAAGUUAAAAUAAGGCUAGUCCGUUAUCAACUUGAAAAAGUGGC | |
| | ACCGAGUCGGUGCUsmUsmUsmU | |
| gRNA-#39 | mGsmUsmUsCUGUCUUUUGGUCAGGACAACCGUCUAGCUAUAAGU | 838 |
| | GCUGCAGGGUGUGAGAAACUCCUAUUGCUGGACGAUGUCUCUUA | |
| | CGAGGCAUUAGCACUGUGAUGGCUGCUCCCAGCsmCsmUsmG | |
| gRNA-#40 | mGsmUsmUsCUGUCUUUUGGUCAGGACAACCGUCUAGCUAUAAGU | 839 |
| | GCUGCAGGGUGUGAGAAACUCCUAUUGCUGGACGAUGUCUCUUA | |
| | CGAGGCAUUAGCACGCAGGAUGGCUUCUCAUCGsmUsmCsmU | |
| gRNA-#41 | mGsmUsmUsCUGUCUUUUGGUCAGGACAACCGUCUAGCUAUAAGU | 840 |
| | GCUGCAGGGUGUGAGAAACUCCUAUUGCUGGACGAUGUCUCUUA | |
| | CGAGGCAUUAGCACUCUAGAGAGAUUAGAGCAUsmCsmGsmG | |
| gRNA-#42 | mGsmUsmUsCUGUCUUUUGGUCAGGACAACCGUCUAGCUAUAAGU | 841 |
| | GCUGCAGGGUGUGAGAAACUCCUAUUGCUGGACGAUGUCUCUUA | |
| | CGAGGCAUUAGCACGUUGACUAAGUCAAUAAUCsmAsmGsmA | |
| gRNA-#43 | mGsmUsmUsCUGUCUUUUGGUCAGGACAACCGUCUAGCUAUAAGU | 842 |
| | GCUGCAGGGUGUGAGAAACUCCUAUUGCUGGACGAUGUCUCUUA | |
| | CGAGGCAUUAGCACUAUACUCACUUCUCCUGAGsmCsmUsmA | |
| gRNA-#44 | mGsmAsmAsGUGAGUAUAAAAGCCCCGUUUUAGAGCUAGAAAUAG | 843 |
| | CAAGUUAAAAUAAGGCUAGUCCGUUAUCAACUUGAAAAAGUGGC | |
| | ACCGAGUCGGUGCUsmUsmUsmU | |
| gRNA-#45 | mGsmAsmCsAAGGUUCAUAUUUGUAUGUUUUAGAGCUAGAAAUAG | 844 |
| | CAAGUUAAAAUAAGGCUAGUCCGUUAUCAACUUGAAAAAGUGGC | |
| | ACCGAGUCGGUGCUsmUsmUsmU | |
| gRNA-#46 | mGsmAsmGsUAUAAAAGCCCCAGGCUGUUUUAGAGCUAGAAAUAG | 845 |
| | CAAGUUAAAAUAAGGCUAGUCCGUUAUCAACUUGAAAAAGUGGC | |
| | ACCGAGUCGGUGCUsmUsmUsmU | |
| gRNA-#47 | mGsmAsmGsUGGACUUCUGUGAUGGCGUUUUAGAGCUAGAAAUAG | 846 |
| | CAAGUUAAAAUAAGGCUAGUCCGUUAUCAACUUGAAAAAGUGGC | |
| | ACCGAGUCGGUGCUsmUsmUsmU | |
| gRNA-#48 | mGsmAsmUsGGCUGCUCCCAGCCUGGGUUUUAGAGCUAGAAAUAG | 847 |
| | CAAGUUAAAAUAAGGCUAGUCCGUUAUCAACUUGAAAAAGUGGC | |
| | ACCGAGUCGGUGCUsmUsmUsmU | |
| gRNA-#49 | mGsmCsmAsGCCUAGCUCAGGAGAAGGUUUUAGAGCUAGAAAUAG | 848 |
| | CAAGUUAAAAUAAGGCUAGUCCGUUAUCAACUUGAAAAAGUGGC | |
| | ACCGAGUCGGUGCUsmUsmUsmU | |
| gRNA-#50 | mGsmCsmUsGCUUAUCCCUGCCAAUCGUUUUAGAGCUAGAAAUAG | 849 |
| | CAAGUUAAAAUAAGGCUAGUCCGUUAUCAACUUGAAAAAGUGGC | |
| | ACCGAGUCGGUGCUsmUsmUsmU | |
| gRNA-#51 | mGsmGsmGsAUAAGCAGCCUAGCUCAGUUUUAGAGCUAGAAAUAG | 850 |
| | CAAGUUAAAAUAAGGCUAGUCCGUUAUCAACUUGAAAAAGUGGC | |
| | ACCGAGUCGGUGCUsmUsmUsmU | |
| gRNA-#52 | mGsmGsmUsUUGCAGUCAGAUUGGCAGUUUUAGAGCUAGAAAUAG | 851 |
| | CAAGUUAAAAUAAGGCUAGUCCGUUAUCAACUUGAAAAAGUGGC | |
| | ACCGAGUCGGUGCUsmUsmUsmU | |
| gRNA-#53 | mGsmUsmUsACUUAUUCUCUCUUUGUGUUUUAGAGCUAGAAAUAG | 852 |
| | CAAGUUAAAAUAAGGCUAGUCCGUUAUCAACUUGAAAAAGUGGC | |
| | ACCGAGUCGGUGCUsmUsmUsmU | |
| gRNA-#54 | mGsmUsmUsCUGUCUUUUGGUCAGGACAACCGUCUAGCUAUAAGU | 853 |
| | GCUGCAGGGUGUGAGAAACUCCUAUUGCUGGACGAUGUCUCUUA | |
| | CGAGGCAUUAGCACCUUAUUCUCUCUUUGUUGAsmCsmUsmA | |
| gRNA-#55 | mGsmUsmUsCUGUCUUUUGGUCAGGACAACCGUCUAGCUAUAAGU | 854 |
| | GCUGCAGGGUGUGAGAAACUCCUAUUGCUGGACGAUGUCUCUUA | |
| | CGAGGCAUUAGCACAUAUUUGUAUGGGUUACUUsmAsmUsmU | |
| gRNA-#56 | mGsmUsmUsCUGUCUUUUGGUCAGGACAACCGUCUAGCUAUAAGU | 855 |
| | GCUGCAGGGUGUGAGAAACUCCUAUUGCUGGACGAUGUCUCUUA | |
| | CGAGGCAUUAGCACACUAAGUCAAUAAUCAGAAsmUsmCsmA | |
| gRNA-#57 | mGsmUsmUsUGCAGUCAGAUUGGCAGGUUUUAGAGCUAGAAAUAG | 856 |
| | CAAGUUAAAAUAAGGCUAGUCCGUUAUCAACUUGAAAAAGUGGC | |
| | ACCGAGUCGGUGCUsmUsmUsmU | |
| gRNA-#58 | mGsmUsmUsCUGUCUUUUGGUCAGGACAACCGUCUAGCUAUAAGU | 857 |
| | GCUGCAGGGUGUGAGAAACUCCUAUUGCUGGACGAUGUCUCUUA | |
| | CGAGGCAUUAGCACGCAGUCAGAUUGGCAGGGAsmUsmAsmA | |
| gRNA-#59 | mUsmAsmCsAAAUAUGAACCUUGUCUGUUUUAGAGCUAGAAAUAG | 858 |
| | CAAGUUAAAAUAAGGCUAGUCCGUUAUCAACUUGAAAAAGUGGC | |
| | ACCGAGUCGGUGCUsmUsmUsmU | |
| gRNA-#60 | mUsmAsmCsUCACUUCUCCUGAGCUAGUUUUAGAGCUAGAAAUAG | 859 |
| | CAAGUUAAAAUAAGGCUAGUCCGUUAUCAACUUGAAAAAGUGGC | |
| | ACCGAGUCGGUGCUsmUsmUsmU | |
| gRNA-#61 | mUsmAsmUsAAAAGCCCCAGGCUGGGGUUUUAGAGCUAGAAAUAG | 860 |
| | CAAGUUAAAAUAAGGCUAGUCCGUUAUCAACUUGAAAAAGUGGC | |
| | ACCGAGUCGGUGCUsmUsmUsmU | |
| gRNA-#62 | mUsmCsmAsCUUCUCCUGAGCUAGGCGUUUUAGAGCUAGAAAUAG | 861 |
| | CAAGUUAAAAUAAGGCUAGUCCGUUAUCAACUUGAAAAAGUGGC | |
| | ACCGAGUCGGUGCUsmUsmUsmU | |
| gRNA-#63 | mUsmCsmAsGAUUGGCAGGGAUAAGCGUUUUAGAGCUAGAAAUAG | 862 |
| | CAAGUUAAAAUAAGGCUAGUCCGUUAUCAACUUGAAAAAGUGGC | |
| | ACCGAGUCGGUGCUsmUsmUsmU | |
| gRNA-#64 | mUsmCsmAsGGAGAAGUGAGUAUAAAGUUUUAGAGCUAGAAAUAG | 863 |
| | CAAGUUAAAAUAAGGCUAGUCCGUUAUCAACUUGAAAAAGUGGC | |
| | ACCGAGUCGGUGCUsmUsmUsmU | |
| gRNA-#65 | mUsmCsmUsGACUGCAAACCUGCUGAUGUUUUAGUACUCUGUAAU | 864 |
| | GAAAAUUACAGAAUCUACUAAAACAAGGCAAAAUGCCGUGUUUA | |
| | UCUCGUCAACUUGUUGGCGAGAUsmUsmUsmU | |
| gRNA-#66 | mUsmGsmAsGCUAGGCUGCUUAUCCCGUUUUAGAGCUAGAAAUAG | 865 |
| | CAAGUUAAAAUAAGGCUAGUCCGUUAUCAACUUGAAAAAGUGGC | |
| | ACCGAGUCGGUGCUsmUsmUsmU | |
| gRNA-#67 | mUsmGsmCsCAAUCUGACUGCAAACCGUUUUAGAGCUAGAAAUAG | 866 |
| | CAAGUUAAAAUAAGGCUAGUCCGUUAUCAACUUGAAAAAGUGGC | |
| | ACCGAGUCGGUGCUsmUsmUsmU | |
| gRNA-#68 | mUsmGsmCsUCUAAUCUCUCUAGACAGUUUUAGAGCUAGAAAUAG | 867 |
| | CAAGUUAAAAUAAGGCUAGUCCGUUAUCAACUUGAAAAAGUGGC | |
| | ACCGAGUCGGUGCUsmUsmUsmU | |
| gRNA-#69 | mUsmGsmUsGAUGGCUGCUCCCAGCCGUUUUAGAGCUAGAAAUAG | 868 |
| | CAAGUUAAAAUAAGGCUAGUCCGUUAUCAACUUGAAAAAGUGGC | |
| | ACCGAGUCGGUGCUsmUsmUsmU | |
| gRNA-#70 | mUsmUsmGsGCAGGGAUAAGCAGCCUGUUUUAGAGCUAGAAAUAG | 869 |
| | CAAGUUAAAAUAAGGCUAGUCCGUUAUCAACUUGAAAAAGUGGC | |
| | ACCGAGUCGGUGCUsmUsmUsmU | |
| gRNA-#71 | mUsmUsmUsUAUACUCACUUCUCCUGGUUUUAGAGCUAGAAAUAG | 870 |
| | CAAGUUAAAAUAAGGCUAGUCCGUUAUCAACUUGAAAAAGUGGC | |
| | ACCGAGUCGGUGCUsmUsmUsmU | |

Lowercase m indicates 2′-O-methylated nucleobases (e.g., mA, mC, mG, mU), and “s” indicates phosphorothioates.

TABLE 2A
Exemplary Spacer and Target Site Sequences. One of skill in the art will understand that some of the
target site sequences correspond to a reverse-complement to the above-provided transthyretin
polynucleotide sequence; i.e., the target sequences may correspond to either strand of a dsDNA
molecule encoding a transthyretin polynucleotide. Further, it is to be understood that a C base can
be targeted by a cytidine deaminase and that an A base can be targeted by an adenine deaminase.

| | | SEQ | Target | Target site sequence (target bases | SEQ | |
|---|---|---|---|---|---|---|
| | | ID | site | for base editing are in bold and | ID | Target |
| sgRNA | Spacer sequence | NO | ID | underlined) | NO | Base(s) |
| sgRNA_361 | UAUAGGAAAACCAGUGAGUC | 408 | TSBTx2602 | TATAGGAAAACCAGTGAGTC | 425 | 4A |
| sgRNA_362 | UACUCACCUCUGCAUGCUCA | 409 | TSBTx2603 | TACTCACCTCTGCATGCTCA | 426 | 6A, 7C |
| sgRNA_363 | ACUCACCUCUGCAUGCUCAU | 410 | TSBTx2604 | ACTCACCTCTGCATGCTCAT | 427 | 5A, 6C |
| sgRNA_364 | UACCACCUAUGAGAGAAGAC | 411 | TSBTx2605 | TACCACCTATGAGAGAAGAC | 428 | 7C |
| sgRNA_365 | AUACUCACCUCUGCAUGCUCA | 412 | TSBTx2606 | ATACTCACCTCTGCATGCTCA | 429 | 7A, 8C |
| sgRNA_366 | ACUGGUUUUCCUAUAAGGUGU | 413 | TSBTx2607 | ACTGGTTTTCCTATAAGGTGT | 430 | 11C |
| sgRNA_367 | UUGGCAGGAUGGCUUCUCAUCG | 414 | TSBTx2608 | TTGGCAGGATGGCTTCTCATCG | 431 | 6A, 9A |
| sgRNA_368 | UCCUAUAAGGUGUGAAAGUCUG | 415 | TSBTx2609 | GTTTTCCTATAAGGTGTGAAAGTCTG | 432 | |
| sgRNA_369 | UGAGCCCAUGCAGCUCUCCAGA | 416 | TSBTx2610 | GTTGTGAGCCCATGCAGCTCTCCAGA | 433 | |
| sgRNA_370 | CUCCUCAGUUGUGAGCCCAUGC | 417 | TSBTx2611 | ATTCCTCCTCAGTTGTGAGCCCATGC | 434 | |
| sgRNA_371 | GUAGAAGGGAUAUACAAAGUGG | 418 | TSBTx2612 | ATTTGTAGAAGGGATATACAAAGTGG | 435 | |
| sgRNA_372 | CCACUUUGUAUAUCCCUUCUAC | 419 | TSBTx2613 | ATTTCCACTTTGTATATCCCTTCTAC | 436 | |
| sgRNA_373 | GGUGUCUAUUUCCACUUUGUAU | 420 | TSBTx2614 | ATTTGGTGTCTATTTCCACTTTGTAT | 437 | |
| sgRNA_374 | CAUGAGCAUGCAGAGGUGAGUA | 421 | TSBTx2615 | ATTCCATGAGCATGCAGAGGTGAGTA | 438 | |
| sgRNA_375 | GGCUAUCGUCACCAAUCCCA | 422 | | GGCTATCGTCACCAATCCCA | 439 | 5A |
| sgRNA_376 | GCUAUCGUCACCAAUCCCAA | 423 | | GCTATCGTCACCAATCCCAA | 440 | 4A |
| sgRNA_377 | GGCUAUCGUCACCAAUCCCA | 424 | | GGCTATCGTCACCAATCCCA | 441 | 5A |

TABLE 2B
Exemplary Spacer and Target Site Sequences.

| | | SEQ ID | | SEQ ID |
|---|---|---|---|---|
| gRNA_Name | Spacer Sequence | NO: | Target Site Sequence | NO: |
| gRNA1747 | AAGAGAGAAUAAGUAACCCAU | 453 | AAGAGAGAATAAGTAACCCAT | 500 |
| gRNA1748 | AAGCAGCCUAGCUCAGGAGAA | 454 | AAGCAGCCTAGCTCAGGAGAA | 501 |
| gRNA1749 | AAGUCCACUCAUUCUUGGCA | 455 | AAGTCCACTCATTCTTGGCA | 502 |
| gRNA1750 | ACGAUGAGAAGCCAUCCUGCC | 456 | ACGATGAGAAGCCATCCTGCC | 503 |
| gRNA1751 | AGACAAGGUUCAUAUUUGUA | 457 | AGACAAGGTTCATATTTGTA | 504 |
| gRNA1752 | AGGCUGGGAGCAGCCAUCAC | 458 | AGGCTGGGAGCAGCCATCAC | 505 |
| gRNA1753 | AUAAGUAACCCAUACAAAUA | 459 | ATAAGTAACCCATACAAATA | 506 |
| gRNA1754 | AUACUCACUUCUCCUGAGCU | 460 | ATACTCACTTCTCCTGAGCT | 507 |
| gRNA1755 | AUUAUUGACUUAGUCAACAA | 461 | ATTATTGACTTAGTCAACAA | 508 |
| gRNA1756 | CAAAUAUGAACCUUGUCUAG | 462 | CAAATATGAACCTTGTCTAG | 509 |
| gRNA1757 | CAGAAGUCCACUCAUUCUUGG | 463 | CAGAAGTCCACTCATTCTTGG | 510 |
| gRNA1758 | CAGGCUGGGAGCAGCCAUCAC | 464 | CAGGCTGGGAGCAGCCATCAC | 511 |
| gRNA1759 | CCAUCCUGCCAAGAAUGAGU | 465 | CCATCCTGCCAAGAATGAGT | 512 |
| gRNA1760 | CCUGCUGAUUCUGAUUAUUGA | 466 | CCTGCTGATTCTGATTATTGA | 513 |
| gRNA1761 | CGAUGCUCUAAUCUCUCUAGA | 467 | CGATGCTCTAATCTCTCTAGA | 514 |
| gRNA1762 | CUAAGUCAAUAAUCAGAAUCA | 468 | CTAAGTCAATAATCAGAATCA | 515 |
| gRNA1763 | CUAGACAAGGUUCAUAUUUGU | 469 | CTAGACAAGGTTCATATTTGT | 516 |
| gRNA1764 | GAACCUUGUCUAGAGAGAUU | 470 | GAACCTTGTCTAGAGAGATT | 517 |
| gRNA1765 | GAAGUCCACUCAUUCUUGGC | 471 | GAAGTCCACTCATTCTTGGC | 518 |
| gRNA1766 | GAAUCAGCAGGUUUGCAGUC | 472 | GAATCAGCAGGTTTGCAGTC | 519 |
| gRNA1767 | GAAUGAGUGGACUUCUGUGA | 473 | GAATGAGTGGACTTCTGTGA | 520 |
| gRNA1768 | GACUGCAAACCUGCUGAUUC | 474 | GACTGCAAACCTGCTGATTC | 521 |
| gRNA1769 | GACUUAGUCAACAAAGAGAGA | 475 | GACTTAGTCAACAAAGAGAGA | 522 |
| gRNA1770 | GAUAAGCAGCCUAGCUCAGG | 476 | GATAAGCAGCCTAGCTCAGG | 523 |
| gRNA1771 | GAUGAGAAGCCAUCCUGCCA | 477 | GATGAGAAGCCATCCTGCCA | 524 |
| gRNA1772 | GCCAUCCUGCCAAGAAUGAG | 478 | GCCATCCTGCCAAGAATGAG | 525 |
| gRNA1773 | GCUUUUAUACUCACUUCUCC | 479 | GCTTTTATACTCACTTCTCC | 526 |
| gRNA1774 | GGAUAAGCAGCCUAGCUCAGG | 480 | GGATAAGCAGCCTAGCTCAGG | 527 |
| gRNA1775 | GUCUAGAGAGAUUAGAGCAU | 481 | GTCTAGAGAGATTAGAGCAT | 528 |
| gRNA1776 | GUGAUGGCUGCUCCCAGCCU | 482 | GTGATGGCTGCTCCCAGCCT | 529 |
| gRNA1777 | UACUUAUUCUCUCUUUGUUGA | 483 | TACTTATTCTCTCTTTGTTGA | 530 |
| gRNA1778 | UAUUCUCUCUUUGUUGACUAA | 484 | TATTCTCTCTTTGTTGACTAA | 531 |
| gRNA1779 | UAUUGACUUAGUCAACAAAG | 485 | TATTGACTTAGTCAACAAAG | 532 |
| gRNA1780 | UAUUGACUUAGUCAACAAAGA | 486 | TATTGACTTAGTCAACAAAGA | 533 |
| gRNA1781 | UCAGAAUCAGCAGGUUUGCAG | 487 | TCAGAATCAGCAGGTTTGCAG | 534 |
| gRNA1782 | UCCACUCAUUCUUGGCAGGA | 488 | TCCACTCATTCTTGGCAGGA | 535 |
| gRNA1783 | UCUCUCUUUGUUGACUAAGUC | 489 | TCTCTCTTTGTTGACTAAGTC | 536 |
| gRNA1784 | UGAGAAGCCAUCCUGCCAAGA | 490 | TGAGAAGCCATCCTGCCAAGA | 537 |
| gRNA1785 | UGAGCUAGGCUGCUUAUCCCU | 491 | TGAGCTAGGCTGCTTATCCCT | 538 |
| gRNA1786 | UGAGUAUAAAAGCCCCAGGC | 492 | TGAGTATAAAAGCCCCAGGC | 539 |
| gRNA1787 | UGAUGGCUGCUCCCAGCCUG | 493 | TGATGGCTGCTCCCAGCCTG | 540 |
| gRNA1788 | UGCCAAGAAUGAGUGGACUUC | 494 | TGCCAAGAATGAGTGGACTTC | 541 |
| gRNA1789 | UGCCAAUCUGACUGCAAACCU | 495 | TGCCAATCTGACTGCAAACCT | 542 |
| gRNA1790 | UGUUGACUAAGUCAAUAAUC | 496 | TGTTGACTAAGTCAATAATC | 543 |
| gRNA1791 | UUGACUUAGUCAACAAAGAG | 497 | TTGACTTAGTCAACAAAGAG | 544 |
| gRNA1792 | UUUGUUGACUAAGUCAAUAAU | 498 | TTTGTTGACTAAGTCAATAAT | 545 |
| gRNA1746 | AACCUGCUGAUUCUGAUUAU | 499 | AACCTGCTGATTCTGATTAT | 546 |
| gRNA1594 | CAACUUACCCAGAGGCAAAU | 551 | CAACTTACCCAGAGGCAAAT | 566 |
| gRNA1595 | AAUGGCUCCCAGGUGUCAUC | 552 | AATGGCTCCCAGGTGTCATC | 567 |
| gRNA1596 | GGCUCCCAGGUGUCAUCAGC | 553 | GGCTCCCAGGTGTCATCAGC | 568 |
| gRNA1597 | CUCUCAUAGGUGGUAUUCAC | 554 | CTCTCATAGGTGGTATTCAC | 569 |
| gRNA1598 | UAUAGGAAAACCAGUGAGUC | 555 | TATAGGAAAACCAGTGAGTC | 570 |
| gRNA1599 | UACUCACCUCUGCAUGCUCA | 556 | TACTCACCTCTGCATGCTCA | 571 |
| gRNA1600 | GCAACUUACCCAGAGGCAAA | 557 | GCAACTTACCCAGAGGCAAA | 572 |
| gRNA1601 | UCUGUAUACUCACCUCUGCA | 558 | TCTGTATACTCACCTCTGCA | 573 |
| gRNA1602 | GAAACACUCACCGUAGGGCC | 559 | GAAACACTCACCGTAGGGCC | 574 |
| gRNA1603 | CUCUACACCCAGGGCACCGG | 560 | CTCTACACCCAGGGCACCGG | 575 |
| gRNA1604 | ACACCUUAUAGGAAAACCAG | 561 | ACACCTTATAGGAAAACCAG | 576 |
| gRNA1605 | AUAGGAAAACCAGUGAGUCU | 562 | ATAGGAAAACCAGTGAGTCT | 577 |
| gRNA1606 | ACUCACCUCUGCAUGCUCAU | 563 | ACTCACCTCTGCATGCTCAT | 578 |
| gRNA1607 | CUCACCGUAGGGCCAGCCUC | 564 | CTCACCGTAGGGCCAGCCTC | 579 |
| gRNA-#1 | AAAAGCCCCAGGCUGGGAGC | 611 | AAAAGCCCCAGGCTGGGAGC | 682 |
| gRNA-#2 | AAGUGAGUAUAAAAGCCCCA | 612 | AAGTGAGTATAAAAGCCCCA | 683 |
| gRNA-#3 | AAUAAUCAGAAUCAGCAGGUU | 613 | AATAATCAGAATCAGCAGGTT | 684 |
| gRNA-#4 | AAUAUGAACCUUGUCUAGAG | 614 | AATATGAACCTTGTCTAGAG | 685 |
| gRNA-#5 | AAUGAGUGGACUUCUGUGAU | 615 | AATGAGTGGACTTCTGTGAT | 686 |
| gRNA-#6 | ACAAAUAUGAACCUUGUCUAG | 616 | ACAAATATGAACCTTGTCTAG | 687 |
| gRNA-#7 | ACAGAAGUCCACUCAUUCUU | 617 | ACAGAAGTCCACTCATTCTT | 688 |
| gRNA-#8 | ACCUUGUCUAGAGAGAUUAG | 618 | ACCTTGTCTAGAGAGATTAG | 689 |
| gRNA-#9 | AGAAGCCAUCCUGCCAAGAA | 619 | AGAAGCCATCCTGCCAAGAA | 690 |
| gRNA-#10 | AGCAGGUUUGCAGUCAGAUU | 620 | AGCAGGTTTGCAGTCAGATT | 691 |
| gRNA-#11 | AGGGAUAAGCAGCCUAGCUC | 621 | AGGGATAAGCAGCCTAGCTC | 692 |
| gRNA-#12 | AGGUUUGCAGUCAGAUUGGC | 622 | AGGTTTGCAGTCAGATTGGC | 693 |
| gRNA-#13 | AGUAUAAAAGCCCCAGGCUG | 623 | AGTATAAAAGCCCCAGGCTG | 694 |
| gRNA-#14 | AGUCAAUAAUCAGAAUCAGC | 624 | AGTCAATAATCAGAATCAGC | 695 |
| gRNA-#15 | AUAAUCAGAAUCAGCAGGUU | 625 | ATAATCAGAATCAGCAGGTT | 696 |
| gRNA-#16 | UUGACUUAGUCAACAAAGAGAG | 626 | TTGACTTAGTCAACAAAGAGAG | 697 |
| gRNA-#17 | UCUCUUUGUUGACUAAGUCAAU | 627 | TCTCTTTGTTGACTAAGTCAAT | 698 |
| gRNA-#18 | UGAUUAUUGACUUAGUCAACAA | 628 | TGATTATTGACTTAGTCAACAA | 699 |
| gRNA-#19 | UUGGCAGGAUGGCUUCUCAUCG | 629 | TTGGCAGGATGGCTTCTCATCG | 700 |
| (sgRNA_367) | | | | |
| gRNA-#20 | ACUUAGUCAACAAAGAGAGAAU | 630 | ACTTAGTCAACAAAGAGAGAAT | 701 |
| gRNA-#21 | GCAGGGAUAAGCAGCCUAGCUC | 631 | GCAGGGATAAGCAGCCTAGCTC | 702 |
| gRNA-#22 | GUAUGGGUUACUUAUUCUCUCU | 632 | GTATGGGTTACTTATTCTCTCT | 703 |
| gRNA-#23 | CAAGAAUGAGUGGACUUCUG | 633 | CAAGAATGAGTGGACTTCTG | 704 |
| gRNA-#24 | CAAUCUGACUGCAAACCUGC | 634 | CAATCTGACTGCAAACCTGC | 705 |
| gRNA-#25 | CACAGAAGUCCACUCAUUCU | 635 | CACAGAAGTCCACTCATTCT | 706 |
| gRNA-#26 | CAGACGAUGAGAAGCCAUCC | 636 | CAGACGATGAGAAGCCATCC | 707 |
| gRNA-#27 | CAGCAGGUUUGCAGUCAGAU | 637 | CAGCAGGTTTGCAGTCAGAT | 708 |
| gRNA-#28 | CAGGAUGGCUUCUCAUCGUC | 638 | CAGGATGGCTTCTCATCGTC | 709 |
| gRNA-#29 | CAGGUUUGCAGUCAGAUUGGC | 639 | CAGGTTTGCAGTCAGATTGGC | 710 |
| gRNA-#30 | CAGUCAGAUUGGCAGGGAUA | 640 | CAGTCAGATTGGCAGGGATA | 711 |
| gRNA-#31 | CCACUCAUUCUUGGCAGGAU | 641 | CCACTCATTCTTGGCAGGAT | 712 |
| gRNA-#32 | CUAAGUCAAUAAUCAGAAUC | 642 | CTAAGTCAATAATCAGAATC | 713 |
| gRNA-#33 | GUCAACAAAGAGAGAAUAAGUA | 643 | GTCAACAAAGAGAGAATAAGTA | 714 |
| gRNA-#34 | CUUAUCCCUGCCAAUCUGAC | 644 | CTTATCCCTGCCAATCTGAC | 715 |
| gRNA-#35 | UCCCUGCCAAUCUGACUGCAAA | 645 | TCCCTGCCAATCTGACTGCAAA | 716 |
| gRNA-#36 | UUCUCUCUUUGUUGACUAAGUC | 646 | TTCTCTCTTTGTTGACTAAGTC | 717 |
| gRNA-#37 | UCCUGAGCUAGGCUGCUUAUCC | 647 | TCCTGAGCTAGGCTGCTTATCC | 718 |
| gRNA-#38 | CUUCUGUGAUGGCUGCUCCC | 648 | CTTCTGTGATGGCTGCTCCC | 719 |
| gRNA-#39 | UGUGAUGGCUGCUCCCAGCCUG | 649 | TGTGATGGCTGCTCCCAGCCTG | 720 |
| gRNA-#40 | GCAGGAUGGCUUCUCAUCGUCU | 650 | GCAGGATGGCTTCTCATCGTCT | 721 |
| gRNA-#41 | UCUAGAGAGAUUAGAGCAUCGG | 651 | TCTAGAGAGATTAGAGCATCGG | 722 |
| gRNA-#42 | GUUGACUAAGUCAAUAAUCAGA | 652 | GTTGACTAAGTCAATAATCAGA | 723 |
| gRNA-#43 | UAUACUCACUUCUCCUGAGCUA | 653 | TATACTCACTTCTCCTGAGCTA | 724 |
| gRNA-#44 | GAAGUGAGUAUAAAAGCCCC | 654 | GAAGTGAGTATAAAAGCCCC | 725 |
| gRNA-#45 | GACAAGGUUCAUAUUUGUAU | 655 | GACAAGGTTCATATTTGTAT | 726 |
| gRNA-#46 | GAGUAUAAAAGCCCCAGGCU | 656 | GAGTATAAAAGCCCCAGGCT | 727 |
| gRNA-#47 | GAGUGGACUUCUGUGAUGGC | 657 | GAGTGGACTTCTGTGATGGC | 728 |
| gRNA-#48 | GAUGGCUGCUCCCAGCCUGG | 658 | GATGGCTGCTCCCAGCCTGG | 729 |
| gRNA-#49 | GCAGCCUAGCUCAGGAGAAG | 659 | GCAGCCTAGCTCAGGAGAAG | 730 |
| gRNA-#50 | GCUGCUUAUCCCUGCCAAUC | 660 | GCTGCTTATCCCTGCCAATC | 731 |
| gRNA-#51 | GGGAUAAGCAGCCUAGCUCA | 661 | GGGATAAGCAGCCTAGCTCA | 732 |
| gRNA-#52 | GGUUUGCAGUCAGAUUGGCA | 662 | GGTTTGCAGTCAGATTGGCA | 733 |
| gRNA-#53 | GUUACUUAUUCUCUCUUUGU | 663 | GTTACTTATTCTCTCTTTGT | 734 |
| gRNA-#54 | CUUAUUCUCUCUUUGUUGACUA | 664 | CTTATTCTCTCTTTGTTGACTA | 735 |
| gRNA-#55 | AUAUUUGUAUGGGUUACUUAUU | 665 | ATATTTGTATGGGTTACTTATT | 736 |
| gRNA-#56 | ACUAAGUCAAUAAUCAGAAUCA | 666 | ACTAAGTCAATAATCAGAATCA | 737 |
| gRNA-#57 | GUUUGCAGUCAGAUUGGCAG | 667 | GTTTGCAGTCAGATTGGCAG | 738 |
| gRNA-#58 | GCAGUCAGAUUGGCAGGGAUAA | 668 | GCAGTCAGATTGGCAGGGATAA | 739 |
| gRNA-#59 | UACAAAUAUGAACCUUGUCU | 669 | TACAAATATGAACCTTGTCT | 740 |
| gRNA-#60 | UACUCACUUCUCCUGAGCUA | 670 | TACTCACTTCTCCTGAGCTA | 741 |
| gRNA-#61 | UAUAAAAGCCCCAGGCUGGG | 671 | TATAAAAGCCCCAGGCTGGG | 742 |
| gRNA-#62 | UCACUUCUCCUGAGCUAGGC | 672 | TCACTTCTCCTGAGCTAGGC | 743 |
| gRNA-#63 | UCAGAUUGGCAGGGAUAAGC | 673 | TCAGATTGGCAGGGATAAGC | 744 |
| gRNA-#64 | UCAGGAGAAGUGAGUAUAAA | 674 | TCAGGAGAAGTGAGTATAAA | 745 |
| gRNA-#65 | UCUGACUGCAAACCUGCUGAU | 675 | TCTGACTGCAAACCTGCTGAT | 746 |
| gRNA-#66 | UGAGCUAGGCUGCUUAUCCC | 676 | TGAGCTAGGCTGCTTATCCC | 747 |
| gRNA-#67 | UGCCAAUCUGACUGCAAACC | 677 | TGCCAATCTGACTGCAAACC | 748 |
| gRNA-#68 | UGCUCUAAUCUCUCUAGACA | 678 | TGCTCTAATCTCTCTAGACA | 749 |
| gRNA-#69 | UGUGAUGGCUGCUCCCAGCC | 679 | TGTGATGGCTGCTCCCAGCC | 750 |
| gRNA-#70 | UUGGCAGGGAUAAGCAGCCU | 680 | TTGGCAGGGATAAGCAGCCT | 751 |
| gRNA-#71 | UUUUAUACUCACUUCUCCUG | 681 | TTTTATACTCACTTCTCCTG | 752 |

TABLE 2C
Exemplary human TTR target site sequences and base editor + guide RNA combinations.

| gRNA | | Cas | Editor | Editing | | SEQ ID |
|---|---|---|---|---|---|---|
| Name | Editor Name | Name | Alias | Strategy | Target Site + PAM Sequence | NO |
| gRNA1594 | CBE_NGC_20 nt_4- | spCas9 | spCas9 NGC | Splice Site | CAACTTACCCAGAGGCAAATGGC | 583 |
| | 9_009 | | CBE | | | |
| gRNA1594 | ABE_NGC_20 nt_3- | spCas9 | spCas9 NGC | Splice Site | CAACTTACCCAGAGGCAAATGGC | 583 |
| | 9_008 | | ABE | | | |
| gRNA1594 | ABE_NGC_20 nt_3- | spCas9 | spCas9 NGC | Splice Site | CAACTTACCCAGAGGCAAATGGC | 583 |
| | 12_020 | | IBE | | | |
| gRNA1595 | CBE_NGC_20 nt_4- | spCas9 | spCas9 NGC | Stop Codon | AATGGCTCCCAGGTGTCATCAGC | 584 |
| | 9_009 | | CBE | | | |
| gRNA1596 | CBE_NGC_20 nt_4- | spCas9 | spCas9 NGC | Stop Codon | GGCTCCCAGGTGTCATCAGCAGC | 585 |
| | 9_009 | | CBE | | | |
| gRNA1597 | ABE_NGC_20 nt_3- | spCas9 | spCas9 NGC | Splice Site | CTCTCATAGGTGGTATTCACAGC | 586 |
| | 9_008 | | ABE | | | |
| gRNA1597 | ABE_NGC_20 nt_3- | spCas9 | spCas9 NGC | Splice Site | CTCTCATAGGTGGTATTCACAGC | 586 |
| | 12_020 | | IBE | | | |
| gRNA1598 | ABE_NGG_20 nt_3- | spCas9 | spCas9 IBE | Splice Site | TATAGGAAAACCAGTGAGTCTGG | 587 |
| | 12_018 | | | | | |
| gRNA1599 | ABE_NGG_20 nt_3- | spCas9 | spCas9 IBE | Splice Site | TACTCACCTCTGCATGCTCATGG | 588 |
| | 12_018 | | | | | |
| gRNA1600 | ABE_NGG_20 nt_3- | spCas9 | spCas9 IBE | Splice Site | GCAACTTACCCAGAGGCAAATGG | 589 |
| | 12_018 | | | | | |
| gRNA1601 | ABE_NGC_20 nt_3- | spCas9 | spCas9 NGC | Splice Site | TCTGTATACTCACCTCTGCATGC | 590 |
| | 12_020 | | IBE | | | |
| gRNA1602 | ABE_NGC_20 nt_3- | spCas9 | spCas9 NGC | Splice Site | GAAACACTCACCGTAGGGCCAGC | 591 |
| | 12_020 | | IBE | | | |
| gRNA1603 | ABE_NGA_20 nt_3- | spCas9 | spCas9 VRQR | Splice Site | CTCTACACCCAGGGCACCGGTGA | 592 |
| | 12_019 | | IBE | | | |
| gRNA1604 | ABE_NGA_20 nt_3- | spCas9 | spCas9 VRQR | Splice Site | ACACCTTATAGGAAAACCAGTGA | 593 |
| | 12_019 | | IBE | | | |
| gRNA1605 | ABE_NGA_20 nt_3- | spCas9 | spCas9 VRQR | Splice Site | ATAGGAAAACCAGTGAGTCTGGA | 594 |
| | 12_019 | | IBE | | | |
| gRNA1606 | ABE_NGA_20 nt_3- | spCas9 | spCas9 VRQR | Splice Site | ACTCACCTCTGCATGCTCATGGA | 595 |
| | 12_019 | | IBE | | | |
| gRNA1607 | ABE_NGA_20 nt_3- | spCas9 | spCas9 VRQR | Splice Site | CTCACCGTAGGGCCAGCCTCAGA | 596 |
| | 12_019 | | IBE | | | |
| gRNA1746 | ABE_NGA_20 nt_3- | TTR | spCas9 | spCas9 | AACCTGCTGATTCTGATTATTGA | 871 |
| | 9_005 | Promoter | | VRQR | | |
| | | | | ABE | | |
| gRNA1746 | CBE_NGA_20 nt_4- | TTR | spCas9 | spCas9 | AACCTGCTGATTCTGATTATTGA | 872 |
| | 9_006 | Promoter | | VRQR | | |
| | | | | CBE | | |
| gRNA1746 | ABE_NGA_20 nt_3- | TTR | spCas9 | spCas9 | AACCTGCTGATTCTGATTATTGA | 873 |
| | 12_019 | Promoter | | VRQR IBE | | |
| gRNA1747 | ABE_NNRRT_21 nt_5- | TTR | saCas9 | saCas9 | AAGAGAGAATAAGTAACCCATACAAA | 874 |
| | 14_014 | Promoter | | KKH ABE | T | |
| gRNA1747 | CBE_NNRRT_21 nt_3- | TTR | saCas9 | saCas9 | AAGAGAGAATAAGTAACCCATACAAA | 875 |
| | 12_015 | Promoter | | KKH CBE | T | |
| gRNA1748 | ABE_NNGRRT_21 nt_5- | TTR | saCas9 | saCas9 | AAGCAGCCTAGCTCAGGAGAAGTGAG | 876 |
| | 14_011 | Promoter | | ABE | T | |
| gRNA1748 | CBE_NNGRRT_21 nt_3- | TTR | saCas9 | saCas9 | AAGCAGCCTAGCTCAGGAGAAGTGAG | 877 |
| | 12_012 | Promoter | | CBE | T | |
| gRNA1749 | ABE_NGA_20 nt_3- | TTR | spCas9 | spCas9 | AAGTCCACTCATTCTTGGCAGGA | 878 |
| | 9_005 | Promoter | | VRQR | | |
| | | | | ABE | | |
| gRNA1749 | CBE_NGA_20 nt_4- | TTR | spCas9 | spCas9 | AAGTCCACTCATTCTTGGCAGGA | 879 |
| | 9_006 | Promoter | | VRQR | | |
| | | | | CBE | | |
| gRNA1749 | ABE_NGA_20 nt_3- | TTR | spCas9 | spCas9 | AAGTCCACTCATTCTTGGCAGGA | 880 |
| | 12_019 | Promoter | | VRQR IBE | | |
| gRNA1750 | ABE_NNGRRT_21 nt_5- | TTR | saCas9 | saCas9 | ACGATGAGAAGCCATCCTGCCAAGAA | 881 |
| | 14_011 | Promoter | | ABE | T | |
| gRNA1750 | CBE_NNGRRT_21 nt_3- | TTR | saCas9 | saCas9 | ACGATGAGAAGCCATCCTGCCAAGAA | 882 |
| | 12_012 | Promoter | | CBE | T | |
| gRNA1751 | ABE_NGG_20 nt_3- | TTR | spCas9 | spCas9 | AGACAAGGTTCATATTTGTATGG | 883 |
| | 9_002 | Promoter | | ABE | | |
| gRNA1751 | CBE_NGG_20 nt_4- | TTR | spCas9 | spCas9 | AGACAAGGTTCATATTTGTATGG | 884 |
| | 9_003 | Promoter | | CBE | | |
| gRNA1751 | ABE_NGG_20 nt_3- | TTR | spCas9 | spCas9 IBE | AGACAAGGTTCATATTTGTATGG | 885 |
| | 12_018 | Promoter | | | | |
| gRNA1752 | ABE_NGA_20 nt_3- | TTR | spCas9 | spCas9 | AGGCTGGGAGCAGCCATCACAGA | 886 |
| | 9_005 | Promoter | | VRQR | | |
| | | | | ABE | | |
| gRNA1752 | CBE_NGA_20 nt_4- | TTR | spCas9 | spCas9 | AGGCTGGGAGCAGCCATCACAGA | 887 |
| | 9_006 | Promoter | | VRQR | | |
| | | | | CBE | | |
| gRNA1752 | ABE_NGA_20 nt_3- | TTR | spCas9 | spCas9 | AGGCTGGGAGCAGCCATCACAGA | 888 |
| | 12_019 | Promoter | | VRQR IBE | | |
| gRNA1753 | ABE_NGA_20 nt_3- | TTR | spCas9 | spCas9 | ATAAGTAACCCATACAAATATGA | 889 |
| | 9_005 | Promoter | | VRQR | | |
| | | | | ABE | | |
| gRNA1753 | CBE_NGA_20 nt_4- | TTR | spCas9 | spCas9 | ATAAGTAACCCATACAAATATGA | 890 |
| | 9_006 | Promoter | | VRQR | | |
| | | | | CBE | | |
| gRNA1753 | ABE_NGA_20 nt_3- | TTR | spCas9 | spCas9 | ATAAGTAACCCATACAAATATGA | 891 |
| | 12_019 | Promoter | | VRQR IBE | | |
| gRNA1754 | ABE_NGG_20 nt_3- | TTR | spCas9 | spCas9 | ATACTCACTTCTCCTGAGCTAGG | 892 |
| | 9_002 | Promoter | | ABE | | |
| gRNA1754 | CBE_NGG_20 nt_4- | TTR | spCas9 | spCas9 | ATACTCACTTCTCCTGAGCTAGG | 893 |
| | 9_003 | Promoter | | CBE | | |
| gRNA1754 | ABE_NGG_20 nt_3- | TTR | spCas9 | spCas9 IBE | ATACTCACTTCTCCTGAGCTAGG | 894 |
| | 12_018 | Promoter | | | | |
| gRNA1755 | ABE_NGA_20 nt_3- | TTR | spCas9 | spCas9 | ATTATTGACTTAGTCAACAAAGA | 895 |
| | 9_005 | Promoter | | VRQR | | |
| | | | | ABE | | |
| gRNA1755 | CBE_NGA_20 nt_4- | TTR | spCas9 | spCas9 | ATTATTGACTTAGTCAACAAAGA | 896 |
| | 9_006 | Promoter | | VRQR | | |
| | | | | CBE | | |
| gRNA1755 | ABE_NGA_20 nt_3- | TTR | spCas9 | spCas9 | ATTATTGACTTAGTCAACAAAGA | 897 |
| | 12_019 | Promoter | | VRQR IBE | | |
| gRNA1756 | ABE_NGA_20 nt_3- | TTR | spCas9 | spCas9 | CAAATATGAACCTTGTCTAGAGA | 898 |
| | 9_005 | Promoter | | VRQR | | |
| | | | | ABE | | |
| gRNA1756 | CBE_NGA_20 nt_4- | TTR | spCas9 | spCas9 | CAAATATGAACCTTGTCTAGAGA | 899 |
| | 9_006 | Promoter | | VRQR | | |
| | | | | CBE | | |
| gRNA1756 | ABE_NGA_20 nt_3- | TTR | spCas9 | spCas9 | CAAATATGAACCTTGTCTAGAGA | 900 |
| | 12_019 | Promoter | | VRQR IBE | | |
| gRNA1757 | ABE_NNGRRT_21 nt_5- | TTR | saCas9 | saCas9 | CAGAAGTCCACTCATTCTTGGCAGGAT | 901 |
| | 14_011 | Promoter | | ABE | | |
| gRNA1757 | CBE_NNGRRT_21 nt_3- | TTR | saCas9 | saCas9 | CAGAAGTCCACTCATTCTTGGCAGGAT | 902 |
| | 12_012 | Promoter | | CBE | | |
| gRNA1758 | ABE_NNRRT_21 nt_5- | TTR | saCas9 | saCas9 | CAGGCTGGGAGCAGCCATCACAGAAG | 903 |
| | 14_014 | Promoter | | KKH ABE | T | |
| gRNA1758 | CBE_NNRRT_21 nt_3- | TTR | saCas9 | saCas9 | CAGGCTGGGAGCAGCCATCACAGAAG | 904 |
| | 12_015 | Promoter | | KKH CBE | T | |
| gRNA1759 | ABE_NGA_20 nt_3- | TTR | spCas9 | spCas9 | CCATCCTGCCAAGAATGAGTGGA | 905 |
| | 9_005 | Promoter | | VRQR | | |
| | | | | ABE | | |
| gRNA1759 | CBE_NGA_20 nt_4- | TTR | spCas9 | spCas9 | CCATCCTGCCAAGAATGAGTGGA | 906 |
| | 9_006 | Promoter | | VRQR | | |
| | | | | CBE | | |
| gRNA1759 | ABE_NGA_20 nt_3- | TTR | spCas9 | spCas9 | CCATCCTGCCAAGAATGAGTGGA | 907 |
| | 12_019 | Promoter | | VRQR IBE | | |
| gRNA1760 | ABE_NNRRT_21 nt_5- | TTR | saCas9 | saCas9 | CCTGCTGATTCTGATTATTGACTTAGT | 908 |
| | 14_014 | Promoter | | KKH ABE | | |
| gRNA1760 | CBE_NNNRRT_21 nt_3- | TTR | saCas9 | saCas9 | CCTGCTGATTCTGATTATTGACTTAGT | 909 |
| | 12_015 | Promoter | | KKH CBE | | |
| gRNA1761 | ABE_NNNRRT_21 nt_5- | TTR | saCas9 | saCas9 | CGATGCTCTAATCTCTCTAGACAAGGT | 910 |
| | 14_014 | Promoter | | KKH ABE | | |
| gRNA1761 | CBE_NNNRRT_21 nt_3- | TTR | saCas9 | saCas9 | CGATGCTCTAATCTCTCTAGACAAGGT | 911 |
| | 12_015 | Promoter | | KKH CBE | | |
| gRNA1762 | ABE_NNNRRT_21 nt_5- | TTR | saCas9 | saCas9 | CTAAGTCAATAATCAGAATCAGCAGGT | 912 |
| | 14_014 | Promoter | | KKH ABE | | |
| gRNA1762 | CBE_NNRRT_21 nt_3- | TTR | saCas9 | saCas9 | CTAAGTCAATAATCAGAATCAGCAGGT | 913 |
| | 12_015 | Promoter | | KKH CBE | | |
| gRNA1763 | ABE_NNGRRT_21 nt_5- | TTR | saCas9 | saCas9 | CTAGACAAGGTTCATATTTGTATGGGT | 914 |
| | 14_011 | Promoter | | ABE | | |
| gRNA1763 | CBE_NNGRRT_21 nt_3- | TTR | saCas9 | saCas9 | CTAGACAAGGTTCATATTTGTATGGGT | 915 |
| | 12_012 | Promoter | | CBE | | |
| gRNA1764 | ABE_NGA_20 nt_3- | TTR | spCas9 | spCas9 | GAACCTTGTCTAGAGAGATTAGA | 916 |
| | 9_005 | Promoter | | VRQR | | |
| | | | | ABE | | |
| gRNA1764 | CBE_NGA_20 nt_4- | TTR | spCas9 | spCas9 | GAACCTTGTCTAGAGAGATTAGA | 917 |
| | 9_006 | Promoter | | VRQR | | |
| | | | | CBE | | |
| gRNA1764 | ABE_NGA_20 nt_3- | TTR | spCas9 | spCas9 | GAACCTTGTCTAGAGAGATTAGA | 918 |
| | 12_019 | Promoter | | VRQR IBE | | |
| gRNA1765 | ABE_NGG_20 nt_3- | TTR | spCas9 | spCas9 | GAAGTCCACTCATTCTTGGCAGG | 919 |
| | 9_002 | Promoter | | ABE | | |
| gRNA1765 | CBE_NGG_20 nt_4- | TTR | spCas9 | spCas9 | GAAGTCCACTCATTCTTGGCAGG | 920 |
| | 9_003 | Promoter | | CBE | | |
| gRNA1765 | ABE_NGG_20 nt_3- | TTR | spCas9 | spCas9 IBE | GAAGTCCACTCATTCTTGGCAGG | 921 |
| | 12_018 | Promoter | | | | |
| gRNA1766 | ABE_NGA_20 nt_3- | TTR | spCas9 | spCas9 | GAATCAGCAGGTTTGCAGTCAGA | 922 |
| | 9_005 | Promoter | | VRQR | | |
| | | | | ABE | | |
| gRNA1766 | CBE_NGA_20 nt_4- | TTR | spCas9 | spCas9 | GAATCAGCAGGTTTGCAGTCAGA | 923 |
| | 9_006 | Promoter | | VRQR | | |
| | | | | CBE | | |
| gRNA1766 | ABE_NGA_20 nt_3- | TTR | spCas9 | spCas9 | GAATCAGCAGGTTTGCAGTCAGA | 924 |
| | 12_019 | Promoter | | VRQR IBE | | |
| gRNA1767 | ABE_NGG_20 nt_3- | TTR | spCas9 | spCas9 | GAATGAGTGGACTTCTGTGATGG | 925 |
| | 9_002 | Promoter | | ABE | | |
| gRNA1767 | CBE_NGG_20 nt_4- | TTR | spCas9 | spCas9 | GAATGAGTGGACTTCTGTGATGG | 926 |
| | 9_003 | Promoter | | CBE | | |
| gRNA1767 | ABE_NGG_20 nt_3- | TTR | spCas9 | spCas9 IBE | GAATGAGTGGACTTCTGTGATGG | 927 |
| | 12_018 | Promoter | | | | |
| gRNA1768 | ABE_NGA_20 nt_3- | TTR | spCas9 | spCas9 | GACTGCAAACCTGCTGATTCTGA | 928 |
| | 9_005 | Promoter | | VRQR | | |
| | | | | ABE | | |
| gRNA1768 | CBE_NGA_20 nt_4- | TTR | spCas9 | spCas9 | GACTGCAAACCTGCTGATTCTGA | 929 |
| | 9_006 | Promoter | | VRQR | | |
| | | | | CBE | | |
| gRNA1768 | ABE_NGA_20 nt_3- | TTR | spCas9 | spCas9 | GACTGCAAACCTGCTGATTCTGA | 930 |
| | 12_019 | Promoter | | VRQR IBE | | |
| gRNA1769 | ABE_NNRRT_21 nt_5- | TTR | saCas9 | saCas9 | GACTTAGTCAACAAAGAGAGAATAAG | 931 |
| | 14_014 | Promoter | | KKH ABE | T | |
| gRNA1769 | CBE_NNRRT_21 nt_3- | TTR | saCas9 | saCas9 | GACTTAGTCAACAAAGAGAGAATAAG | 932 |
| | 12_015 | Promoter | | KKH CBE | T | |
| gRNA1770 | ABE_NGA_20 nt_3- | TTR | spCas9 | spCas9 | GATAAGCAGCCTAGCTCAGGAGA | 933 |
| | 9_005 | Promoter | | VRQR | | |
| | | | | ABE | | |
| gRNA1770 | CBE_NGA_20 nt_4- | TTR | spCas9 | spCas9 | GATAAGCAGCCTAGCTCAGGAGA | 934 |
| | 9_006 | Promoter | | VRQR | | |
| | | | | CBE | | |
| gRNA1770 | ABE_NGA_20 nt_3- | TTR | spCas9 | spCas9 | GATAAGCAGCCTAGCTCAGGAGA | 935 |
| | 12_019 | Promoter | | VRQR IBE | | |
| gRNA1771 | ABE_NGA_20 nt_3- | TTR | spCas9 | spCas9 | GATGAGAAGCCATCCTGCCAAGA | 936 |
| | 9_005 | Promoter | | VRQR | | |
| | | | | ABE | | |
| gRNA1771 | CBE_NGA_20 nt_4- | TTR | spCas9 | spCas9 | GATGAGAAGCCATCCTGCCAAGA | 937 |
| | 9_006 | Promoter | | VRQR | | |
| | | | | CBE | | |
| gRNA1771 | ABE_NGA_20 nt_3- | TTR | spCas9 | spCas9 | GATGAGAAGCCATCCTGCCAAGA | 938 |
| | 12_019 | Promoter | | VRQR IBE | | |
| gRNA1772 | ABE_NGG_20 nt_3- | TTR | spCas9 | spCas9 | GCCATCCTGCCAAGAATGAGTGG | 939 |
| | 9_002 | Promoter | | ABE | | |
| gRNA1772 | CBE_NGG_20 nt_4- | TTR | spCas9 | spCas9 | GCCATCCTGCCAAGAATGAGTGG | 940 |
| | 9_003 | Promoter | | CBE | | |
| gRNA1772 | ABE_NGG_20 nt_3- | TTR | spCas9 | spCas9 IBE | GCCATCCTGCCAAGAATGAGTGG | 941 |
| | 12_018 | Promoter | | | | |
| gRNA1773 | ABE_NGA_20 nt_3- | TTR | spCas9 | spCas9 | GCTTTTATACTCACTTCTCCTGA | 942 |
| | 9_005 | Promoter | | VRQR | | |
| | | | | ABE | | |
| gRNA1773 | CBE_NGA_20 nt_4- | TTR | spCas9 | spCas9 | GCTTTTATACTCACTTCTCCTGA | 943 |
| | 9_006 | Promoter | | VRQR | | |
| | | | | CBE | | |
| gRNA1773 | ABE_NGA_20 nt_3- | TTR | spCas9 | spCas9 | GCTTTTATACTCACTTCTCCTGA | 944 |
| | 12_019 | Promoter | | VRQR IBE | | |
| gRNA1774 | ABE_NNRRT_21 nt_5- | TTR | saCas9 | saCas9 | GGATAAGCAGCCTAGCTCAGGAGAAG | 945 |
| | 14_014 | Promoter | | KKH ABE | T | |
| gRNA1774 | CBE_NNRRT_21 nt_3- | TTR | saCas9 | saCas9 | GGATAAGCAGCCTAGCTCAGGAGAAG | 946 |
| | 12_015 | Promoter | | KKH CBE | T | |
| gRNA1775 | ABE_NGG_20 nt_3- | TTR | spCas9 | spCas9 | GTCTAGAGAGATTAGAGCATCGG | 947 |
| | 9_002 | Promoter | | ABE | | |
| gRNA1775 | CBE_NGG_20 nt_4- | TTR | spCas9 | spCas9 | GTCTAGAGAGATTAGAGCATCGG | 948 |
| | 9_003 | Promoter | | CBE | | |
| gRNA1775 | ABE_NGG_20 nt_3- | TTR | spCas9 | spCas9 IBE | GTCTAGAGAGATTAGAGCATCGG | 949 |
| | 12_018 | Promoter | | | | |
| gRNA1776 | ABE_NGG_20 nt_3- | TTR | spCas9 | spCas9 | GTGATGGCTGCTCCCAGCCTGGG | 950 |
| | 9_002 | Promoter | | ABE | | |
| gRNA1776 | CBE_NGG_20 nt_4- | TTR | spCas9 | spCas9 | GTGATGGCTGCTCCCAGCCTGGG | 951 |
| | 9_003 | Promoter | | CBE | | |
| gRNA1776 | ABE_NGG_20 nt_3- | TTR | spCas9 | spCas9 IBE | GTGATGGCTGCTCCCAGCCTGGG | 952 |
| | 12_018 | Promoter | | | | |
| gRNA1777 | ABE_NNRRT_21 nt_5- | TTR | saCas9 | saCas9 | TACTTATTCTCTCTTTGTTGACTAAGT | 953 |
| | 14_014 | Promoter | | KKH ABE | | |
| gRNA1777 | CBE_NNRRT_21 nt_3- | TTR | saCas9 | saCas9 | TACTTATTCTCTCTTTGTTGACTAAGT | 954 |
| | 12_015 | Promoter | | KKH CBE | | |
| gRNA1778 | ABE_NNRRT_21 nt_5- | TTR | saCas9 | saCas9 | TATTCTCTCTTTGTTGACTAAGTCAAT | 955 |
| | 14_014 | Promoter | | KKH ABE | | |
| gRNA1778 | CBE_NNRRT_21 nt_3- | TTR | saCas9 | saCas9 | TATTCTCTCTTTGTTGACTAAGTCAAT | 956 |
| | 12_015 | Promoter | | KKH CBE | | |
| gRNA1779 | ABE_NGA_20 nt_3- | TTR | spCas9 | spCas9 | TATTGACTTAGTCAACAAAGAGA | 957 |
| | 9_005 | Promoter | | VRQR | | |
| | | | | ABE | | |
| gRNA1779 | CBE_NGA_20 nt_4- | TTR | spCas9 | spCas9 | TATTGACTTAGTCAACAAAGAGA | 958 |
| | 9_006 | Promoter | | VRQR | | |
| | | | | CBE | | |
| gRNA1779 | ABE_NGA_20 nt_3- | TTR | spCas9 | spCas9 | TATTGACTTAGTCAACAAAGAGA | 959 |
| | 12_019 | Promoter | | VRQR IBE | | |
| gRNA1780 | ABE_NNGRRT_21 nt_5- | TTR | saCas9 | saCas9 | TATTGACTTAGTCAACAAAGAGAGAAT | 960 |
| | 14_011 | Promoter | | ABE | | |
| gRNA1780 | CBE_NNGRRT_21 nt_3- | TTR | saCas9 | saCas9 | TATTGACTTAGTCAACAAAGAGAGAAT | 961 |
| | 12_012 | Promoter | | CBE | | |
| gRNA1781 | ABE_NNRRT_21 nt_5- | TTR | saCas9 | saCas9 | TCAGAATCAGCAGGTTTGCAGTCAGAT | 962 |
| | 14_014 | Promoter | | KKH ABE | | |
| gRNA1781 | CBE_NNRRT_21 nt_3- | TTR | saCas9 | saCas9 | TCAGAATCAGCAGGTTTGCAGTCAGAT | 963 |
| | 12_015 | Promoter | | KKH CBE | | |
| gRNA1782 | ABE_NGG_20 nt_3- | TTR | spCas9 | spCas9 | TCCACTCATTCTTGGCAGGATGG | 964 |
| | 9_002 | Promoter | | ABE | | |
| gRNA1782 | CBE_NGG_20 nt_4- | TTR | spCas9 | spCas9 | TCCACTCATTCTTGGCAGGATGG | 965 |
| | 9_003 | Promoter | | CBE | | |
| gRNA1782 | ABE_NGG_20 nt_3- | TTR | spCas9 | spCas9 IBE | TCCACTCATTCTTGGCAGGATGG | 966 |
| | 12_018 | Promoter | | | | |
| gRNA1783 | ABE_NNRRT_21 nt_5- | TTR | saCas9 | saCas9 | TCTCTCTTTGTTGACTAAGTCAATAAT | 967 |
| | 14_014 | Promoter | | KKH ABE | | |
| gRNA1783 | CBE_NNRRT_21 nt_3- | TTR | saCas9 | saCas9 | TCTCTCTTTGTTGACTAAGTCAATAAT | 968 |
| | 12_015 | Promoter | | KKH CBE | | |
| gRNA1784 | ABE_NNGRRT_21 nt_5- | TTR | saCas9 | saCas9 | TGAGAAGCCATCCTGCCAAGAATGAGT | 969 |
| | 14_011 | Promoter | | ABE | | |
| gRNA1784 | CBE_NNGRRT_21 nt_3- | TTR | saCas9 | saCas9 | TGAGAAGCCATCCTGCCAAGAATGAGT | 970 |
| | 12_012 | Promoter | | CBE | | |
| gRNA1785 | ABE_NNRRT_21 nt_5- | TTR | saCas9 | saCas9 | TGAGCTAGGCTGCTTATCCCTGCCAAT | 971 |
| | 14_014 | Promoter | | KKH ABE | | |
| gRNA1785 | CBE_NNRRT_21 nt_3- | TTR | saCas9 | saCas9 | TGAGCTAGGCTGCTTATCCCTGCCAAT | 972 |
| | 12_015 | Promoter | | KKH CBE | | |
| gRNA1786 | ABE_NGG_20 nt_3- | TTR | spCas9 | spCas9 | TGAGTATAAAAGCCCCAGGCTGG | 973 |
| | 9_002 | Promoter | | ABE | | |
| gRNA1786 | CBE_NGG_20 nt_4- | TTR | spCas9 | spCas9 | TGAGTATAAAAGCCCCAGGCTGG | 974 |
| | 9_003 | Promoter | | CBE | | |
| gRNA1786 | ABE_NGG_20 nt_3- | TTR | spCas9 | spCas9 IBE | TGAGTATAAAAGCCCCAGGCTGG | 975 |
| | 12_018 | Promoter | | | | |
| gRNA1787 | ABE_NGG_20 nt_3- | TTR | spCas9 | spCas9 | TGATGGCTGCTCCCAGCCTGGGG | 976 |
| | 9_002 | Promoter | | ABE | | |
| gRNA1787 | CBE_NGG_20 nt_4- | TTR | spCas9 | spCas9 | TGATGGCTGCTCCCAGCCTGGGG | 977 |
| | 9_003 | Promoter | | CBE | | |
| gRNA1787 | ABE_NGG_20 nt_3- | TTR | spCas9 | spCas9 IBE | TGATGGCTGCTCCCAGCCTGGGG | 978 |
| | 12_018 | Promoter | | | | |
| gRNA1788 | ABE_NNRRT_21 nt_5- | TTR | saCas9 | saCas9 | TGCCAAGAATGAGTGGACTTCTGTGAT | 979 |
| | 14_014 | Promoter | | KKH ABE | | |
| gRNA1788 | CBE_NNRRT_21 nt_3- | TTR | saCas9 | saCas9 | TGCCAAGAATGAGTGGACTTCTGTGAT | 980 |
| | 12_015 | Promoter | | KKH CBE | | |
| gRNA1789 | ABE_NNRRT_21 nt_5- | TTR | saCas9 | saCas9 | TGCCAATCTGACTGCAAACCTGCTGAT | 981 |
| | 14_014 | Promoter | | KKH ABE | | |
| gRNA1789 | CBE_NNNRRT_21 nt_3- | TTR | saCas9 | saCas9 | TGCCAATCTGACTGCAAACCTGCTGAT | 982 |
| | 12_015 | Promoter | | KKH CBE | | |
| gRNA1790 | ABE_NGA_20 nt_3- | TTR | spCas9 | spCas9 | TGTTGACTAAGTCAATAATCAGA | 983 |
| | 9_005 | Promoter | | VRQR | | |
| | | | | ABE | | |
| gRNA1790 | CBE_NGA_20 nt_4- | TTR | spCas9 | spCas9 | TGTTGACTAAGTCAATAATCAGA | 984 |
| | 9_006 | Promoter | | VRQR | | |
| | | | | CBE | | |
| gRNA1790 | ABE_NGA_20 nt_3- | TTR | spCas9 | spCas9 | TGTTGACTAAGTCAATAATCAGA | 985 |
| | 12_019 | Promoter | | VRQR IBE | | |
| gRNA1791 | ABE_NGA_20 nt_3- | TTR | spCas9 | spCas9 | TTGACTTAGTCAACAAAGAGAGA | 986 |
| | 9_005 | Promoter | | VRQR | | |
| | | | | ABE | | |
| gRNA1791 | CBE_NGA_20 nt_4- | TTR | spCas9 | spCas9 | TTGACTTAGTCAACAAAGAGAGA | 987 |
| | 9_006 | Promoter | | VRQR | | |
| | | | | CBE | | |
| gRNA1791 | ABE_NGA_20 nt_3- | TTR | spCas9 | spCas9 | TTGACTTAGTCAACAAAGAGAGA | 988 |
| | 12_019 | Promoter | | VRQR IBE | | |
| gRNA1792 | ABE_NNGRRT_21 nt_5- | TTR | saCas9 | saCas9 | TTTGTTGACTAAGTCAATAATCAGAAT | 989 |
| | 14_011 | Promoter | | ABE | | |
| gRNA1792 | CBE_NNGRRT_21 nt_3- | TTR | saCas9 | saCas9 | TTTGTTGACTAAGTCAATAATCAGAAT | 990 |
| | 12_012 | Promoter | | CBE | | |
| gRNA-#1 | ABE_NGC_20 nt_3- | TTR | spCas9 | spCas9 | AAAAGCCCCAGGCTGGGAGCAGC | 991 |
| | 9_008 | Promoter | | NGC ABE | | |
| gRNA-#1 | CBE_NGC_20 nt_4- | TTR | spCas9 | spCas9 | AAAAGCCCCAGGCTGGGAGCAGC | 992 |
| | 9_009 | Promoter | | NGC CBE | | |
| gRNA-#1 | ABE_NGC_20 nt_3- | TTR | spCas9 | spCas9 | AAAAGCCCCAGGCTGGGAGCAGC | 993 |
| | 12_020 | Promoter | | NGC IBE | | |
| gRNA-#2 | ABE_NGC_20 nt_3- | TTR | spCas9 | spCas9 | AAGTGAGTATAAAAGCCCCAGGC | 994 |
| | 9_008 | Promoter | | NGC ABE | | |
| gRNA-#2 | CBE_NGC_20 nt_4- | TTR | spCas9 | spCas9 | AAGTGAGTATAAAAGCCCCAGGC | 995 |
| | 9_009 | Promoter | | NGC CBE | | |
| gRNA-#2 | ABE_NGC_20 nt_3- | TTR | spCas9 | spCas9 | AAGTGAGTATAAAAGCCCCAGGC | 996 |
| | 12_020 | Promoter | | NGC IBE | | |
| gRNA-#3 | ABE_NNNRRT_21 nt_5- | TTR | saCas9 | saCas9 | AATAATCAGAATCAGCAGGTTTGCAGT | 997 |
| | 14_014 | Promoter | | KKH ABE | | |
| gRNA-#3 | CBE_NNRRT_21 nt_3- | TTR | saCas9 | saCas9 | AATAATCAGAATCAGCAGGTTTGCAGT | 998 |
| | 12_015 | Promoter | | KKH CBE | | |
| gRNA-#4 | CBE_NGA_20 nt_4- | TTR | spCas9 | spCas9 | AATATGAACCTTGTCTAGAGAGA | 999 |
| | 9_006 | Promoter | | VRQR | | |
| | | | | CBE | | |
| gRNA-#4 | ABE_NGA_20 nt_3- | TTR | spCas9 | spCas9 | AATATGAACCTTGTCTAGAGAGA | 1000 |
| | 9_005 | Promoter | | VRQR | | |
| | | | | ABE | | |
| gRNA-#4 | ABE_NGA_20 nt_3- | TTR | spCas9 | spCas9 | AATATGAACCTTGTCTAGAGAGA | 1001 |
| | 12_019 | Promoter | | VRQR IBE | | |
| gRNA-#5 | ABE_NGC_20 nt_3- | TTR | spCas9 | spCas9 | AATGAGTGGACTTCTGTGATGGC | 1002 |
| | 9_008 | Promoter | | NGC ABE | | |
| gRNA-#5 | CBE_NGC_20 nt_4- | TTR | spCas9 | spCas9 | AATGAGTGGACTTCTGTGATGGC | 1003 |
| | 9_009 | Promoter | | NGC CBE | | |
| gRNA-#5 | ABE_NGC_20 nt_3- | TTR | spCas9 | spCas9 | AATGAGTGGACTTCTGTGATGGC | 1004 |
| | 12_020 | Promoter | | NGC IBE | | |
| gRNA-#6 | ABE_NNRRT_21 nt_5- | TTR | saCas9 | saCas9 | ACAAATATGAACCTTGTCTAGAGAGAT | 1005 |
| | 14_014 | Promoter | | KKH ABE | | |
| gRNA-#6 | CBE_NNRRT_21 nt_3- | TTR | saCas9 | saCas9 | ACAAATATGAACCTTGTCTAGAGAGAT | 1006 |
| | 12_015 | Promoter | | KKH CBE | | |
| gRNA-#7 | ABE_NGC_20 nt_3- | TTR | spCas9 | spCas9 | ACAGAAGTCCACTCATTCTTGGC | 1007 |
| | 9_008 | Promoter | | NGC ABE | | |
| gRNA-#7 | CBE_NGC_20 nt_4- | TTR | spCas9 | spCas9 | ACAGAAGTCCACTCATTCTTGGC | 1008 |
| | 9_009 | Promoter | | NGC CBE | | |
| gRNA-#7 | ABE_NGC_20 nt_3- | TTR | spCas9 | spCas9 | ACAGAAGTCCACTCATTCTTGGC | 1009 |
| | 12_020 | Promoter | | NGC IBE | | |
| gRNA-#8 | ABE_NGC_20 nt_3- | TTR | spCas9 | spCas9 | ACCTTGTCTAGAGAGATTAGAGC | 1010 |
| | 9_008 | Promoter | | NGC ABE | | |
| gRNA-#8 | CBE_NGC_20 nt_4- | TTR | spCas9 | spCas9 | ACCTTGTCTAGAGAGATTAGAGC | 1011 |
| | 9_009 | Promoter | | NGC CBE | | |
| gRNA-#8 | ABE_NGC_20 nt_3- | TTR | spCas9 | spCas9 | ACCTTGTCTAGAGAGATTAGAGC | 1012 |
| | 12_020 | Promoter | | NGC IBE | | |
| gRNA-#9 | CBE_NGA_20 nt_4- | TTR | spCas9 | spCas9 | AGAAGCCATCCTGCCAAGAATGA | 1013 |
| | 9_006 | Promoter | | VRQR | | |
| | | | | CBE | | |
| gRNA-#9 | ABE_NGA_20 nt_3- | TTR | spCas9 | spCas9 | AGAAGCCATCCTGCCAAGAATGA | 1014 |
| | 9_005 | Promoter | | VRQR | | |
| | | | | ABE | | |
| gRNA-#9 | ABE_NGA_20 nt_3- | TTR | spCas9 | spCas9 | AGAAGCCATCCTGCCAAGAATGA | 1015 |
| | 12_019 | Promoter | | VRQR IBE | | |
| gRNA-#10 | ABE_NGC_20 nt_3- | TTR | spCas9 | spCas9 | AGCAGGTTTGCAGTCAGATTGGC | 1016 |
| | 9_008 | Promoter | | NGC ABE | | |
| gRNA-#10 | CBE_NGC_20 nt_4- | TTR | spCas9 | spCas9 | AGCAGGTTTGCAGTCAGATTGGC | 1017 |
| | 9_009 | Promoter | | NGC CBE | | |
| gRNA-#10 | ABE_NGC_20 nt_3- | TTR | spCas9 | spCas9 | AGCAGGTTTGCAGTCAGATTGGC | 1018 |
| | 12_020 | Promoter | | NGC IBE | | |
| gRNA-#11 | ABE_NGG_20 nt_3- | TTR | spCas9 | spCas9 | AGGGATAAGCAGCCTAGCTCAGG | 1019 |
| | 9_002 | Promoter | | ABE | | |
| gRNA-#11 | CBE_NGG_20 nt_4- | TTR | spCas9 | spCas9 | AGGGATAAGCAGCCTAGCTCAGG | 1020 |
| | 9_003 | Promoter | | CBE | | |
| gRNA-#11 | ABE_NGG_20 nt_3- | TTR | spCas9 | spCas9 IBE | AGGGATAAGCAGCCTAGCTCAGG | 1021 |
| | 12_018 | Promoter | | | | |
| gRNA-#12 | ABE_NGG_20 nt_3- | TTR | spCas9 | spCas9 | AGGTTTGCAGTCAGATTGGCAGG | 1022 |
| | 9_002 | Promoter | | ABE | | |
| gRNA-#12 | CBE_NGG_20 nt_4- | TTR | spCas9 | spCas9 | AGGTTTGCAGTCAGATTGGCAGG | 1023 |
| | 9_003 | Promoter | | CBE | | |
| gRNA-#12 | ABE_NGG_20 nt_3- | TTR | spCas9 | spCas9 IBE | AGGTTTGCAGTCAGATTGGCAGG | 1024 |
| | 12_018 | Promoter | | | | |
| gRNA-#13 | CBE_NGA_20 nt_4- | TTR | spCas9 | spCas9 | AGTATAAAAGCCCCAGGCTGGGA | 1025 |
| | 9_006 | Promoter | | VRQR | | |
| | | | | CBE | | |
| gRNA-#13 | ABE_NGA_20 nt_3- | TTR | spCas9 | spCas9 | AGTATAAAAGCCCCAGGCTGGGA | 1026 |
| | 9_005 | Promoter | | VRQR | | |
| | | | | ABE | | |
| gRNA-#13 | ABE_NGA_20 nt_3- | TTR | spCas9 | spCas9 | AGTATAAAAGCCCCAGGCTGGGA | 1027 |
| | 12_019 | Promoter | | VRQR IBE | | |
| gRNA-#14 | ABE_NGG_20 nt_3- | TTR | spCas9 | spCas9 | AGTCAATAATCAGAATCAGCAGG | 1028 |
| | 9_002 | Promoter | | ABE | | |
| gRNA-#14 | CBE_NGG_20 nt_4- | TTR | spCas9 | spCas9 | AGTCAATAATCAGAATCAGCAGG | 1029 |
| | 9_003 | Promoter | | CBE | | |
| gRNA-#14 | ABE_NGG_20 nt_3- | TTR | spCas9 | spCas9 IBE | AGTCAATAATCAGAATCAGCAGG | 1030 |
| | 12_018 | Promoter | | | | |
| gRNA-#15 | ABE_NGC_20 nt_3- | TTR | spCas9 | spCas9 | ATAATCAGAATCAGCAGGTTTGC | 1031 |
| | 9_008 | Promoter | | NGC ABE | | |
| gRNA-#15 | CBE_NGC_20 nt_4- | TTR | spCas9 | spCas9 | ATAATCAGAATCAGCAGGTTTGC | 1032 |
| | 9_009 | Promoter | | NGC CBE | | |
| gRNA-#15 | ABE_NGC_20 nt_3- | TTR | spCas9 | spCas9 | ATAATCAGAATCAGCAGGTTTGC | 1033 |
| | 12_020 | Promoter | | NGC IBE | | |
| gRNA-#16 | ABE_VTTN_22 nt_5- | TTR | cas12b | cas12b | ATTATTGACTTAGTCAACAAAGAGAG | 1034 |
| | 9_017 | Promoter | | ABE | | |
| gRNA-#17 | ABE_VTTN_22 nt_5- | TTR | cas12b | cas12b | ATTCTCTCTTTGTTGACTAAGTCAAT | 1035 |
| | 9_017 | Promoter | | ABE | | |
| gRNA-#18 | ABE_VTTN_22 nt_5- | TTR | cas12b | cas12b | ATTCTGATTATTGACTTAGTCAACAA | 1036 |
| | 9_017 | Promoter | | ABE | | |
| gRNA-#19 | ABE_VTTN_22 nt_5- | TTR | cas12b | cas12b | ATTCTTGGCAGGATGGCTTCTCATCG | 1037 |
| (sgRNA_ | 9_017 | Promoter | | ABE | | |
| 367) | | | | | | |
| gRNA-#20 | ABE_VTTN_22 nt_5- | TTR | cas12b | cas12b | ATTGACTTAGTCAACAAAGAGAGAAT | 1038 |
| | 9_017 | Promoter | | ABE | | |
| gRNA-#21 | ABE_VTTN_22 nt_5- | TTR | cas12b | cas12b | ATTGGCAGGGATAAGCAGCCTAGCTC | 1039 |
| | 9_017 | Promoter | | ABE | | |
| gRNA-#22 | ABE_VTTN_22 nt_5- | TTR | cas12b | cas12b | ATTTGTATGGGTTACTTATTCTCTCT | 1040 |
| | 9_017 | Promoter | | ABE | | |
| gRNA-#23 | CBE_NGA_20 nt_4- | TTR | spCas9 | spCas9 | CAAGAATGAGTGGACTTCTGTGA | 1041 |
| | 9_006 | Promoter | | VRQR | | |
| | | | | CBE | | |
| gRNA-#23 | ABE_NGA_20 nt_3- | TTR | spCas9 | spCas9 | CAAGAATGAGTGGACTTCTGTGA | 1042 |
| | 9_005 | Promoter | | VRQR | | |
| | | | | ABE | | |
| gRNA-#23 | ABE_NGA_20 nt_3- | TTR | spCas9 | spCas9 | CAAGAATGAGTGGACTTCTGTGA | 1043 |
| | 12_019 | Promoter | | VRQR IBE | | |
| gRNA-#24 | CBE_NGA_20 nt_4- | TTR | spCas9 | spCas9 | CAATCTGACTGCAAACCTGCTGA | 1044 |
| | 9_006 | Promoter | | VRQR | | |
| | | | | CBE | | |
| gRNA-#24 | ABE_NGA_20 nt_3- | TTR | spCas9 | spCas9 | CAATCTGACTGCAAACCTGCTGA | 1045 |
| | 9_005 | Promoter | | VRQR | | |
| | | | | ABE | | |
| gRNA-#24 | ABE_NGA_20 nt_3- | TTR | spCas9 | spCas9 | CAATCTGACTGCAAACCTGCTGA | 1046 |
| | 12_019 | Promoter | | VRQR IBE | | |
| gRNA-#25 | ABE_NGG_20 nt_3- | TTR | spCas9 | spCas9 | CACAGAAGTCCACTCATTCTTGG | 1047 |
| | 9_002 | Promoter | | ABE | | |
| gRNA-#25 | CBE_NGG_20 nt_4- | TTR | spCas9 | spCas9 | CACAGAAGTCCACTCATTCTTGG | 1048 |
| | 9_003 | Promoter | | CBE | | |
| gRNA-#25 | ABE_NGG_20 nt_3- | TTR | spCas9 | spCas9 IBE | CACAGAAGTCCACTCATTCTTGG | 1049 |
| | 12_018 | Promoter | | | | |
| gRNA-#26 | ABE_NGC_20 nt_3- | TTR | spCas9 | spCas9 | CAGACGATGAGAAGCCATCCTGC | 1050 |
| | 9_008 | Promoter | | NGC ABE | | |
| gRNA-#26 | CBE_NGC_20 nt_4- | TTR | spCas9 | spCas9 | CAGACGATGAGAAGCCATCCTGC | 1051 |
| | 9_009 | Promoter | | NGC CBE | | |
| gRNA-#26 | ABE_NGC_20 nt_3- | TTR | spCas9 | spCas9 | CAGACGATGAGAAGCCATCCTGC | 1052 |
| | 12_020 | Promoter | | NGC IBE | | |
| gRNA-#27 | ABE_NGG_20 nt_3- | TTR | spCas9 | spCas9 | CAGCAGGTTTGCAGTCAGATTGG | 1053 |
| | 9_002 | Promoter | | ABE | | |
| gRNA-#27 | CBE_NGG_20 nt_4- | TTR | spCas9 | spCas9 | CAGCAGGTTTGCAGTCAGATTGG | 1054 |
| | 9_003 | Promoter | | CBE | | |
| gRNA-#27 | ABE_NGG_20 nt_3- | TTR | spCas9 | spCas9 IBE | CAGCAGGTTTGCAGTCAGATTGG | 1055 |
| | 12_018 | Promoter | | | | |
| gRNA-#28 | ABE_NGC_20 nt_3- | TTR | spCas9 | spCas9 | CAGGATGGCTTCTCATCGTCTGC | 1056 |
| | 9_008 | Promoter | | NGC ABE | | |
| gRNA-#28 | CBE_NGC_20 nt_4- | TTR | spCas9 | spCas9 | CAGGATGGCTTCTCATCGTCTGC | 1057 |
| | 9_009 | Promoter | | NGC CBE | | |
| gRNA-#28 | ABE_NGC_20 nt_3- | TTR | spCas9 | spCas9 | CAGGATGGCTTCTCATCGTCTGC | 1058 |
| | 12_020 | Promoter | | NGC IBE | | |
| gRNA-#29 | ABE_NNGRRT_21 nt_5- | TTR | saCas9 | saCas9 | CAGGTTTGCAGTCAGATTGGCAGGGAT | 1059 |
| | 14_011 | Promoter | | ABE | | |
| gRNA-#29 | CBE_NNGRRT_21 nt_3- | TTR | saCas9 | saCas9 | CAGGTTTGCAGTCAGATTGGCAGGGAT | 1060 |
| | 12_012 | Promoter | | CBE | | |
| gRNA-#30 | ABE_NGC_20 nt_3- | TTR | spCas9 | spCas9 | CAGTCAGATTGGCAGGGATAAGC | 1061 |
| | 9_008 | Promoter | | NGC ABE | | |
| gRNA-#30 | CBE_NGC_20 nt_4- | TTR | spCas9 | spCas9 | CAGTCAGATTGGCAGGGATAAGC | 1062 |
| | 9_009 | Promoter | | NGC_CBE | | |
| gRNA-#30 | ABE_NGC_20 nt_3- | TTR | spCas9 | spCas9 | CAGTCAGATTGGCAGGGATAAGC | 1063 |
| | 12_020 | Promoter | | NGC IBE | | |
| gRNA-#31 | ABE_NGC_20 nt_3- | TTR | spCas9 | spCas9 | CCACTCATTCTTGGCAGGATGGC | 1064 |
| | 9_008 | Promoter | | NGC ABE | | |
| gRNA-#31 | CBE_NGC_20 nt_4- | TTR | spCas9 | spCas9 | CCACTCATTCTTGGCAGGATGGC | 1065 |
| | 9_009 | Promoter | | NGC CBE | | |
| gRNA-#31 | ABE_NGC_20 nt_3- | TTR | spCas9 | spCas9 | CCACTCATTCTTGGCAGGATGGC | 1066 |
| | 12_020 | Promoter | | NGC IBE | | |
| gRNA-#32 | ABE_NGC_20 nt_3- | TTR | spCas9 | spCas9 | CTAAGTCAATAATCAGAATCAGC | 1067 |
| | 9_008 | Promoter | | NGC ABE | | |
| gRNA-#32 | CBE_NGC_20 nt_4- | TTR | spCas9 | spCas9 | CTAAGTCAATAATCAGAATCAGC | 1068 |
| | 9_009 | Promoter | | NGC CBE | | |
| gRNA-#32 | ABE_NGC_20 nt_3- | TTR | spCas9 | spCas9 | CTAAGTCAATAATCAGAATCAGC | 1069 |
| | 12_020 | Promoter | | NGC IBE | | |
| gRNA-#33 | ABE_VTTN_22 nt_5- | TTR | cas12b | cas12b | CTTAGTCAACAAAGAGAGAATAAGTA | 1070 |
| | 9_017 | Promoter | | ABE | | |
| gRNA-#34 | ABE_NGC_20 nt_3- | TTR | spCas9 | spCas9 | CTTATCCCTGCCAATCTGACTGC | 1071 |
| | 9_008 | Promoter | | NGC ABE | | |
| gRNA-#34 | CBE_NGC_20 nt_4- | TTR | spCas9 | spCas9 | CTTATCCCTGCCAATCTGACTGC | 1072 |
| | 9_009 | Promoter | | NGC CBE | | |
| gRNA-#34 | ABE_NGC_20 nt_3- | TTR | spCas9 | spCas9 | CTTATCCCTGCCAATCTGACTGC | 1073 |
| | 12_020 | Promoter | | NGC IBE | | |
| gRNA-#35 | ABE_VTTN_22 nt_5- | TTR | cas12b | cas12b | CTTATCCCTGCCAATCTGACTGCAAA | 1074 |
| | 9_017 | Promoter | | ABE | | |
| gRNA-#36 | ABE_VTTN_22 nt_5- | TTR | cas12b | cas12b | CTTATTCTCTCTTTGTTGACTAAGTC | 1075 |
| | 9_017 | Promoter | | ABE | | |
| gRNA-#37 | ABE_VTTN_22 nt_5- | TTR | cas12b | cas12b | CTTCTCCTGAGCTAGGCTGCTTATCC | 1076 |
| | 9_017 | Promoter | | ABE | | |
| gRNA-#38 | ABE_NGC_20 nt_3- | TTR | spCas9 | spCas9 | CTTCTGTGATGGCTGCTCCCAGC | 1077 |
| | 9_008 | Promoter | | NGC ABE | | |
| gRNA-#38 | CBE_NGC_20 nt_4- | TTR | spCas9 | spCas9 | CTTCTGTGATGGCTGCTCCCAGC | 1078 |
| | 9_009 | Promoter | | NGC CBE | | |
| gRNA-#38 | ABE_NGC_20 nt_3- | TTR | spCas9 | spCas9 | CTTCTGTGATGGCTGCTCCCAGC | 1079 |
| | 12_020 | Promoter | | NGC IBE | | |
| gRNA-#39 | ABE_VTTN_22 nt_5- | TTR | cas12b | cas12b | CTTCTGTGATGGCTGCTCCCAGCCTG | 1080 |
| | 9_017 | Promoter | | ABE | | |
| gRNA-#40 | ABE_VTTN_22 nt_5- | TTR | cas12b | cas12b | CTTGGCAGGATGGCTTCTCATCGTCT | 1081 |
| | 9_017 | Promoter | | ABE | | |
| gRNA-#41 | ABE_VTTN_22 nt_5- | TTR | cas12b | cas12b | CTTGTCTAGAGAGATTAGAGCATCGG | 1082 |
| | 9_017 | Promoter | | ABE | | |
| gRNA-#42 | ABE_VTTN_22 nt_5- | TTR | cas12b | cas12b | CTTTGTTGACTAAGTCAATAATCAGA | 1083 |
| | 9_017 | Promoter | | ABE | | |
| gRNA-#43 | ABE_VTTN_22 nt_5- | TTR | cas12b | cas12b | CTTTTATACTCACTTCTCCTGAGCTA | 1084 |
| | 9_017 | Promoter | | ABE | | |
| gRNA-#44 | ABE_NGG_20 nt_3- | TTR | spCas9 | spCas9 | GAAGTGAGTATAAAAGCCCCAGG | 1085 |
| | 9_002 | Promoter | | ABE | | |
| gRNA-#44 | CBE_NGG_20 nt_4- | TTR | spCas9 | spCas9 | GAAGTGAGTATAAAAGCCCCAGG | 1086 |
| | 9_003 | Promoter | | CBE | | |
| gRNA-#44 | ABE_NGG_20 nt_3- | TTR | spCas9 | spCas9 IBE | GAAGTGAGTATAAAAGCCCCAGG | 1087 |
| | 12_018 | Promoter | | | | |
| gRNA-#45 | ABE_NGG_20 nt_3- | TTR | spCas9 | spCas9 | GACAAGGTTCATATTTGTATGGG | 1088 |
| | 9_002 | Promoter | | ABE | | |
| gRNA-#45 | CBE_NGG_20 nt_4- | TTR | spCas9 | spCas9 | GACAAGGTTCATATTTGTATGGG | 1089 |
| | 9_003 | Promoter | | CBE | | |
| gRNA-#45 | ABE_NGG_20 nt_3- | TTR | spCas9 | spCas9 IBE | GACAAGGTTCATATTTGTATGGG | 1090 |
| | 12_018 | Promoter | | | | |
| gRNA-#46 | ABE_NGG_20 nt_3- | TTR | spCas9 | spCas9 | GAGTATAAAAGCCCCAGGCTGGG | 1091 |
| | 9_002 | Promoter | | ABE | | |
| gRNA-#46 | CBE_NGG_20 nt_4- | TTR | spCas9 | spCas9 | GAGTATAAAAGCCCCAGGCTGGG | 1092 |
| | 9_003 | Promoter | | CBE | | |
| gRNA-#46 | ABE_NGG_20 nt_3- | TTR | spCas9 | spCas9 IBE | GAGTATAAAAGCCCCAGGCTGGG | 1093 |
| | 12_018 | Promoter | | | | |
| gRNA-#47 | ABE_NGC_20 nt_3- | TTR | spCas9 | spCas9 | GAGTGGACTTCTGTGATGGCTGC | 1094 |
| | 9_008 | Promoter | | NGC ABE | | |
| gRNA-#47 | CBE_NGC_20 nt_4- | TTR | spCas9 | spCas9 | GAGTGGACTTCTGTGATGGCTGC | 1095 |
| | 9_009 | Promoter | | NGC CBE | | |
| gRNA-#47 | ABE_NGC_20 nt_3- | TTR | spCas9 | spCas9 | GAGTGGACTTCTGTGATGGCTGC | 1096 |
| | 12_020 | Promoter | | NGC IBE | | |
| gRNA-#48 | ABE_NGC_20 nt_3- | TTR | spCas9 | spCas9 | GATGGCTGCTCCCAGCCTGGGGC | 1097 |
| | 9_008 | Promoter | | NGC ABE | | |
| gRNA-#48 | CBE_NGC_20 nt_4- | TTR | spCas9 | spCas9 | GATGGCTGCTCCCAGCCTGGGGC | 1098 |
| | 9_009 | Promoter | | NGC CBE | | |
| gRNA-#48 | ABE_NGC_20 nt_3- | TTR | spCas9 | spCas9 | GATGGCTGCTCCCAGCCTGGGGC | 1099 |
| | 12_020 | Promoter | | NGC IBE | | |
| gRNA-#48 | CBE_NGA_20 nt_4- | TTR | spCas9 | spCas9 | GCAGCCTAGCTCAGGAGAAGTGA | 1100 |
| | 9_006 | Promoter | | VRQR | | |
| | | | | CBE | | |
| gRNA-#48 | ABE_NGA_20 nt_3- | TTR | spCas9 | spCas9 | GCAGCCTAGCTCAGGAGAAGTGA | 1101 |
| | 9_005 | Promoter | | VRQR | | |
| | | | | ABE | | |
| gRNA-#48 | ABE_NGA_20 nt_3- | TTR | spCas9 | spCas9 | GCAGCCTAGCTCAGGAGAAGTGA | 1102 |
| | 12_019 | Promoter | | VRQR IBE | | |
| gRNA-#50 | CBE_NGA_20 nt_4- | TTR | spCas9 | spCas9 | GCTGCTTATCCCTGCCAATCTGA | 1103 |
| | 9_006 | Promoter | | VRQR | | |
| | | | | CBE | | |
| gRNA-#50 | ABE_NGA_20 nt_3- | TTR | spCas9 | spCas9 | GCTGCTTATCCCTGCCAATCTGA | 1104 |
| | 9_005 | Promoter | | VRQR | | |
| | | | | ABE | | |
| gRNA-#50 | ABE_NGA_20 nt_3- | TTR | spCas9 | spCas9 | GCTGCTTATCCCTGCCAATCTGA | 1105 |
| | 12_019 | Promoter | | VRQR IBE | | |
| gRNA-#51 | CBE_NGA_20 nt_4- | TTR | spCas9 | spCas9 | GGGATAAGCAGCCTAGCTCAGGA | 1106 |
| | 9_006 | Promoter | | VRQR | | |
| | | | | CBE | | |
| gRNA-#51 | ABE_NGA_20 nt_3- | TTR | spCas9 | spCas9 | GGGATAAGCAGCCTAGCTCAGGA | 1107 |
| | 9_005 | Promoter | | VRQR | | |
| | | | | ABE | | |
| gRNA-#51 | ABE_NGA_20 nt_3- | TTR | spCas9 | spCas9 | GGGATAAGCAGCCTAGCTCAGGA | 1108 |
| | 12_019 | Promoter | | VRQR IBE | | |
| gRNA-#52 | ABE_NGG_20 nt_3- | TTR | spCas9 | spCas9 | GGTTTGCAGTCAGATTGGCAGGG | 1109 |
| | 9_002 | Promoter | | ABE | | |
| gRNA-#52 | CBE_NGG_20 nt_4- | TTR | spCas9 | spCas9 | GGTTTGCAGTCAGATTGGCAGGG | 1110 |
| | 9_003 | Promoter | | CBE | | |
| gRNA-#52 | ABE_NGG_20 nt_3- | TTR | spCas9 | spCas9 IBE | GGTTTGCAGTCAGATTGGCAGGG | 1111 |
| | 12_018 | Promoter | | | | |
| gRNA-#53 | CBE_NGA_20 nt_4- | TTR | spCas9 | spCas9 | GTTACTTATTCTCTCTTTGTTGA | 1112 |
| | 9_006 | Promoter | | VRQR | | |
| | | | | CBE | | |
| gRNA-#53 | ABE_NGA_20 nt_3- | TTR | spCas9 | spCas9 | GTTACTTATTCTCTCTTTGTTGA | 1113 |
| | 9_005 | Promoter | | VRQR | | |
| | | | | ABE | | |
| gRNA-#53 | ABE_NGA_20 nt_3- | TTR | spCas9 | spCas9 | GTTACTTATTCTCTCTTTGTTGA | 1114 |
| | 12_019 | Promoter | | VRQR IBE | | |
| gRNA-#54 | ABE_VTTN_22 nt_5- | TTR | cas12b | cas12b | GTTACTTATTCTCTCTTTGTTGACTA | 1115 |
| | 9_017 | Promoter | | ABE | | |
| gRNA-#55 | ABE_VTTN_22 nt_5- | TTR | cas12b | cas12b | GTTCATATTTGTATGGGTTACTTATT | 1116 |
| | 9_017 | Promoter | | ABE | | |
| gRNA-#56 | ABE_VTTN_22 nt_5- | TTR | cas12b | cas12b | GTTGACTAAGTCAATAATCAGAATCA | 1117 |
| | 9_017 | Promoter | | ABE | | |
| gRNA-#57 | CBE_NGA_20 nt_4- | TTR | spCas9 | spCas9 | GTTTGCAGTCAGATTGGCAGGGA | 1118 |
| | 9_006 | Promoter | | VRQR | | |
| | | | | CBE | | |
| gRNA-#57 | ABE_NGA_20 nt_3- | TTR | spCas9 | spCas9 | GTTTGCAGTCAGATTGGCAGGGA | 1119 |
| | 9_005 | Promoter | | VRQR | | |
| | | | | ABE | | |
| gRNA-#57 | ABE_NGA_20 nt_3- | TTR | spCas9 | spCas9 | GTTTGCAGTCAGATTGGCAGGGA | 1120 |
| | 12_019 | Promoter | | VRQR IBE | | |
| gRNA-#58 | ABE_VTTN_22 nt_5- | TTR | cas12b | cas12b | GTTTGCAGTCAGATTGGCAGGGATAA | 1121 |
| | 9_017 | Promoter | | ABE | | |
| gRNA-#59 | CBE_NGA_20 nt_4- | TTR | spCas9 | spCas9 | TACAAATATGAACCTTGTCTAGA | 1122 |
| | 9_006 | Promoter | | VRQR | | |
| | | | | CBE | | |
| gRNA-#59 | ABE_NGA_20 nt_3- | TTR | spCas9 | spCas9 | TACAAATATGAACCTTGTCTAGA | 1123 |
| | 9_005 | Promoter | | VRQR | | |
| | | | | ABE | | |
| gRNA-#59 | ABE_NGA_20 nt_3- | TTR | spCas9 | spCas9 | TACAAATATGAACCTTGTCTAGA | 1124 |
| | 12_019 | Promoter | | VRQR IBE | | |
| gRNA-#60 | ABE_NGC_20 nt_3- | TTR | spCas9 | spCas9 | TACTCACTTCTCCTGAGCTAGGC | 1125 |
| | 9_008 | Promoter | | NGC ABE | | |
| gRNA-#60 | CBE_NGC_20 nt_4- | TTR | spCas9 | spCas9 | TACTCACTTCTCCTGAGCTAGGC | 1126 |
| | 9_009 | Promoter | | NGC CBE | | |
| gRNA-#60 | ABE_NGC_20 nt_3- | TTR | spCas9 | spCas9 | TACTCACTTCTCCTGAGCTAGGC | 1127 |
| | 12_020 | Promoter | | NGC IBE | | |
| gRNA-#61 | ABE_NGC_20 nt_3- | TTR | spCas9 | spCas9 | TATAAAAGCCCCAGGCTGGGAGC | 1128 |
| | 9_008 | Promoter | | NGC ABE | | |
| gRNA-#61 | CBE_NGC_20 nt_4- | TTR | spCas9 | spCas9 | TATAAAAGCCCCAGGCTGGGAGC | 1129 |
| | 9_009 | Promoter | | NGC CBE | | |
| gRNA-#61 | ABE_NGC_20 nt_3- | TTR | spCas9 | spCas9 | TATAAAAGCCCCAGGCTGGGAGC | 1130 |
| | 12_020 | Promoter | | NGC IBE | | |
| gRNA-#62 | ABE_NGC_20 nt_3- | TTR | spCas9 | spCas9 | TCACTTCTCCTGAGCTAGGCTGC | 1131 |
| | 9_008 | Promoter | | NGC ABE | | |
| gRNA-#62 | CBE_NGC_20 nt_4- | TTR | spCas9 | spCas9 | TCACTTCTCCTGAGCTAGGCTGC | 1132 |
| | 9_009 | Promoter | | NGC CBE | | |
| gRNA-#62 | ABE_NGC_20 nt_3- | TTR | spCas9 | spCas9 | TCACTTCTCCTGAGCTAGGCTGC | 1133 |
| | 12_020 | Promoter | | NGC IBE | | |
| gRNA-#63 | ABE_NGC_20 nt_3- | TTR | spCas9 | spCas9 | TCAGATTGGCAGGGATAAGCAGC | 1134 |
| | 9_008 | Promoter | | NGC ABE | | |
| gRNA-#63 | CBE_NGC_20 nt_4- | TTR | spCas9 | spCas9 | TCAGATTGGCAGGGATAAGCAGC | 1135 |
| | 9_009 | Promoter | | NGC CBE | | |
| gRNA-#63 | ABE_NGC_20 nt_3- | TTR | spCas9 | spCas9 | TCAGATTGGCAGGGATAAGCAGC | 1136 |
| | 12_020 | Promoter | | NGC IBE | | |
| gRNA-#64 | ABE_NGC_20 nt_3- | TTR | spCas9 | spCas9 | TCAGGAGAAGTGAGTATAAAAGC | 1137 |
| | 9_008 | Promoter | | NGC ABE | | |
| gRNA-#64 | CBE_NGC_20 nt_4- | TTR | spCas9 | spCas9 | TCAGGAGAAGTGAGTATAAAAGC | 1138 |
| | 9_009 | Promoter | | NGC CBE | | |
| gRNA-#64 | ABE_NGC_20 nt_3- | TTR | spCas9 | spCas9 | TCAGGAGAAGTGAGTATAAAAGC | 1139 |
| | 12_020 | Promoter | | NGC IBE | | |
| gRNA-#65 | ABE_NNRRT_21 nt_5- | TTR | saCas9 | saCas9 | TCTGACTGCAAACCTGCTGATTCTGAT | 1140 |
| | 14_014 | Promoter | | KKH ABE | | |
| gRNA-#65 | CBE_NNRRT_21 nt_3- | TTR | saCas9 | saCas9 | TCTGACTGCAAACCTGCTGATTCTGAT | 1141 |
| | 12_015 | Promoter | | KKH CBE | | |
| gRNA-#66 | ABE_NGC_20 nt_3- | TTR | spCas9 | spCas9 | TGAGCTAGGCTGCTTATCCCTGC | 1142 |
| | 9_008 | Promoter | | NGC ABE | | |
| gRNA-#66 | CBE_NGC_20 nt_4- | TTR | spCas9 | spCas9 | TGAGCTAGGCTGCTTATCCCTGC | 1143 |
| | 9_009 | Promoter | | NGC CBE | | |
| gRNA-#66 | ABE_NGC_20 nt_3- | TTR | spCas9 | spCas9 | TGAGCTAGGCTGCTTATCCCTGC | 1144 |
| | 12_020 | Promoter | | NGC IBE | | |
| gRNA-#67 | ABE_NGC_20 nt_3- | TTR | spCas9 | spCas9 | TGCCAATCTGACTGCAAACCTGC | 1145 |
| | 9_008 | Promoter | | NGC ABE | | |
| gRNA-#67 | CBE_NGC_20 nt_4- | TTR | spCas9 | spCas9 | TGCCAATCTGACTGCAAACCTGC | 1146 |
| | 9_009 | Promoter | | NGC CBE | | |
| gRNA-#67 | ABE_NGC_20 nt_3- | TTR | spCas9 | spCas9 | TGCCAATCTGACTGCAAACCTGC | 1147 |
| | 12_020 | Promoter | | NGC IBE | | |
| gRNA-#68 | ABE_NGG_20 nt_3- | TTR | spCas9 | spCas9 | TGCTCTAATCTCTCTAGACAAGG | 1148 |
| | 9_002 | Promoter | | ABE | | |
| gRNA-#68 | CBE_NGG_20 nt_4- | TTR | spCas9 | spCas9 | TGCTCTAATCTCTCTAGACAAGG | 1149 |
| | 9_003 | Promoter | | CBE | | |
| gRNA-#68 | ABE_NGG_20 nt_3- | TTR | spCas9 | spCas9 IBE | TGCTCTAATCTCTCTAGACAAGG | 1150 |
| | 12_018 | Promoter | | | | |
| gRNA-#69 | ABE_NGG_20 nt_3- | TTR | spCas9 | spCas9 | TGTGATGGCTGCTCCCAGCCTGG | 1151 |
| | 9_002 | Promoter | | ABE | | |
| gRNA-#69 | CBE_NGG_20 nt_4- | TTR | spCas9 | spCas9 | TGTGATGGCTGCTCCCAGCCTGG | 1152 |
| | 9_003 | Promoter | | CBE | | |
| gRNA-#69 | ABE_NGG_20 nt_3- | TTR | spCas9 | spCas9 IBE | TGTGATGGCTGCTCCCAGCCTGG | 1153 |
| | 12_018 | Promoter | | | | |
| gRNA-#70 | ABE_NGC_20 nt_3- | TTR | spCas9 | spCas9 | TTGGCAGGGATAAGCAGCCTAGC | 1154 |
| | 9_008 | Promoter | | NGC ABE | | |
| gRNA-#70 | CBE_NGC_20 nt_4- | TTR | spCas9 | spCas9 | TTGGCAGGGATAAGCAGCCTAGC | 1155 |
| | 9_009 | Promoter | | NGC CBE | | |
| gRNA-#70 | ABE_NGC_20 nt_3- | TTR | spCas9 | spCas9 | TTGGCAGGGATAAGCAGCCTAGC | 1156 |
| | 12_020 | Promoter | | NGC IBE | | |
| gRNA-#71 | ABE_NGC_20 nt_3- | TTR | spCas9 | spCas9 | TTTTATACTCACTTCTCCTGAGC | 1157 |
| | 9_008 | Promoter | | NGC ABE | | |
| gRNA-#71 | CBE_NGC_20 nt_4- | TTR | spCas9 | spCas9 | TTTTATACTCACTTCTCCTGAGC | 1158 |
| | 9_009 | Promoter | | NGC CBE | | |
| gRNA-#71 | ABE_NGC_20 nt_3- | TTR | spCas9 | spCas9 | TTTTATACTCACTTCTCCTGAGC | 1159 |
| | 12_020 | Promoter | | NGC IBE | | |
| | | Human | | | | Target |
| gRNA | | Chromosome | Start | | | Base |
| Name | PAM | Location | Site | End Site | Strand | Position(s) |
| gRNA1594 | GGC | chr18 | 31593011 | 31593034 | − | 8 |
| gRNA1594 | GGC | chr18 | 31593011 | 31593034 | − | 7 |
| gRNA1594 | GGC | chr18 | 31593011 | 31593034 | − | 7 |
| gRNA1595 | AGC | chr18 | 31592994 | 31593017 | − | 9 |
| gRNA1596 | AGC | chr18 | 31592991 | 31593014 | − | 7, 6 |
| gRNA1597 | AGC | chr18 | 31598558 | 31598581 | + | 8 |
| gRNA1597 | AGC | chr18 | 31598558 | 31598581 | + | 8 |
| gRNA1598 | TGG | chr18 | 31595114 | 31595137 | + | 4 |
| gRNA1599 | TGG | chr18 | 31595239 | 31595262 | − | 6 |
| gRNA1600 | TGG | chr18 | 31593012 | 31593035 | − | 8 |
| gRNA1601 | TGC | chr18 | 31595245 | 31595268 | − | 12 |
| gRNA1602 | AGC | chr18 | 31591959 | 31591982 | − | 10 |
| gRNA1603 | TGA | chr18 | 31592883 | 31592906 | + | 11 |
| gRNA1604 | TGA | chr18 | 31595108 | 31595131 | + | 10 |
| gRNA1605 | GGA | chr18 | 31595115 | 31595138 | + | 3 |
| gRNA1606 | GGA | chr18 | 31595238 | 31595261 | − | 5 |
| gRNA1607 | AGA | chr18 | 31591953 | 31591976 | − | 4 |
| gRNA1746 | TGA | chr18 | 31591776 | 31591799 | − | |
| gRNA1746 | TGA | chr18 | 31591776 | 31591799 | − | |
| gRNA1746 | TGA | chr18 | 31591776 | 31591799 | − | |
| gRNA1747 | ACAAAT | chr18 | 31591738 | 31591765 | − | |
| gRNA1747 | ACAAAT | chr18 | 31591738 | 31591765 | − | |
| gRNA1748 | GTGAGT | chr18 | 31591820 | 31591847 | + | |
| gRNA1748 | GTGAGT | chr18 | 31591820 | 31591847 | + | |
| gRNA1749 | GGA | chr18 | 31591880 | 31591903 | + | |
| gRNA1749 | GGA | chr18 | 31591880 | 31591903 | + | |
| gRNA1749 | GGA | chr18 | 31591880 | 31591903 | + | |
| gRNA1750 | AAGAAT | chr18 | 31591890 | 31591917 | − | |
| gRNA1750 | AAGAAT | chr18 | 31591890 | 31591917 | − | |
| gRNA1751 | TGG | chr18 | 31591725 | 31591748 | + | |
| gRNA1751 | TGG | chr18 | 31591725 | 31591748 | + | |
| gRNA1751 | TGG | chr18 | 31591725 | 31591748 | + | |
| gRNA1752 | AGA | chr18 | 31591858 | 31591881 | + | |
| gRNA1752 | AGA | chr18 | 31591858 | 31591881 | + | |
| gRNA1752 | AGA | chr18 | 31591858 | 31591881 | + | |
| gRNA1753 | TGA | chr18 | 31591734 | 31591757 | − | |
| gRNA1753 | TGA | chr18 | 31591734 | 31591757 | − | |
| gRNA1753 | TGA | chr18 | 31591734 | 31591757 | − | |
| gRNA1754 | AGG | chr18 | 31591826 | 31591849 | − | |
| gRNA1754 | AGG | chr18 | 31591826 | 31591849 | − | |
| gRNA1754 | AGG | chr18 | 31591826 | 31591849 | − | |
| gRNA1755 | AGA | chr18 | 31591761 | 31591784 | − | |
| gRNA1755 | AGA | chr18 | 31591761 | 31591784 | − | |
| gRNA1755 | AGA | chr18 | 31591761 | 31591784 | − | |
| gRNA1756 | AGA | chr18 | 31591720 | 31591743 | − | |
| gRNA1756 | AGA | chr18 | 31591720 | 31591743 | − | |
| gRNA1756 | AGA | chr18 | 31591720 | 31591743 | − | |
| gRNA1757 | CAGGAT | chr18 | 31591877 | 31591904 | + | |
| gRNA1757 | CAGGAT | chr18 | 31591877 | 31591904 | + | |
| gRNA1758 | AGAAGT | chr18 | 31591857 | 31591884 | + | |
| gRNA1758 | AGAAGT | chr18 | 31591857 | 31591884 | + | |
| gRNA1759 | GGA | chr18 | 31591883 | 31591906 | − | |
| gRNA1759 | GGA | chr18 | 31591883 | 31591906 | − | |
| gRNA1759 | GGA | chr18 | 31591883 | 31591906 | − | |
| gRNA1760 | CTTAGT | chr18 | 31591770 | 31591797 | − | |
| gRNA1760 | CTTAGT | chr18 | 31591770 | 31591797 | − | |
| gRNA1761 | CAAGGT | chr18 | 31591707 | 31591734 | + | |
| gRNA1761 | CAAGGT | chr18 | 31591707 | 31591734 | + | |
| gRNA1762 | GCAGGT | chr18 | 31591771 | 31591798 | + | |
| gRNA1762 | GCAGGT | chr18 | 31591771 | 31591798 | + | |
| gRNA1763 | ATGGGT | chr18 | 31591723 | 31591750 | + | |
| gRNA1763 | ATGGGT | chr18 | 31591723 | 31591750 | + | |
| gRNA1764 | AGA | chr18 | 31591713 | 31591736 | − | |
| gRNA1764 | AGA | chr18 | 31591713 | 31591736 | − | |
| gRNA1764 | AGA | chr18 | 31591713 | 31591736 | − | |
| gRNA1765 | AGG | chr18 | 31591879 | 31591902 | + | |
| gRNA1765 | AGG | chr18 | 31591879 | 31591902 | + | |
| gRNA1765 | AGG | chr18 | 31591879 | 31591902 | + | |
| gRNA1766 | AGA | chr18 | 31591786 | 31591809 | + | |
| gRNA1766 | AGA | chr18 | 31591786 | 31591809 | + | |
| gRNA1766 | AGA | chr18 | 31591786 | 31591809 | + | |
| gRNA1767 | TGG | chr18 | 31591871 | 31591894 | − | |
| gRNA1767 | TGG | chr18 | 31591871 | 31591894 | − | |
| gRNA1767 | TGG | chr18 | 31591871 | 31591894 | − | |
| gRNA1768 | TGA | chr18 | 31591783 | 31591806 | − | |
| gRNA1768 | TGA | chr18 | 31591783 | 31591806 | − | |
| gRNA1768 | TGA | chr18 | 31591783 | 31591806 | − | |
| gRNA1769 | ATAAGT | chr18 | 31591751 | 31591778 | − | |
| gRNA1769 | ATAAGT | chr18 | 31591751 | 31591778 | − | |
| gRNA1770 | AGA | chr18 | 31591817 | 31591840 | + | |
| gRNA1770 | AGA | chr18 | 31591817 | 31591840 | + | |
| gRNA1770 | AGA | chr18 | 31591817 | 31591840 | + | |
| gRNA1771 | AGA | chr18 | 31591892 | 31591915 | − | |
| gRNA1771 | AGA | chr18 | 31591892 | 31591915 | − | |
| gRNA1771 | AGA | chr18 | 31591892 | 31591915 | − | |
| gRNA1772 | TGG | chr18 | 31591884 | 31591907 | − | |
| gRNA1772 | TGG | chr18 | 31591884 | 31591907 | − | |
| gRNA1772 | TGG | chr18 | 31591884 | 31591907 | − | |
| gRNA1773 | TGA | chr18 | 31591832 | 31591855 | − | |
| gRNA1773 | TGA | chr18 | 31591832 | 31591855 | − | |
| gRNA1773 | TGA | chr18 | 31591832 | 31591855 | − | |
| gRNA1774 | AGAAGT | chr18 | 31591816 | 31591843 | + | |
| gRNA1774 | AGAAGT | chr18 | 31591816 | 31591843 | + | |
| gRNA1775 | CGG | chr18 | 31591706 | 31591729 | − | |
| gRNA1775 | CGG | chr18 | 31591706 | 31591729 | − | |
| gRNA1775 | CGG | chr18 | 31591706 | 31591729 | − | |
| gRNA1776 | GGG | chr18 | 31591855 | 31591878 | − | |
| gRNA1776 | GGG | chr18 | 31591855 | 31591878 | − | |
| gRNA1776 | GGG | chr18 | 31591855 | 31591878 | − | |
| gRNA1777 | CTAAGT | chr18 | 31591750 | 31591777 | + | |
| gRNA1777 | CTAAGT | chr18 | 31591750 | 31591777 | + | |
| gRNA1778 | GTCAAT | chr18 | 31591754 | 31591781 | + | |
| gRNA1778 | GTCAAT | chr18 | 31591754 | 31591781 | + | |
| gRNA1779 | AGA | chr18 | 31591759 | 31591782 | − | |
| gRNA1779 | AGA | chr18 | 31591759 | 31591782 | − | |
| gRNA1779 | AGA | chr18 | 31591759 | 31591782 | − | |
| gRNA1780 | GAGAAT | chr18 | 31591755 | 31591782 | − | |
| gRNA1780 | GAGAAT | chr18 | 31591755 | 31591782 | − | |
| gRNA1781 | TCAGAT | chr18 | 31591783 | 31591810 | + | |
| gRNA1781 | TCAGAT | chr18 | 31591783 | 31591810 | + | |
| gRNA1782 | TGG | chr18 | 31591883 | 31591906 | + | |
| gRNA1782 | TGG | chr18 | 31591883 | 31591906 | + | |
| gRNA1782 | TGG | chr18 | 31591883 | 31591906 | + | |
| gRNA1783 | AATAAT | chr18 | 31591757 | 31591784 | + | |
| gRNA1783 | AATAAT | chr18 | 31591757 | 31591784 | + | |
| gRNA1784 | ATGAGT | chr18 | 31591886 | 31591913 | − | |
| gRNA1784 | ATGAGT | chr18 | 31591886 | 31591913 | − | |
| gRNA1785 | GCCAAT | chr18 | 31591808 | 31591835 | − | |
| gRNA1785 | GCCAAT | chr18 | 31591808 | 31591835 | − | |
| gRNA1786 | TGG | chr18 | 31591842 | 31591865 | + | |
| gRNA1786 | TGG | chr18 | 31591842 | 31591865 | + | |
| gRNA1786 | TGG | chr18 | 31591842 | 31591865 | + | |
| gRNA1787 | GGG | chr18 | 31591854 | 31591877 | − | |
| gRNA1787 | GGG | chr18 | 31591854 | 31591877 | − | |
| gRNA1787 | GGG | chr18 | 31591854 | 31591877 | − | |
| gRNA1788 | TGTGAT | chr18 | 31591873 | 31591900 | − | |
| gRNA1788 | TGTGAT | chr18 | 31591873 | 31591900 | − | |
| gRNA1789 | GCTGAT | chr18 | 31591788 | 31591815 | − | |
| gRNA1789 | GCTGAT | chr18 | 31591788 | 31591815 | − | |
| gRNA1790 | AGA | chr18 | 31591765 | 31591788 | + | |
| gRNA1790 | AGA | chr18 | 31591765 | 31591788 | + | |
| gRNA1790 | AGA | chr18 | 31591765 | 31591788 | + | |
| gRNA1791 | AGA | chr18 | 31591757 | 31591780 | − | |
| gRNA1791 | AGA | chr18 | 31591757 | 31591780 | − | |
| gRNA1791 | AGA | chr18 | 31591757 | 31591780 | − | |
| gRNA1792 | CAGAAT | chr18 | 31591763 | 31591790 | + | |
| gRNA1792 | CAGAAT | chr18 | 31591763 | 31591790 | + | |
| gRNA-#1 | AGC | chr18 | 31591849 | 31591872 | + | |
| gRNA-#1 | AGC | chr18 | 31591849 | 31591872 | + | |
| gRNA-#1 | AGC | chr18 | 31591849 | 31591872 | + | |
| gRNA-#2 | GGC | chr18 | 31591839 | 31591862 | + | |
| gRNA-#2 | GGC | chr18 | 31591839 | 31591862 | + | |
| gRNA-#2 | GGC | chr18 | 31591839 | 31591862 | + | |
| gRNA-#3 | TGCAGT | chr18 | 31591778 | 31591805 | + | |
| gRNA-#3 | TGCAGT | chr18 | 31591778 | 31591805 | + | |
| gRNA-#4 | AGA | chr18 | 31591718 | 31591741 | − | |
| gRNA-#4 | AGA | chr18 | 31591718 | 31591741 | − | |
| gRNA-#4 | AGA | chr18 | 31591718 | 31591741 | − | |
| gRNA-#5 | GGC | chr18 | 31591870 | 31591893 | − | |
| gRNA-#5 | GGC | chr18 | 31591870 | 31591893 | − | |
| gRNA-#5 | GGC | chr18 | 31591870 | 31591893 | − | |
| gRNA-#6 | AGAGAT | chr18 | 31591717 | 31591744 | − | |
| gRNA-#6 | AGAGAT | chr18 | 31591717 | 31591744 | − | |
| gRNA-#7 | GGC | chr18 | 31591876 | 31591899 | + | |
| gRNA-#7 | GGC | chr18 | 31591876 | 31591899 | + | |
| gRNA-#7 | GGC | chr18 | 31591876 | 31591899 | + | |
| gRNA-#8 | AGC | chr18 | 31591711 | 31591734 | − | |
| gRNA-#8 | AGC | chr18 | 31591711 | 31591734 | − | |
| gRNA-#8 | AGC | chr18 | 31591711 | 31591734 | − | |
| gRNA-#9 | TGA | chr18 | 31591888 | 31591911 | − | |
| gRNA-#9 | TGA | chr18 | 31591888 | 31591911 | − | |
| gRNA-#9 | TGA | chr18 | 31591888 | 31591911 | − | |
| gRNA-#10 | GGC | chr18 | 31591791 | 31591814 | + | |
| gRNA-#10 | GGC | chr18 | 31591791 | 31591814 | + | |
| gRNA-#10 | GGC | chr18 | 31591791 | 31591814 | + | |
| gRNA-#11 | AGG | chr18 | 31591814 | 31591837 | + | |
| gRNA-#11 | AGG | chr18 | 31591814 | 31591837 | + | |
| gRNA-#11 | AGG | chr18 | 31591814 | 31591837 | + | |
| gRNA-#12 | AGG | chr18 | 31591794 | 31591817 | + | |
| gRNA-#12 | AGG | chr18 | 31591794 | 31591817 | + | |
| gRNA-#12 | AGG | chr18 | 31591794 | 31591817 | + | |
| gRNA-#13 | GGA | chr18 | 31591844 | 31591867 | + | |
| gRNA-#13 | GGA | chr18 | 31591844 | 31591867 | + | |
| gRNA-#13 | GGA | chr18 | 31591844 | 31591867 | + | |
| gRNA-#14 | AGG | chr18 | 31591774 | 31591797 | + | |
| gRNA-#14 | AGG | chr18 | 31591774 | 31591797 | + | |
| gRNA-#14 | AGG | chr18 | 31591774 | 31591797 | + | |
| gRNA-#15 | TGC | chr18 | 31591779 | 31591802 | + | |
| gRNA-#15 | TGC | chr18 | 31591779 | 31591802 | + | |
| gRNA-#15 | TGC | chr18 | 31591779 | 31591802 | + | |
| gRNA-#16 | ATTA | chr18 | 31591758 | 31591784 | − | |
| gRNA-#17 | ATTC | chr18 | 31591755 | 31591781 | + | |
| gRNA-#18 | ATTC | chr18 | 31591764 | 31591790 | − | |
| gRNA-#19 | ATTC | chr18 | 31591890 | 31591916 | + | |
| gRNA-#20 | ATTG | chr18 | 31591755 | 31591781 | − | |
| gRNA-#21 | ATTG | chr18 | 31591808 | 31591834 | + | |
| gRNA-#22 | ATTT | chr18 | 31591738 | 31591764 | + | |
| gRNA-#23 | TGA | chr18 | 31591874 | 31591897 | − | |
| gRNA-#23 | TGA | chr18 | 31591874 | 31591897 | − | |
| gRNA-#23 | TGA | chr18 | 31591874 | 31591897 | − | |
| gRNA-#24 | TGA | chr18 | 31591789 | 31591812 | − | |
| gRNA-#24 | TGA | chr18 | 31591789 | 31591812 | − | |
| gRNA-#24 | TGA | chr18 | 31591789 | 31591812 | − | |
| gRNA-#25 | TGG | chr18 | 31591875 | 31591898 | + | |
| gRNA-#25 | TGG | chr18 | 31591875 | 31591898 | + | |
| gRNA-#25 | TGG | chr18 | 31591875 | 31591898 | + | |
| gRNA-#26 | TGC | chr18 | 31591897 | 31591920 | − | |
| gRNA-#26 | TGC | chr18 | 31591897 | 31591920 | − | |
| gRNA-#26 | TGC | chr18 | 31591897 | 31591920 | − | |
| gRNA-#27 | TGG | chr18 | 31591790 | 31591813 | + | |
| gRNA-#27 | TGG | chr18 | 31591790 | 31591813 | + | |
| gRNA-#27 | TGG | chr18 | 31591790 | 31591813 | + | |
| gRNA-#28 | TGC | chr18 | 31591898 | 31591921 | + | |
| gRNA-#28 | TGC | chr18 | 31591898 | 31591921 | + | |
| gRNA-#28 | TGC | chr18 | 31591898 | 31591921 | + | |
| gRNA-#29 | AGGGAT | chr18 | 31591793 | 31591820 | + | |
| gRNA-#29 | AGGGAT | chr18 | 31591793 | 31591820 | + | |
| gRNA-#30 | AGC | chr18 | 31591801 | 31591824 | + | |
| gRNA-#30 | AGC | chr18 | 31591801 | 31591824 | + | |
| gRNA-#30 | AGC | chr18 | 31591801 | 31591824 | + | |
| gRNA-#31 | GGC | chr18 | 31591884 | 31591907 | + | |
| gRNA-#31 | GGC | chr18 | 31591884 | 31591907 | + | |
| gRNA-#31 | GGC | chr18 | 31591884 | 31591907 | + | |
| gRNA-#32 | AGC | chr18 | 31591771 | 31591794 | + | |
| gRNA-#32 | AGC | chr18 | 31591771 | 31591794 | + | |
| gRNA-#32 | AGC | chr18 | 31591771 | 31591794 | + | |
| gRNA-#33 | CTTA | chr18 | 31591750 | 31591776 | − | |
| gRNA-#34 | TGC | chr18 | 31591800 | 31591823 | − | |
| gRNA-#34 | TGC | chr18 | 31591800 | 31591823 | − | |
| gRNA-#34 | TGC | chr18 | 31591800 | 31591823 | − | |
| gRNA-#35 | CTTA | chr18 | 31591797 | 31591823 | − | |
| gRNA-#36 | CTTA | chr18 | 31591752 | 31591778 | + | |
| gRNA-#37 | CTTC | chr18 | 31591816 | 31591842 | − | |
| gRNA-#38 | AGC | chr18 | 31591860 | 31591883 | − | |
| gRNA-#38 | AGC | chr18 | 31591860 | 31591883 | − | |
| gRNA-#38 | AGC | chr18 | 31591860 | 31591883 | − | |
| gRNA-#39 | CTTC | chr18 | 31591857 | 31591883 | − | |
| gRNA-#40 | CTTG | chr18 | 31591893 | 31591919 | + | |
| gRNA-#41 | CTTG | chr18 | 31591706 | 31591732 | − | |
| gRNA-#42 | CTTT | chr18 | 31591762 | 31591788 | + | |
| gRNA-#43 | CTTT | chr18 | 31591828 | 31591854 | − | |
| gRNA-#44 | AGG | chr18 | 31591838 | 31591861 | + | |
| gRNA-#44 | AGG | chr18 | 31591838 | 31591861 | + | |
| gRNA-#44 | AGG | chr18 | 31591838 | 31591861 | + | |
| gRNA-#45 | GGG | chr18 | 31591726 | 31591749 | + | |
| gRNA-#45 | GGG | chr18 | 31591726 | 31591749 | + | |
| gRNA-#45 | GGG | chr18 | 31591726 | 31591749 | + | |
| gRNA-#46 | GGG | chr18 | 31591843 | 31591866 | + | |
| gRNA-#46 | GGG | chr18 | 31591843 | 31591866 | + | |
| gRNA-#46 | GGG | chr18 | 31591843 | 31591866 | + | |
| gRNA-#47 | TGC | chr18 | 31591867 | 31591890 | − | |
| gRNA-#47 | TGC | chr18 | 31591867 | 31591890 | − | |
| gRNA-#47 | TGC | chr18 | 31591867 | 31591890 | − | |
| gRNA-#48 | GGC | chr18 | 31591853 | 31591876 | − | |
| gRNA-#48 | GGC | chr18 | 31591853 | 31591876 | − | |
| gRNA-#48 | GGC | chr18 | 31591853 | 31591876 | − | |
| gRNA-#48 | TGA | chr18 | 31591822 | 31591845 | + | |
| gRNA-#48 | TGA | chr18 | 31591822 | 31591845 | + | |
| gRNA-#48 | TGA | chr18 | 31591822 | 31591845 | + | |
| gRNA-#50 | TGA | chr18 | 31591804 | 31591827 | − | |
| gRNA-#50 | TGA | chr18 | 31591804 | 31591827 | − | |
| gRNA-#50 | TGA | chr18 | 31591804 | 31591827 | − | |
| gRNA-#51 | GGA | chr18 | 31591815 | 31591838 | + | |
| gRNA-#51 | GGA | chr18 | 31591815 | 31591838 | + | |
| gRNA-#51 | GGA | chr18 | 31591815 | 31591838 | + | |
| gRNA-#52 | GGG | chr18 | 31591795 | 31591818 | + | |
| gRNA-#52 | GGG | chr18 | 31591795 | 31591818 | + | |
| gRNA-#52 | GGG | chr18 | 31591795 | 31591818 | + | |
| gRNA-#53 | TGA | chr18 | 31591748 | 31591771 | + | |
| gRNA-#53 | TGA | chr18 | 31591748 | 31591771 | + | |
| gRNA-#53 | TGA | chr18 | 31591748 | 31591771 | + | |
| gRNA-#54 | GTTA | chr18 | 31591748 | 31591774 | + | |
| gRNA-#55 | GTTC | chr18 | 31591732 | 31591758 | + | |
| gRNA-#56 | GTTG | chr18 | 31591766 | 31591792 | + | |
| gRNA-#57 | GGA | chr18 | 31591796 | 31591819 | + | |
| gRNA-#57 | GGA | chr18 | 31591796 | 31591819 | + | |
| gRNA-#57 | GGA | chr18 | 31591796 | 31591819 | + | |
| gRNA-#58 | GTTT | chr18 | 31591796 | 31591822 | + | |
| gRNA-#59 | AGA | chr18 | 31591722 | 31591745 | − | |
| gRNA-#59 | AGA | chr18 | 31591722 | 31591745 | − | |
| gRNA-#59 | AGA | chr18 | 31591722 | 31591745 | − | |
| gRNA-#60 | GGC | chr18 | 31591825 | 31591848 | − | |
| gRNA-#60 | GGC | chr18 | 31591825 | 31591848 | − | |
| gRNA-#60 | GGC | chr18 | 31591825 | 31591848 | − | |
| gRNA-#61 | AGC | chr18 | 31591846 | 31591869 | + | |
| gRNA-#61 | AGC | chr18 | 31591846 | 31591869 | + | |
| gRNA-#61 | AGC | chr18 | 31591846 | 31591869 | + | |
| gRNA-#62 | TGC | chr18 | 31591822 | 31591845 | − | |
| gRNA-#62 | TGC | chr18 | 31591822 | 31591845 | − | |
| gRNA-#62 | TGC | chr18 | 31591822 | 31591845 | − | |
| gRNA-#63 | AGC | chr18 | 31591804 | 31591827 | + | |
| gRNA-#63 | AGC | chr18 | 31591804 | 31591827 | + | |
| gRNA-#63 | AGC | chr18 | 31591804 | 31591827 | + | |
| gRNA-#64 | AGC | chr18 | 31591832 | 31591855 | + | |
| gRNA-#64 | AGC | chr18 | 31591832 | 31591855 | + | |
| gRNA-#64 | AGC | chr18 | 31591832 | 31591855 | + | |
| gRNA-#65 | TCTGAT | chr18 | 31591782 | 31591809 | − | |
| gRNA-#65 | TCTGAT | chr18 | 31591782 | 31591809 | − | |
| gRNA-#66 | TGC | chr18 | 31591812 | 31591835 | − | |
| gRNA-#66 | TGC | chr18 | 31591812 | 31591835 | − | |
| gRNA-#66 | TGC | chr18 | 31591812 | 31591835 | − | |
| gRNA-#67 | TGC | chr18 | 31591792 | 31591815 | − | |
| gRNA-#67 | TGC | chr18 | 31591792 | 31591815 | − | |
| gRNA-#67 | TGC | chr18 | 31591792 | 31591815 | − | |
| gRNA-#68 | AGG | chr18 | 31591710 | 31591733 | + | |
| gRNA-#68 | AGG | chr18 | 31591710 | 31591733 | + | |
| gRNA-#68 | AGG | chr18 | 31591710 | 31591733 | + | |
| gRNA-#69 | TGG | chr18 | 31591856 | 31591879 | − | |
| gRNA-#69 | TGG | chr18 | 31591856 | 31591879 | − | |
| gRNA-#69 | TGG | chr18 | 31591856 | 31591879 | − | |
| gRNA-#70 | AGC | chr18 | 31591809 | 31591832 | + | |
| gRNA-#70 | AGC | chr18 | 31591809 | 31591832 | + | |
| gRNA-#70 | AGC | chr18 | 31591809 | 31591832 | + | |
| gRNA-#71 | AGC | chr18 | 31591830 | 31591853 | − | |
| gRNA-#71 | AGC | chr18 | 31591830 | 31591853 | − | |
| gRNA-#71 | AGC | chr18 | 31591830 | 31591853 | − | |

The spacer sequences in Table 2A corresponding to sgRNAs sgRNA_361, sgRNA_362, sgRNA_363, sgRNA_364, sgRNA_365, sgRNA_366, and sgRNA_367 can be used for targeting a base editor to alter a nucleobase of a splice site of the transthyretin polynucleotide. The spacer sequences in Table 2A corresponding to sgRNAs sgRNA_368, sgRNA_369, sgRNA_370, sgRNA_371, sgRNA_372, sgRNA_373, and sgRNA_374 can be used for targeting an endonuclease to a transthyretin (TTR) polynucleotide sequence. The three spacer sequences in Table 2 corresponding to sgRNA_375, sgRNA_376, and sgRNA_377 can be used to alter a nucleobase of a transthyretin (TTR) polynucleotide. The alteration of the nucleobase can result in an alteration of an isoleucine (I) to a valine (V) (e.g., to correct a V122I mutation in a transthyretin polypeptide encoded by the transthyretin polynucleotide). In embodiments, a transthyretin polynucleotide can be edited using the following combinations of base editors and sgRNA sequences (see Tables 1 and 2A): ABE8.8 and sgRNA_361; ABE8.8 and sgRNA_362; ABE8.8-VRQR and sgRNA_363; BE4-VRQR and sgRNA_363; BE4-VRQR and sgRNA_364; saABE8.8 and sgRNA_365; saBE4 and sgRNA_365; saBE4-KKH and sgRNA_366, ABE-bhCas12b and sgRNA_367; spCas9-ABE and sgRNA_375; spCas9-VRQR-ABE and sgRNA_376; or saCas9-ABE and sgRNA_377. The PAM sequence of spCas9-ABE can be AGG. The PAM sequence of spCas9-VRQR-ABE can be GGA. The PAM sequence of saCas9-ABE can be AGGAAT.

In certain embodiments, the fusion proteins provided herein comprise one or more features that improve the base editing activity of the fusion proteins. For example, any of the fusion proteins provided herein may comprise a Cas9 domain that has reduced nuclease activity. In some embodiments, any of the fusion proteins provided herein may have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-methylated) strand opposite the targeted nucleobase. Mutation of the catalytic residue (e.g., D10 to A10) prevents cleavage of the edited strand containing the targeted A residue. Such Cas9 variants can generate a single-strand DNA break (nick) at a specific location based on the gRNA-defined target sequence, leading to repair of the non-edited strand, ultimately resulting in a nucleobase change on the non-edited strand.

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase or cytidine deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain can comprise an endonuclease or an exonuclease. An endonuclease can cleave a single strand of a double-stranded nucleic acid or both strands of a double-stranded nucleic acid molecule. In some embodiments, a nuclease domain of a polynucleotide programmable nucleotide binding domain can cut zero, one, or two strands of a target polynucleotide.

Non-limiting examples of a polynucleotide programmable nucleotide binding domain which can be incorporated into a base editor include a CRISPR protein-derived domain, a restriction nuclease, a meganuclease, TAL nuclease (TALEN), and a zinc finger nuclease (ZFN). In some embodiments, a base editor comprises a polynucleotide programmable nucleotide binding domain comprising a natural or modified protein or portion thereof which via a bound guide nucleic acid is capable of binding to a nucleic acid sequence during CRISPR (i.e., Clustered Regularly Interspaced Short Palindromic Repeats)-mediated modification of a nucleic acid. Such a protein is referred to herein as a “CRISPR protein.” Accordingly, disclosed herein is a base editor comprising a polynucleotide programmable nucleotide binding domain comprising all or a portion of a CRISPR protein (i.e. a base editor comprising as a domain all or a portion of a CRISPR protein, also referred to as a “CRISPR protein-derived domain” of the base editor). A CRISPR protein-derived domain incorporated into a base editor can be modified compared to a wild-type or natural version of the CRISPR protein. For example, as described below a CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2c1 (e.g., SEQ ID NO: 236), Cas12c/C2c3, Cas12d/CasY, Cas12e/CasX, Cas12g, Cas12h, Cas12i, and Cas12j/Cas(D, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect, to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof. In some embodiments, a CRISPR protein-derived domain of a base editor can include all or a portion of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Cas9 nuclease sequences and structures are well known to those of skill in the art (See, e.g., “Complete genome sequence of an Ml strain of Streptococcus pyogenes.” Ferretti et al., Proc. Natl. Acad. Sci. U.S.A. 98:4658-4663(2001); “CRISPR RNA maturation by trans-encoded small RNA and host factor RNase III.” Deltcheva E., et al., Nature 471:602-607(2011); and “A programmable dual-RNA-guided DNA endonuclease in adaptive bacterial immunity.” Jinek M., et al., Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

### High Fidelity Cas9 Domains

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 237. In some embodiments, high fidelity Cas9 domains are engineered Cas9 domains comprising one or more mutations that decrease electrostatic interactions between the Cas9 domain and the sugar-phosphate backbone of a DNA, relative to a corresponding wild-type Cas9 domain. High fidelity Cas9 domains that have decreased electrostatic interactions with the sugar-phosphate backbone of DNA have less off-target effects. In some embodiments, the Cas9 domain (e.g., a wild type Cas9 domain (SEQ ID NOs: 201 and 204)) comprises one or more mutations that decrease the association between the Cas9 domain and the sugar-phosphate backbone of a DNA. In some embodiments, a Cas9 domain comprises one or more mutations that decreases the association between the Cas9 domain and the sugar-phosphate backbone of DNA by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, or at least 70%.

In some embodiments, any of the Cas9 fusion proteins provided herein comprise one or more of a D10A, N497X, a R661X, a Q695X, and/or a Q926X mutation, or a corresponding mutation in any of the amino acid sequences provided herein, wherein X is any amino acid. In some embodiments, the high fidelity Cas9 enzyme is SpCas9(K855A), eSpCas9(1.1), SpCas9-HF1, or hyper accurate Cas9 variant (HypaCas9). In some embodiments, the modified Cas9 eSpCas9(1.1) contains alanine substitutions that weaken the interactions between the HNH/RuvC groove and the non-target DNA strand, preventing strand separation and cutting at off-target sites. Similarly, SpCas9-HF1 lowers off-target editing through alanine substitutions that disrupt Cas9's interactions with the DNA phosphate backbone. HypaCas9 contains mutations (SpCas9 N692A/M694A/Q695A/H698A) in the REC3 domain that increase Cas9 proofreading and target discrimination. All three high fidelity enzymes generate less off-target editing than wildtype Cas9.

Cas9 Domains with Reduced Exclusivity

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. This may limit the ability to edit desired bases within a genome. In some embodiments, the base editing fusion proteins provided herein may need to be placed at a precise location, for example a region comprising a target base that is upstream of the PAM. See e.g., Komor, A. C., et al., “Programmable editing of a target base in genomic DNA without double-stranded DNA cleavage” Nature 533, 420-424 (2016), the entire contents of which are hereby incorporated by reference. Exemplary polypeptide sequences for spCas9 proteins capable of binding a PAM sequence are provided in the Sequence Listing as SEQ ID NOs: 201, 205, and 238-241 Accordingly, in some embodiments, any of the fusion proteins provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

### Nickases

In some embodiments, the polynucleotide programmable nucleotide binding domain can comprise a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). In some embodiments, a nickase can be derived from a fully catalytically active (e.g., natural) form of a polynucleotide programmable nucleotide binding domain by introducing one or more mutations into the active polynucleotide programmable nucleotide binding domain. For example, where a polynucleotide programmable nucleotide binding domain comprises a nickase domain derived from Cas9, the Cas9-derived nickase domain can include a D10A mutation and a histidine at position 840. In such embodiments, the residue H840 retains catalytic activity and can thereby cleave a single strand of the nucleic acid duplex. In another example, a Cas9-derived nickase domain can comprise an H840A mutation, while the amino acid residue at position 10 remains a D. In some embodiments, a nickase can be derived from a fully catalytically active (e.g., natural) form of a polynucleotide programmable nucleotide binding domain by removing all or a portion of a nuclease domain that is not required for the nickase activity. For example, where a polynucleotide programmable nucleotide binding domain comprises a nickase domain derived from Cas9, the Cas9-derived nickase domain can comprise a deletion of all or a portion of the RuvC domain or the HNH domain.

In some embodiments, wild-type Cas9 corresponds to, or comprises the following amino acid sequence:

(SEQ ID NO: 201)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKERGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRENASLGTYHDLLKI
IKDKDFLDNEENEDILEDIVLTLTLFEDREMIEERLKTYAHLEDDKVMKQ
LKRRRYTGWGRLSRKLINGIRDKQSGKTILDFLKSDGFANRNEMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDERKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD (single underline: HNH domain;
double underline: RuvC domain).

In some embodiments, the strand of a nucleic acid duplex target polynucleotide sequence that is cleaved by a base editor comprising a nickase domain (e.g., Cas9-derived nickase domain, Cas12-derived nickase domain) is the strand that is not edited by the base editor (i.e., the strand that is cleaved by the base editor is opposite to a strand comprising a base to be edited). In other embodiments, a base editor comprising a nickase domain (e.g., Cas9-derived nickase domain, Cas12-derived nickase domain) can cleave the strand of a DNA molecule which is being targeted for editing. In such embodiments, the non-targeted strand is not cleaved.

In some embodiments, a Cas9 nuclease has an inactive (e.g., an inactivated) DNA cleavage domain, that is, the Cas9 is a nickase, referred to as an “nCas9” protein (for “nickase” Cas9). The Cas9 nickase may be a Cas9 protein that is capable of cleaving only one strand of a duplexed nucleic acid molecule (e.g., a duplexed DNA molecule). In some embodiments the Cas9 nickase cleaves the target strand of a duplexed nucleic acid molecule, meaning that the Cas9 nickase cleaves the strand that is base paired to (complementary to) a gRNA (e.g., an sgRNA) that is bound to the Cas9. In some embodiments, a Cas9 nickase comprises a D10A mutation and has a histidine at position 840. In some embodiments the Cas9 nickase cleaves the non-target, non-base-edited strand of a duplexed nucleic acid molecule, meaning that the Cas9 nickase cleaves the strand that is not base paired to a gRNA (e.g., an sgRNA) that is bound to the Cas9. In some embodiments, a Cas9 nickase comprises an H840A mutation and has an aspartic acid residue at position 10, or a corresponding mutation. In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field, and are within the scope of this disclosure.

The amino acid sequence of an exemplary catalytically Cas9 nickase (nCas9) is as follows:

(SEQ ID NO: 205)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESELVEEDKKHERHPIFGNIVDEVAYHEKYPTIYHLRKKLVDSTDKAD
LRLIYLALAHMIKERGHFLIEGDLNPDNSDVDKLFIQLVQTYNQLFEENP
INASGVDAKAILSARLSKSRRLENLIAQLPGEKKNGLFGNLIALSLGLTP
NFKSNEDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTEDNGSIPHQIHLGELHAILRRQEDFYPFLKDNREKIEKILTFRIPY
YVGPLARGNSRFAWMTRKSEETITPWNFEEVVDKGASAQSFIERMTNEDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDELDNEENEDILEDIVLTLTLFEDREMIEERLKTYAHLFDDKVMKQ
LKRRRYTGWGRLSRKLINGIRDKQSGKTILDELKSDGFANRNEMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDERKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

The Cas9 nuclease has two functional endonuclease domains: RuvC and HNH. Cas9 undergoes a conformational change upon target binding that positions the nuclease domains to cleave opposite strands of the target DNA. The end result of Cas9-mediated DNA cleavage is a double-strand break (DSB) within the target DNA (˜3-4 nucleotides upstream of the PAM sequence). The resulting DSB is then repaired by one of two general repair pathways: (1) the efficient but error-prone non-homologous end joining (NHEJ) pathway; or (2) the less efficient but high-fidelity homology directed repair (HDR) pathway.

The “efficiency” of non-homologous end joining (NHEJ) and/or homology directed repair (HDR) can be calculated by any convenient method. For example, in some embodiments, efficiency can be expressed in terms of percentage of successful HDR. For example, a surveyor nuclease assay can be used to generate cleavage products and the ratio of products to substrate can be used to calculate the percentage. For example, a surveyor nuclease enzyme can be used that directly cleaves DNA containing a newly integrated restriction sequence as the result of successful HDR. More cleaved substrate indicates a greater percent HDR (a greater efficiency of HDR). As an illustrative example, a fraction (percentage) of HDR can be calculated using the following equation [(cleavage products)/(substrate plus cleavage products)] (e.g., (b+c)/(a+b+c), where “a” is the band intensity of DNA substrate and “b” and “c” are the cleavage products).

In some embodiments, efficiency can be expressed in terms of percentage of successful NHEJ. For example, a T7 endonuclease I assay can be used to generate cleavage products and the ratio of products to substrate can be used to calculate the percentage NHEJ. T7 endonuclease I cleaves mismatched heteroduplex DNA which arises from hybridization of wild-type and mutant DNA strands (NHEJ generates small random insertions or deletions (indels) at the site of the original break). More cleavage indicates a greater percent NHEJ (a greater efficiency of NHEJ). As an illustrative example, a fraction (percentage) of NHEJ can be calculated using the following equation: (1−(1−(b+c)/(a+b+c))^{1/2})×100, where “a” is the band intensity of DNA substrate and “b” and “c” are the cleavage products (Ran et. al., Cell. 2013 Sep. 12; 154(6):1380-9; and Ran et al., Nat Protoc. 2013 November; 8(11): 2281-2308).

The NHEJ repair pathway is the most active repair mechanism, and it frequently causes small nucleotide insertions or deletions (indels) at the DSB site. The randomness of NHEJ-mediated DSB repair has important practical implications because a population of cells expressing Cas9 and a gRNA or a guide polynucleotide can result in a diverse array of mutations. In most embodiments, NHEJ gives rise to small indels in the target DNA that result in amino acid deletions, insertions, or frameshift mutations leading to premature stop codons within the open reading frame (ORF) of the targeted gene. The ideal end result is a loss-of-function mutation within the targeted gene.

While NHEJ-mediated DSB repair often disrupts the open reading frame of the gene, homology directed repair (HDR) can be used to generate specific nucleotide changes ranging from a single nucleotide change to large insertions like the addition of a fluorophore or tag.

In order to utilize HDR for gene editing, a DNA repair template containing the desired sequence can be delivered into the cell type of interest with the gRNA(s) and Cas9 or Cas9 nickase. The repair template can contain the desired edit as well as additional homologous sequence immediately upstream and downstream of the target (termed left & right homology arms). The length of each homology arm can be dependent on the size of the change being introduced, with larger insertions requiring longer homology arms. The repair template can be a single-stranded oligonucleotide, double-stranded oligonucleotide, or a double-stranded DNA plasmid. The efficiency of HDR is generally low (<10% of modified alleles) even in cells that express Cas9, gRNA and an exogenous repair template. The efficiency of HDR can be enhanced by synchronizing the cells, since HDR takes place during the S and G2 phases of the cell cycle. Chemically or genetically inhibiting genes involved in NHEJ can also increase HDR frequency.

In some embodiments, Cas9 is a modified Cas9. A given gRNA targeting sequence can have additional sites throughout the genome where partial homology exists. These sites are called off-targets and need to be considered when designing a gRNA. In addition to optimizing gRNA design, CRISPR specificity can also be increased through modifications to Cas9. Cas9 generates double-strand breaks (DSBs) through the combined activity of two nuclease domains, RuvC and HNH. Cas9 nickase, a D10A mutant of SpCas9, retains one nuclease domain and generates a DNA nick rather than a DSB. The nickase system can also be combined with HDR-mediated gene editing for specific gene edits.

### Catalytically Dead Nucleases

Also provided herein are base editors comprising a polynucleotide programmable nucleotide binding domain which is catalytically dead (i.e., incapable of cleaving a target polynucleotide sequence). Herein the terms “catalytically dead” and “nuclease dead” are used interchangeably to refer to a polynucleotide programmable nucleotide binding domain which has one or more mutations and/or deletions resulting in its inability to cleave a strand of a nucleic acid. In some embodiments, a catalytically dead polynucleotide programmable nucleotide binding domain base editor can lack nuclease activity as a result of specific point mutations in one or more nuclease domains. For example, in the case of a base editor comprising a Cas9 domain, the Cas9 can comprise both a D10A mutation and an H840A mutation. Such mutations inactivate both nuclease domains, thereby resulting in the loss of nuclease activity. In other embodiments, a catalytically dead polynucleotide programmable nucleotide binding domain can comprise one or more deletions of all or a portion of a catalytic domain (e.g., RuvC1 and/or HNH domains). In further embodiments, a catalytically dead polynucleotide programmable nucleotide binding domain comprises a point mutation (e.g., D10A or H840A) as well as a deletion of all or a portion of a nuclease domain. dCas9 domains are known in the art and described, for example, in Qi et al., “Repurposing CRISPR as an RNA-guided platform for sequence-specific control of gene expression.” Cell. 2013; 152(5):1173-83, the entire contents of which are incorporated herein by reference.

Additional suitable nuclease-inactive dCas9 domains will be apparent to those of skill in the art based on this disclosure and knowledge in the field, and are within the scope of this disclosure. Such additional exemplary suitable nuclease-inactive Cas9 domains include, but are not limited to, D10A/H840A, D10A/D839A/H840A, and D10A/D839A/H840A/N863A mutant domains (See, e.g., Prashant et al., CAS9 transcriptional activators for target specificity screening and paired nickases for cooperative genome engineering. Nature Biotechnology. 2013; 31(9): 833-838, the entire contents of which are incorporated herein by reference).

In some embodiments, dCas9 corresponds to, or comprises in part or in whole, a Cas9 amino acid sequence having one or more mutations that inactivate the Cas9 nuclease activity. In some embodiments, the nuclease-inactive dCas9 domain comprises a D10X mutation and a H840X mutation of the amino acid sequence set forth herein, or a corresponding mutation in any of the amino acid sequences provided herein, wherein X is any amino acid change. In some embodiments, the nuclease-inactive dCas9 domain comprises a D10A mutation and a H840A mutation of the amino acid sequence set forth herein, or a corresponding mutation in any of the amino acid sequences provided herein. In some embodiments, a nuclease-inactive Cas9 domain comprises the amino acid sequence set forth in Cloning vector pPlatTET-gRNA2 (Accession No. BAV54124).

In some embodiments, a variant Cas9 protein can cleave the complementary strand of a guide target sequence but has reduced ability to cleave the non-complementary strand of a double stranded guide target sequence. For example, the variant Cas9 protein can have a mutation (amino acid substitution) that reduces the function of the RuvC domain. As a non-limiting example, in some embodiments, a variant Cas9 protein has a D10A (aspartate to alanine at amino acid position 10) and can therefore cleave the complementary strand of a double stranded guide target sequence but has reduced ability to cleave the non-complementary strand of a double stranded guide target sequence (thus resulting in a single strand break (SSB) instead of a double strand break (DSB) when the variant Cas9 protein cleaves a double stranded target nucleic acid) (see, for example, Jinek et al., Science. 2012 Aug. 17; 337(6096):816-21).

In some embodiments, a variant Cas9 protein can cleave the non-complementary strand of a double stranded guide target sequence but has reduced ability to cleave the complementary strand of the guide target sequence. For example, the variant Cas9 protein can have a mutation (amino acid substitution) that reduces the function of the HNH domain (RuvC/HNH/RuvC domain motifs). As a non-limiting example, in some embodiments, the variant Cas9 protein has an H840A (histidine to alanine at amino acid position 840) mutation and can therefore cleave the non-complementary strand of the guide target sequence but has reduced ability to cleave the complementary strand of the guide target sequence (thus resulting in a SSB instead of a DSB when the variant Cas9 protein cleaves a double stranded guide target sequence). Such a Cas9 protein has a reduced ability to cleave a guide target sequence (e.g., a single stranded guide target sequence) but retains the ability to bind a guide target sequence (e.g., a single stranded guide target sequence).

As another non-limiting example, in some embodiments, the variant Cas9 protein harbors W476A and W1126A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some embodiments, the variant Cas9 protein harbors P475A, W476A, N477A, D1125A, WI 126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some embodiments, the variant Cas9 protein harbors H840A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors H840A, D10A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, the variant Cas9 has restored catalytic His residue at position 840 in the Cas9 HNH domain (A840H).

As another non-limiting example, in some embodiments, the variant Cas9 protein harbors, H840A, P475A, W476A, N477A, D1125A, W 1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors D10A, H840A, P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, when a variant Cas9 protein harbors W476A and W1126A mutations or when the variant Cas9 protein harbors P475A, W476A, N477A, D1125A, W1126A, and D1127A mutations, the variant Cas9 protein does not bind efficiently to a PAM sequence. Thus, in some such embodiments, when such a variant Cas9 protein is used in a method of binding, the method does not require a PAM sequence. In other words, in some embodiments, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted). Also, mutations other than alanine substitutions are suitable.

In some embodiments, a variant Cas9 protein that has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the variant Cas9 protein can still bind to target DNA in a site-specific manner (because it is still guided to a target DNA sequence by a guide RNA) as long as it retains the ability to interact with the guide RNA.

In some embodiments, the variant Cas protein can be spCas9, spCas9-VRQR, spCas9-VRER, xCas9 (sp), saCas9, saCas9-KKH, spCas9-MQKSER, spCas9-LRKIQK, or spCas9-LRVSQL.

In some embodiments, the Cas9 domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 comprises a N579A mutation, or a corresponding mutation in any of the amino acid sequences provided in the Sequence Listing submitted herewith.

In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT or a NNGRRV PAM sequence. In some embodiments, the SaCas9 domain comprises one or more of a E781X, a N967X, and a R1014X mutation, or a corresponding mutation in any of the amino acid sequences provided herein, wherein X is any amino acid. In some embodiments, the SaCas9 domain comprises one or more of a E781K, a N967K, and a R1014H mutation, or one or more corresponding mutation in any of the amino acid sequences provided herein. In some embodiments, the SaCas9 domain comprises a E781K, a N967K, or a R1014H mutation, or corresponding mutations in any of the amino acid sequences provided herein.

In some embodiments, one of the Cas9 domains present in the fusion protein may be replaced with a guide nucleotide sequence-programmable DNA-binding protein domain that has no requirements for a PAM sequence. In some embodiments, the Cas9 is an SaCas9. Residue A579 of SaCas9 can be mutated from N579 to yield a SaCas9 nickase. Residues K781, K967, and H1014 can be mutated from E781, N967, and R1014 to yield a SaKKH Cas9.

In some embodiments, a modified SpCas9 including amino acid substitutions D1135M, S1136Q, G1218K, E1219F, A1322R, D1332A, R1335E, and T1337R (SpCas9-MQKFRAER) and having specificity for the altered PAM 5′-NGC-3′ was used.

Alternatives to S. pyogenes Cas9 can include RNA-guided endonucleases from the Cpf1 family that display cleavage activity in mammalian cells. CRISPR from Prevotella and Francisella 1 (CRISPR/Cpf1) is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations. Unlike Cas9 nucleases, the result of Cpf1-mediated DNA cleavage is a double-strand break with a short 3′ overhang. Cpf1 's staggered cleavage pattern can open up the possibility of directional gene transfer, analogous to traditional restriction enzyme cloning, which can increase the efficiency of gene editing. Like the Cas9 variants and orthologues described above, Cpf1 can also expand the number of sites that can be targeted by CRISPR to AT-rich regions or AT-rich genomes that lack the NGG PAM sites favored by SpCas9. The Cpf1 locus contains a mixed alpha/beta domain, a RuvC-I followed by a helical region, a RuvC-II and a zinc finger-like domain. The Cpf1 protein has a RuvC-like endonuclease domain that is similar to the RuvC domain of Cas9.

Furthermore, Cpf1, unlike Cas9, does not have a HNH endonuclease domain, and the N-terminal of Cpf1 does not have the alpha-helical recognition lobe of Cas9. Cpf1 CRISPR-Cas domain architecture shows that Cpf1 is functionally unique, being classified as Class 2, type V CRISPR system. The Cpf1 loci encode Cas1, Cas2 and Cas4 proteins that are more similar to types I and III than type II systems. Functional Cpf1 does not require the trans-activating CRISPR RNA (tracrRNA), therefore, only CRISPR (crRNA) is required. This benefits genome editing because Cpf1 is not only smaller than Cas9, but also it has a smaller sgRNA molecule (approximately half as many nucleotides as Cas9). The Cpf1-crRNA complex cleaves target DNA or RNA by identification of a protospacer adjacent motif 5′-YTN-3′ or 5′-TTN-3′ in contrast to the G-rich PAM targeted by Cas9. After identification of PAM, Cpf1 introduces a sticky-end-like DNA double-stranded break having an overhang of 4 or 5 nucleotides.

In some embodiments, the Cas9 is a Cas9 variant having specificity for an altered PAM sequence. In some embodiments, the Additional Cas9 variants and PAM sequences are described in Miller, S. M., et al. Continuous evolution of SpCas9 variants compatible with non-G PAMs, Nat. Biotechnol. (2020), the entirety of which is incorporated herein by reference. in some embodiments, a Cas9 variate have no specific PAM requirements. In some embodiments, a Cas9 variant, e.g. a SpCas9 variant has specificity for a NRNH PAM, wherein R is A or G and H is A, C, or T. In some embodiments, the SpCas9 variant has specificity for a PAM sequence AAA, TAA, CAA, GAA, TAT, GAT, or CAC. In some embodiments, the SpCas9 variant comprises an amino acid substitution at position 1114, 1134, 1135, 1137, 1139, 1151, 1180, 1188, 1211, 1218, 1219, 1221, 1249, 1256, 1264, 1290, 1318, 1317, 1320, 1321, 1323, 1332, 1333, 1335, 1337, or 1339 or a corresponding position thereof. In some embodiments, the SpCas9 variant comprises an amino acid substitution at position 1114, 1135, 1218, 1219, 1221, 1249, 1320, 1321, 1323, 1332, 1333, 1335, or 1337 or a corresponding position thereof. In some embodiments, the SpCas9 variant comprises an amino acid substitution at position 1114, 1134, 1135, 1137, 1139, 1151, 1180, 1188, 1211, 1219, 1221, 1256, 1264, 1290, 1318, 1317, 1320, 1323, 1333 or a corresponding position thereof. In some embodiments, the SpCas9 variant comprises an amino acid substitution at position 1114, 1131, 1135, 1150, 1156, 1180, 1191, 1218, 1219, 1221, 1227, 1249, 1253, 1286, 1293, 1320, 1321, 1332, 1335, 1339 or a corresponding position thereof. In some embodiments, the SpCas9 variant comprises an amino acid substitution at position 1114, 1127, 1135, 1180, 1207, 1219, 1234, 1286, 1301, 1332, 1335, 1337, 1338, 1349 or a corresponding position thereof. Exemplary amino acid substitutions and PAM specificity of SpCas9 variants are shown in Tables 3A-3D)

TABLE 3A
SpCas9 Variants and PAM specificity

| | SpCas9 amino acid position | | | | | | | | | | | | |
|---|---|---|---|---|---|---|---|---|---|---|---|---|---|
| | 1114 | 1135 | 1218 | 1219 | 1221 | 1249 | 1320 | 1321 | 1323 | 1332 | 1333 | 1335 | 1337 |
| PAM | R | D | G | E | Q | P | A | P | A | D | R | R | T |
| AAA | | N | | V | H | | | | | | G | | |
| AAA | | N | | V | H | | | | | | G | | |
| AAA | | | | V | | | | | | | G | | |
| TAA | G | N | | V | | | | | | | I | | |
| TAA | | N | | V | | | | | | | I | | A |
| TAA | G | N | | V | | | | | | | I | | A |
| CAA | | | | V | | | | | | | K | | |
| CAA | | N | | V | | | | | | | K | | |
| CAA | | N | | V | | | | | | | K | | |
| GAA | | | | V | H | | V | | | | K | | |
| GAA | | N | | V | | | V | | | | K | | |
| GAA | | | | V | H | | V | | | | K | | |
| TAT | | | S | V | H | S | | S | | | | L | |
| TAT | | | S | V | H | S | | S | | | | L | |
| TAT | | | S | V | H | S | | S | | | | L | |
| GAT | | | | V | | | | | | | I | | |
| GAT | | | | V | | | | | D | | | Q | |
| GAT | | | | V | | | | | D | | | Q | |
| CAC | | | | V | | | | | | N | | Q | N |
| CAC | | N | | V | | | | | | | | Q | N |
| CAC | | | | V | | | | | | N | | Q | N |

TABLE 3B
SpCas9 Variants and PAM specificity

| | SpCas9 amino acid position | | | | | | | | | | | | | | | | | | |
|---|---|---|---|---|---|---|---|---|---|---|---|---|---|---|---|---|---|---|---|
| | 1114 | 1134 | 1135 | 1137 | 1139 | 1151 | 1180 | 1188 | 1211 | 1219 | 1221 | 1256 | 1264 | 1290 | 1318 | 1317 | 1320 | 1323 | 1333 |
| PAM | R | F | D | P | V | K | D | K | K | E | Q | Q | H | V | L | N | A | A | R |
| GAA | | | | | | | | | | V | H | | | | | | V | | K |
| GAA | | | N | S | | | | | | V | | | | | | | V | D | K |
| GAA | | | N | | | | | | | V | H | | Y | | | | V | | K |
| CAA | | | N | | | | | | | V | H | | Y | | | | V | | K |
| CAA | G | | N | S | | | | | | V | H | | Y | | | | V | | K |
| CAA | | | N | | | | | R | | V | H | | | | | | V | | K |
| CAA | | | N | | | | G | | R | V | H | | Y | | | | V | | K |
| CAA | | | N | | | | | | | V | H | | Y | | | | V | | K |
| AAA | | | N | | | | G | | | V | H | R | Y | | | | V | D | K |
| CAA | G | | N | | | | G | | | V | H | | Y | | | | V | D | K |
| CAA | | L | N | | | | G | | | V | H | | Y | | | T | V | D | K |
| TAA | G | | N | | | | G | | | V | H | | Y | G | S | | V | D | K |
| TAA | G | | N | | | E | G | | | V | H | | Y | | S | | V | | K |
| TAA | G | | N | | | | G | | | V | H | | Y | | S | | V | D | K |
| TAA | G | | N | | | | G | | R | V | H | | | | | | V | | K |
| TAA | | | N | | | | G | | R | V | H | | Y | | | | V | | K |
| TAA | G | | N | | A | | G | | | V | H | | | | | | V | | K |
| TAA | G | | N | | | | | | | V | H | | | | | | V | | K |

TABLE 3C
SpCas9 Variants and PAM specificity

| | SpCas9 amino acid position | | | | | | | | | | | | | | | | | | | |
|---|---|---|---|---|---|---|---|---|---|---|---|---|---|---|---|---|---|---|---|---|
| | 1114 | 1131 | 1135 | 1150 | 1156 | 1180 | 1191 | 1218 | 1219 | 1221 | 1227 | 1249 | 1253 | 1286 | 1293 | 1320 | 1321 | 1332 | 1335 | 1339 |
| PAM | R | Y | D | E | K | D | K | G | E | Q | A | P | E | N | A | A | P | D | R | T |
| SacB. | | | N | | | | N | | V | H | | | | | | V | S | | L | |
| TAT | | | | | | | | | | | | | | | | | | | | |
| SacB. | | | N | | | | | S | V | H | | S | | | | | S | G | L | |
| TAT | | | | | | | | | | | | | | | | | | | | |
| AAT | | | N | | | | | S | V | H | V | S | | K | T | | S | G | L | I |
| TAT | G | | N | | | G | | S | V | H | | S | K | | | | S | G | L | |
| TAT | G | | N | | | G | | S | V | H | | S | | | | | S | G | L | |
| TAT | G | C | N | | | G | | S | V | H | | S | | | | | S | G | L | |
| TAT | G | C | N | | | G | | S | V | H | | S | | | | | S | G | L | |
| TAT | G | C | N | | | G | | S | V | H | | S | | | | | S | G | L | |
| TAT | G | C | N | | E | G | | S | V | H | | S | | | | | S | G | L | |
| TAT | G | C | N | V | | G | | S | V | H | | S | | | | | S | G | L | |
| TAT | | C | N | | | G | | S | V | H | | S | | | | | S | G | L | |
| TAT | G | C | N | | | G | | S | V | H | | S | | | | | S | G | L | |

TABLE 3D
SpCas9 Variants and PAM specificity

| | SpCas9 amino acid position | | | | | | | | | | | | | |
|---|---|---|---|---|---|---|---|---|---|---|---|---|---|---|
| | 1114 | 1127 | 1135 | 1180 | 1207 | 1219 | 1234 | 1286 | 1301 | 1332 | 1335 | 1337 | 1338 | 1349 |
| PAM | R | D | D | D | E | E | N | N | P | D | R | T | S | H |
| SacB.CAC | | | N | | | V | | | | N | Q | N | | |
| AAC | G | | N | | | V | | | | N | Q | N | | |
| AAC | G | | N | | | V | | | | N | Q | N | | |
| TAC | G | | N | | | V | | | | N | Q | N | | |
| TAC | G | | N | | | V | | H | | N | Q | N | | |
| TAC | G | | N | | G | V | D | H | | N | Q | N | | |
| TAC | G | | N | | | V | | | | N | Q | N | | |
| TAC | G | G | N | E | | V | | H | | N | Q | N | | |
| TAC | G | | N | | | V | | H | | N | Q | N | | |
| TAC | G | | N | | | V | | | | N | Q | N | T | R |

Further exemplary Cas9 (e.g., SaCas9) polypeptides with modified PAM recognition are described in Kleinstiver, et al. “Broadening the targeting range of Staphylococcus aureus CRISPR-Cas9 by modifying PAM recognition,” Nature Biotechnology, 33:1293-1298 (2015) DOI: 10.1038/nbt.3404, the disclosure of which is incorporated herein by reference in its entirety for all purposes. In some embodiments, a Cas9 variant (e.g., a SaCas9 variant) comprising one or more of the alterations E782K, N929R, N968K, and/or R1015H has specificity for, or is associated with increased editing activities relative to a reference polypeptide (e.g., SaCas9) at an NNNRRT or NNHRRT PAM sequence, where N represents any nucleotide, H represents any nucleotide other than G (i.e., “not G”), and R represents a purine. In embodiments, the Cas9 variant (e.g., a SaCas9 variant) comprises the alterations E782K, N968K, and R1015H or the alterations E782K, K929R, and R1015H.

In some embodiments, the nucleic acid programmable DNA binding protein (napDNAbp) is a single effector of a microbial CRISPR-Cas system. Single effectors of microbial CRISPR-Cas systems include, without limitation, Cas9, Cpf1, Cas12b/C2c1, and Cas12c/C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (Cas12b/C2c1, and Cas12c/C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, Cas12b/C2c1, and Cas12c/C2c3, contain RuvC-like endonuclease domains related to Cpf1. A third system contains an effector with two predicated HEPN RNase domains. Production of mature CRISPR RNA is tracrRNA-independent, unlike production of CRISPR RNA by Cas12b/C2c1. Cas12b/C2c1 depends on both CRISPR RNA and tracrRNA for DNA cleavage.

In some embodiments, the napDNAbp is a circular permutant (e.g., SEQ ID NO: 242).

The crystal structure of Alicyclobaccillus acidoterrastris Cas12b/C2c1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with Cas12b/C2c1-mediated cleavage resulting in a staggered seven-nucleotide break of target DNA. Structural comparisons between Cas12b/C2c1 ternary complexes and previously identified Cas9 and Cpf1 counterparts demonstrate the diversity of mechanisms used by CRISPR-Cas9 systems.

In some embodiments, the nucleic acid programmable DNA binding protein (napDNAbp) of any of the fusion proteins provided herein may be a Cas12b/C2c1, or a Cas12c/C2c3 protein. In some embodiments, the napDNAbp is a Cas12b/C2c1 protein. In some embodiments, the napDNAbp is a Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12b/C2c1 or Cas12c/C2c3 protein. In some embodiments, the napDNAbp is a naturally-occurring Cas12b/C2c1 or Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of the napDNAbp sequences provided herein. It should be appreciated that Cas12b/C2c1 or Cas12c/C2c3 from other bacterial species may also be used in accordance with the present disclosure.

In some embodiments, a napDNAbp refers to Cas12c. In some embodiments, the Cas12c protein is a Cas12c1 (SEQ ID NO: 243) or a variant of Cas12c1. In some embodiments, the Cas12 protein is a Cas12c2 (SEQ ID NO: 244) or a variant of Cas12c2. In some embodiments, the Cas12 protein is a Cas12c protein from Oleiphilus sp. H10009 (i.e., OspCas12c; SEQ ID NO: 245) or a variant of OspCas12c. These Cas12c molecules have been described in Yan et al., “Functionally Diverse Type V CRISPR-Cas Systems,” Science, 2019 Jan. 4; 363: 88-91; the entire contents of which is hereby incorporated by reference. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12c1, Cas12c2, or OspCas12c protein. In some embodiments, the napDNAbp is a naturally-occurring Cas12c1, Cas12c2, or OspCas12c protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any Cas12c1, Cas12c2, or OspCas12c protein described herein. It should be appreciated that Cas12c1, Cas12c2, or OspCas12c from other bacterial species may also be used in accordance with the present disclosure.

In some embodiments, a napDNAbp refers to Cas12g, Cas12h, or Cas12i, which have been described in, for example, Yan et al., “Functionally Diverse Type V CRISPR-Cas Systems,” Science, 2019 Jan. 4; 363: 88-91; the entire contents of each is hereby incorporated by reference. Exemplary Cas12g, Cas12h, and Cas12i polypeptide sequences are provided in the Sequence Listing as SEQ ID NOs: 246-249. By aggregating more than 10 terabytes of sequence data, new classifications of Type V Cas proteins were identified that showed weak similarity to previously characterized Class V protein, including Cas12g, Cas12h, and Cas12i. In some embodiments, the Cas12 protein is a Cas12g or a variant of Cas12g. In some embodiments, the Cas12 protein is a Cas12h or a variant of Cas12h. In some embodiments, the Cas12 protein is a Cas12i or a variant of Cas12i. It should be appreciated that other RNA-guided DNA binding proteins may be used as a napDNAbp, and are within the scope of this disclosure. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12g, Cas12h, or Cas12i protein. In some embodiments, the napDNAbp is a naturally-occurring Cas12g, Cas12h, or Cas12i protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any Cas12g, Cas12h, or Cas12i protein described herein. It should be appreciated that Cas12g, Cas12h, or Cas12i from other bacterial species may also be used in accordance with the present disclosure. In some embodiments, the Cas12i is a Cas12i1 or a Cas12i2.

In some embodiments, the nucleic acid programmable DNA binding protein (napDNAbp) of any of the fusion proteins provided herein may be a Cas12j/CasΦ protein. Cas12j/CasΦ is described in Pausch et al., “CRISPR-CasΦ from huge phages is a hypercompact genome editor,” Science, 17 Jul. 2020, Vol. 369, Issue 6501, pp. 333-337, which is incorporated herein by reference in its entirety. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12j/CasΦ protein. In some embodiments, the napDNAbp is a naturally-occurring Cas12j/CasΦ protein. In some embodiments, the napDNAbp is a nuclease inactive (“dead”) Cas12j/CasΦ protein. It should be appreciated that Cas12j/CasΦ from other species may also be used in accordance with the present disclosure.

Fusion Proteins with Internal Insertion

Provided herein are fusion proteins comprising a heterologous polypeptide fused to a nucleic acid programmable nucleic acid binding protein, for example, a napDNAbp. A heterologous polypeptide can be a polypeptide that is not found in the native or wild-type napDNAbp polypeptide sequence. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine of adenosine deaminase) or a functional fragment thereof. For example, a fusion protein can comprise a deaminase flanked by an N-terminal fragment and a C-terminal fragment of a Cas9 or Cas12 (e.g., Cas12b/C2c1), polypeptide. In some embodiments, the cytidine deaminase is an APOBEC deaminase (e.g., APOBEC1). In some embodiments, the adenosine deaminase is a TadA (e.g., TadA*7.10 or TadA*8). In some embodiments, the TadA is a TadA*8 or a TadA*9. TadA sequences (e.g., TadA7.10 or TadA*8) as described herein are suitable deaminases for the above-described fusion proteins.

In some embodiments, the fusion protein comprises the structure:
NH2-[N-terminal fragment of a napDNAbp]-[deaminase]-[C-terminal fragment of a napDNAbp]-COOH;
NH2-[N-terminal fragment of a Cas9]-[adenosine deaminase]-[C-terminal fragment of a Cas9]-COOH;
NH2-[N-terminal fragment of a Cas12]-[adenosine deaminase]-[C-terminal fragment of a Cas12]-COOH;
NH2-[N-terminal fragment of a Cas9]-[cytidine deaminase]-[C-terminal fragment of a Cas9]-COOH;
NH2-[N-terminal fragment of a Cas12]-[cytidine deaminase]-[C-terminal fragment of a Cas12]-COOH;
   wherein each instance of “]-[” is an optional linker.

The deaminase can be a circular permutant deaminase. For example, the deaminase can be a circular permutant adenosine deaminase. In some embodiments, the deaminase is a circular permutant TadA, circularly permutated at amino acid residue 116, 136, or 65 as numbered in the TadA reference sequence.

The fusion protein can comprise more than one deaminase. The fusion protein can comprise, for example, 1, 2, 3, 4, 5 or more deaminases. In some embodiments, the fusion protein comprises one or two deaminase. The two or more deaminases in a fusion protein can be an adenosine deaminase, a cytidine deaminase, or a combination thereof. The two or more deaminases can be homodimers or heterodimers. The two or more deaminases can be inserted in tandem in the napDNAbp. In some embodiments, the two or more deaminases may not be in tandem in the napDNAbp.

In some embodiments, the napDNAbp in the fusion protein is a Cas9 polypeptide or a fragment thereof. The Cas9 polypeptide can be a variant Cas9 polypeptide. In some embodiments, the Cas9 polypeptide is a Cas9 nickase (nCas9) polypeptide or a fragment thereof. In some embodiments, the Cas9 polypeptide is a nuclease dead Cas9 (dCas9) polypeptide or a fragment thereof. The Cas9 polypeptide in a fusion protein can be a full-length Cas9 polypeptide. In some cases, the Cas9 polypeptide in a fusion protein may not be a full length Cas9 polypeptide. The Cas9 polypeptide can be truncated, for example, at a N-terminal or C-terminal end relative to a naturally-occurring Cas9 protein. The Cas9 polypeptide can be a circularly permuted Cas9 protein. The Cas9 polypeptide can be a fragment, a portion, or a domain of a Cas9 polypeptide, that is still capable of binding the target polynucleotide and a guide nucleic acid sequence.

In some embodiments, the Cas9 polypeptide is a Streptococcus pyogenes Cas9 (SpCas9), Staphylococcus aureus Cas9 (SaCas9), Streptococcus thermophilus 1 Cas9 (St1Cas9), or fragments or variants of any of the Cas9 polypeptides described herein.

In some embodiments, the fusion protein comprises an adenosine deaminase domain and a cytidine deaminase domain inserted within a Cas9. In some embodiments, an adenosine deaminase is fused within a Cas9 and a cytidine deaminase is fused to the C-terminus. In some embodiments, an adenosine deaminase is fused within Cas9 and a cytidine deaminase fused to the N-terminus. In some embodiments, a cytidine deaminase is fused within Cas9 and an adenosine deaminase is fused to the C-terminus. In some embodiments, a cytidine deaminase is fused within Cas9 and an adenosine deaminase fused to the N-terminus.

Exemplary structures of a fusion protein with an adenosine deaminase and a cytidine deaminase and a Cas9 are provided as follows:
NH2-[Cas9(adenosine deaminase)]-[cytidine deaminase]-COOH;
NH2-[cytidine deaminase]-[Cas9(adenosine deaminase)]-COOH;
NH2-[Cas9(cytidine deaminase)]-[adenosine deaminase]-COOH; or
NH2-[adenosine deaminase]-[Cas9(cytidine deaminase)]-COOH.

In some embodiments, the “-” used in the general architecture above indicates the presence of an optional linker.

In various embodiments, the catalytic domain has DNA modifying activity (e.g., deaminase activity), such as adenosine deaminase activity. In some embodiments, the adenosine deaminase is a TadA (e.g., TadA*7.10). In some embodiments, the TadA is a TadA*8. In some embodiments, a TadA*8 is fused within Cas9 and a cytidine deaminase is fused to the C-terminus. In some embodiments, a TadA*8 is fused within Cas9 and a cytidine deaminase fused to the N-terminus. In some embodiments, a cytidine deaminase is fused within Cas9 and a TadA*8 is fused to the C-terminus. In some embodiments, a cytidine deaminase is fused within Cas9 and a TadA*8 fused to the N-terminus. Exemplary structures of a fusion protein with a TadA*8 and a cytidine deaminase and a Cas9 are provided as follows:
NH2-[Cas9(TadA*8)]-[cytidine deaminase]-COOH;
NH2-[cytidine deaminase]-[Cas9(TadA*8)]-COOH;
NH2-[Cas9(cytidine deaminase)]-[TadA*8]-COOH; or
NH2-[TadA*8]-[Cas9(cytidine deaminase)]-COOH.

In some embodiments, the “-” used in the general architecture above indicates the presence of an optional linker.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase) can be inserted into a napDNAbp without compromising function of the deaminase (e.g., base editing activity) or the napDNAbp (e.g., ability to bind to target nucleic acid and guide nucleic acid). A deaminase (e.g., adenosine deaminase, cytidine deaminase, or adenosine deaminase and cytidine deaminase) can be inserted in the napDNAbp at, for example, a disordered region or a region comprising a high temperature factor or B-factor as shown by crystallographic studies. Regions of a protein that are less ordered, disordered, or unstructured, for example solvent exposed regions and loops, can be used for insertion without compromising structure or function. A deaminase (e.g., adenosine deaminase, cytidine deaminase, or adenosine deaminase and cytidine deaminase) can be inserted in the napDNAbp in a flexible loop region or a solvent-exposed region. In some embodiments, the deaminase (e.g., adenosine deaminase, cytidine deaminase, or adenosine deaminase and cytidine deaminase) is inserted in a flexible loop of the Cas9 or the Cas12b/C2c1 polypeptide.

In some embodiments, the insertion location of a deaminase (e.g., adenosine deaminase, cytidine deaminase, or adenosine deaminase and cytidine deaminase) is determined by B-factor analysis of the crystal structure of Cas9 polypeptide. In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). B-factor or temperature factor can indicate the fluctuation of atoms from their average position (for example, as a result of temperature-dependent atomic vibrations or static disorder in a crystal lattice). A high B-factor (e.g., higher than average B-factor) for backbone atoms can be indicative of a region with relatively high local mobility. Such a region can be used for inserting a deaminase without compromising structure or function. A deaminase (e.g., adenosine deaminase, cytidine deaminase, or adenosine deaminase and cytidine deaminase) can be inserted at a location with a residue having a Cα atom with a B-factor that is 50%, 60%, 70%, 80%, 90%, 100%, 110%, 120%, 130%, 140%, 150%, 160%, 170%, 180%, 190%, 200%, or greater than 200% more than the average B-factor for the total protein. A deaminase (e.g., adenosine deaminase, cytidine deaminase, or adenosine deaminase and cytidine deaminase) can be inserted at a location with a residue having a Cα atom with a B-factor that is 50%, 60%, 70%, 80%, 90%, 100%, 110%, 120%, 130%, 140%, 150%, 160%, 170%, 180%, 190%,200% or greater than 200% more than the average B-factor for a Cas9 protein domain comprising the residue. Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

A heterologous polypeptide (e.g., deaminase) can be inserted in the napDNAbp at an amino acid residue selected from the group consisting of: 768, 791, 792, 1015, 1016, 1022, 1023, 1026, 1029, 1040, 1052, 1054, 1067, 1068, 1069, 1246, 1247, and 1248 as numbered in the above Cas9 reference sequence, or a corresponding amino acid residue in another Cas9 polypeptide. In some embodiments, the heterologous polypeptide is inserted between amino acid positions 768-769, 791-792, 792-793, 1015-1016, 1022-1023, 1026-1027, 1029-1030, 1040-1041, 1052-1053, 1054-1055, 1067-1068, 1068-1069, 1247-1248, or 1248-1249 as numbered in the above Cas9 reference sequence or corresponding amino acid positions thereof. In some embodiments, the heterologous polypeptide is inserted between amino acid positions 769-770, 792-793, 793-794, 1016-1017, 1023-1024, 1027-1028, 1030-1031, 1041-1042, 1053-1054, 1055-1056, 1068-1069, 1069-1070, 1248-1249, or 1249-1250 as numbered in the above Cas9 reference sequence or corresponding amino acid positions thereof. In some embodiments, the heterologous polypeptide replaces an amino acid residue selected from the group consisting of: 768, 791, 792, 1015, 1016, 1022, 1023, 1026, 1029, 1040, 1052, 1054, 1067, 1068, 1069, 1246, 1247, and 1248 as numbered in the above Cas9 reference sequence, or a corresponding amino acid residue in another Cas9 polypeptide. It should be understood that the reference to the above Cas9 reference sequence with respect to insertion positions is for illustrative purposes. The insertions as discussed herein are not limited to the Cas9 polypeptide sequence of the above Cas9 reference sequence, but include insertion at corresponding locations in variant Cas9 polypeptides, for example a Cas9 nickase (nCas9), nuclease dead Cas9 (dCas9), a Cas9 variant lacking a nuclease domain, a truncated Cas9, or a Cas9 domain lacking partial or complete HNH domain.

A heterologous polypeptide (e.g., deaminase) can be inserted in the napDNAbp at an amino acid residue selected from the group consisting of: 768, 792, 1022, 1026, 1040, 1068, and 1247 as numbered in the above Cas9 reference sequence, or a corresponding amino acid residue in another Cas9 polypeptide. In some embodiments, the heterologous polypeptide is inserted between amino acid positions 768-769, 792-793, 1022-1023, 1026-1027, 1029-1030, 1040-1041, 1068-1069, or 1247-1248 as numbered in the above Cas9 reference sequence or corresponding amino acid positions thereof. In some embodiments, the heterologous polypeptide is inserted between amino acid positions 769-770, 793-794, 1023-1024, 1027-1028, 1030-1031, 1041-1042, 1069-1070, or 1248-1249 as numbered in the above Cas9 reference sequence or corresponding amino acid positions thereof. In some embodiments, the heterologous polypeptide replaces an amino acid residue selected from the group consisting of: 768, 792, 1022, 1026, 1040, 1068, and 1247 as numbered in the above Cas9 reference sequence, or a corresponding amino acid residue in another Cas9 polypeptide.

A heterologous polypeptide (e.g., deaminase) can be inserted in the napDNAbp at an amino acid residue as described herein, or a corresponding amino acid residue in another Cas9 polypeptide. In an embodiment, a heterologous polypeptide (e.g., deaminase) can be inserted in the napDNAbp at an amino acid residue selected from the group consisting of: 1002, 1003, 1025, 1052-1056, 1242-1247, 1061-1077, 943-947, 686-691, 569-578, 530-539, and 1060-1077 as numbered in the above Cas9 reference sequence, or a corresponding amino acid residue in another Cas9 polypeptide. The deaminase (e.g., adenosine deaminase, cytidine deaminase, or adenosine deaminase and cytidine deaminase) can be inserted at the N-terminus or the C-terminus of the residue or replace the residue. In some embodiments, the deaminase (e.g., adenosine deaminase, cytidine deaminase, or adenosine deaminase and cytidine deaminase) is inserted at the C-terminus of the residue.

In some embodiments, an adenosine deaminase (e.g., TadA) is inserted at an amino acid residue selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, an adenosine deaminase (e.g., TadA) is inserted in place of residues 792-872, 792-906, or 2-791 as numbered in the above Cas9 reference sequence, or a corresponding amino acid residue in another Cas9 polypeptide. In some embodiments, the adenosine deaminase is inserted at the N-terminus of an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, the adenosine deaminase is inserted at the C-terminus of an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, the adenosine deaminase is inserted to replace an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide.

In some embodiments, a cytidine deaminase (e.g., APOBEC1) is inserted at an amino acid residue selected from the group consisting of: 1016, 1023, 1029, 1040, 1069, and 1247 as numbered in the above Cas9 reference sequence, or a corresponding amino acid residue in another Cas9 polypeptide. In some embodiments, the cytidine deaminase is inserted at the N-terminus of an amino acid selected from the group consisting of: 1016, 1023, 1029, 1040, 1069, and 1247 as numbered in the above Cas9 reference sequence, or a corresponding amino acid residue in another Cas9 polypeptide. In some embodiments, the cytidine deaminase is inserted at the C-terminus of an amino acid selected from the group consisting of: 1016, 1023, 1029, 1040, 1069, and 1247 as numbered in the above Cas9 reference sequence, or a corresponding amino acid residue in another Cas9 polypeptide. In some embodiments, the cytidine deaminase is inserted to replace an amino acid selected from the group consisting of: 1016, 1023, 1029, 1040, 1069, and 1247 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 768 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 791 or is inserted at amino acid residue 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 791 or is inserted at the N-terminus of amino acid 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid 791 or is inserted at the N-terminus of amino acid 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid 791, or is inserted to replace amino acid 792, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1016 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1022, or is inserted at amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1022 or is inserted at the N-terminus of amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1022 or is inserted at the C-terminus of amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1022, or is inserted to replace amino acid residue 1023, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1026, or is inserted at amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1026 or is inserted at the N-terminus of amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1026 or is inserted at the C-terminus of amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1026, or is inserted to replace amino acid residue 1029, as numbered in the above Cas9 reference sequence, or corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1040 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1052, or is inserted at amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1052 or is inserted at the N-terminus of amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1052 or is inserted at the C-terminus of amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1052, or is inserted to replace amino acid residue 1054, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1067, or is inserted at amino acid residue 1068, or is inserted at amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1067 or is inserted at the N-terminus of amino acid residue 1068 or is inserted at the N-terminus of amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1067 or is inserted at the C-terminus of amino acid residue 1068 or is inserted at the C-terminus of amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1067, or is inserted to replace amino acid residue 1068, or is inserted to replace amino acid residue 1069, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1246, or is inserted at amino acid residue 1247, or is inserted at amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1246 or is inserted at the N-terminus of amino acid residue 1247 or is inserted at the N-terminus of amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1246 or is inserted at the C-terminus of amino acid residue 1247 or is inserted at the C-terminus of amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1246, or is inserted to replace amino acid residue 1247, or is inserted to replace amino acid residue 1248, as numbered in the above Cas9 reference sequence, or a corresponding amino acid residue in another Cas9 polypeptide.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. In some embodiments, the deleted region corresponds to residues 792-872 as numbered in the above Cas9 reference sequence, or a corresponding amino acid residue in another Cas9 polypeptide. In some embodiments, the deleted region corresponds to residues 792-906 as numbered in the above Cas9 reference sequence, or a corresponding amino acid residue in another Cas9 polypeptide. In some embodiments, the deleted region corresponds to residues 2-791 as numbered in the above Cas9 reference sequence, or a corresponding amino acid residue in another Cas9 polypeptide. In some embodiments, the deleted region corresponds to residues 1017-1069 as numbered in the above Cas9 reference sequence, or corresponding amino acid residues thereof.

Exemplary internal fusions base editors are provided in Table 4 below:

TABLE 4
Insertion loci in Cas9 proteins, where “IBE”
represents “Internal Base Editor”

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC 1, RuvC II, RuvC III, Rec1, Rec2, PI, or HNH.

In some embodiments, the Cas9 polypeptide lacks one or more domains selected from the group consisting of RuvC I, RuvC II, RuvC III, Rec1, Rec2, PI, or HNH domain. In some embodiments, the Cas9 polypeptide lacks a nuclease domain. In some embodiments, the Cas9 polypeptide lacks an HNH domain. In some embodiments, the Cas9 polypeptide lacks a portion of the HNH domain such that the Cas9 polypeptide has reduced or abolished HNH activity. In some embodiments, the Cas9 polypeptide comprises a deletion of the nuclease domain, and the deaminase is inserted to replace the nuclease domain. In some embodiments, the HNH domain is deleted and the deaminase is inserted in its place. In some embodiments, one or more of the RuvC domains is deleted and the deaminase is inserted in its place.

A fusion protein comprising a heterologous polypeptide can be flanked by a N-terminal and a C-terminal fragment of a napDNAbp. In some embodiments, the fusion protein comprises a deaminase flanked by a N-terminal fragment and a C-terminal fragment of a Cas9 polypeptide. The N terminal fragment or the C terminal fragment can bind the target polynucleotide sequence. The C-terminus of the N terminal fragment or the N-terminus of the C terminal fragment can comprise a part of a flexible loop of a Cas9 polypeptide. The C-terminus of the N terminal fragment or the N-terminus of the C terminal fragment can comprise a part of an alpha-helix structure of the Cas9 polypeptide. The N-terminal fragment or the C-terminal fragment can comprise a DNA binding domain. The N-terminal fragment or the C-terminal fragment can comprise a RuvC domain. The N-terminal fragment or the C-terminal fragment can comprise an HNH domain. In some embodiments, neither of the N-terminal fragment and the C-terminal fragment comprises an HNH domain.

In some embodiments, the C-terminus of the N terminal Cas9 fragment comprises an amino acid that is in proximity to a target nucleobase when the fusion protein deaminates the target nucleobase. In some embodiments, the N-terminus of the C terminal Cas9 fragment comprises an amino acid that is in proximity to a target nucleobase when the fusion protein deaminates the target nucleobase. The insertion location of different deaminases can be different in order to have proximity between the target nucleobase and an amino acid in the C-terminus of the N terminal Cas9 fragment or the N-terminus of the C terminal Cas9 fragment. For example, the insertion position of an deaminase can be at an amino acid residue selected from the group consisting of 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide.

The N-terminal Cas9 fragment of a fusion protein (i.e. the N-terminal Cas9 fragment flanking the deaminase in a fusion protein) can comprise the N-terminus of a Cas9 polypeptide. The N-terminal Cas9 fragment of a fusion protein can comprise a length of at least about: 100, 200, 300, 400, 500, 600, 700, 800, 900, 1000, 1100, 1200, or 1300 amino acids. The N-terminal Cas9 fragment of a fusion protein can comprise a sequence corresponding to amino acid residues: 1-56, 1-95, 1-200, 1-300, 1-400, 1-500, 1-600, 1-700, 1-718, 1-765, 1-780, 1-906, 1-918, or 1-1100 as numbered in the above Cas9 reference sequence, or a corresponding amino acid residue in another Cas9 polypeptide. The N-terminal Cas9 fragment can comprise a sequence comprising at least: 85%, at least 90%, at least 91%, at least 92%, at least 93%, at least 94%, at least 95%, at least 96%, at least 97%, at least 98%, at least 99%, or at least 99.5% sequence identity to amino acid residues: 1-56, 1-95, 1-200, 1-300, 1-400, 1-500, 1-600, 1-700, 1-718, 1-765, 1-780, 1-906, 1-918, or 1-1100 as numbered in the above Cas9 reference sequence, or a corresponding amino acid residue in another Cas9 polypeptide.

The C-terminal Cas9 fragment of a fusion protein (i.e. the C-terminal Cas9 fragment flanking the deaminase in a fusion protein) can comprise the C-terminus of a Cas9 polypeptide. The C-terminal Cas9 fragment of a fusion protein can comprise a length of at least about: 100, 200, 300, 400, 500, 600, 700, 800, 900, 1000, 1100, 1200, or 1300 amino acids. The C-terminal Cas9 fragment of a fusion protein can comprise a sequence corresponding to amino acid residues: 1099-1368, 918-1368, 906-1368, 780-1368, 765-1368, 718-1368, 94-1368, or 56-1368 as numbered in the above Cas9 reference sequence, or a corresponding amino acid residue in another Cas9 polypeptide. The N-terminal Cas9 fragment can comprise a sequence comprising at least: 85%, at least 90%, at least 91%, at least 92%, at least 93%, at least 94%, at least 95%, at least 96%, at least 97%, at least 98%, at least 99%, or at least 99.5% sequence identity to amino acid residues: 1099-1368, 918-1368, 906-1368, 780-1368, 765-1368, 718-1368, 94-1368, or 56-1368 as numbered in the above Cas9 reference sequence, or a corresponding amino acid residue in another Cas9 polypeptide.

The N-terminal Cas9 fragment and C-terminal Cas9 fragment of a fusion protein taken together may not correspond to a full-length naturally occurring Cas9 polypeptide sequence, for example, as set forth in the above Cas9 reference sequence.

The fusion protein described herein can effect targeted deamination with reduced deamination at non-target sites (e.g., off-target sites), such as reduced genome wide spurious deamination. The fusion protein described herein can effect targeted deamination with reduced bystander deamination at non-target sites. The undesired deamination or off-target deamination can be reduced by at least 30%, at least 40%, at least 50%, at least 60%, at least 70%, at least 80%, at least 90%, at least 95%, or at least 99% compared with, for example, an end terminus fusion protein comprising the deaminase fused to a N terminus or a C terminus of a Cas9 polypeptide. The undesired deamination or off-target deamination can be reduced by at least one-fold, at least two-fold, at least three-fold, at least four-fold, at least five-fold, at least tenfold, at least fifteen fold, at least twenty fold, at least thirty fold, at least forty fold, at least fifty fold, at least 60 fold, at least 70 fold, at least 80 fold, at least 90 fold, or at least hundred fold, compared with, for example, an end terminus fusion protein comprising the deaminase fused to a N terminus or a C terminus of a Cas9 polypeptide.

In some embodiments, the deaminase (e.g., adenosine deaminase, cytidine deaminase, or adenosine deaminase and cytidine deaminase) of the fusion protein deaminates no more than two nucleobases within the range of an R-loop. In some embodiments, the deaminase of the fusion protein deaminates no more than three nucleobases within the range of the R-loop. In some embodiments, the deaminase of the fusion protein deaminates no more than 2, 3, 4, 5, 6, 7, 8, 9, or 10 nucleobases within the range of the R-loop. An R-loop is a three-stranded nucleic acid structure including a DNA:RNA hybrid, a DNA:DNA or an RNA: RNA complementary structure and the associated with single-stranded DNA. As used herein, an R-loop may be formed when a target polynucleotide is contacted with a CRISPR complex or a base editing complex, wherein a portion of a guide polynucleotide, e.g. a guide RNA, hybridizes with and displaces with a portion of a target polynucleotide, e.g. a target DNA. In some embodiments, an R-loop comprises a hybridized region of a spacer sequence and a target DNA complementary sequence. An R-loop region may be of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobase pairs in length. In some embodiments, the R-loop region is about 20 nucleobase pairs in length. It should be understood that, as used herein, an R-loop region is not limited to the target DNA strand that hybridizes with the guide polynucleotide. For example, editing of a target nucleobase within an R-loop region may be to a DNA strand that comprises the complementary strand to a guide RNA, or may be to a DNA strand that is the opposing strand of the strand complementary to the guide RNA. In some embodiments, editing in the region of the R-loop comprises editing a nucleobase on non-complementary strand (protospacer strand) to a guide RNA in a target DNA sequence.

The fusion protein described herein can effect target deamination in an editing window different from canonical base editing. In some embodiments, a target nucleobase is from about 1 to about 20 bases upstream of a PAM sequence in the target polynucleotide sequence. In some embodiments, a target nucleobase is from about 2 to about 12 bases upstream of a PAM sequence in the target polynucleotide sequence. In some embodiments, a target nucleobase is from about 1 to 9 base pairs, about 2 to 10 base pairs, about 3 to 11 base pairs, about 4 to 12 base pairs, about 5 to 13 base pairs, about 6 to 14 base pairs, about 7 to 15 base pairs, about 8 to 16 base pairs, about 9 to 17 base pairs, about 10 to 18 base pairs, about 11 to 19 base pairs, about 12 to 20 base pairs, about 1 to 7 base pairs, about 2 to 8 base pairs, about 3 to 9 base pairs, about 4 to 10 base pairs, about 5 to 11 base pairs, about 6 to 12 base pairs, about 7 to 13 base pairs, about 8 to 14 base pairs, about 9 to 15 base pairs, about 10 to 16 base pairs, about 11 to 17 base pairs, about 12 to 18 base pairs, about 13 to 19 base pairs, about 14 to 20 base pairs, about 1 to 5 base pairs, about 2 to 6 base pairs, about 3 to 7 base pairs, about 4 to 8 base pairs, about 5 to 9 base pairs, about 6 to 10 base pairs, about 7 to 11 base pairs, about 8 to 12 base pairs, about 9 to 13 base pairs, about 10 to 14 base pairs, about 11 to 15 base pairs, about 12 to 16 base pairs, about 13 to 17 base pairs, about 14 to 18 base pairs, about 15 to 19 base pairs, about 16 to 20 base pairs, about 1 to 3 base pairs, about 2 to 4 base pairs, about 3 to 5 base pairs, about 4 to 6 base pairs, about 5 to 7 base pairs, about 6 to 8 base pairs, about 7 to 9 base pairs, about 8 to 10 base pairs, about 9 to 11 base pairs, about 10 to 12 base pairs, about 11 to 13 base pairs, about 12 to 14 base pairs, about 13 to 15 base pairs, about 14 to 16 base pairs, about 15 to 17 base pairs, about 16 to 18 base pairs, about 17 to 19 base pairs, about 18 to 20 base pairs away or upstream of the PAM sequence. In some embodiments, a target nucleobase is about 1, 2, 3, 4, 5, 6, 7, 8, 9, 10, 11, 12, 13, 14, 15, 16, 17, 18, 19, 20, or more base pairs away from or upstream of the PAM sequence. In some embodiments, a target nucleobase is about 1, 2, 3, 4, 5, 6, 7, 8, or 9 base pairs upstream of the PAM sequence. In some embodiments, a target nucleobase is about 2, 3, 4, or 6 base pairs upstream of the PAM sequence.

The fusion protein can comprise more than one heterologous polypeptide. For example, the fusion protein can additionally comprise one or more UGI domains and/or one or more nuclear localization signals. The two or more heterologous domains can be inserted in tandem. The two or more heterologous domains can be inserted at locations such that they are not in tandem in the NapDNAbp.

A fusion protein can comprise a linker between the deaminase and the napDNAbp polypeptide. The linker can be a peptide or a non-peptide linker. For example, the linker can be an XTEN, (GGGS)n (SEQ ID NO: 250), (GGGGS)n (SEQ ID NO: 251), (G)n, (EAAAK)n (SEQ ID NO: 252), (GGS)n, SGSETPGTSESATPES (SEQ ID NO: 253). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase, but does not comprise a linker between the C-terminal Cas9 fragment and the deaminase. In some embodiments, the fusion protein comprises a linker between the C-terminal Cas9 fragment and the deaminase, but does not comprise a linker between the N-terminal Cas9 fragment and the deaminase.

In some embodiments, the napDNAbp in the fusion protein is a Cas12 polypeptide, e.g., Cas12b/C2c1, or a fragment thereof. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 254) or GSSGSETPGTSESATPESSG (SEQ ID NO: 255). In other embodiments, the linker is a rigid linker. In other embodiments of the above aspects, the linker is encoded by

(SEQ ID NO: 256)
GGAGGCTCTGGAGGAAGC
or
(SEQ ID NO: 257)
GGCTCTTCTGGATCTGAAACACCTGGCACAAGCGAGAGCGCCACCCCTGA
GAGCTCTGGC.

Fusion proteins comprising a heterologous catalytic domain flanked by N- and C-terminal fragments of a Cas12 polypeptide are also useful for base editing in the methods as described herein. Fusion proteins comprising Cas12 and one or more deaminase domains, e.g., adenosine deaminase, or comprising an adenosine deaminase domain flanked by Cas12 sequences are also useful for highly specific and efficient base editing of target sequences. In an embodiment, a chimeric Cas12 fusion protein contains a heterologous catalytic domain (e.g., adenosine deaminase, cytidine deaminase, or adenosine deaminase and cytidine deaminase) inserted within a Cas12 polypeptide. In some embodiments, the fusion protein comprises an adenosine deaminase domain and a cytidine deaminase domain inserted within a Cas12. In some embodiments, an adenosine deaminase is fused within Cas12 and a cytidine deaminase is fused to the C-terminus. In some embodiments, an adenosine deaminase is fused within Cas12 and a cytidine deaminase fused to the N-terminus. In some embodiments, a cytidine deaminase is fused within Cas12 and an adenosine deaminase is fused to the C-terminus. In some embodiments, a cytidine deaminase is fused within Cas12 and an adenosine deaminase fused to the N-terminus. Exemplary structures of a fusion protein with an adenosine deaminase and a cytidine deaminase and a Cas12 are provided as follows:
NH2-[Cas12(adenosine deaminase)]-[cytidine deaminase]-COOH;
NH2-[cytidine deaminase]-[Cas12(adenosine deaminase)]-COOH;
NH2-[Cas12(cytidine deaminase)]-[adenosine deaminase]-COOH; or
NH2-[adenosine deaminase]-[Cas12(cytidine deaminase)]-COOH;

In some embodiments, the “-” used in the general architecture above indicates the presence of an optional linker.

In various embodiments, the catalytic domain has DNA modifying activity (e.g., deaminase activity), such as adenosine deaminase activity. In some embodiments, the adenosine deaminase is a TadA (e.g., TadA*7.10). In some embodiments, the TadA is a TadA*8. In some embodiments, a TadA*8 is fused within Cas12 and a cytidine deaminase is fused to the C-terminus. In some embodiments, a TadA*8 is fused within Cas12 and a cytidine deaminase fused to the N-terminus. In some embodiments, a cytidine deaminase is fused within Cas12 and a TadA*8 is fused to the C-terminus. In some embodiments, a cytidine deaminase is fused within Cas12 and a TadA*8 fused to the N-terminus. Exemplary structures of a fusion protein with a TadA*8 and a cytidine deaminase and a Cas12 are provided as follows:
N-[Cas12(TadA*8)]-[cytidine deaminase]-C;
N-[cytidine deaminase]-[Cas12(TadA*8)]-C;
N-[Cas12(cytidine deaminase)]-[TadA*8]-C; or
N-[TadA*8]-[Cas12(cytidine deaminase)]-C.

In some embodiments, the “-” used in the general architecture above indicates the presence of an optional linker.

In other embodiments, the fusion protein contains one or more catalytic domains. In other embodiments, at least one of the one or more catalytic domains is inserted within the Cas12 polypeptide or is fused at the Cas12 N-terminus or C-terminus. In other embodiments, at least one of the one or more catalytic domains is inserted within a loop, an alpha helix region, an unstructured portion, or a solvent accessible portion of the Cas12 polypeptide. In other embodiments, the Cas12 polypeptide is Cas12a, Cas12b, Cas12c, Cas12d, Cas12e, Cas12g, Cas12h, Cas12i, or Cas12j/CasΦ. In other embodiments, the Cas12 polypeptide has at least about 85% amino acid sequence identity to Bacillus hisashii Cas12b, Bacillus thermoamylovorans Cas12b, Bacillus sp. V3-13 Cas12b, or Alicyclobacillus acidiphilus Cas12b (SEQ ID NO: 258). In other embodiments, the Cas12 polypeptide has at least about 90% amino acid sequence identity to Bacillus hisashii Cas12b (SEQ ID NO: 259), Bacillus thermoamylovorans Cas12b, Bacillus sp. V3-13 Cas12b, or Alicyclobacillus acidiphilus Cas12b. In other embodiments, the Cas12 polypeptide has at least about 95% amino acid sequence identity to Bacillus hisashii Cas12b, Bacillus thermoamylovorans Cas12b (SEQ ID NO: 260), Bacillus sp. V3-13 Cas12b (SEQ ID NO: 261), or Alicyclobacillus acidiphilus Cas12b. In other embodiments, the Cas12 polypeptide contains or consists essentially of a fragment of Bacillus hisashii Cas12b, Bacillus thermoamylovorans Cas12b, Bacillus sp. V3-13 Cas12b, or Alicyclobacillus acidiphilus Cas12b. In embodiments, the Cas12 polypeptide contains BvCas12b (V4), which in some embodiments is expressed as 5′ mRNA Cap-5′ UTR-bhCas12b-STOP sequence-3′ UTR-120polyA tail (SEQ ID NOs: 262-264).

In other embodiments, the catalytic domain is inserted between amino acid positions 153-154, 255-256, 306-307, 980-981, 1019-1020, 534-535, 604-605, or 344-345 of BhCas12b or a corresponding amino acid residue of Cas12a, Cas12c, Cas12d, Cas12e, Cas12g, Cas12h, Cas12i, or Cas12j/CasΦ. In other embodiments, the catalytic domain is inserted between amino acids P153 and S154 of BhCas12b. In other embodiments, the catalytic domain is inserted between amino acids K255 and E256 of BhCas12b. In other embodiments, the catalytic domain is inserted between amino acids D980 and G981 of BhCas12b. In other embodiments, the catalytic domain is inserted between amino acids K1019 and L1020 of BhCas12b. In other embodiments, the catalytic domain is inserted between amino acids F534 and P535 of BhCas12b. In other embodiments, the catalytic domain is inserted between amino acids K604 and G605 of BhCas12b. In other embodiments, the catalytic domain is inserted between amino acids H344 and F345 of BhCas12b. In other embodiments, catalytic domain is inserted between amino acid positions 147 and 148, 248 and 249, 299 and 300, 991 and 992, or 1031 and 1032 of BvCas12b or a corresponding amino acid residue of Cas12a, Cas12c, Cas12d, Cas12e, Cas12g, Cas12h, Cas12i, or Cas12j/CasΦ. In other embodiments, the catalytic domain is inserted between amino acids P147 and D148 of BvCas12b. In other embodiments, the catalytic domain is inserted between amino acids G248 and G249 of BvCas12b. In other embodiments, the catalytic domain is inserted between amino acids P299 and E300 of BvCas12b. In other embodiments, the catalytic domain is inserted between amino acids G991 and E992 of BvCas12b. In other embodiments, the catalytic domain is inserted between amino acids K1031 and M1032 of BvCas12b. In other embodiments, the catalytic domain is inserted between amino acid positions 157 and 158, 258 and 259, 310 and 311, 1008 and 1009, or 1044 and 1045 of AaCas12b or a corresponding amino acid residue of Cas12a, Cas12c, Cas12d, Cas12e, Cas12g, Cas12h, Cas12i, or Cas12j/CasΦ. In other embodiments, the catalytic domain is inserted between amino acids P157 and G158 of AaCas12b. In other embodiments, the catalytic domain is inserted between amino acids V258 and G259 of AaCas12b. In other embodiments, the catalytic domain is inserted between amino acids D310 and P311 of AaCas12b. In other embodiments, the catalytic domain is inserted between amino acids G1008 and E1009 of AaCas12b. In other embodiments, the catalytic domain is inserted between amino acids G1044 and K1045 at of AaCas12b.

In other embodiments, the fusion protein contains a nuclear localization signal (e.g., a bipartite nuclear localization signal). In other embodiments, the amino acid sequence of the nuclear localization signal is MAPKKKRKVGIHGVPAA (SEQ ID NO: 265). In other embodiments of the above aspects, the nuclear localization signal is encoded by the following sequence: ATGGCCCCAAAGAAGAAGCGGAAGGTCGGTATCCACGGAGTCCCAGCAGCC (SEQ ID NO: 266). In other embodiments, the Cas12b polypeptide contains a mutation that silences the catalytic activity of a RuvC domain. In other embodiments, the Cas12b polypeptide contains D574A, D829A and/or D952A mutations. In other embodiments, the fusion protein further contains a tag (e.g., an influenza hemagglutinin tag).

In some embodiments, the fusion protein comprises a napDNAbp domain (e.g., Cas12-derived domain) with an internally fused nucleobase editing domain (e.g., all or a portion of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 5 below.

TABLE 5
Insertion loci in Cas12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

By way of nonlimiting example, an adenosine deaminase (e.g., TadA*8.13) may be inserted into a BhCas12b to produce a fusion protein (e.g., TadA*8.13-BhCas12b) that effectively edits a nucleic acid sequence.

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 267-312.

In some embodiments, adenosine base editors were generated to insert TadA or variants thereof into the Cas9 polypeptide at the identified positions.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. Adenosine deaminase is capable of deaminating (i.e., removing an amine group) adenine of a deoxyadenosine residue in deoxyribonucleic acid (DNA). In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In certain embodiments, a base editor comprising an adenosine deaminase can deaminate a target A of a polynucleotide comprising RNA. For example, the base editor can comprise an adenosine deaminase domain capable of deaminating a target A of an RNA polynucleotide and/or a DNA-RNA hybrid polynucleotide. In an embodiment, an adenosine deaminase incorporated into a base editor comprises all or a portion of adenosine deaminase acting on RNA (ADAR, e.g., ADAR1 or ADAR2) or tRNA (ADAT). A base editor comprising an adenosine deaminase domain can also be capable of deaminating an A nucleobase of a DNA polynucleotide. In an embodiment an adenosine deaminase domain of a base editor comprises all or a portion of an ADAT comprising one or more mutations which permit the ADAT to deaminate a target A in DNA. For example, the base editor can comprise all or a portion of an ADAT from Escherichia coli (EcTadA) comprising one or more of the following mutations: D108N, A106V, D147Y, E155V, L84F, H123Y, 1156F, or a corresponding mutation in another adenosine deaminase. Exemplary ADAT homolog polypeptide sequences are provided in the Sequence Listing as SEQ ID NOs: 4 and 313-319.

The adenosine deaminase can be derived from any suitable organism (e.g., E. coli). In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

It should be appreciated that any of the mutations provided herein (e.g., based on the TadA reference sequence) can be introduced into other adenosine deaminases, such as E. coli TadA (ecTadA), S. aureus TadA (saTadA), or other adenosine deaminases (e.g., bacterial adenosine deaminases). It would be apparent to the skilled artisan that additional deaminases may similarly be aligned to identify homologous amino acid residues that can be mutated as provided herein. Thus, any of the mutations identified in the TadA reference sequence can be made in other adenosine deaminases (e.g., ecTada) that have homologous amino acid residues. It should also be appreciated that any of the mutations provided herein can be made individually or in any combination in the TadA reference sequence or another adenosine deaminase.

In some embodiments, the adenosine deaminase comprises a D108X mutation in the TadA reference sequence, or a corresponding mutation in another adenosine deaminase, where X indicates any amino acid other than the corresponding amino acid in the wild-type adenosine deaminase. In some embodiments, the adenosine deaminase comprises a D108G, D108N, D108V, D108A, or D108Y mutation in TadA reference sequence, or a corresponding mutation in another adenosine deaminase. It should be appreciated, however, that additional deaminases may similarly be aligned to identify homologous amino acid residues that can be mutated as provided herein.

In some embodiments, the adenosine deaminase comprises an A106X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A106V mutation in TadA reference sequence, or a corresponding mutation in another adenosine deaminase (e.g., ecTadA).

In some embodiments, the adenosine deaminase comprises a E155X mutation in TadA reference sequence, or a corresponding mutation in another adenosine deaminase, where the presence of X indicates any amino acid other than the corresponding amino acid in the wild-type adenosine deaminase. In some embodiments, the adenosine deaminase comprises a E155D, E155G, or E155V mutation in TadA reference sequence, or a corresponding mutation in another adenosine deaminase (e.g., ecTadA).

In some embodiments, the adenosine deaminase comprises a D147X mutation in TadA reference sequence, or a corresponding mutation in another adenosine deaminase, where the presence of X indicates any amino acid other than the corresponding amino acid in the wild-type adenosine deaminase. In some embodiments, the adenosine deaminase comprises a D147Y, mutation in TadA reference sequence, or a corresponding mutation in another adenosine deaminase (e.g., ecTadA).

In some embodiments, the adenosine deaminase comprises an A106X, E155X, or D147X, mutation in the TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an E155D, E155G, or E155V mutation. In some embodiments, the adenosine deaminase comprises a D147Y.

It should also be appreciated that any of the mutations provided herein may be made individually or in any combination in ecTadA or another adenosine deaminase. For example, an adenosine deaminase may contain a D108N, a A106V, a E155V, and/or a D147Y mutation in TadA reference sequence, or a corresponding mutation in another adenosine deaminase (e.g., ecTadA). In some embodiments, an adenosine deaminase comprises the following group of mutations (groups of mutations are separated by a “;”) in TadA reference sequence, or corresponding mutations in another adenosine deaminase: D108N and A106V; D108N and E155V; D108N and D147Y; A106V and E155V; A106V and D147Y; E155V and D147Y; D108N, A106V, and E155V; D108N, A106V, and D147Y; D108N, E155V, and D147Y; A106V, E155V, and D147Y; and D108N, A106V, E155V, and D147Y. It should be appreciated, however, that any combination of corresponding mutations provided herein may be made in an adenosine deaminase (e.g., ecTadA).

In some embodiments, the adenosine deaminase comprises a combination of mutations in a TadA reference sequence (e.g., TadA*7.10), or corresponding mutations in another adenosine deaminase: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; or L36H+I76Y+V82G+Y147D+F149Y+Q154S+N157K+D167N.

In some embodiments, the adenosine deaminase comprises one or more of a H8X, T17X, L18X, W23X, L34X, W45X, R51X, A56X, E59X, E85X, M94X, I95X, V102X, F104X, A106X, R107X, D108X, K110X, M118X, N127X, A138X, F149X, M151X, R153X, Q154X, I156X, and/or K157X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H8Y, T17S, L18E, W23L, L34S, W45L, R51H, A56E, or A56S, E59G, E85K, or E85G, M94L, I95L, V102A, F104L, A106V, R107C, or R107H, or R107P, D108G, or D108N, or D108V, or D108A, or D108Y, K110I, M118K, N127S, A138V, F149Y, M151V, R153C, Q154L, I156D, and/or K157R mutation in TadA reference sequence, or one or more corresponding mutations in another adenosine deaminase.

In some embodiments, the adenosine deaminase comprises one or more of a H8X, D108X, and/or N127X mutation in TadA reference sequence, or one or more corresponding mutations in another adenosine deaminase, where X indicates the presence of any amino acid. In some embodiments, the adenosine deaminase comprises one or more of a H8Y, D108N, and/or N127S mutation in TadA reference sequence, or one or more corresponding mutations in another adenosine deaminase.

In some embodiments, the adenosine deaminase comprises one or more of H8X, R26X, M61X, L68X, M70X, A106X, D108X, A109X, N127X, D147X, R152X, Q154X, E155X, K161X, Q163X, and/or T166X mutation in TadA reference sequence, or one or more corresponding mutations in another adenosine deaminase, where X indicates the presence of any amino acid other than the corresponding amino acid in the wild-type adenosine deaminase. In some embodiments, the adenosine deaminase comprises one or more of H8Y, R26W, M61I, L68Q, M70V, A106T, D108N, A109T, N127S, D147Y, R152C, Q154H or Q154R, E155G or E155V or E155D, K161Q, Q163H, and/or T166P mutation in TadA reference sequence, or one or more corresponding mutations in another adenosine deaminase.

In some embodiments, the adenosine deaminase comprises one, two, three, four, five, or six mutations selected from the group consisting of H8X, D108X, N127X, D147X, R152X, and Q154X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, M61X, M70X, D108X, N127X, Q154X, E155X, and Q163X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, or five, mutations selected from the group consisting of H8X, D108X, N127X, E155X, and T166X in TadA reference sequence, or a corresponding mutation or mutations in another adenosine deaminase (e.g., ecTadA), where X indicates the presence of any amino acid other than the corresponding amino acid in the wild-type adenosine deaminase.

In some embodiments, the adenosine deaminase comprises one, two, three, four, five, or six mutations selected from the group consisting of H8X, AT06X, and D108X, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, R26X, L68X, D108X, N127X, D147X, and E155X, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of H8X, R126X, L68X, D108X, N127X, D147X, and E155X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, D108X, A109X, N127X, and E155X in TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, or six mutations selected from the group consisting of H8Y, D108N, N127S, D147Y, R152C, and Q154H in TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, M61I, M70V, D108N, N127S, Q154R, E155G and Q163H in TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, N127S, E155V, and T166P in TadA reference sequence, or a corresponding mutation or mutations in another adenosine deaminase (e.g., ecTadA). In some embodiments, the adenosine deaminase comprises one, two, three, four, five, or six mutations selected from the group consisting of H8Y, A106T, D108N, N127S, E155D, and K161Q in TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, R26W, L68Q, D108N, N127S, D147Y, and E155V in TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, A109T, N127S, and E155G in TadA reference sequence, or a corresponding mutation or mutations in another adenosine deaminase (e.g., ecTadA).

In some embodiments, the adenosine deaminase comprises one or more of the or one or more corresponding mutations in another adenosine deaminase. In some embodiments, the adenosine deaminase comprises a D108N, D108G, or D108V mutation in TadA reference sequence, or corresponding mutations in another adenosine deaminase. In some embodiments, the adenosine deaminase comprises a A106V and D108N mutation in TadA reference sequence, or corresponding mutations in another adenosine deaminase. In some embodiments, the adenosine deaminase comprises R107C and D108N mutations in TadA reference sequence, or corresponding mutations in another adenosine deaminase. In some embodiments, the adenosine deaminase comprises a H8Y, D108N, N127S, D147Y, and Q154H mutation in TadA reference sequence, or corresponding mutations in another adenosine deaminase. In some embodiments, the adenosine deaminase comprises a H8Y, D108N, N127S, D147Y, and E155V mutation in TadA reference sequence, or corresponding mutations in another adenosine deaminase. In some embodiments, the adenosine deaminase comprises a D108N, D147Y, and E155V mutation in TadA reference sequence, or corresponding mutations in another adenosine deaminase. In some embodiments, the adenosine deaminase comprises a H8Y, D108N, and N127S mutation in TadA reference sequence, or corresponding mutations in another adenosine deaminase. In some embodiments, the adenosine deaminase comprises a A106V, D108N, D147Y, and E155V mutation in TadA reference sequence, or corresponding mutations in another adenosine deaminase (e.g., ecTadA).

In some embodiments, the adenosine deaminase comprises one or more of S2X, H8X, I49X, L84X, H123X, N127X, I156X, and/or K160X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of S2A, H8Y, I49F, L84F, H123Y, N127S, I156F, and/or K160S mutation in TadA reference sequence, or one or more corresponding mutations in another adenosine deaminase (e.g., ecTadA).

In some embodiments, the adenosine deaminase comprises an L84X mutation adenosine deaminase, where X indicates any amino acid other than the corresponding amino acid in the wild-type adenosine deaminase. In some embodiments, the adenosine deaminase comprises an L84F mutation in TadA reference sequence, or a corresponding mutation in another adenosine deaminase (e.g., ecTadA).

In some embodiments, the adenosine deaminase comprises an H123X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an H123Y mutation in TadA reference sequence, or a corresponding mutation in another adenosine deaminase.

In some embodiments, the adenosine deaminase comprises an I156X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an I156F mutation in TadA reference sequence, or a corresponding mutation in another adenosine deaminase.

In some embodiments, the adenosine deaminase comprises one, two, three, four, five, six, or seven mutations selected from the group consisting of L84X, A106X, D108X, H123X, D147X, E155X, and I156X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, or six mutations selected from the group consisting of S2X, I49X, A106X, D108X, D147X, and E155X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, A106X, D108X, N127X, and K160X in TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of L84F, A106V, D108N, H123Y, D147Y, E155V, and I156F in TadA reference sequence, or a corresponding mutation or mutations in another adenosine deaminase. In some embodiments, the adenosine deaminase comprises one, two, three, four, five, or six mutations selected from the group consisting of S2A, I49F, A106V, D108N, D147Y, and E155V in TadA reference sequence.

In some embodiments, the adenosine deaminase comprises one, two, three, four, or five mutations selected from the group consisting of H8Y, A106T, D108N, N127S, and K160S in TadA reference sequence, or a corresponding mutation or mutations in another adenosine deaminase.

In some embodiments, the adenosine deaminase comprises one or more of a E25X, R26X, R107X, A142X, and/or A143X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of E25M, E25D, E25A, E25R, E25V, E25S, E25Y, R26G, R26N, R26Q, R26C, R26L, R26K, R107P, R107K, R107A, R107N, R107W, R107H, R107S, A142N, A142D, A142G, A143D, A143G, A143E, A143L, A143W, A143M, A143S, A143Q, and/or A143R mutation in TadA reference sequence, or one or more corresponding mutations in another adenosine deaminase. In some embodiments, the adenosine deaminase comprises one or more of the mutations described herein corresponding to TadA reference sequence, or one or more corresponding mutations in another adenosine deaminase.

In some embodiments, the adenosine deaminase comprises an E25X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an E25M, E25D, E25A, E25R, E25V, E25S, or E25Y mutation in TadA reference sequence, or a corresponding mutation in another adenosine deaminase (e.g., ecTadA).

In some embodiments, the adenosine deaminase comprises an R26X mutation in TadA reference sequence, or a corresponding mutation in another adenosine deaminase, where X indicates any amino acid other than the corresponding amino acid in the wild-type adenosine deaminase. In some embodiments, the adenosine deaminase comprises R26G, R26N, R26Q, R26C, R26L, or R26K mutation in TadA reference sequence, or a corresponding mutation in another adenosine deaminase (e.g., ecTadA).

In some embodiments, the adenosine deaminase comprises an R107X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an R107P, R107K, R107A, R107N, R107W, R107H, or R107S mutation in TadA reference sequence, or a corresponding mutation in another adenosine deaminase (e.g., ecTadA).

In some embodiments, the adenosine deaminase comprises an A142X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A142N, A142D, A142G, mutation in TadA reference sequence, or a corresponding mutation in another adenosine deaminase (e.g., ecTadA).

In some embodiments, the adenosine deaminase comprises an A143X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A143D, A143G, A143E, A143L, A143W, A143M, A143S, A143Q, and/or A143R mutation in TadA reference sequence, or a corresponding mutation in another adenosine deaminase (e.g., ecTadA).

In some embodiments, the adenosine deaminase comprises one or more of a H36X, N37X, P48X, I49X, R51X, M70X, N72X, D77X, E134X, S146X, Q154X, K157X, and/or K161X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H36L, N37T, N37S, P48T, P48L, I49V, R51H, R51L, M70L, N72S, D77G, E134G, S146R, S146C, Q154H, K157N, and/or K161T mutation in TadA reference sequence, or one or more corresponding mutations in another adenosine deaminase (e.g., ecTadA).

In some embodiments, the adenosine deaminase comprises an H36X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an H36L mutation in TadA reference sequence, or a corresponding mutation in another adenosine deaminase.

In some embodiments, the adenosine deaminase comprises an N37X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an N37T or N37S mutation in TadA reference sequence, or a corresponding mutation in another adenosine deaminase.

In some embodiments, the adenosine deaminase comprises an P48X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an P48T or P48L mutation in TadA reference sequence, or a corresponding mutation in another adenosine deaminase.

In some embodiments, the adenosine deaminase comprises an R51X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an R51H or R51L mutation in TadA reference sequence, or a corresponding mutation in another adenosine deaminase.

In some embodiments, the adenosine deaminase comprises an S146X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an S146R or S146C mutation in TadA reference sequence, or a corresponding mutation in another adenosine deaminase.

In some embodiments, the adenosine deaminase comprises an K157X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a K157N mutation in TadA reference sequence, or a corresponding mutation in another adenosine deaminase.

In some embodiments, the adenosine deaminase comprises an P48X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a P48S, P48T, or P48A mutation in TadA reference sequence, or a corresponding mutation in another adenosine deaminase.

In some embodiments, the adenosine deaminase comprises an A142X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a A142N mutation in TadA reference sequence, or a corresponding mutation in another adenosine deaminase.

In some embodiments, the adenosine deaminase comprises an W23X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a W23R or W23L mutation in TadA reference sequence, or a corresponding mutation in another adenosine deaminase.

In some embodiments, the adenosine deaminase comprises an R152X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a R152P or R52H mutation in TadA reference sequence, or a corresponding mutation in another adenosine deaminase.

In one embodiment, the adenosine deaminase may comprise the mutations H36L, R51L, L84F, A106V, D108N, H123Y, S146C, D147Y, E155V, I156F, and K157N. In some embodiments, the adenosine deaminase comprises the following combination of mutations relative to TadA reference sequence, where each mutation of a combination is separated by a “_” and each combination of mutations is between parentheses:
(A106V_D108N),
(R107C_D108N),
(H8Y_D108N_N127S_D147Y_Q154H),
(H8Y_D108N_N127S_D147Y_E155V),
(D108N_D147Y_E155V),
(H8Y_D108N_N127S),
(H8Y_D108N_N127S_D147Y_Q154H),
(A106V_D108N_D147Y_E155V),
(D108Q_D147Y_E155V),
(D108M_D147Y_E155V),
(D108L_D147Y_E155V),
(D108K_D147Y_E155V),
(D108I_D147Y_E155V),
(D108F_D147Y_E155V),
(A106V_D108N_D147Y),
(A106V_D108M_D147Y_E155V),
(E59A_A106V_D108N_D147Y_E155V),
(E59A cat dead_A106V_D108N_D147Y_E155V),
(L84F_A106V_D108N_H123Y_D147Y_E155V_I156Y),
(L84F_A106V_D108N_H123Y_D147Y_E155V_I156F),
(D103A_D104N),
(G22P_D103A_D104N),
(D103A_D104N_S138A),
(R26G_L84F_A106V_R107H_D108N_H123Y_A142N_A143D_D147Y_E155V_I156F),
(E25G_R26G_L84F_A106V_R107H_D108N_H123Y_A142N_A143D_D147Y_E155V_I156F),
(E25D_R26G_L84F_A106V_R107K_D108N_H123Y_A142N_A143G_D147Y_E155V_I156F),
(R26Q_L84F_A106V_D108N_H123Y_A142N_D147Y_E155V_I156F),
(E25M_R26G_L84F_A106V_R107P_D108N_H123Y_A142N_A143D_D147Y_E155V_I156F),
(R26C_L84F_A106V_R107H_D108N_H123Y_A142N_D147Y_E155V_I156F),
(L84F_A106V_D108N_H123Y_A142N_A143L_D147Y_E155V_I156F),
(R26G_L84F_A106V_D108N_H123Y_A142N_D147Y_E155V_I156F),
(E25A_R26G_L84F_A106V_R107N_D108N_H123Y_A142N_A143E_D147Y_E155V_I156F),
(R26G_L84F_A106V_R107H_D108N_H123Y_A142N_A143D_D147Y_E155V_I156F),
(A106V_D108N_A142N_D147Y_E155V),
(R26G_A106V_D108N_A142N_D147Y_E155V),
(E25D_R26G_A106V_R107K_D108N_A142N_A143G_D147Y_E155V),
(R26G_A106V_D108N_R107H_A142N_A143D_D147Y_E155V),
(E25D_R26G_A106V_D108N_A142N_D147Y_E155V),
(A106V_R107K_D108N_A142N_D147Y_E155V),
(A106V_D108N_A142N_A143G_D147Y_E155V),
(A106V_D108N_A142N_A143L_D147Y_E155V),
(H36L_R51L_L84F_A106V_D108N_H123Y_S146C_D147Y_E155V_I156F_K157N),
(N37T_P48T_M70L_L84F_A106V_D108N_H123Y_D147Y_149V_E155V_1156F),
(N37S_L84F_A106V_D108N_H123Y_D147Y_E155V_I156F_K161T),
(H36L_L84F_A106V_D108N_H123Y_D147Y_Q154H_E155V_I156F),
(N72S_L84F_A106V_D108N_H123Y_S146R_D147Y_E155V_I156F),
(H36L_P48L_L84F_A106V_D108N_H123Y_E134G_D147Y_E155V_I156F),
(H36L_L84F_A106V_D108N_H123Y_D147Y_E155V_I156F_K157N)
(H36L_L84F_A106V_D108N_H123Y_S146C_D147Y_E155V_I156F),
(L84F_A106V_D108N_H123Y_S146R_D147Y_E155V_I156F_K161T),
(N37S_R51H_D77G_L84F_A106V_D108N_H123Y_D147Y_E155V_I156F),
(R51L_L84F_A106V_D108N_H123Y_D147Y_E155V_I156F_K157N),
(D24G_Q71R_L84F_H96L_A106V_D108N_H123Y_D147Y_E155V_I156F_K160E),
(H36L_G67V_L84F_A106V_D108N_H123Y_S146T_D147Y_E155V_I156F),
(Q71L_L84F_A106V_D108N_H123Y_L137M_A143E_D147Y_E155V_I156F),
(E25G_L84F_A106V_D108N_H123Y_D147Y_E155V_I156F_Q159L),
(L84F_A91T_F104I_A106V_D108N_H123Y_D147Y_E155V_I156F),
(N72D_L84F_A106V_D108N_H123Y_G125A_D147Y_E155V_1156F),
(P48S_L84F_S97C_A106V_D108N_H123Y_D147Y_E155V_I156F),
(W23G_L84F_A106V_D108N_H123Y_D147Y_E155V_I156F),
(D24G_P48L_Q71R_L84F_A106V_D108N_H123Y_D147Y_E155V_I156F_Q159L),
(L84F_A106V_D108N_H123Y_A142N_D147Y_E155V_I156F),
(H36L_R51L_L84F_A106V_D108N_H123Y_A142N_S146C_D147Y_E155V_I156F_K157N),
(N37S_L84F_A106V_D108N_H123Y_A142N_D147Y_E155V_I156F_K161T),
(L84F_A106V_D108N_D147Y_E155V_I156F),
(R51L_L84F_A106V_D108N_H123Y_S146C_D147Y_E155V_I156F_K157N_K161T),
(L84F_A106V_D108N_H123Y_S146C_D147Y_E155V_I156F_K161T),
(L84F_A106V_D108N_H123Y_S146C_D147Y_E155V_I156F_K157N_K160E_K161T),
(L84F_A106V_D108N_H123Y_S146C_D147Y_E155V_I156F_K157N_K160E),
(R74Q_L84F_A106V_D108N_H123Y_D147Y_E155V_I156F),
(R74A_L84F_A106V_D108N_H123Y_D147Y_E155V_I156F),
(L84F_A106V_D108N_H123Y_D147Y_E155V_I156F),
(R74Q_L84F_A106V_D108N_H123Y_D147Y_E155V_I156F),
(L84F_R98Q_A106V_D108N_H123Y_D147Y_E155V_I156F),
(L84F_A106V_D108N_H123Y_R129Q_D147Y_E155V_I156F),
(P48S_L84F_A106V_D108N_H123Y_A142N_D147Y_E155V_I156F),
(P48S_A142N),
(P48T_I49V_L84F_A106V_D108N_H123Y_A142N_D147Y_E155V_I156F_L157N),
(P48T_I49V_A142N),
(H36L_P48S_R51L_L84F_A106V_D108N_H123Y_S146C_D147Y_E155V_I156F_K157N),
(H36L_P48S_R51L_L84F_A106V_D108N_H123Y_S146C_A142N_D147Y_E155V_I156F
(H36L_P48T_I49V_R51L_L84F_A106V_D108N_H123Y_S146C_D147Y_E155V_I156F_K157N),
(H36L_P48T_I49V_R51L_L84F_A106V_D108N_H123Y_A142N_S146C_D147Y_E155V_I156F_K157N),
(H36L_P48A_R51L_L84F_A106V_D108N_H123Y_S146C_D147Y_E155V_I156F_K157N),
(H36L_P48A_R51L_L84F_A106V_D108N_H123Y_A142N_S146C_D147Y_E155V_I156F_K157N),
(H36L_P48A_R51L_L84F_A106V_D108N_H123Y_S146C_A142N_D147Y_E155V_I156F_K157N),
(W23L_H36L_P48A_R51L_L84F_A106V_D108N_H123Y_S146C_D147Y_E155V_I156F_K157N),
(W23R_H36L_P48A_R51L_L84F_A106V_D108N_H123Y_S146C_D147Y_E155V_I156F_K157N),
(W23L_H36L_P48A_R51L_L84F_A106V_D108N_H123Y_S146R_D147Y_E155V_I156F_K161T),
(H36L_P48A_R51L_L84F_A106V_D108N_H123Y_S146C_D147Y_R152H_E155V_I156F_K157N),
(H36L_P48A_R51L_L84F_A106V_D108N_H123Y_S146C_D147Y_R152P_E155V_I156F_K157N),
(W23L_H36L_P48A_R51L_L84F_A106V_D108N_H123Y_S146C_D147Y_R152P_E155V_I156F_K157N),
(W23L_H36L_P48A_R51L_L84F_A106V_D108N_H123Y_A142A_S146C_D147Y_E155V_I156F_K157N),
(W23L_H36L_P48A_R51L_L84F_A106V_D108N_H123Y_A142A_S146C_D147Y_R152P_E155V_1156F_K157N),
(W23L_H36L_P48A_R51L_L84F_A106V_D108N_H123Y_S146R_D147Y_E155V_1156F_K161T),
(W23R_H36L_P48A_R51L_L84F_A106V_D108N_H123Y_S146C_D147Y_R152P_E155V_I156F_K157N), (H36L_P48A_R51L_L84F_A106V_D108N_H123Y_A142N_S146C_D147Y_R152P_E155 V_1156F_K157N).

In some embodiments, the TadA deaminase is a TadA variant. In some embodiments, the TadA variant is TadA*7.10. In particular embodiments, the fusion proteins comprise a single TadA*7.10 domain (e.g., provided as a monomer). In other embodiments, the fusion protein comprises TadA*7.10 and TadA(wt), which are capable of forming heterodimers. In one embodiment, a fusion protein of the invention comprises a wild-type TadA linked to TadA*7.10, which is linked to Cas9 nickase.

In some embodiments, TadA*7.10 comprises at least one alteration. In some embodiments, the adenosine deaminase comprises an alteration in the following sequence:

TadA*7.10
(SEQ ID NO: 4)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD

In some embodiments, TadA*7.10 comprises an alteration at amino acid 82 and/or 166. In particular embodiments, TadA*7.10 comprises one or more of the following alterations: Y147T, Y147R, Q154S, Y123H, V82S, T166R, and/or Q154R. In other embodiments, a variant of TadA*7.10 comprises a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R.

In some embodiments, a variant of TadA*7.10 comprises one or more of alterations selected from the group of L36H, I76Y, V82G, Y147T, Y147D, F149Y, Q154S, N157K, and/or D167N. In some embodiments, a variant of TadA*7.10 comprises V82G, Y147T/D, Q154S, and one or more of L36H, I76Y, F149Y, N157K, and D167N. In other embodiments, a variant of TadA*7.10 comprises a combination of alterations selected from the group of: V82G+Y147T +Q154S; I76Y+V82G+Y147T+Q154S; L36H+V82G+Y147T+Q154S+N157K; V82G +Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N.

In some embodiments, an adenosine deaminase variant (e.g., TadA*8) comprises a deletion. In some embodiments, an adenosine deaminase variant comprises a deletion of the C terminus. In particular embodiments, an adenosine deaminase variant comprises a deletion of the C terminus beginning at residue 149, 150, 151, 152, 153, 154, 155, 156, and 157, relative to TadA*7.10, the TadA reference sequence, or a corresponding mutation in another TadA.

In other embodiments, an adenosine deaminase variant (e.g., TadA*8) is a monomer comprising one or more of the following alterations: Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant (TadA*8) is a monomer comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, the adenosine deaminase variant is a homodimer comprising two adenosine deaminase domains (e.g., TadA*8) each having one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a homodimer comprising two adenosine deaminase domains (e.g., TadA*8) each having a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R +Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor of the disclosure comprising an adenosine deaminase variant (e.g., TadA*8) monomer comprising one or more of the following alterations: R26C, V88A, A109S, T111R, D119N, H122N, Y147D, F149Y, T166I and/or D167N, relative to TadA*7.10, the TadA reference sequence, or a corresponding mutation in another TadA. In other embodiments, the adenosine deaminase variant (TadA*8) monomer comprises a combination of alterations selected from the group of: R26C+A109S+T111R+D119N+H122N+Y147D+F149Y+T166I +D167N; V88A+A109S+T111R+D119N+H122N+F149Y+T166I +D167N; R26C+A109S+T111R+D119N+H122N+F149Y+T166I +D167N; V88A+T111R+D119N+F149Y; and A109S+T111R+D119N+H122N+Yl47D +F149Y+T166I+D167N, relative to TadA*7.10, the TadA reference sequence, or a corresponding mutation in another TadA.

In some embodiments, an adenosine deaminase variant (e.g., MSP828) is a monomer comprising one or more of the following alterations L36H, I76Y, V82G, Yl47T, Yl47D, F149Y, Q154S, N157K, and/or D167N, relative to TadA*7.10, the TadA reference sequence, or a corresponding mutation in another TadA. In some embodiments, an adenosine deaminase variant (e.g., MSP828) is a monomer comprising V82G, Yl47T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant (TadA variant) is a monomer comprising a combination of alterations selected from the group of: V82G+Yl47T+Q154S; I76Y+V82G+Yl47T+Q154S; L36H+V82G+Yl47T +Q154S+N157K; V82G+Yl47D+F149Y+Q154S+D167N; L36H+V82G+Yl47D+F149Y+Q154S+N157K+D167N; L36H+I76Y+V82G+Yl47T+Q154S+N157K; I76Y +V82G+Yl47D+F149Y+Q154S+D167N; L36H+I76Y+V82G+Yl47D+F149Y+Q154S+N157K+D167N, relative to TadA*7.10, the TadA reference sequence, or a corresponding mutation in another TadA.

In other embodiments, the adenosine deaminase variant is a heterodimer of a wild-type adenosine deaminase domain and an adenosine deaminase variant domain (e.g., TadA*8) comprising one or more of the following alterations Yl47T, Yl47R, Q154S, Y123H, V82S, T166R, and/or Q154R, relative to TadA*7.10, the TadA reference sequence, or a corresponding mutation in another TadA. In other embodiments, the adenosine deaminase variant is a heterodimer of a wild-type adenosine deaminase domain and an adenosine deaminase variant domain (e.g., TadA*8) comprising a combination of alterations selected from the group of: Yl47T+Q154R; Yl47T+Q154S; Yl47R+Q154S; V82S+Q154S; V82S+Yl47R; V82S+Q154R; V82S+Y123H; I76Y+V82S; V82S+Y123H+Yl47T; V82S+Y123H+Yl47R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor of the disclosure comprising an adenosine deaminase variant (e.g., TadA*8) homodimer comprising two adenosine deaminase domains (e.g., TadA*8) each having one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the adenosine deaminase variant is a homodimer comprising two adenosine deaminase domains (e.g., TadA*8) each having a combination of alterations selected from the group of: R26C+A109S+T111R+D119N+H122N+Y147D+F149Y+T166I+D167N; V88A+A109S+T111R+D119N+H122N+F149Y+T166I +D167N; R26C+A109S+T111R+D119N+H122N+F149Y+T166I+D167N; V88A+T111R+D119N+F149Y; and A109S+T111R+D119N+H122N+Yl47D+F149Y+T166I+D167N, relative to TadA*7.10, the TadA reference sequence, or a corresponding mutation in another TadA.

In some embodiments, an adenosine deaminase variant is a homodimer comprising two adenosine deaminase domains (e.g., TadA*7.10) each having one or more of the following alterations L36H, I76Y, V82G, Yl47T, Yl47D, F149Y, Q154S, N157K, and/or D167N, relative to TadA*7.10, the TadA reference sequence, or a corresponding mutation in another TadA. In some embodiments, an adenosine deaminase variant is a homodimer comprising two adenosine deaminase variant domains (e.g., MSP828) each having the following alterations V82G, Yl47T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant is a homodimer comprising two adenosine deaminase domains (e.g., TadA*7.10) each having a combination of alterations selected from the group of: V82G+Yl47T+Q154S; I76Y+V82G+Yl47T+Q154S; L36H+V82G+Yl47T +Q154S+N157K; V82G+Yl47D+F149Y+Q154S+D167N; L36H+V82G+Yl47D+F149Y+Q154S+N157K+D167N; L36H+I76Y+V82G+Yl47T+Q154S+N157K; I76Y +V82G+Yl47D+F149Y+Q154S+D167N; L36H+I76Y+V82G+Yl47D+F149Y+Q154S+N157K+D167N, relative to TadA*7.10, the TadA reference sequence, or a corresponding mutation in another TadA.

In other embodiments, the adenosine deaminase variant is a heterodimer of a TadA*7.10 domain and an adenosine deaminase variant domain (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a heterodimer of a TadA*7.10 domain and an adenosine deaminase variant domain (e.g., TadA*8) comprising a combination of alterations selected from the group of Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R +Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor comprises a heterodimer of a wild-type adenosine deaminase domain and an adenosine deaminase variant domain (e.g., TadA*8) comprising one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the base editor comprises a heterodimer of a wild-type adenosine deaminase domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I +D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In other embodiments, the adenosine deaminase variant is a heterodimer of a wild-type adenosine deaminase domain and an adenosine deaminase variant domain (e.g., TadA*7.10) comprising one or more of the following alterations L36H, I76Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant is a heterodimer comprising a wild-type adenosine deaminase domain and an adenosine deaminase variant domain (e.g., MSP828) having the following alterations V82G, Y147T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant is a heterodimer of a wild-type adenosine deaminase domain and an adenosine deaminase variant domain (e.g., TadA*7.10) comprising a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G +Y147T+Q154S+N157K; I76Y+V82G+Y147D+F149Y+Q154S+D167N; L36H+I76Y+V82G+Y147D+F149Y+Q154S+N157K+D167N, relative to TadA*7.10, the TadA reference sequence, or a corresponding mutation in another TadA.

In other embodiments, the adenosine deaminase variant is a heterodimer of a TadA*7.10 domain and an adenosine deaminase variant domain (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a heterodimer of a TadA*7.10 domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R +Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, an adenosine deaminase is a TadA*8. In one embodiment, an adenosine deaminase is a TadA*8 that comprises or consists essentially of the following sequence or a fragment thereof having adenosine deaminase activity:

(SEQ ID NO: 320)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCTFFR
MPRQVFNAQKKAQSSTD

In some embodiments, the TadA*8 is truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8.

In some embodiments the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24.

In other embodiments, a base editor of the disclosure comprising an adenosine deaminase variant (e.g., TadA*8) monomer comprising one or more of the following alterations: R26C, V88A, A109S, T111R, D119N, H122N, Y147D, F149Y, T166I and/or D167N, relative to TadA*7.10, the TadA reference sequence, or a corresponding mutation in another TadA. In other embodiments, the adenosine deaminase variant (TadA*8) monomer comprises a combination of alterations selected from the group of: R26C+A109S+T11R+D119N+H122N+Y147D+F149Y+T166I +D167N; V88A+A109S+T111R+D119N+H122N+F149Y+T166I +D167N; R26C+A109S+T111R+D119N+H122N+F149Y+T166I +D167N; V88A+T111R+D119N+F149Y; and A109S+T111R+D119N+H122N+Y147D +F149Y+T166I+D167N, relative to TadA*7.10, the TadA reference sequence, or a corresponding mutation in another TadA.

In other embodiments, a base editor comprises a heterodimer of a wild-type adenosine deaminase domain and an adenosine deaminase variant domain (e.g., TadA*8) comprising one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the base editor comprises a heterodimer of a wild-type adenosine deaminase domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I +D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In other embodiments, a base editor comprises a heterodimer of a TadA*7.10 domain and an adenosine deaminase variant domain (e.g., TadA*8) comprising one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the base editor comprises a heterodimer of a TadA*7.10 domain and an adenosine deaminase variant domain (e.g., TadA*8) comprising a combination of alterations selected from the group of: R26C+A109S+T111R+D119N+H122N+Y147D+F149Y+T166I +D167N; V88A+A109S+T111R+D119N+H122N+F149Y+T166I +D167N; R26C+A109S+T111R+D119N+H122N+F149Y+T166I+D167N; V88A+T111R+D119N+F149Y; andA109S+T111R+D119N+H122N+Y147D+F149Y+T166I +D167N, relative to TadA*7.10, the TadA reference sequence, or a corresponding mutation in another TadA.

In other embodiments, the adenosine deaminase variant is a heterodimer of a TadA*7.10 domain and an adenosine deaminase variant domain (e.g., TadA*7.10) comprising one or more of the following alterations L36H, I76Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant is a heterodimer comprising a TadA*7.10 domain and an adenosine deaminase variant domain (e.g., MSP828) having the following alterations V82G, Y147T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant is a heterodimer of a TadA*7.10 domain and an adenosine deaminase variant domain (e.g., TadA*7.10) comprising a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D +F149Y+Q154S+D167N; L36H+I76Y+V82G+Y147D+F149Y+Q154S+N157K+D167N, relative to TadA*7.10, the TadA reference sequence, or a corresponding mutation in another TadA.

In some embodiments, the TadA*8 is a variant as shown in Table 6. Table 6 shows certain amino acid position numbers in the TadA amino acid sequence and the amino acids present in those positions in the TadA-7.10 adenosine deaminase. Table 6 also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e.

TABLE 6
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA-7.10 | R | V | A | T | D | H | Y | F | T | D |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 6.1. Table 6.1 shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 6.1
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In one embodiment, a fusion protein of the invention comprises a wild-type TadA is linked to an adenosine deaminase variant described herein (e.g., TadA*8), which is linked to Cas9 nickase. In particular embodiments, the fusion proteins comprise a single TadA*8 domain (e.g., provided as a monomer). In other embodiments, the fusion protein comprises TadA*8 and TadA(wt), which are capable of forming heterodimers.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

In particular embodiments, a TadA*8 comprises one or more mutations at any of the following positions shown in bold. In other embodiments, a TadA*8 comprises one or more mutations at any of the positions shown with underlining:

| (SEQ ID NO: 4) | |
|---|---|
| MSEVEFSHEY WMRHALTLAK RARDEREVPV GAVLVLNNRV IGEGWNRAIG | 50 |
| LHDPTAHAEI MALRQGGLVM QNYRLIDATL YVTFEPCVMC AGAMIHSRIG | 100 |
| RVVFGVRNAK TGAAGSLMDV LHYPGMNHRV EITEGILADE CAALLCYFFR | 150 |
| MPRQVFNAQK KAQSSTD | |

For example, the TadA*8 comprises alterations at amino acid position 82 and/or 166 (e.g., V82S, T166R) alone or in combination with any one or more of the following Y147T, Y147R, Q154S, Y123H, and/or Q154R, relative to TadA*7.10, the TadA reference sequence, or a corresponding mutation in another TadA. In particular embodiments, a combination of alterations is selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R +Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In some embodiments, the TadA*8 is truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8.

In one embodiment, a fusion protein of the invention comprises a wild-type TadA is linked to an adenosine deaminase variant described herein (e.g., TadA*8), which is linked to Cas9 nickase. In particular embodiments, the fusion proteins comprise a single TadA*8 domain (e.g., provided as a monomer). In other embodiments, the base editor comprises TadA*8 and TadA(wt), which are capable of forming heterodimers.

In particular embodiments, the fusion proteins comprise a single (e.g., provided as a monomer) TadA*8. In some embodiments, the TadA*8 is linked to a Cas9 nickase. In some embodiments, the fusion proteins of the invention comprise as a heterodimer of a wild-type TadA (TadA(wt)) linked to a TadA*8. In other embodiments, the fusion proteins of the invention comprise as a heterodimer of a TadA*7.10 linked to a TadA*8. In some embodiments, the base editor is ABE8 comprising a TadA*8 variant monomer. In some embodiments, the base editor is ABE8 comprising a heterodimer of a TadA*8 and a TadA(wt). In some embodiments, the base editor is ABE8 comprising a heterodimer of a TadA*8 and TadA*7.10. In some embodiments, the base editor is ABE8 comprising a heterodimer of a TadA*8. In some embodiments, the TadA*8 is selected from Table 6, 12 or 13. In some embodiments, the ABE8 is selected from Table 12, 13 or 15.

In some embodiments, the adenosine deaminase is a TadA*9 variant. In some embodiments, the adenosine deaminase is a TadA*9 variant selected from the variants described below and with reference to the following sequence (termed TadA*7.10):

(SEQ ID NO: 4)

MSEVEFSHEY WMRHALTLAK RARDEREVPV GAVLVLNNRV
IGEGWNRAIG LHDPTAHAEI MALRQGGLVM QNYRLIDATL
YVTFEPCVMC AGAMIHSRIG RVVFGVRNAK TGAAGSLMDV
LHYPGMNHRV EITEGILADE CAALLCYFFR MPRQVFNAQK
KAQSSTD

In some embodiments, an adenosine deaminase comprises one or more of the following alterations: R21N, R23H, E25F, N38G, L51W, P54C, M70V, Q71M, N72K, Y73S, V82T, M94V, P124W, T133K, D139L, D139M, C146R, and A158K. The one or more alternations are shown in the sequence above in underlining and bold font.

In some embodiments, an adenosine deaminase comprises one or more of the following combinations of alterations: V82S+Q154R+Y147R; V82S+Q154R+Y123H; V82S+Q154R+Y147R+Y123H; Q154R+Y147R+Y123H+I76Y+V82S; V82S+I76Y; V82S+Y147R; V82S+Y147R+Y123H; V82S+Q154R+Y123H; Q154R+Y147R+Y123H+I76Y; V82S+Y147R; V82S+Y147R+Y123H; V82S+Q154R+Y123H; V82S+Q154R+Y147R; V82S+Q154R+Y147R; Q154R+Y147R+Y123H+I76Y; Q154R+Y147R+Y123H+I76Y+V82S; I76Y_V82S_Y123H_Y147R_Q154R; Y147R+Q154R+H123H; and V82S+Q154R.

In some embodiments, an adenosine deaminase comprises one or more of the following combinations of alterations: E25F+V82S+Y123H, T133K+Y147R+Q154R; E25F+V82S +Y123H+Y147R+Q154R; L51W+V82S+Y123H+C146R+Y147R+Q154R; Y73S+V82S+Y123H+Y147R+Q154R; P54C+V82S+Y123H+Y147R+Q154R; N38G+V82T +Y123H+Y147R+Q154R; N72K+V82S+Y123H+D139L+Y147R+Q154R; E25F+V82S+Y123H+D139M+Y147R+Q154R; Q71M+V82S+Y123H+Y147R+Q154R; E25F+V82S+Y123H+T133K+Y147R+Q154R; E25F+V82S+Y123H+Y147R+Q154R; V82S+Y123H+P124W+Y147R+Q154R; L51W+V82S+Y123H+C146R+Y147R+Q154R; P54C+V82S+Y123H+Y147R+Q154R; Y73S+V82S+Y123H+Y147R +Q154R; N38G+V82T+Y123H+Y147R+Q154R; R23H+V82S+Y123H+Y147R+Q154R; R21N+V82S+Y123H+Y147R+Q154R; V82S+Y123H+Y147R+Q154R+A158K; N72K+V82S+Y123H+D139L+Y147R+Q154R; E25F+V82S+Y123H+D139M+Y147R+Q154R; and M70V+V82S+M94V+Y123H+Y147R+Q154R

In some embodiments, an adenosine deaminase comprises one or more of the following combinations of alterations: Q71M+V82S+Y123H+Y147R+Q154R; E25F+I76Y+V82S +Y123H+Y147R+Q154R; I76Y+V82T+Y123H+Y147R+Q154R; N38G+I76Y+V82S+Y123H+Y147R+Q154R; R23H+I76Y+V82S+Y123H+Y147R+Q154R; P54C +I76Y+V82S+Y123H+Y147R+Q154R; R21N+I76Y+V82S+Y123H+Y147R+Q154R; 176Y+V82S+Y123H+D139M+Y147R+Q154R; Y73S+I76Y+V82S+Y123H +Y147R+Q154R; E25F+I76Y+V82S+Y123H+Y147R+Q154R; I76Y+V82T+Y123H +Y147R+Q154R; N38G+I76Y+V82S+Y123H+Y147R+Q154R; R23H+I76Y+V82S +Y123H+Y147R+Q154R; P54C+I76Y+V82S+Y123H+Y147R+Q154R; R21N+I76Y +V82S+Y123H+Y147R+Q154R; I76Y+V82S+Y123H+D139M+Y147R+Q154R; Y73S+I76Y+V82S+Y123H+Y147R+Q154R; and V82S+Q154R; N72K_V82S+Y123H +Y147R+Q154R; Q71M_V82S+Y123H+Y147R+Q154R; V82S+Y123H+T133K+Y147R+Q154R; V82S+Y123H+T133K+Y147R+Q154R+A158K; M70V+Q71M+N72K+V82S+Y123H+Y147R+Q154R; N72K_V82S+Y123H+Y147R+Q154R; Q71M_V82S+Y123H+Y147R+Q154R; M70V+V82S+M94V+Y123H+Y147R+Q154R; V82S+Y123H+T133K+Y147R+Q154R; V82S+Y123H+T133K+Y147R+Q154R+A158K; and M70V+Q71M+N72K+V82S+Y123H+Y147R+Q154R. In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation, e.g., Y73S and Y72S and D139M and D138M.

In some embodiments, the TadA*9 variant comprises the alterations described in Table 16 as described herein. In some embodiments, the TadA*9 variant is a monomer. In some embodiments, the TadA*9 variant is a heterodimer with a wild-type TadA adenosine deaminase. In some embodiments, the TadA*9 variant is a heterodimer with another TadA variant (e.g., TadA*8, TadA*9). Additional details of TadA*9 adenosine deaminases are described in International PCT Application No. PCT/2020/049975, which is incorporated herein by reference for its entirety.

Any of the mutations provided herein and any additional mutations (e.g., based on the ecTadA amino acid sequence) can be introduced into any other adenosine deaminases. Any of the mutations provided herein can be made individually or in any combination in TadA reference sequence or another adenosine deaminase (e.g., ecTadA).

Details of A to G nucleobase editing proteins are described in International PCT Application No. PCT/2017/045381 (WO2018/027078) and Gaudelli, N. M., et al., “Programmable base editing of A•T to G•C in genomic DNA without DNA cleavage” Nature, 551, 464-471 (2017), the entire contents of which are hereby incorporated by reference.

### C to T Editing

In some embodiments, a base editor disclosed herein comprises a fusion protein comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T:A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids. Typically, a cytidine deaminase catalyzes a C nucleobase that is positioned in the context of a single-stranded portion of a polynucleotide. In some embodiments, the entire polynucleotide comprising a target C can be single-stranded. For example, a cytidine deaminase incorporated into the base editor can deaminate a target C in a single-stranded RNA polynucleotide. In other embodiments, a base editor comprising a cytidine deaminase domain can act on a double-stranded polynucleotide, but the target C can be positioned in a portion of the polynucleotide which at the time of the deamination reaction is in a single-stranded state. For example, in embodiments where the NAGPB domain comprises a Cas9 domain, several nucleotides can be left unpaired during formation of the Cas9-gRNA-target DNA complex, resulting in formation of a Cas9 “R-loop complex”. These unpaired nucleotides can form a bubble of single-stranded DNA that can serve as a substrate for a single-strand specific nucleotide deaminase enzyme (e.g., cytidine deaminase).

In some embodiments, a cytidine deaminase of a base editor can comprise all or a portion of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase. In some embodiments, a deaminase incorporated into a base editor comprises all or a portion of an APOBEC1 deaminase. In some embodiments, a deaminase incorporated into a base editor comprises all or a portion of APOBEC2 deaminase. In some embodiments, a deaminase incorporated into a base editor comprises all or a portion of is an APOBEC3 deaminase. In some embodiments, a deaminase incorporated into a base editor comprises all or a portion of an APOBEC3A deaminase. In some embodiments, a deaminase incorporated into a base editor comprises all or a portion of APOBEC3B deaminase. In some embodiments, a deaminase incorporated into a base editor comprises all or a portion of APOBEC3C deaminase. In some embodiments, a deaminase incorporated into a base editor comprises all or a portion of APOBEC3D deaminase. In some embodiments, a deaminase incorporated into a base editor comprises all or a portion of APOBEC3E deaminase. In some embodiments, a deaminase incorporated into a base editor comprises all or a portion of APOBEC3F deaminase. In some embodiments, a deaminase incorporated into a base editor comprises all or a portion of APOBEC3G deaminase. In some embodiments, a deaminase incorporated into a base editor comprises all or a portion of APOBEC3H deaminase. In some embodiments, a deaminase incorporated into a base editor comprises all or a portion of APOBEC4 deaminase. In some embodiments, a deaminase incorporated into a base editor comprises all or a portion of activation-induced deaminase (AID). In some embodiments a deaminase incorporated into a base editor comprises all or a portion of cytidine deaminase 1 (CDA1). It should be appreciated that a base editor can comprise a deaminase from any suitable organism (e.g., a human or a rat). In some embodiments, a deaminase domain of a base editor is from a human, chimpanzee, gorilla, monkey, cow, dog, rat, or mouse. In some embodiments, the deaminase domain of the base editor is derived from rat (e.g., rat APOBEC1). In some embodiments, the deaminase domain of the base editor is human APOBEC1. In some embodiments, the deaminase domain of the base editor is pmCDA1.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins described herein, for example by making point mutations in the deaminase domain, affect the processivity of the fusion proteins (e.g., base editors). For example, mutations that reduce, but do not eliminate, the catalytic activity of a deaminase domain within a base editing fusion protein can make it less likely that the deaminase domain will catalyze the deamination of a residue adjacent to a target residue, thereby narrowing the deamination window. The ability to narrow the deamination window can prevent unwanted deamination of residues adjacent to specific target residues, which can decrease or prevent off-target effects.

For example, in some embodiments, an APOBEC deaminase incorporated into a base editor can comprise one or more mutations selected from the group consisting of H121X, H122X, R126X, R126X, R118X, W90X, W90X, and R132X of rAPOBEC1, or one or more corresponding mutations in another APOBEC deaminase, wherein X is any amino acid. In some embodiments, an APOBEC deaminase incorporated into a base editor can comprise one or more mutations selected from the group consisting of H121R, H122R, R126A, R126E, R118A, W90A, W90Y, and R132E of rAPOBEC1, or one or more corresponding mutations in another APOBEC deaminase.

In some embodiments, an APOBEC deaminase incorporated into a base editor can comprise one or more mutations selected from the group consisting of D316X, D317X, R320X, R320X, R313X, W285X, W285X, R326X of hAPOBEC3G, or one or more corresponding mutations in another APOBEC deaminase, wherein X is any amino acid. In some embodiments, any of the fusion proteins provided herein comprise an APOBEC deaminase comprising one or more mutations selected from the group consisting of D316R, D317R, R320A, R320E, R313A, W285A, W285Y, R326E of hAPOBEC3G, or one or more corresponding mutations in another APOBEC deaminase.

In some embodiments, an APOBEC deaminase incorporated into a base editor can comprise a H121R and a H122R mutation of rAPOBEC1, or one or more corresponding mutations in another APOBEC deaminase. In some embodiments an APOBEC deaminase incorporated into a base editor can comprise an APOBEC deaminase comprising a R126A mutation of rAPOBEC1, or one or more corresponding mutations in another APOBEC deaminase. In some embodiments, an APOBEC deaminase incorporated into a base editor can comprise an APOBEC deaminase comprising a R126E mutation of rAPOBEC1, or one or more corresponding mutations in another APOBEC deaminase. In some embodiments, an APOBEC deaminase incorporated into a base editor can comprise an APOBEC deaminase comprising a R118A mutation of rAPOBEC1, or one or more corresponding mutations in another APOBEC deaminase. In some embodiments, an APOBEC deaminase incorporated into a base editor can comprise an APOBEC deaminase comprising a W90A mutation of rAPOBEC1, or one or more corresponding mutations in another APOBEC deaminase. In some embodiments, an APOBEC deaminase incorporated into a base editor can comprise an APOBEC deaminase comprising a W90Y mutation of rAPOBEC1, or one or more corresponding mutations in another APOBEC deaminase. In some embodiments, an APOBEC deaminase incorporated into a base editor can comprise an APOBEC deaminase comprising a R132E mutation of rAPOBEC1, or one or more corresponding mutations in another APOBEC deaminase. In some embodiments an APOBEC deaminase incorporated into a base editor can comprise an APOBEC deaminase comprising a W90Y and a R126E mutation of rAPOBEC1, or one or more corresponding mutations in another APOBEC deaminase. In some embodiments, an APOBEC deaminase incorporated into a base editor can comprise an APOBEC deaminase comprising a R126E and a R132E mutation of rAPOBEC1, or one or more corresponding mutations in another APOBEC deaminase. In some embodiments, an APOBEC deaminase incorporated into a base editor can comprise an APOBEC deaminase comprising a W90Y and a R132E mutation of rAPOBEC1, or one or more corresponding mutations in another APOBEC deaminase. In some embodiments, an APOBEC deaminase incorporated into a base editor can comprise an APOBEC deaminase comprising a W90Y, R126E, and R132E mutation of rAPOBEC1, or one or more corresponding mutations in another APOBEC deaminase.

In some embodiments, an APOBEC deaminase incorporated into a base editor can comprise an APOBEC deaminase comprising a D316R and a D317R mutation of hAPOBEC3G, or one or more corresponding mutations in another APOBEC deaminase. In some embodiments, any of the fusion proteins provided herein comprise an APOBEC deaminase comprising a R320A mutation of hAPOBEC3G, or one or more corresponding mutations in another APOBEC deaminase. In some embodiments, an APOBEC deaminase incorporated into a base editor can comprise an APOBEC deaminase comprising a R320E mutation of hAPOBEC3G, or one or more corresponding mutations in another APOBEC deaminase. In some embodiments, an APOBEC deaminase incorporated into a base editor can comprise an APOBEC deaminase comprising a R313A mutation of hAPOBEC3G, or one or more corresponding mutations in another APOBEC deaminase. In some embodiments, an APOBEC deaminase incorporated into a base editor can comprise an APOBEC deaminase comprising a W285A mutation of hAPOBEC3G, or one or more corresponding mutations in another APOBEC deaminase. In some embodiments, an APOBEC deaminase incorporated into a base editor can comprise an APOBEC deaminase comprising a W285Y mutation of hAPOBEC3G, or one or more corresponding mutations in another APOBEC deaminase. In some embodiments, an APOBEC deaminase incorporated into a base editor can comprise an APOBEC deaminase comprising a R326E mutation of hAPOBEC3G, or one or more corresponding mutations in another APOBEC deaminase. In some embodiments, an APOBEC deaminase incorporated into a base editor can comprise an APOBEC deaminase comprising a W285Y and a R320E mutation of hAPOBEC3G, or one or more corresponding mutations in another APOBEC deaminase. In some embodiments, an APOBEC deaminase incorporated into a base editor can comprise an APOBEC deaminase comprising a R320E and a R326E mutation of hAPOBEC3G, or one or more corresponding mutations in another APOBEC deaminase. In some embodiments, an APOBEC deaminase incorporated into a base editor can comprise an APOBEC deaminase comprising a W285Y and a R326E mutation of hAPOBEC3G, or one or more corresponding mutations in another APOBEC deaminase. In some embodiments, an APOBEC deaminase incorporated into a base editor can comprise an APOBEC deaminase comprising a W285Y, R320E, and R326E mutation of hAPOBEC3G, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion of an APOBEC1 deaminase.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Cytidine Deaminases

In some embodiments, the fusion proteins of the invention comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

The disclosure provides any deaminase domains with a certain percent identity plus any of the mutations or combinations thereof described herein. In some embodiments, the cytidine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the cytidine deaminases provided herein. In some embodiments, the cytidine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

A fusion protein of the invention second protein comprises two or more nucleic acid editing domains.

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, and then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. See e.g., “Complete genome sequence of an M1 strain of Streptococcus pyogenes.” Ferretti, J. J. et al., Natl. Acad. Sci. U.S.A. 98:4658-4663(2001); “CRISPR RNA maturation by trans-encoded small RNA and host factor RNase III.” Deltcheva E. et al., Nature 471:602-607(2011); and “Programmable dual-RNA-guided DNA endonuclease in adaptive bacterial immunity.” Jinek M.et al, Science 337:816-821(2012), the entire contents of each of which are incorporated herein by reference).

The PAM sequence can be any PAM sequence known in the art. Suitable PAM sequences include, but are not limited to, NGG, NGA, NGC, NGN, NGT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

In an embodiment, a guide polynucleotide described herein can be RNA or DNA. In one embodiment, the guide polynucleotide is a gRNA. An RNA/Cas complex can assist in “guiding” a Cas protein to a target DNA. Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M. et al., Science 337:816-821(2012), the entire contents of which is hereby incorporated by reference.

In some embodiments, the guide polynucleotide is at least one single guide RNA (“sgRNA” or “gNRA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

In some embodiments, the guide polynucleotide is at least one tracrRNA. In some embodiments, the guide polynucleotide does not require PAM sequence to guide the polynucleotide-programmable DNA-binding domain (e.g., Cas9 or Cpf1) to the target nucleotide sequence.

A guide polynucleotide may include natural or non-natural (or unnatural) nucleotides (e.g., peptide nucleic acid or nucleotide analogs). In some cases, the targeting region of a guide nucleic acid sequence can be at least 15, 16, 17, 18, 19, 20, 21, 22, 23, 24, 25, 26, 27, 28, 29, or 30 nucleotides in length. A targeting region of a guide nucleic acid can be between 10-30 nucleotides in length, or between 15-25 nucleotides in length, or between 15-20 nucleotides in length.

In some embodiments, the base editor provided herein utilizes one or more guide polynucleotide (e.g., multiple gRNA). In some embodiments, a single guide polynucleotide is utilized for different base editors described herein. For example, a single guide polynucleotide can be utilized for a cytidine base editor and an adenosine base editor.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 321-331. Thus, a skilled artisan can change the genomic target of the Cas protein specificity is partially determined by how specific the gRNA targeting sequence is for the genomic target compared to the rest of the genome.

In other embodiments, a guide polynucleotide can comprise both the polynucleotide targeting portion of the nucleic acid and the scaffold portion of the nucleic acid in a single molecule (i.e., a single-molecule guide nucleic acid). For example, a single-molecule guide polynucleotide can be a single guide RNA (sgRNA or gRNA). Herein the term guide polynucleotide sequence contemplates any single, dual or multi-molecule nucleic acid capable of interacting with and directing a base editor to a target polynucleotide sequence.

Typically, a guide polynucleotide (e.g., crRNA/trRNA complex or a gRNA) comprises a “polynucleotide-targeting segment” that includes a sequence capable of recognizing and binding to a target polynucleotide sequence, and a “protein-binding segment” that stabilizes the guide polynucleotide within a polynucleotide programmable nucleotide binding domain component of a base editor. In some embodiments, the polynucleotide targeting segment of the guide polynucleotide recognizes and binds to a DNA polynucleotide, thereby facilitating the editing of a base in DNA. In other cases, the polynucleotide targeting segment of the guide polynucleotide recognizes and binds to an RNA polynucleotide, thereby facilitating the editing of a base in RNA. Herein a “segment” refers to a section or region of a molecule, e.g., a contiguous stretch of nucleotides in the guide polynucleotide. A segment can also refer to a region/section of a complex such that a segment can comprise regions of more than one molecule. For example, where a guide polynucleotide comprises multiple nucleic acid molecules, the protein-binding segment of can include all or a portion of multiple separate molecules that are for instance hybridized along a region of complementarity. In some embodiments, a protein-binding segment of a DNA-targeting RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and can include regions of RNA molecules that are of any total length and can include regions with complementarity to other molecules.

The guide polynucleotides can be synthesized chemically, synthesized enzymatically, or a combination thereof. For example, the gRNA can be synthesized using standard phosphoramidite-based solid-phase synthesis methods. Alternatively, the gRNA can be synthesized in vitro by operably linking DNA encoding the gRNA to a promoter control sequence that is recognized by a phage RNA polymerase. Examples of suitable phage promoter sequences include T7, T3, SP6 promoter sequences, or variations thereof. In embodiments in which the gRNA comprises two separate molecules (e.g., crRNA and tracrRNA), the crRNA can be chemically synthesized and the tracrRNA can be enzymatically synthesized. A gRNA molecule can be transcribed in vitro.

A guide polynucleotide may be expressed, for example, by a DNA that encodes the gRNA, e.g., a DNA vector comprising a sequence encoding the gRNA. The gRNA may be encoded alone or together with an encoded base editor. Such DNA sequences may be introduced into an expression system, e.g., a cell, together or separately. For example, DNA sequences encoding a polynucleotide programmable nucleotide binding domain and a gRNA may be introduced into a cell, each DNA sequence can be part of a separate molecule (e.g., one vector containing the polynucleotide programmable nucleotide binding domain coding sequence and a second vector containing the gRNA coding sequence) or both can be part of a same molecule (e.g., one vector containing coding (and regulatory) sequence for both the polynucleotide programmable nucleotide binding domain and the gRNA). An RNA can be transcribed from a synthetic DNA molecule, e.g., a gBlocks® gene fragment.

A gRNA or a guide polynucleotide can comprise three regions: a first region at the 5′ end that can be complementary to a target site in a chromosomal sequence, a second internal region that can form a stem loop structure, and a third 3′ region that can be single-stranded. A first region of each gRNA can also be different such that each gRNA guides a fusion protein to a specific target site. Further, second and third regions of each gRNA can be identical in all gRNAs.

A first region of a gRNA or a guide polynucleotide can be complementary to sequence at a target site in a chromosomal sequence such that the first region of the gRNA can base pair with the target site. In some cases, a first region of a gRNA can comprise from or from about 10 nucleotides to 25 nucleotides (i.e., from 10 nucleotides to nucleotides; or from about 10 nucleotides to about 25 nucleotides; or from 10 nucleotides to about 25 nucleotides; or from about 10 nucleotides to 25 nucleotides) or more. For example, a region of base pairing between a first region of a gRNA and a target site in a chromosomal sequence can be or can be about 10, 11, 12, 13, 14, 15, 16, 17, 18, 19, 20, 22, 23, 24, 25, or more nucleotides in length. Sometimes, a first region of a gRNA can be or can be about 19, 20, or 21 nucleotides in length.

A gRNA or a guide polynucleotide can also comprise a second region that forms a secondary structure. For example, a secondary structure formed by a gRNA can comprise a stem (or hairpin) and a loop. A length of a loop and a stem can vary. For example, a loop can range from or from about 3 to 10 nucleotides in length, and a stem can range from or from about 6 to 20 base pairs in length. A stem can comprise one or more bulges of 1 to 10 or about 10 nucleotides. The overall length of a second region can range from or from about 16 to 60 nucleotides in length. For example, a loop can be or can be about 4 nucleotides in length and a stem can be or can be about 12 base pairs.

A gRNA or a guide polynucleotide can also comprise a third region at the 3′ end that can be essentially single-stranded. For example, a third region is sometimes not complementarity to any chromosomal sequence in a cell of interest and is sometimes not complementarity to the rest of a gRNA. Further, the length of a third region can vary. A third region can be more than or more than about 4 nucleotides in length. For example, the length of a third region can range from or from about 5 to 60 nucleotides in length.

A gRNA or a guide polynucleotide can target any exon or intron of a gene target. In some cases, a guide can target exon 1 or 2 of a gene, in other cases; a guide can target exon 3 or 4 of a gene. In some embodiments, a composition comprises multiple gRNAs that all target the same exon or multiple gRNAs that target different exons. An exon and/or an intron of a gene can be targeted.

A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

Methods for selecting, designing, and validating guide polynucleotides, e.g., gRNAs and targeting sequences are described herein and known to those skilled in the art. For example, to minimize the impact of potential substrate promiscuity of a deaminase domain in the nucleobase editor system (e.g., an AID domain), the number of residues that could unintentionally be targeted for deamination (e.g., off-target C residues that could potentially reside on single strand DNA within the target nucleic acid locus) may be minimized. In addition, software tools can be used to optimize the gRNAs corresponding to a target nucleic acid sequence, e.g., to minimize total off-target activity across the genome. For example, for each possible targeting domain choice using S. pyogenes Cas9, all off-target sequences (preceding selected PAMs, e.g., NAG or NGG) may be identified across the genome that contain up to certain number (e.g., 1, 2, 3, 4, 5, 6, 7, 8, 9, or 10) of mismatched base-pairs. First regions of gRNAs complementary to a target site can be identified, and all first regions (e.g., crRNAs) can be ranked according to its total predicted off-target score; the top-ranked targeting domains represent those that are likely to have the greatest on-target and the least off-target activity. Candidate targeting gRNAs can be functionally evaluated by using methods known in the art and/or as set forth herein.

As a non-limiting example, target DNA hybridizing sequences in crRNAs of a gRNA for use with Cas9s may be identified using a DNA sequence searching algorithm. gRNA design is carried out using custom gRNA design software based on the public tool cas-offinder as described in Bae S., Park J., & Kim J.-S. Cas-OFFinder: A fast and versatile algorithm that searches for potential off-target sites of Cas9 RNA-guided endonucleases. Bioinformatics 30, 1473-1475 (2014). This software scores guides after calculating their genome-wide off-target propensity. Typically matches ranging from perfect matches to 7 mismatches are considered for guides ranging in length from 17 to 24. Once the off-target sites are computationally-determined, an aggregate score is calculated for each guide and summarized in a tabular output using a web-interface. In addition to identifying potential target sites adjacent to PAM sequences, the software also identifies all PAM adjacent sequences that differ by 1, 2, 3 or more than 3 nucleotides from the selected target sites. Genomic DNA sequences for a target nucleic acid sequence, e.g., a target gene may be obtained and repeat elements may be screened using publicly available tools, for example, the RepeatMasker program. RepeatMasker searches input DNA sequences for repeated elements and regions of low complexity. The output is a detailed annotation of the repeats present in a given query sequence.

Following identification, first regions of gRNAs, e.g., crRNAs, are ranked into tiers based on their distance to the target site, their orthogonality and presence of 5′ nucleotides for close matches with relevant PAM sequences (for example, a 5′ G based on identification of close matches in the human genome containing a relevant PAM e.g., NGG PAM for S. pyogenes, NNGRRT or NNGRRV PAM for S. aureus). As used herein, orthogonality refers to the number of sequences in the human genome that contain a minimum number of mismatches to the target sequence. A “high level of orthogonality” or “good orthogonality” may, for example, refer to 20-mer targeting domains that have no identical sequences in the human genome besides the intended target, nor any sequences that contain one or two mismatches in the target sequence. Targeting domains with good orthogonality may be selected to minimize off-target DNA cleavage.

A gRNA can then be introduced into a cell or embryo as an RNA molecule or a non-RNA nucleic acid molecule, e.g., DNA molecule. In one embodiment, a DNA encoding a gRNA is operably linked to promoter control sequence for expression of the gRNA in a cell or embryo of interest. A RNA coding sequence can be operably linked to a promoter sequence that is recognized by RNA polymerase III (Pol III). Plasmid vectors that can be used to express gRNA include, but are not limited to, px330 vectors and px333 vectors. In some cases, a plasmid vector (e.g., px333 vector) can comprise at least two gRNA-encoding DNA sequences. Further, a vector can comprise additional expression control sequences (e.g., enhancer sequences, Kozak sequences, polyadenylation sequences, transcriptional termination sequences, etc.), selectable marker sequences (e.g., GFP or antibiotic resistance genes such as puromycin), origins of replication, and the like. A DNA molecule encoding a gRNA can also be linear. A DNA molecule encoding a gRNA or a guide polynucleotide can also be circular.

In some embodiments, a reporter system is used for detecting base-editing activity and testing candidate guide polynucleotides. In some embodiments, a reporter system comprises a reporter gene based assay where base editing activity leads to expression of the reporter gene. For example, a reporter system may include a reporter gene comprising a deactivated start codon, e.g., a mutation on the template strand from 3′-TAC-5′ to 3′-CAC-5′. Upon successful deamination of the target C, the corresponding mRNA will be transcribed as 5′-AUG-3′ instead of 5′-GUG-3′, enabling the translation of the reporter gene. Suitable reporter genes will be apparent to those of skill in the art. Non-limiting examples of reporter genes include gene encoding green fluorescence protein (GFP), red fluorescence protein (RFP), luciferase, secreted alkaline phosphatase (SEAP), or any other gene whose expression are detectable and apparent to those skilled in the art. The reporter system can be used to test many different gRNAs, e.g., in order to determine which residue(s) with respect to the target DNA sequence the respective deaminase will target. sgRNAs that target non-template strand can also be tested in order to assess off-target effects of a specific base editing protein, e.g., a Cas9 deaminase fusion protein. In some embodiments, such gRNAs can be designed such that the mutated start codon will not be base-paired with the gRNA. The guide polynucleotides can comprise standard ribonucleotides, modified ribonucleotides (e.g., pseudouridine), ribonucleotide isomers, and/or ribonucleotide analogs. In some embodiments, the guide polynucleotide can comprise at least one detectable label. The detectable label can be a fluorophore (e.g., FAM, TMR, Cy3, Cy5, Texas Red, Oregon Green, Alexa Fluors, Halo tags, or suitable fluorescent dye), a detection tag (e.g., biotin, digoxigenin, and the like), quantum dots, or gold particles.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and are preferably separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In a particular embodiment, the chemical modifications are 2′-O-methyl (2′-OMe) modifications. The modified guide RNAs may improve saCas9 efficacy and also specificity. The effect of an individual modification varies based on the position and combination of chemical modifications used as well as the inter- and intramolecular interactions with other modified nucleotides. By way of example, S-cEt has been used to improve oligonucleotide intramolecular folding.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises four modified nucleosides at the 5′ end and four modified nucleosides at the 3′ end of the guide. In some embodiments, the modified nucleoside comprises a 2′ O-methyl or a phosphorothioate.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications (“heavy mods”). Such heavy mods can increase base editing ˜2 fold in vivo or in vitro. For such modifications, mN=2′-OMe; Ns=phosphorothioate (PS), where “N” represents the any nucleotide, as would be understood by one having skill in the art. In some cases, a nucleotide (N) may contain two modifications, for example, both a 2′-OMe and a PS modification. For example, a nucleotide with a phosphorothioate and 2′ OMe is denoted as “mNs;” when there are two modifications next to each other, the notation is “mNsmNs.

In some embodiments of the modified gRNA, the gRNA comprises one or more chemical modifications selected from the group consisting of 2′-O-methyl (2′-OMe), phosphorothioate (PS), 2′-O-methyl thioPACE (MSP), 2′-O-methyl-PACE (MP), 2′-O-methyl thioPACE (MSP), 2′-fluoro RNA (2′-F-RNA), and constrained ethyl (S-cEt). In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

In some cases, a gRNA or a guide polynucleotide can comprise modifications. A modification can be made at any location of a gRNA or a guide polynucleotide. More than one modification can be made to a single gRNA or a guide polynucleotide. A gRNA or a guide polynucleotide can undergo quality control after a modification. In some cases, quality control can include PAGE, HPLC, MS, or any combination thereof.

A modification of a gRNA or a guide polynucleotide can be a substitution, insertion, deletion, chemical modification, physical modification, stabilization, purification, or any combination thereof.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencer 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a modification is permanent. In other cases, a modification is transient. In some cases, multiple modifications are made to a gRNA or a guide polynucleotide. A gRNA or a guide polynucleotide modification can alter physiochemical properties of a nucleotide, such as their conformation, polarity, hydrophobicity, chemical reactivity, base-pairing interactions, or any combination thereof.

A guide polynucleotide can be transferred into a cell by transfecting the cell with an isolated gRNA or a plasmid DNA comprising a sequence coding for the guide RNA and a promoter. A gRNA or a guide polynucleotide can also be transferred into a cell in other way, such as using virus-mediated gene delivery. A gRNA or a guide polynucleotide can be isolated. For example, a gRNA can be transfected in the form of an isolated RNA into a cell or organism. A gRNA can be prepared by in vitro transcription using any in vitro transcription system known in the art. A gRNA can be transferred to a cell in the form of isolated RNA rather than in the form of plasmid comprising encoding sequence for a gRNA.

A modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a gRNA or a guide polynucleotide. A modification can also enhance biological activity. In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

In some embodiments, the guide RNA is designed to disrupt a splice site (i.e., a splice acceptor (SA) or a splice donor (SD). In some embodiments, the guide RNA is designed such that the base editing results in a premature STOP codon.

### Protospacer Adjacent Motif

The term “protospacer adjacent motif (PAM)” or PAM-like motif refers to a 2-6 base pair DNA sequence immediately following the DNA sequence targeted by the Cas9 nuclease in the CRISPR bacterial adaptive immune system. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence is essential for target binding, but the exact sequence depends on a type of Cas protein. The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence. A PAM site is a nucleotide sequence in proximity to a target polynucleotide sequence. Some aspects of the disclosure provide for base editors comprising all or a portion of CRISPR proteins that have different PAM specificities.

For example, typically Cas9 proteins, such as Cas9 from S. pyogenes (spCas9), require a canonical NGG PAM sequence to bind a particular nucleic acid region, where the “N” in “NGG” is adenine (A), thymine (T), guanine (G), or cytosine (C), and the G is guanine. A PAM can be CRISPR protein-specific and can be different between different base editors comprising different CRISPR protein-derived domains. A PAM can be 5′ or 3′ of a target sequence. A PAM can be upstream or downstream of a target sequence. A PAM can be 1, 2, 3, 4, 5, 6, 7, 8, 9, 10 or more nucleotides in length. Often, a PAM is between 2-6 nucleotides in length.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 7 below.

TABLE 7
Cas9 proteins and corresponding PAM sequences

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-MQKSER | NGCG |
| spCas9-MQKSER | NGCN |
| spCas9-LRKIQK | NGTN |
| spCas9-LRVSQK | NGTN |
| spCas9-LRVSQL | NGTN |
| spCas9-MQKFRAER | NGC |
| Cpf1 | 5′ (TTTV) |
| SpyMac | 5′-NAA-3′ |

In some embodiments, the PAM is NGC. In some embodiments, the NGC PAM is recognized by a Cas9 variant. In some embodiments, the NGC PAM variant includes one or more amino acid substitutions selected from D1135M, S1136Q, G1218K, E1219F, A1322R, D1332A, R1335E, and T1337R (collectively termed “MQKFRAER”).

In some embodiments, the PAM is NGT. In some embodiments, the NGT PAM is recognized by a Cas9 variant. In some embodiments, the NGT PAM variant is generated through targeted mutations at one or more residues 1335, 1337, 1135, 1136, 1218, and/or 1219. In some embodiments, the NGT PAM variant is created through targeted mutations at one or more residues 1219, 1335, 1337, 1218. In some embodiments, the NGT PAM variant is created through targeted mutations at one or more residues 1135, 1136, 1218, 1219, and 1335. In some embodiments, the NGT PAM variant is selected from the set of targeted mutations provided in Tables 8A and 8B below.

TABLE 8A
NGT PAM Variant Mutations at residues 1219, 1335, 1337, 1218

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |
| 9 | L | L | T | |
| 10 | L | L | R | |
| 11 | L | L | Q | |
| 12 | L | L | L | |
| 13 | F | I | T | |
| 14 | F | I | R | |
| 15 | F | I | Q | |
| 16 | F | I | L | |
| 17 | F | G | C | |
| 18 | H | L | N | |
| 19 | F | G | C | A |
| 20 | H | L | N | V |
| 21 | L | A | W | |
| 22 | L | A | F | |
| 23 | L | A | Y | |
| 24 | I | A | W | |
| 25 | I | A | F | |
| 26 | I | A | Y | |

TABLE 8B
NGT PAM Variant Mutations at residues
1135, 1136, 1218, 1219, and 1335

| Variant | D1135L | S1136R | G1218S | E1219V | R1335Q |
|---|---|---|---|---|---|
| 27 | G | | | | |
| 28 | V | | | | |
| 29 | I | | | | |
| 30 | | A | | | |
| 31 | | W | | | |
| 32 | | H | | | |
| 33 | | K | | | |
| 34 | | | K | | |
| 35 | | | R | | |
| 36 | | | Q | | |
| 37 | | | T | | |
| 38 | | | N | | |
| 39 | | | | I | |
| 40 | | | | A | |
| 41 | | | | N | |
| 42 | | | | Q | |
| 43 | | | | G | |
| 44 | | | | L | |
| 45 | | | | S | |
| 46 | | | | T | |
| 47 | | | | | L |
| 48 | | | | | I |
| 49 | | | | | V |
| 50 | | | | | N |
| 51 | | | | | S |
| 52 | | | | | T |
| 53 | | | | | F |
| 54 | | | | | Y |
| 55 | N1286Q | I1331F | | | |

In some embodiments, the NGT PAM variant is selected from variant 5, 7, 28, 31, or 36 in Table 8A and Table 8B. In some embodiments, the variants have improved NGT PAM recognition.

In some embodiments, the NGT PAM variants have mutations at residues 1219, 1335, 1337, and/or 1218. In some embodiments, the NGT PAM variant is selected with mutations for improved recognition from the variants provided in Table 9 below.

TABLE 9
NGT PAM Variant Mutations at residues
1219, 1335, 1337, and 1218

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |

In some embodiments, the NGT PAM is selected from the variants provided in Table 10 below.

TABLE 10
NGT PAM variants

| | NGTN | | | | | | | |
|---|---|---|---|---|---|---|---|---|
| | variant | D1135 | S1136 | G1218 | E1219 | A1322R | R1335 | T1337 |
| Variant 1 | LRKIQK | L | R | K | I | — | Q | K |
| Variant 2 | LRSVQK | L | R | S | V | — | Q | K |
| Variant 3 | LRSVQL | L | R | S | V | — | Q | L |
| Variant 4 | LRKIRQK | L | R | K | I | R | Q | K |
| Variant 5 | LRSVRQK | L | R | S | V | R | Q | K |
| Variant 6 | LRSVRQL | L | R | S | V | R | Q | L |

In some embodiments the NGTN variant is variant 1. In some embodiments, the NGTN variant is variant 2. In some embodiments, the NGTN variant is variant 3. In some embodiments, the NGTN variant is variant 4. In some embodiments, the NGTN variant is variant 5. In some embodiments, the NGTN variant is variant 6.

In some embodiments, the Cas9 domain is a Cas9 domain from Streptococcus pyogenes (SpCas9). In some embodiments, the SpCas9 domain is a nuclease active SpCas9, a nuclease inactive SpCas9 (SpCas9d), or a SpCas9 nickase (SpCas9n). In some embodiments, the SpCas9 comprises a D9X mutation, or a corresponding mutation in any of the amino acid sequences provided herein, wherein X is any amino acid except for D. In some embodiments, the SpCas9 comprises a D9A mutation, or a corresponding mutation in any of the amino acid sequences provided herein. In some embodiments, the SpCas9 domain, the SpCas9d domain, or the SpCas9n domain can bind to a nucleic acid sequence having a non-canonical PAM. In some embodiments, the SpCas9 domain, the SpCas9d domain, or the SpCas9n domain can bind to a nucleic acid sequence having an NGG, a NGA, or a NGCG PAM sequence.

In some embodiments, the SpCas9 domain comprises one or more of a D1135X, a R1335X, and a T1337X mutation, or a corresponding mutation in any of the amino acid sequences provided herein, wherein X is any amino acid. In some embodiments, the SpCas9 domain comprises one or more of a D1135E, R1335Q, and T1337R mutation, or a corresponding mutation in any of the amino acid sequences provided herein. In some embodiments, the SpCas9 domain comprises a D1135E, a R1335Q, and a T1337R mutation, or corresponding mutations in any of the amino acid sequences provided herein. In some embodiments, the SpCas9 domain comprises one or more of a D1135X, a R1335X, and a T1337X mutation, or a corresponding mutation in any of the amino acid sequences provided herein, wherein X is any amino acid. In some embodiments, the SpCas9 domain comprises one or more of a D1135V, a R1335Q, and a T1337R mutation, or a corresponding mutation in any of the amino acid sequences provided herein. In some embodiments, the SpCas9 domain comprises a D1135V, a R1335Q, and a T1337R mutation, or corresponding mutations in any of the amino acid sequences provided herein. In some embodiments, the SpCas9 domain comprises one or more of a D1135X, a G1218X, a R1335X, and a T1337X mutation, or a corresponding mutation in any of the amino acid sequences provided herein, wherein X is any amino acid. In some embodiments, the SpCas9 domain comprises one or more of a D1135V, a G1218R, a R1335Q, and a T1337R mutation, or a corresponding mutation in any of the amino acid sequences provided herein. In some embodiments, the SpCas9 domain comprises a D1135V, a G1218R, a R1335Q, and a T1337R mutation, or corresponding mutations in any of the amino acid sequences provided herein.

In some examples, a PAM recognized by a CRISPR protein-derived domain of a base editor disclosed herein can be provided to a cell on a separate oligonucleotide to an insert (e.g., an AAV insert) encoding the base editor. In such embodiments, providing PAM on a separate oligonucleotide can allow cleavage of a target sequence that otherwise would not be able to be cleaved, because no adjacent PAM is present on the same polynucleotide as the target sequence.

In an embodiment, S. pyogenes Cas9 (SpCas9) can be used as a CRISPR endonuclease for genome engineering. However, others can be used. In some embodiments, a different endonuclease can be used to target certain genomic targets. In some embodiments, synthetic SpCas9-derived variants with non-NGG PAM sequences can be used. Additionally, other Cas9 orthologues from various species have been identified and these “non-SpCas9s” can bind a variety of PAM sequences that can also be useful for the present disclosure. For example, the relatively large size of SpCas9 (approximately 4 kb coding sequence) can lead to plasmids carrying the SpCas9 cDNA that cannot be efficiently expressed in a cell. Conversely, the coding sequence for Staphylococcus aureus Cas9 (SaCas9) is approximately 1 kilobase shorter than SpCas9, possibly allowing it to be efficiently expressed in a cell. Similar to SpCas9, the SaCas9 endonuclease is capable of modifying target genes in mammalian cells in vitro and in mice in vivo. In some embodiments, a Cas protein can target a different PAM sequence. In some embodiments, a target gene can be adjacent to a Cas9 PAM, 5′-NGG, for example. In other embodiments, other Cas9 orthologs can have different PAM requirements. For example, other PAMs such as those of S. thermophilus (5′-NNAGAA for CRISPRI and 5′-NGGNG for CRISPR3) and Neisseria meningitidis (5′-NNNNGATT) can also be found adjacent to a target gene.

In some embodiments, for a S. pyogenes system, a target gene sequence can precede (i.e., be 5′ to) a 5′-NGG PAM, and a 20-nt guide RNA sequence can base pair with an opposite strand to mediate a Cas9 cleavage adjacent to a PAM. In some embodiments, an adjacent cut can be or can be about 3 base pairs upstream of a PAM. In some embodiments, an adjacent cut can be or can be about 10 base pairs upstream of a PAM. In some embodiments, an adjacent cut can be or can be about 0-20 base pairs upstream of a PAM. For example, an adjacent cut can be next to, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 base pairs upstream of a PAM. An adjacent cut can also be downstream of a PAM by 1 to 30 base pairs. The sequences of exemplary SpCas9 proteins capable of binding a PAM sequence follow:

In some embodiments, engineered SpCas9 variants are capable of recognizing protospacer adjacent motif (PAM) sequences flanked by a 3′ H (non-G PAM) (see Tables 3A-3D). In some embodiments, the SpCas9 variants recognize NRNH PAMs (where R is A or G and H is A, C or T). In some embodiments, the non-G PAM is NRRH, NRTH, or NRCH (see e.g., Miller, S. M., et al. Continuous evolution of SpCas9 variants compatible with non-G PAMs, Nat. Biotechnol. (2020), the contents of which is incorporated herein by reference in its entirety).

In some embodiments, the Cas9 domain is a recombinant Cas9 domain. In some embodiments, the recombinant Cas9 domain is a SpyMacCas9 domain. In some embodiments, the SpyMacCas9 domain is a nuclease active SpyMacCas9, a nuclease inactive SpyMacCas9 (SpyMacCas9d), or a SpyMacCas9 nickase (SpyMacCas9n). In some embodiments, the SaCas9 domain, the SaCas9d domain, or the SaCas9n domain can bind to a nucleic acid sequence having a non-canonical PAM. In some embodiments, the SpyMacCas9 domain, the SpCas9d domain, or the SpCas9n domain can bind to a nucleic acid sequence having a NAA PAM sequence.

The sequence of an exemplary Cas9 A homolog of Spy Cas9 in Streptococcus macacae with native 5′-NAAN-3′ PAM specificity is known in the art and described, for example, by Jakimo et al., (Chatterjee, et al., “A Cas9 with PAM recognition for adenine dinucleotides”, Nature Communications, vol. 11, article no. 2474 (2020)), and is in the Sequence Listing as SEQ ID NO: 241.

In some embodiments, a variant Cas9 protein harbors, H840A, P475A, W476A, N477A, D1125A, W1126A, and D1218A mutations such that the polypeptide has a reduced ability to cleave a target DNA or RNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors D10A, H840A, P475A, W476A, N477A, D1125A, W1126A, and D1218A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, when a variant Cas9 protein harbors W476A and WI 126A mutations or when the variant Cas9 protein harbors P475A, W476A, N477A, D1125A, W1126A, and D1218A mutations, the variant Cas9 protein does not bind efficiently to a PAM sequence. Thus, in some such cases, when such a variant Cas9 protein is used in a method of binding, the method does not require a PAM sequence. In other words, in some embodiments, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted). Also, mutations other than alanine substitutions are suitable.

In some embodiments, a CRISPR protein-derived domain of a base editor can comprise all or a portion of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556(7699), 57-63; Miller et al., “Continuous evolution of SpCas9 variants compatible with non-G PAMs” Nat. Biotechnol., 2020 April; 38(4):471-481; the entire contents of each are hereby incorporated by reference.

Fusion Proteins Comprising a NapDNAbp and a Cytidine Deaminase and/or Adenosine Deaminase

Some aspects of the disclosure provide fusion proteins comprising a Cas9 domain or other nucleic acid programmable DNA binding protein (e.g., Cas12) and one or more cytidine deaminase or adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion protein comprises the following domains A-C, A-D, or A-E:
NH₂-[A-B-C]-COOH;
NH₂-[A-B-C-D]-COOH; or
NH₂-[A-B-C-D-E]-COOH;
   wherein A and C or A, C, and E, each comprises one or more of the following:
an adenosine deaminase domain or an active fragment thereof,
a cytidine deaminase domain or an active fragment thereof, and
wherein B or B and D, each comprises one or more domains having nucleic acid sequence specific binding activity.

In some embodiments, the fusion protein comprises the following structure:
NH₂-[Aₙ-Bₒ-Cₙ]-COOH;
NH₂-[Aₙ-Bₒ-Cₙ-Dₒ]-COOH; or
NH₂-[Aₙ-Bₒ-Cₚ-Dₒ-E]-COOH;
   wherein A and C or A, C, and E, each comprises one or more of the following:
an adenosine deaminase domain or an active fragment thereof,
a cytidine deaminase domain or an active fragment thereof, and wherein n is an integer: 1, 2, 3, 4, or 5, wherein p is an integer: 0, 1, 2, 3, 4, or 5; wherein q is an integer 0, 1, 2, 3, 4, or 5; and wherein B or B and D each comprises a domain having nucleic acid sequence specific binding activity; and wherein o is an integer: 1, 2, 3, 4, or 5.

For example, and without limitation, in some embodiments, the fusion protein comprises the structure:
NH2-[adenosine deaminase]-[Cas9 domain]-COOH;
NH2-[Cas9 domain]-[adenosine deaminase]-COOH;
NH2-[cytidine deaminase]-[Cas9 domain]-COOH;
NH2-[Cas9 domain]-[cytidine deaminase]-COOH;
NH2-[cytidine deaminase]-[Cas9 domain]-[adenosine deaminase]-COOH;
NH2-[adenosine deaminase]-[Cas9 domain]-[cytidine deaminase]-COOH;
NH2-[adenosine deaminase]-[cytidine deaminase]-[Cas9 domain]-COOH;
NH2-[cytidine deaminase]-[adenosine deaminase]-[Cas9 domain]-COOH;
NH2-[Cas9 domain]-[adenosine deaminase]-[cytidine deaminase]-COOH; or
NH2-[Cas9 domain]-[cytidine deaminase]-[adenosine deaminase]-COOH.

In some embodiments, any of the Cas12 domains or Cas12 proteins provided herein may be fused with any of the cytidine or adenosine deaminases provided herein. For example, and without limitation, in some embodiments, the fusion protein comprises the structure:
NH2-[adenosine deaminase]-[Cas12 domain]-COOH;
NH2-[Cas12 domain]-[adenosine deaminase]-COOH;
NH2-[cytidine deaminase]-[Cas12 domain]-COOH;
NH2-[Cas12 domain]-[cytidine deaminase]-COOH;
NH2-[cytidine deaminase]-[Cas12 domain]-[adenosine deaminase]-COOH;
NH2-[adenosine deaminase]-[Cas12 domain]-[cytidine deaminase]-COOH;
NH2-[adenosine deaminase]-[cytidine deaminase]-[Cas12 domain]-COOH;
NH2-[cytidine deaminase]-[adenosine deaminase]-[Cas12 domain]-COOH;
NH2-[Cas12 domain]-[adenosine deaminase]-[cytidine deaminase]-COOH; or
NH2-[Cas12 domain]-[cytidine deaminase]-[adenosine deaminase]-COOH.

In some embodiments, the adenosine deaminase is a TadA*8. Exemplary fusion protein structures include the following:
NH2-[TadA*8]-[Cas9 domain]-COOH;
NH2-[Cas9 domain]-[TadA*8]-COOH;
NH2-[TadA*8]-[Cas12 domain]-COOH; or
NH2-[Cas12 domain]-[TadA*8]-COOH.

In some embodiments, the adenosine deaminase of the fusion protein comprises a TadA*8 and a cytidine deaminase and/or an adenosine deaminase. In some embodiments, the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24.

Exemplary fusion protein structures include the following:
NH2-[TadA*8]-[Cas9/Cas12]-[adenosine deaminase]-COOH;
NH2-[adenosine deaminase]-[Cas9/Cas12]-[TadA*8]-COOH;
NH2-[TadA*8]-[Cas9/Cas12]-[cytidine deaminase]-COOH; or
NH2-[cytidine deaminase]-[Cas9/Cas12]-[TadA*8]-COOH.

In some embodiments, the adenosine deaminase of the fusion protein comprises a TadA*9 and a cytidine deaminase and/or an adenosine deaminase. Exemplary fusion protein structures include the following:
NH2-[TadA*9]-[Cas9/Cas12]-[adenosine deaminase]-COOH;
NH2-[adenosine deaminase]-[Cas9/Cas12]-[TadA*9]-COOH;
NH2-[TadA*9]-[Cas9/Cas12]-[cytidine deaminase]-COOH; or
NH2-[cytidine deaminase]-[Cas9/Cas12]-[TadA*9]-COOH.

In some embodiments, the fusion protein can comprise a deaminase flanked by an N-terminal fragment and a C-terminal fragment of a Cas9 or Cas12 polypeptide. In some embodiments, the fusion protein comprises a cytidine deaminase flanked by an N-terminal fragment and a C-terminal fragment of a Cas9 or Cas12 polypeptide. In some embodiments, the fusion protein comprises an adenosine deaminase flanked by an N-terminal fragment and a C-terminal fragment of a Cas9 or Cas 12 polypeptide.

In some embodiments, the fusion proteins comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, the “-” used in the general architecture above indicates the presence of an optional linker. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins of the present disclosure may comprise one or more additional features. For example, in some embodiments, the fusion protein may comprise inhibitors, cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

Exemplary, yet nonlimiting, fusion proteins are described in International PCT Application Nos. PCT/2017/044935, PCT/US2019/044935, and PCT/US2020/016288, each of which is incorporated herein by reference for its entirety.

### Fusion Proteins Comprising a Nuclear Localiazation Sequence (NLS)

In some embodiments, the fusion proteins provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, an NLS comprises the amino acid sequence PKKKRKVEGADKRTADGSEFESPKKKRKV (SEQ ID NO: 332), KRTADGSEFESPKKKRKV (SEQ ID NO: 194), KRPAATKKAGQAKKKK (SEQ ID NO: 195), KKTELQTTNAENKTKKL (SEQ ID NO: 196), KRGINDRNFWRGENGRKTR (SEQ ID NO: 197), RKSGKIAAIVVKRPRKPKKKRKV (SEQ ID NO: 333), or MDSLLMNRRKFLYQFKNVRWAKGRRETYLC (SEQ ID NO: 200).

In some embodiments, the fusion proteins comprising a cytidine or adenosine deaminase, a Cas9 domain, and an NLS do not comprise a linker sequence. In some embodiments, linker sequences between one or more of the domains or proteins (e.g., cytidine or adenosine deaminase, Cas9 domain or NLS) are present. In some embodiments, a linker is present between the cytidine deaminase and adenosine deaminase domains and the napDNAbp. In some embodiments, the “-” used in the general architecture below indicates the presence of an optional linker. In some embodiments, the cytidine deaminase and adenosine deaminase and the napDNAbp are fused via any of the linkers provided herein. For example, in some embodiments the cytidine deaminase and adenosine deaminase and the napDNAbp are fused via any of the linkers provided herein.

In some embodiments, the general architecture of exemplary napDNAbp (e.g., Cas9 or Cas12) fusion proteins with a cytidine or adenosine deaminase and a napDNAbp (e.g., Cas9 or Cas12) domain comprises any one of the following structures, where NLS is a nuclear localization sequence (e.g., any NLS provided herein), NH₂ is the N-terminus of the fusion protein, and COOH is the C-terminus of the fusion protein:
NH₂-NLS-[cytidine deaminase]-[napDNAbp domain]-COOH;
NH₂-NLS [napDNAbp domain]-[cytidine deaminase]-COOH;
NH₂-[cytidine deaminase]-[napDNAbp domain]-NLS-COOH;
NH₂-[napDNAbp domain]-[cytidine deaminase]-NLS-COOH;
NH₂-NLS-[adenosine deaminase]-[napDNAbp domain]-COOH;
NH₂-NLS [napDNAbp domain]-[adenosine deaminase]-COOH;
NH₂-[adenosine deaminase]-[napDNAbp domain]-NLS-COOH;
NH₂-[napDNAbp domain]-[adenosine deaminase]-NLS-COOH;
NH₂-NLS-[cytidine deaminase]-[napDNAbp domain]-[adenosine deaminase]-COOH;
NH₂-NLS-[adenosine deaminase]-[napDNAbp domain]-[cytidine deaminase]-COOH;
NH₂-NLS-[adenosine deaminase] [cytidine deaminase]-[napDNAbp domain]-COOH;
NH₂-NLS-[cytidine deaminase]-[adenosine deaminase]-[napDNAbp domain]-COOH;
NH₂-NLS-[napDNAbp domain]-[adenosine deaminase]-[cytidine deaminase]-COOH;
NH₂-NLS-[napDNAbp domain]-[cytidine deaminase]-[adenosine deaminase]-COOH;
NH₂-[cytidine deaminase]-[napDNAbp domain]-[adenosine deaminase]-NLS-COOH;
NH₂-[adenosine deaminase]-[napDNAbp domain]-[cytidine deaminase]-NLS-COOH;
NH₂-[adenosine deaminase] [cytidine deaminase]-[napDNAbp domain]-NLS-COOH;
NH₂-[cytidine deaminase]-[adenosine deaminase]-[napDNAbp domain]-NLS-COOH;
NH₂-[napDNAbp domain]-[adenosine deaminase]-[cytidine deaminase]-NLS-COOH; or
NH₂-[napDNAbp domain]-[cytidine deaminase]-[adenosine deaminase]-NLS-COOH. In some embodiments, the NLS is present in a linker or the NLS is flanked by linkers, for example described herein. A bipartite NLS comprises two basic amino acid clusters, which are separated by a relatively short spacer sequence (hence bipartite—2 parts, while monopartite NLSs are not). The NLS of nucleoplasmin, KR[PAATKKAGQA]KKKK (SEQ ID NO: 195), is the prototype of the ubiquitous bipartite signal: two clusters of basic amino acids, separated by a spacer of about 10 amino acids. The sequence of an exemplary bipartite NLS follows:

| (SEQ ID NO: 332) | |
|---|---|
| | PKKKRKVEGADKRTADGSEFESPKKKRKV |

A vector that encodes a CRISPR enzyme comprising one or more nuclear localization sequences (NLSs) can be used. For example, there can be or be about 1, 2, 3, 4, 5, 6, 7, 8, 9, 10 NLSs used. A CRISPR enzyme can comprise the NLSs at or near the amino-terminus, about or more than about 1, 2, 3, 4, 5, 6, 7, 8, 9, 10 NLSs at or near the carboxy-terminus, or any combination thereof (e.g., one or more NLS at the amino-terminus and one or more NLS at the carboxy terminus). When more than one NLS is present, each can be selected independently of others, such that a single NLS can be present in more than one copy and/or in combination with one or more other NLSs present in one or more copies.

CRISPR enzymes used in the methods can comprise about 6 NLSs. An NLS is considered near the N- or C-terminus when the nearest amino acid to the NLS is within about 50 amino acids along a polypeptide chain from the N- or C-terminus, e.g., within 1, 2, 3, 4, 5, 10, 15, 20, 25, 30, 40, or 50 amino acids.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor can comprise a nuclease, a nickase, a recombinase, a deaminase, a methyltransferase, a methylase, an acetylase, an acetyltransferase, a transcriptional activator, or a transcriptional repressor domain.

In some embodiments, a base editor can comprise an uracil glycosylase inhibitor (UGI) domain. In some embodiments, cellular DNA repair response to the presence of U: G heteroduplex DNA can be responsible for a decrease in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein comprising a UGI domain.

In some embodiments, a base editor comprises as a domain all or a portion of a double-strand break (DSB) binding protein. For example, a DSB binding protein can include a Gam protein of bacteriophage Mu that can bind to the ends of DSBs and can protect them from degradation. See Komor, A. C., et al., “Improved base excision repair inhibition and bacteriophage Mu Gam protein yields C:G-to-T:A base editors with higher efficiency and product purity” Science Advances 3:eaao4774 (2017), the entire content of which is hereby incorporated by reference.

Additionally, in some embodiments, a Gam protein can be fused to an N terminus of a base editor. In some embodiments, a Gam protein can be fused to a C terminus of a base editor. The Gam protein of bacteriophage Mu can bind to the ends of double strand breaks (DSBs) and protect them from degradation. In some embodiments, using Gam to bind the free ends of DSB can reduce indel formation during the process of base editing. In some embodiments, 174-residue Gam protein is fused to the N terminus of the base editors. See Komor, A. C., et al., “Improved base excision repair inhibition and bacteriophage Mu Gam protein yields C:G-to-T:A base editors with higher efficiency and product purity” Science Advances 3:eaao4774 (2017). In some embodiments, a mutation or mutations can change the length of a base editor domain relative to a wild type domain. For example, a deletion of at least one amino acid in at least one domain can reduce the length of the base editor. In another case, a mutation or mutations do not change the length of a domain relative to a wild type domain. For example, substitutions in any domain does not change the length of the base editor.

Non-limiting examples of such base editors, where the length of all the domains is the same as the wild type domains, can include:
NH2-[nucleobase editing domain]-Linker1-[APOBEC1]-Linker2-[nucleobase editing domain]-COOH;
NH2-[nucleobase editing domain]-Linker1-[APOBEC1]-[nucleobase editing domain]-COOH;
NH2-[nucleobase editing domain]-[APOBEC1]-Linker2-[nucleobase editing domain]-COOH;
NH2-[nucleobase editing domain]-[APOBEC1]-[nucleobase editing domain]-COOH;
NH2-[nucleobase editing domain]-Linker1-[APOBEC1]-Linker2-[nucleobase editing domain]-[UGI]-COOH;
NH2-[nucleobase editing domain]-Linker1-[APOBEC1]-[nucleobase editing domain]-[UGI]-COOH;
NH2-[nucleobase editing domain]-[APOBEC1]-Linker2-[nucleobase editing domain]-[UGI]-COOH;
NH2-[nucleobase editing domain]-[APOBEC1]-[nucleobase editing domain]-[UGI]-COOH;
NH2-[UGI]-[nucleobase editing domain]-Linker1-[APOBEC1]-Linker2-[nucleobase editing domain]-COOH;
NH2-[UGI]-[nucleobase editing domain]-Linker1-[APOBEC1]-[nucleobase editing domain]-COOH;
NH2-[UGI]-[nucleobase editing domain]-[APOBEC1]-Linker2-[nucleobase editing domain]-COOH; or
NH2-[UGI]-[nucleobase editing domain]-[APOBEC1]-[nucleobase editing domain]-COOH.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

In some embodiments, a base editing system as provided herein provides a new approach to genome editing that uses a fusion protein containing a catalytically defective Streptococcus pyogenes Cas9, a deaminase (e.g., cytidine or adenosine deaminase), and an inhibitor of base excision repair to induce programmable, single nucleotide (C→T or A→G) changes in DNA without generating double-strand DNA breaks, without requiring a donor DNA template, and without inducing an excess of stochastic insertions and deletions.

Details of nucleobase editing proteins are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleic acid (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

In some embodiments, the cut single strand (nicked strand) is hybridized to the guide nucleic acid. In some embodiments, the cut single strand is opposite to the strand comprising the first nucleobase. In some embodiments, the base editor comprises a Cas9 domain. In some embodiments, the first base is adenine, and the second base is not a G, C, A, or T. In some embodiments, the second base is inosine.

In some embodiments, a single guide polynucleotide may be utilized to target a deaminase to a target nucleic acid sequence. In some embodiments, a single pair of guide polynucleotides may be utilized to target different deaminases to a target nucleic acid sequence.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fe domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g. an MS2, an MS2 C-5 mutant, or an MS2 F-5 mutant), a non-natural RNA motif, a PP7 opterator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 385, 387, 389, 391-393, or fragments thereof. Non-limiting examples of additional heterologous portions include polynucleotides with at least about 85% sequence identity to any one or more of SEQ ID NOs: 384, 386, 388, 390, or fragments thereof.

A base editor system may further comprise a guide polynucleotide component. It should be appreciated that components of the base editor system may be associated with each other via covalent bonds, noncovalent interactions, or any combination of associations and interactions thereof. In some embodiments, a deaminase domain can be targeted to a target nucleotide sequence by a guide polynucleotide. For example, in some embodiments, the nucleobase editing component of the base editor system (e.g., the deaminase component) comprises an additional heterologous portion or domain (e.g., polynucleotide binding domain such as an RNA or DNA binding protein) that is capable of interacting with, associating with, or capable of forming a complex with a heterologous portion or segment (e.g., a polynucleotide motif), or antigen of a guide polynucleotide. In some embodiments, the additional heterologous portion or domain (e.g., polynucleotide binding domain such as an RNA or DNA binding protein) can be fused or linked to the deaminase domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fe domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g. an MS2, an MS2 C-5 mutant, or an MS2 F-5 mutant), a non-natural RNA motif, a PP7 opterator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 385, 387, 389, 391-393, or fragments thereof. Non-limiting examples of additional heterologous portions include polynucleotides with at least about 85% sequence identity to any one or more of SEQ ID NOs: 384, 386, 388, 390, or fragments thereof.

In some embodiments, a base editor system can further comprise an inhibitor of base excision repair (BER) component. It should be appreciated that components of the base editor system may be associated with each other via covalent bonds, noncovalent interactions, or any combination of associations and interactions thereof. The inhibitor of BER component may comprise a base excision repair inhibitor. In some embodiments, the inhibitor of base excision repair can be a uracil DNA glycosylase inhibitor (UGI). In some embodiments, the inhibitor of base excision repair can be an inosine base excision repair inhibitor. In some embodiments, the inhibitor of base excision repair can be targeted to the target nucleotide sequence by the polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to an inhibitor of base excision repair. In some embodiments, a polynucleotide programmable nucleotide binding domain can be fused or linked to a deaminase domain and an inhibitor of base excision repair. In some embodiments, a polynucleotide programmable nucleotide binding domain can target an inhibitor of base excision repair to a target nucleotide sequence by non-covalently interacting with or associating with the inhibitor of base excision repair. For example, in some embodiments, the inhibitor of base excision repair component comprises an additional heterologous portion or domain that is capable of interacting with, associating with, or capable of forming a complex with a corresponding additional heterologous portion, antigen, or domain that is part of a polynucleotide programmable nucleotide binding domain. In some embodiments, the polynucleotide programming nucleotide binding domain component, and/or a guide polynucleotide (e.g., a guide RNA) complexed therewith, comprises an additional heterologous portion or domain that is capable of interacting with, associating with, or capable of forming a corresponding heterologous portion, antigen, or domain that is part of an inhibitor of base excision repair component. In some embodiments, the inhibitor of base excision repair can be targeted to the target nucleotide sequence by the guide polynucleotide. For example, in some embodiments, the inhibitor of base excision repair comprises an additional heterologous portion or domain (e.g., polynucleotide binding domain such as an RNA or DNA binding protein) that is capable of interacting with, associating with, or capable of forming a complex with a portion or segment (e.g., a polynucleotide motif) of a guide polynucleotide. In some embodiments, the additional heterologous portion or domain of the guide polynucleotide (e.g., polynucleotide binding domain such as an RNA or DNA binding protein) can be fused or linked to the inhibitor of base excision repair. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fe domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g. an MS2, an MS2 C-5 mutant, or an MS2 F-5 mutant), a non-natural RNA motif, a PP7 opterator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 385, 387, 389, 391-393, or fragments thereof. Non-limiting examples of additional heterologous portions include polynucleotides with at least about 85% sequence identity to any one or more of SEQ ID NOs: 384, 386, 388, 390, or fragments thereof.

In some instances, components of the base editing system are associated with one another through the interaction of leucine zipper domains (e.g., SEQ ID NOs: 392 and 393). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and Voß, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. Non-limiting examples of polypeptides that can dimerize and their corresponding dimerizing agents are provided in Table 10.1 below.

TABLE 10.1
Chemically induced dimerization systems.

| | | Dimerizing |
|---|---|---|
| Dimerizing Polypeptides | | agent |
| FKBP | FKBP | FK1012 |
| FKBP | Calcineurin A (CNA) | FK506 |
| FKBP | CyP-Fas | FKCsA |
| FKBP | FRB (FKBP-rapamycin-binding) | Rapamycin |
| | domain of mTOR | |
| GyrB | GyrB | Coumermycin |
| GAI | GID1 (gibberellin insensitive dwarf 1) | Gibberellin |
| ABI | PYL | Abscisic acid |
| ABI | PYRMandi | Mandipropamid |
| SNAP-tag | HaloTag | HaXS |
| eDHFR | HaloTag | TMP-HTag |
| Bcl-xL | Fab (AZ1) | ABT-737 |

In embodiments, the additional heterologous portion is part of a guide RNA molecule. In some instances, the additional heterologous portion contains or is an RNA motif. The RNA motif may be positioned at the 5′ or 3′ end of the guide RNA molecule or various positions of a guide RNA molecule. In embodiments, the RNA motif is positioned within the guide RNA to reduce steric hindrance, optionally where such hindrance is associated with other bulky loops of an RNA scaffold. In some instances, it is advantageous to link the RNA motif is linked to other portions of the guide RNA by way of a linker, where the linker can be about, at least about, or no more than about 1, 2, 3, 4, 5, 6, 7, 8, 9, 10, or more nucleotides in length. Optionally, the linker contains a GC-rich nucleotide sequence. The guide RNA can contain 1, 2, 3, 4, 5, or more copies of the RNA motif, optionally where they are positioned consecutively, and/or optionally where they are each separated from one another by a linker(s). The RNA motif may include any one or more of the polynucleotide modifications described herein. Non-limiting examples of suitable modifications to the RNA motif include 2′ deoxy-2-aminopurine, 2′ ribose-2-aminopurine, phosphorothioate mods, 2′-Omethyl mods, 2′-Fluro mods and LNA mods. Advantageously, the modifications help to increase stability and promote stronger bonds/folding structure of a hairpin(s) formed by the RNA motif.

In some embodiments, the RNA motif is modified to include an extension. In embodiments, the extension contains about, at least about, or no more than about 2, 3, 4, 5, 10, 15, 20, or 25 nucleotides. In some instances, the extension results in an alteration in the length of a stem formed by the RNA motif (e.g., a lengthening or a shortening). It can be advantageous for a stem formed by the RNA motif to be about, at least about, or no more than about 5, 10, 15, 20, 25, 30, 35, 40, 45, 50, 55, 60, 65, 70, 75, 80, 85, 90, 95, or 100 nucleotides in length. In various embodiments, the extension increases flexibility of the RNA motif and/or increases binding with a corresponding RNA motif.

In some embodiments, the base editor inhibits base excision repair (BER) of the edited strand. In some embodiments, the base editor protects or binds the non-edited strand. In some embodiments, the base editor comprises UGI activity. In some embodiments, the base editor comprises a catalytically inactive inosine-specific nuclease. In some embodiments, the base editor comprises nickase activity. In some embodiments, the intended edit of base pair is upstream of a PAM site. In some embodiments, the intended edit of base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edit of base-pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site.

In some embodiments, the method does not require a canonical (e.g., NGG) PAM site. In some embodiments, the nucleobase editor comprises a linker or a spacer. In some embodiments, the linker or spacer is 1-25 amino acids in length. In some embodiments, the linker or spacer is 5-20 amino acids in length. In some embodiments, the linker or spacer is 10, 11, 12, 13, 14, 15, 16, 17, 18, 19, or 20 amino acids in length.

In some embodiments, the base editing fusion proteins provided herein need to be positioned at a precise location, for example, where a target base is placed within a defined region (e.g., a “deamination window”). In some embodiments, a target can be within a 4 base region. In some embodiments, such a defined target region can be approximately 15 bases upstream of the PAM. See Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

In some embodiments, the target region comprises a target window, wherein the target window comprises the target nucleobase pair. In some embodiments, the target window comprises 1-10 nucleotides. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edit of base pair is within the target window. In some embodiments, the target window comprises the intended edit of base pair. In some embodiments, the method is performed using any of the base editors provided herein. In some embodiments, a target window is a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Other exemplary features that can be present in a base editor as disclosed herein are localization sequences, such as cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments, non-limiting exemplary cytidine base editors (CBE) include BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, or saB4E-Gam. BE4 extends the APOBEC1-Cas9n(D10A) linker to 32 amino acids and the Cas9n-UGI linker to 9 amino acids, and appends a second copy of UGI to the C-terminus of the construct with another 9-amino acid linker into a single base editor construct. The base editors saBE3 and saBE4 have the S. pyogenes Cas9n(D10A) replaced with the smaller S. aureus Cas9n(D10A). BE3-Gam, saBE3-Gam, BE4-Gam, and saBE4-Gam have 174 residues of Gam protein fused to the N-terminus of BE3, saBE3, BE4, and saBE4 via the 16 amino acid XTEN linker.

In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises evolved TadA variant. In some embodiments, the ABE is ABE 1.2 (TadA*-XTEN-nCas9-NLS). In some embodiments, TadA* comprises A106V and D108N mutations.

In some embodiments, the ABE is a second-generation ABE. In some embodiments, the ABE is ABE2.1, which comprises additional mutations D147Y and E155V in TadA* (TadA*2.1). In some embodiments, the ABE is ABE2.2, ABE2.1 fused to catalytically inactivated version of human alkyl adenine DNA glycosylase (AAG with E125Q mutation). In some embodiments, the ABE is ABE2.3, ABE2.1 fused to catalytically inactivated version of E. coli Endo V (inactivated with D35A mutation). In some embodiments, the ABE is ABE2.6 which has a linker twice as long (32 amino acids, (SGGS)₂ (SEQ ID NO: 334)-XTEN-(SGGS)₂ (SEQ ID NO: 334)) as the linker in ABE2.1. In some embodiments, the ABE is ABE2.7, which is ABE2.1 tethered with an additional wild-type TadA monomer. In some embodiments, the ABE is ABE2.8, which is ABE2.1 tethered with an additional TadA*2.1 monomer. In some embodiments, the ABE is ABE2.9, which is a direct fusion of evolved TadA (TadA*2.1) to the N-terminus of ABE2.1. In some embodiments, the ABE is ABE2.10, which is a direct fusion of wild-type TadA to the N-terminus of ABE2.1. In some embodiments, the ABE is ABE2.11, which is ABE2.9 with an inactivating E59A mutation at the N-terminus of TadA* monomer. In some embodiments, the ABE is ABE2.12, which is ABE2.9 with an inactivating E59A mutation in the internal TadA* monomer.

In some embodiments, the ABE is a third generation ABE. In some embodiments, the ABE is ABE3.1, which is ABE2.3 with three additional TadA mutations (L84F, H123Y, and 1156F).

In some embodiments, the ABE is a fourth generation ABE. In some embodiments, the ABE is ABE4.3, which is ABE3.1 with an additional TadA mutation A142N (TadA*4.3).

In some embodiments, the ABE is a fifth generation ABE. In some embodiments, the ABE is ABE5.1, which is generated by importing a consensus set of mutations from surviving clones (H36L, R51L, S146C, and K157N) into ABE3.1. In some embodiments, the ABE is ABE5.3, which has a heterodimeric construct containing wild-type E. coli TadA fused to an internal evolved TadA*. In some embodiments, the ABE is ABE5.2, ABE5.4, ABE5.5, ABE5.6, ABE5.7, ABE5.8, ABE5.9, ABE5.10, ABE5.11, ABE5.12, ABE5.13, or ABE5.14, as shown in Table 11 below. In some embodiments, the ABE is a sixth generation ABE. In some embodiments, the ABE is ABE6.1, ABE6.2, ABE6.3, ABE6.4, ABE6.5, or ABE6.6, as shown in Table 11 below. In some embodiments, the ABE is a seventh generation ABE. In some embodiments, the ABE is ABE7.1, ABE7.2, ABE7.3, ABE7.4, ABE7.5, ABE7.6, ABE7.7, ABE7.8, ABE 7.9, or ABE7.10, as shown in Table 11 below.

TABLE 11
Genotypes of ABEs

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| ABE0.1 | W | R | H | N | P | | R | N | L | S | A | D | H | G | A | S | D | R | E | I | K | K |
| ABE0.2 | W | R | H | N | P | | R | N | L | S | A | D | H | G | A | S | D | R | E | I | K | K |
| ABE1.1 | W | R | H | N | P | | R | N | L | S | A | N | H | G | A | S | D | R | E | I | K | K |
| ABE1.2 | W | R | H | N | P | | R | N | L | S | V | N | H | G | A | S | D | R | E | I | K | K |
| ABE2.1 | W | R | H | N | P | | R | N | L | S | V | N | H | G | A | S | Y | R | V | I | K | K |
| ABE2.2 | W | R | H | N | P | | R | N | L | S | V | N | H | G | A | S | Y | R | V | I | K | K |
| ABE2.3 | W | R | H | N | P | | R | N | L | S | V | N | H | G | A | S | Y | R | V | I | K | K |
| ABE2.4 | W | R | H | N | P | | R | N | L | S | V | N | H | G | A | S | Y | R | V | I | K | K |
| ABE2.5 | W | R | H | N | P | | R | N | L | S | V | N | H | G | A | S | Y | R | V | I | K | K |
| ABE2.6 | W | R | H | N | P | | R | N | L | S | V | N | H | G | A | S | Y | R | V | I | K | K |
| ABE2.7 | W | R | H | N | P | | R | N | L | S | V | N | H | G | A | S | Y | R | V | I | K | K |
| ABE2.8 | W | R | H | N | P | | R | N | L | S | V | N | H | G | A | S | Y | R | V | I | K | K |
| ABE2.9 | W | R | H | N | P | | R | N | L | S | V | N | H | G | A | S | Y | R | V | I | K | K |
| ABE2.10 | W | R | H | N | P | | R | N | L | S | V | N | H | G | A | S | Y | R | V | I | K | K |
| ABE2.11 | W | R | H | N | P | | R | N | L | S | V | N | H | G | A | S | Y | R | V | I | K | K |
| ABE2.12 | W | R | H | N | P | | R | N | L | S | V | N | H | G | A | S | Y | R | V | I | K | K |
| ABE3.1 | W | R | H | N | P | | R | N | F | S | V | N | Y | G | A | S | Y | R | V | F | K | K |
| ABE3.2 | W | R | H | N | P | | R | N | F | S | V | N | Y | G | A | S | Y | R | V | F | K | K |
| ABE3.3 | W | R | H | N | P | | R | N | F | S | V | N | Y | G | A | S | Y | R | V | F | K | K |
| ABE3.4 | W | R | H | N | P | | R | N | F | S | V | N | Y | G | A | S | Y | R | V | F | K | K |
| ABE3.5 | W | R | H | N | P | | R | N | F | S | V | N | Y | G | A | S | Y | R | V | F | K | K |
| ABE3.6 | W | R | H | N | P | | R | N | F | S | V | N | Y | G | A | S | Y | R | V | F | K | K |
| ABE3.7 | W | R | H | N | P | | R | N | F | S | V | N | Y | G | A | S | Y | R | V | F | K | K |
| ABE3.8 | W | R | H | N | P | | R | N | F | S | V | N | Y | G | A | S | Y | R | V | F | K | K |
| ABE4.1 | W | R | H | N | P | | R | N | L | S | V | N | H | G | N | S | Y | R | V | I | K | K |
| ABE4.2 | W | G | H | N | P | | R | N | L | S | V | N | H | G | N | S | Y | R | V | I | K | K |
| ABE4.3 | W | R | H | N | P | | R | N | F | S | V | N | Y | G | N | S | Y | R | V | F | K | K |
| ABE5.1 | W | R | L | N | P | | L | N | F | S | V | N | Y | G | A | C | Y | R | V | F | N | K |
| ABE5.2 | W | R | H | S | P | | R | N | F | S | V | N | Y | G | A | S | Y | R | V | F | K | T |
| ABE5.3 | W | R | L | N | P | | L | N | I | S | V | N | Y | G | A | C | Y | R | V | F | N | K |
| ABE5.4 | W | R | H | S | P | | R | N | F | S | V | N | Y | G | A | S | Y | R | V | F | K | T |
| ABE5.5 | W | R | L | N | P | | L | N | F | S | V | N | Y | G | A | C | Y | R | V | F | N | K |
| ABE5.6 | W | R | L | N | P | | L | N | F | S | V | N | Y | G | A | C | Y | R | V | F | N | K |
| ABE5.7 | W | R | L | N | P | | L | N | F | S | V | N | Y | G | A | C | Y | R | V | F | N | K |
| ABE5.8 | W | R | L | N | P | | L | N | F | S | V | N | Y | G | A | C | Y | R | V | F | N | K |
| ABE5.9 | W | R | L | N | P | | L | N | F | S | V | N | Y | G | A | C | Y | R | V | F | N | K |
| ABE5.10 | W | R | L | N | P | | L | N | F | S | V | N | Y | G | A | C | Y | R | V | F | N | K |
| ABE5.11 | W | R | L | N | P | | L | N | F | S | V | N | Y | G | A | C | Y | R | V | F | N | K |
| ABE5.12 | W | R | L | N | P | | L | N | F | S | V | N | Y | G | A | C | Y | R | V | F | N | K |
| ABE5.13 | W | R | H | N | P | | L | D | F | S | V | N | Y | A | A | S | Y | R | V | F | K | K |
| ABE5.14 | W | R | H | N | S | | L | N | F | C | V | N | Y | G | A | S | Y | R | V | F | K | K |
| ABE6.1 | W | R | H | N | S | | L | N | F | S | V | N | Y | G | N | S | Y | R | V | F | K | K |
| ABE6.2 | W | R | H | N | T | V | L | N | F | S | V | N | Y | G | N | S | Y | R | V | F | N | K |
| ABE6.3 | W | R | L | N | S | | L | N | F | S | V | N | Y | G | A | C | Y | R | V | F | N | K |
| ABE6.4 | W | R | L | N | S | | L | N | F | S | V | N | Y | G | N | C | Y | R | V | F | N | K |
| ABE6.5 | W | R | L | N | T | V | L | N | F | S | V | N | Y | G | A | C | Y | R | V | F | N | K |
| ABE6.6 | W | R | L | N | T | V | L | N | F | S | V | N | Y | G | N | C | Y | R | V | F | N | K |
| ABE7.1 | W | R | L | N | A | | L | N | F | S | V | N | Y | G | A | C | Y | R | V | F | N | K |
| ABE7.2 | W | R | L | N | A | | L | N | F | S | V | N | Y | G | N | C | Y | R | V | F | N | K |
| ABE7.3 | L | R | L | N | A | | L | N | F | S | V | N | Y | G | A | C | Y | R | V | F | N | K |
| ABE7.4 | R | R | L | N | A | | L | N | F | S | V | N | Y | G | A | C | Y | R | V | F | N | K |
| ABE7.5 | W | R | L | N | A | | L | N | F | S | V | N | Y | G | A | C | Y | H | V | F | N | K |
| ABE7.6 | W | R | L | N | A | | L | N | I | S | V | N | Y | G | A | C | Y | P | V | F | N | K |
| ABE7.7 | L | R | L | N | A | | L | N | F | S | V | N | Y | G | A | C | Y | P | V | F | N | K |
| ABE7.8 | L | R | L | N | A | | L | N | F | S | V | N | Y | G | N | C | Y | R | V | F | N | K |
| ABE7.9 | L | R | L | N | A | | L | N | F | S | V | N | Y | G | N | C | Y | P | V | F | N | K |
| ABE7.10 | R | R | L | N | A | | L | N | F | S | V | N | Y | G | A | C | Y | P | V | F | N | K |

In embodiments, the ABE8 contains a TadA*8 variant. In some embodiments, the ABE8 has a monomeric construct containing a TadA*8 variant (“ABE8.x-m”). In some embodiments, the ABE8 is ABE8.1-m, which has a monomeric construct containing TadA*7.10 with a Y147T mutation (TadA*8.1). In some embodiments, the ABE8 is ABE8.2-m, which has a monomeric construct containing TadA*7.10 with a Y147R mutation (TadA*8.2). In some embodiments, the ABE8 is ABE8.3-m, which has a monomeric construct containing TadA*7.10 with a Q154S mutation (TadA*8.3). In some embodiments, the ABE8 is ABE8.4-m, which has a monomeric construct containing TadA*7.10 with a Y123H mutation (TadA*8.4). In some embodiments, the ABE8 is ABE8.5-m, which has a monomeric construct containing TadA*7.10 with a V82S mutation (TadA*8.5). In some embodiments, the ABE8 is ABE8.6-m, which has a monomeric construct containing TadA*7.10 with a T166R mutation (TadA*8.6). In some embodiments, the ABE8 is ABE8.7-m, which has a monomeric construct containing TadA*7.10 with a Q154R mutation (TadA*8.7). In some embodiments, the ABE8 is ABE8.8-m, which has a monomeric construct containing TadA*7.10 with Y147R, Q154R, and Y123H mutations (TadA*8.8). In some embodiments, the ABE8 is ABE8.9-m, which has a monomeric construct containing TadA*7.10 with Y147R, Q154R and I76Y mutations (TadA*8.9). In some embodiments, the ABE8 is ABE8.10-m, which has a monomeric construct containing TadA*7.10 with Y147R, Q154R, and T166R mutations (TadA*8.10). In some embodiments, the ABE8 is ABE8.11-m, which has a monomeric construct containing TadA*7.10 with Y147T and Q154R mutations (TadA*8.11). In some embodiments, the ABE8 is ABE8.12-m, which has a monomeric construct containing TadA*7.10 with Y147T and Q154S mutations (TadA*8.12).

In some embodiments, the ABE8 is ABE8.13-m, which has a monomeric construct containing TadA*7.10 with Y123H (Y123H reverted from H123Y), Y147R, Q154R and I76Y mutations (TadA*8.13). In some embodiments, the ABE8 is ABE8.14-m, which has a monomeric construct containing TadA*7.10 with I76Y and V82S mutations (TadA*8.14). In some embodiments, the ABE8 is ABE8.15-m, which has a monomeric construct containing TadA*7.10 with V82S and Y147R mutations (TadA*8.15). In some embodiments, the ABE8 is ABE8.16-m, which has a monomeric construct containing TadA*7.10 with V82S, Y123H (Y123H reverted from H123Y) and Y147R mutations (TadA*8.16). In some embodiments, the ABE8 is ABE8.17-m, which has a monomeric construct containing TadA*7.10 with V82S and Q154R mutations (TadA*8.17). In some embodiments, the ABE8 is ABE8.18-m, which has a monomeric construct containing TadA*7.10 with V82S, Y123H (Y123H reverted from H123Y) and Q154R mutations (TadA*8.18). In some embodiments, the ABE8 is ABE8.19-m, which has a monomeric construct containing TadA*7.10 with V82S, Y123H (Y123H reverted from H123Y), Y147R and Q154R mutations (TadA*8.19). In some embodiments, the ABE8 is ABE8.20-m, which has a monomeric construct containing TadA*7.10 with I76Y, V82S, Y123H (Y123H reverted from H123Y), Y147R and Q154R mutations (TadA*8.20). In some embodiments, the ABE8 is ABE8.21-m, which has a monomeric construct containing TadA*7.10 with Y147R and Q154S mutations (TadA*8.21). In some embodiments, the ABE8 is ABE8.22-m, which has a monomeric construct containing TadA*7.10 with V82S and Q154S mutations (TadA*8.22). In some embodiments, the ABE8 is ABE8.23-m, which has a monomeric construct containing TadA*7.10 with V82S and Y123H (Y123H reverted from H123Y) mutations (TadA*8.23). In some embodiments, the ABE8 is ABE8.24-m, which has a monomeric construct containing TadA*7.10 with V82S, Y123H (Y123H reverted from H123Y), and Y147T mutations (TadA*8.24).

In some embodiments, the ABE8 has a heterodimeric construct containing wild-type E. coli TadA fused to a TadA*8 variant (“ABE8.x-d”). In some embodiments, the ABE8 is ABE8.1-d, which has a heterodimeric construct containing wild-type E. coli TadA fused to TadA*7.10 with aY147T mutation (TadA*8.1). In some embodiments, the ABE8 is ABE8.2-d, which has a heterodimeric construct containing wild-type E. coli TadA fused to TadA*7.10 with a Y147R mutation (TadA*8.2). In some embodiments, the ABE8 is ABE8.3-d, which has a heterodimeric construct containing wild-type E. coli TadA fused to TadA*7.10 with a Q154S mutation (TadA*8.3). In some embodiments, the ABE8 is ABE8.4-d, which has a heterodimeric construct containing wild-type E. coli TadA fused to TadA*7.10 with a Y123H mutation (TadA*8.4). In some embodiments, the ABE8 is ABE8.5-d, which has a heterodimeric construct containing wild-type E. coli TadA fused to TadA*7.10 with a V82S mutation (TadA*8.5). In some embodiments, the ABE8 is ABE8.6-d, which has a heterodimeric construct containing wild-type E. coli TadA fused to TadA*7.10 with a T166R mutation (TadA*8.6). In some embodiments, the ABE8 is ABE8.7-d, which has a heterodimeric construct containing wild-type E. coli TadA fused to TadA*7.10 with a Q154R mutation (TadA*8.7). In some embodiments, the ABE8 is ABE8.8-d, which has a heterodimeric construct containing wild-type E. coli TadA fused to TadA*7.10 with Y147R, Q154R, and Y123H mutations (TadA*8.8). In some embodiments, the ABE8 is ABE8.9-d, which has a heterodimeric construct containing wild-type E. coli TadA fused to TadA*7.10 with Y147R, Q154R and I76Y mutations (TadA*8.9). In some embodiments, the ABE8 is ABE8.10-d, which has a heterodimeric construct containing wild-type E. coli TadA fused to TadA*7.10 with Y147R, Q154R, and T166R mutations (TadA*8.10). In some embodiments, the ABE8 is ABE8.11-d, which has a heterodimeric construct containing wild-type E. coli TadA fused to TadA*7.10 with Y147T and Q154R mutations (TadA*8.11). In some embodiments, the ABE8 is ABE8.12-d, which has heterodimeric construct containing wild-type E. coli TadA fused to TadA*7.10 with Y147T and Q154S mutations (TadA*8.12). In some embodiments, the ABE8 is ABE8.13-d, which has a heterodimeric construct containing wild-type E. coli TadA fused to TadA*7.10 with Y123H (Y123H reverted from H123Y), Y147R, Q154R and I76Y mutations (TadA*8.13). In some embodiments, the ABE8 is ABE8.14-d, which has a heterodimeric construct containing wild-type E. coli TadA fused to TadA*7.10 with I76Y and V82S mutations (TadA*8.14). In some embodiments, the ABE8 is ABE8.15-d, which has a heterodimeric construct containing wild-type E. coli TadA fused to TadA*7.10 with V82S and Y147R mutations (TadA*8.15). In some embodiments, the ABE8 is ABE8.16-d, which has a heterodimeric construct containing wild-type E. coli TadA fused to TadA*7.10 with V82S, Y123H (Y123H reverted from H123Y) and Y147R mutations (TadA*8.16). In some embodiments, the ABE8 is ABE8.17-d, which has a heterodimeric construct containing wild-type E. coli TadA fused to TadA*7.10 with V82S and Q154R mutations (TadA*8.17). In some embodiments, the ABE8 is ABE8.18-d, which has a heterodimeric construct containing wild-type E. coli TadA fused to TadA*7.10 with V82S, Y123H (Y123H reverted from H123Y) and Q154R mutations (TadA*8.18). In some embodiments, the ABE8 is ABE8.19-d, which has a heterodimeric construct containing wild-type E. coli TadA fused to TadA*7.10 with V82S, Y123H (Y123H reverted from H123Y), Y147R and Q154R mutations (TadA*8.19). In some embodiments, the ABE8 is ABE8.20-d, which has a heterodimeric construct containing wild-type E. coli TadA fused to TadA*7.10 with I76Y, V82S, Y123H (Y123H reverted from H123Y), Y147R and Q154R mutations (TadA*8.20). In some embodiments, the ABE8 is ABE8.21-d, which has a heterodimeric construct containing wild-type E. coli TadA fused to TadA*7.10 with Y147R and Q154S mutations (TadA*8.21). In some embodiments, the ABE8 is ABE8.22-d, which has a heterodimeric construct containing wild-type E. coli TadA fused to TadA*7.10 with V82S and Q154S mutations (TadA*8.22). In some embodiments, the ABE8 is ABE8.23-d, which has a heterodimeric construct containing wild-type E. coli TadA fused to TadA*7.10 with V82S and Y123H (Y123H reverted from H123Y) mutations (TadA*8.23). In some embodiments, the ABE8 is ABE8.24-d, which has a heterodimeric construct containing wild-type E. coli TadA fused to TadA*7.10 with V82S, Y123H (Y123H reverted from H123Y), and Y147T mutations (TadA*8.24).

In some embodiments, the ABE8 has a heterodimeric construct containing TadA*7.10 fused to a TadA*8 variant (“ABE8.x-7”). In some embodiments, the ABE8 is ABE8.1-7, which has a heterodimeric construct containing TadA*7.10 fused to TadA*7.10 with a Y147T mutation (TadA*8.1). In some embodiments, the ABE8 is ABE8.2-7, which has a heterodimeric construct containing TadA*7.10 fused to TadA*7.10 with a Y147R mutation (TadA*8.2). In some embodiments, the ABE8 is ABE8.3-7, which has a heterodimeric construct containing TadA*7.10 fused to TadA*7.10 with a Q154S mutation (TadA*8.3). In some embodiments, the ABE8 is ABE8.4-7, which has a heterodimeric construct containing TadA*7.10 fused to TadA*7.10 with aY123H mutation (TadA*8.4). In some embodiments, the ABE8 is ABE8.5-7, which has a heterodimeric construct containing TadA*7.10 fused to TadA*7.10 with a V82S mutation (TadA*8.5). In some embodiments, the ABE8 is ABE8.6-7, which has a heterodimeric construct containing TadA*7.10 fused to TadA*7.10 with a T166R mutation (TadA*8.6). In some embodiments, the ABE8 is ABE8.7-7, which has a heterodimeric construct containing TadA*7.10 fused to TadA*7.10 with a Q154R mutation (TadA*8.7). In some embodiments, the ABE8 is ABE8.8-7, which has a heterodimeric construct containing TadA*7.10 fused to TadA*7.10 with Y147R, Q154R, and Y123H mutations (TadA*8.8). In some embodiments, the ABE8 is ABE8.9-7, which has a heterodimeric construct containing TadA*7.10 fused to TadA*7.10 with Y147R, Q154R and 176Y mutations (TadA*8.9). In some embodiments, the ABE8 is ABE8.10-7, which has a heterodimeric construct containing TadA*7.10 fused to TadA*7.10 with Y147R, Q154R, and T166R mutations (TadA*8.10). In some embodiments, the ABE8 is ABE8.11-7, which has a heterodimeric construct containing TadA*7.10 fused to TadA*7.10 with Y147T and Q154R mutations (TadA*8.11). In some embodiments, the ABE8 is ABE8.12-7, which has a heterodimeric construct containing TadA*7.10 fused to TadA*7.10 with Y147T and Q154S mutations (TadA*8.12). In some embodiments, the ABE8 is ABE8.13-7, which has a heterodimeric construct containing TadA*7.10 fused to TadA*7.10 with Y123H (Y123H reverted from H123Y), Y147R, Q154R and 176Y mutations (TadA*8.13). In some embodiments, the ABE8 is ABE8.14-7, which has a heterodimeric construct containing TadA*7.10 fused to TadA*7.10 with 176Y and V82S mutations (TadA*8.14). In some embodiments, the ABE8 is ABE8.15-7, which has a heterodimeric construct containing TadA*7.10 fused to TadA*7.10 with V82S and Y147R mutations (TadA*8.15). In some embodiments, the ABE8 is ABE8.16-7, which has a heterodimeric construct containing TadA*7.10 fused to TadA*7.10 with V82S, Y123H (Y123H reverted from H123Y) and Y147R mutations (TadA*8.16). In some embodiments, the ABE8 is ABE8.17-7, which has a heterodimeric construct containing TadA*7.10 fused to TadA*7.10 with V82S and Q154R mutations (TadA*8.17). In some embodiments, the ABE8 is ABE8.18-7, which has a heterodimeric construct containing TadA*7.10 fused to TadA*7.10 with V82S, Y123H (Y123H reverted from H123Y) and Q154R mutations (TadA*8.18). In some embodiments, the ABE8 is ABE8.19-7, which has a heterodimeric construct containing TadA*7.10 fused to TadA*7.10 with V82S, Y123H (Y123H reverted from H123Y), Y147R and Q154R mutations (TadA*8.19). In some embodiments, the ABE8 is ABE8.20-7, which has a heterodimeric construct containing TadA*7.10 fused to TadA*7.10 with I76Y, V82S, Y123H (Y123H reverted from H123Y), Y147R and Q154R mutations (TadA*8.20). In some embodiments, the ABE8 is ABE8.21-7, which has a heterodimeric construct containing TadA*7.10 fused to TadA*7.10 with Y147R and Q154S mutations (TadA*8.21). In some embodiments, the ABE8 is ABE8.22-7, which has a heterodimeric construct containing TadA*7.10 fused to TadA*7.10 with V82S and Q154S mutations (TadA*8.22). In some embodiments, the ABE8 is ABE8.23-7, which has a heterodimeric construct containing TadA*7.10 fused to TadA*7.10 with V82S and Y123H (Y123H reverted from H123Y) mutations (TadA*8.23). In some embodiments, the ABE8 is ABE8.24-7, which has a heterodimeric construct containing TadA*7.10 fused to TadA*7.10 with V82S, Y123H (Y123H reverted from H123Y), and Y147T mutations (TadA*8.24 [1] In some embodiments, the ABE is ABE8.1-m, ABE8.2-m, ABE8.3-m, ABE8.4-m, ABE8.5-m, ABE8.6-m, ABE8.7-m, ABE8.8-m, ABE8.9-m, ABE8.10-m, ABE8.11-m, ABE8.12-m, ABE8.13-m, ABE8.14-m, ABE8.15-m, ABE8.16-m, ABE8.17-m, ABE8.18-m, ABE8.19-m, ABE8.20-m, ABE8.21-m, ABE8.22-m, ABE8.23-m, ABE8.24-m, ABE8.1-d, ABE8.2-d, ABE8.3-d, ABE8.4-d, ABE8.5-d, ABE8.6-d, ABE8.7-d, ABE8.8-d, ABE8.9-d, ABE8.10-d, ABE8.11-d, ABE8.12-d, ABE8.13-d, ABE8.14-d, ABE8.15-d, ABE8.16-d, ABE8.17-d, ABE8.18-d, ABE8.19-d, ABE8.20-d, ABE8.21-d, ABE8.22-d, ABE8.23-d, or ABE8.24-d as shown in Table 12 below.

TABLE 12
Adenosine Base Editor 8 (ABE8) Variants

| ABE8 | Adenosine Deaminase | Adenosine Deaminase Description |
|---|---|---|
| ABE8.1-m | TadA*8.1 | Monomer_TadA*7.10 + Y147T |
| ABE8.2-m | TadA*8.2 | Monomer_TadA*7.10 + Y147R |
| ABE8.3-m | TadA*8.3 | Monomer_TadA*7.10 + Q154S |
| ABE8.4-m | TadA*8.4 | Monomer_TadA*7.10 + Y123H |
| ABE8.5-m | TadA*8.5 | Monomer_TadA*7.10 + V82S |
| ABE8.6-m | TadA*8.6 | Monomer_TadA*7.10 + T166R |
| ABE8.7-m | TadA*8.7 | Monomer_TadA*7.10 + Q154R |
| ABE8.8-m | TadA*8.8 | Monomer_TadA*7.10 + Y147R_Q154R_Y123H |
| ABE8.9-m | TadA*8.9 | Monomer_TadA*7.10 + Y147R_Q154R_I76Y |
| ABE8.10-m | TadA*8.10 | Monomer_TadA*7.10 + Y147R_Q154R_T166R |
| ABE8.11-m | TadA*8.11 | Monomer_TadA*7.10 + Y147T_Q154R |
| ABE8.12-m | TadA*8.12 | Monomer_TadA*7.10 + Y147T_Q154S |
| ABE8.13-m | TadA*8.13 | Monomer_TadA*7.10 + |
| | | Y123H_Y147R_Q154R_I76Y |
| ABE8.14-m | TadA*8.14 | Monomer_TadA*7.10 + I76Y_V82S |
| ABE8.15-m | TadA*8.15 | Monomer_TadA*7.10 + V82S_Y147R |
| ABE8.16-m | TadA*8.16 | Monomer_TadA*7.10 + V82S_Y123H_Y147R |
| ABE8.17-m | TadA*8.17 | Monomer_TadA*7.10 + V82S_Q154R |
| ABE8.18-m | TadA*8.18 | Monomer_TadA*7.10 + V82S_Y123H_Q154R |
| ABE8.19-m | TadA*8.19 | Monomer_TadA*7.10 + |
| | | V82S_Y123H_Y147R_Q154R |
| ABE8.20-m | TadA*8.20 | Monomer_TadA*7.10 + |
| | | I76Y_V82S_Y123H_Y147R_Q154R |
| ABE8.21-m | TadA*8.21 | Monomer_TadA*7.10 + Y147R_Q154S |
| ABE8.22-m | TadA*8.22 | Monomer_TadA*7.10 + V82S_Q154S |
| ABE8.23-m | TadA*8.23 | Monomer_TadA*7.10 + V82S_Y123H |
| ABE8.24-m | TadA*8.24 | Monomer_TadA*7.10 + V82S_Y123H_Y147T |
| ABE8.1-d | TadA*8.1 | Heterodimer_(WT) + (TadA*7.10 + Y147T) |
| ABE8.2-d | TadA*8.2 | Heterodimer_(WT) + (TadA*7.10 + Y147R) |
| ABE8.3-d | TadA*8.3 | Heterodimer_(WT) + (TadA*7.10 + Q154S) |
| ABE8.4-d | TadA*8.4 | Heterodimer_(WT) + (TadA*7.10 + Y123H) |
| ABE8.5-d | TadA*8.5 | Heterodimer_(WT) + (TadA*7.10 + V82S) |
| ABE8.6-d | TadA*8.6 | Heterodimer_(WT) + (TadA*7.10 + T166R) |
| ABE8.7-d | TadA*8.7 | Heterodimer_(WT) + (TadA*7.10 + Q154R) |
| ABE8.8-d | TadA*8.8 | Heterodimer_(WT) + (TadA*7.10 + |
| | | Y147R_Q154R_Y123H) |
| ABE8.9-d | TadA*8.9 | Heterodimer_(WT) + (TadA*7.10 + |
| | | Y147R_Q154R_I76Y) |
| ABE8.10-d | TadA*8.10 | Heterodimer_(WT) + (TadA*7.10 + |
| | | Y147R_Q154R_T166R) |
| ABE8.11-d | TadA*8.11 | Heterodimer_(WT) + (TadA*7.10 + Y147T_Q154R) |
| ABE8.12-d | TadA*8.12 | Heterodimer_(WT) + (TadA*7.10 + Y147T_Q154S) |
| ABE8.13-d | TadA*8.13 | Heterodimer_(WT) + (TadA*7.10 + |
| | | Y123H_Y147T_Q154R_I76Y) |
| ABE8.14-d | TadA*8.14 | Heterodimer_(WT) + (TadA*7.10 + I76Y_V82S) |
| ABE8.15-d | TadA*8.15 | Heterodimer_(WT) + (TadA*7.10 + V82S_Y147R) |
| ABE8.16-d | TadA*8.16 | Heterodimer_(WT) + (TadA*7.10 + |
| | | V82S_Y123H_Y147R) |
| ABE8.17-d | TadA*8.17 | Heterodimer_(WT) + (TadA*7.10 + V82S_Q154R) |
| ABE8.18-d | TadA*8.18 | Heterodimer_(WT) + (TadA*7.10 + |
| | | V82S_Y123H_Q154R) |
| ABE8.19-d | TadA*8.19 | Heterodimer_(WT) + (TadA*7.10 + |
| | | V82S_Y123H_Y147R_Q154R) |
| ABE8.20-d | TadA*8.20 | Heterodimer_(WT) + (TadA*7.10 + |
| | | I76Y_V82S_Y123H_Y147R_Q154R) |
| ABE8.21-d | TadA*8.21 | Heterodimer_(WT) + (TadA*7.10 + Y147R_Q154S) |
| ABE8.22-d | TadA*8.22 | Heterodimer_(WT) + (TadA*7.10 + V82S_Q154S) |
| ABE8.23-d | TadA*8.23 | Heterodimer_(WT) + (TadA*7.10 + V82S_Y123H) |
| ABE8.24-d | TadA*8.24 | Heterodimer_(WT) + (TadA*7.10 + |
| | | V82S_Y123H_Y147T) |

In some embodiments, the ABE8 is ABE8a-m, which has a monomeric construct containing TadA*7.10 with R26C, A109S, T111R, D119N, H122N, Y147D, F149Y, T166I, and D167N mutations (TadA*8a). In some embodiments, the ABE8 is ABE8b-m, which has a monomeric construct containing TadA*7.10 with V88A, A109S, T111R, D119N, H122N, F149Y, T166I, and D167N mutations (TadA*8b). In some embodiments, the ABE8 is ABE8c-m, which has a monomeric construct containing TadA*7.10 with R26C, A109S, T111R, D119N, H122N, F149Y, T166I, and D167N mutations (TadA*8c). In some embodiments, the ABE8 is ABE8d-m, which has a monomeric construct containing TadA*7.10 with V88A, T111R, D119N, and F149Y mutations (TadA*8d). In some embodiments, the ABE8 is ABE8e-m, which has a monomeric construct containing TadA*7.10 with A109S, T111R, D119N, H122N, Y147D, F149Y, T166I, and D167N mutations (TadA*8e).

In some embodiments, the ABE8 is ABE8a-d, which has a heterodimeric construct containing wild-type E. coli TadA fused to TadA*7.10 with R26C, A109S, T111R, D119, H122N, Y147D, F149Y, T166I, and D167N mutations (TadA*8a). In some embodiments, the ABE8 is ABE8b-d, which has a heterodimeric construct containing wild-type E. coli TadA fused to TadA*7.10 with V88A, A109S, T111R, D119N, H122N, F149Y, T166I, and D167N mutations (TadA*8b). In some embodiments, the ABE8 is ABE8c-d, which has a heterodimeric construct containing wild-type E. coli TadA fused to TadA*7.10 with R26C, A109S, T111R, D119N, H122N, F149Y, T166I, and D167N mutations (TadA*8c). In some embodiments, the ABE8 is ABE8d-d, which has a heterodimeric construct containing wild-type E. coli TadA fused to TadA*7.10 with V88A, T111R, D119N, and F149Y mutations (TadA*8d). In some embodiments, the ABE8 is ABE8e-d, which has a heterodimeric construct containing wild-type E. coli TadA fused to TadA*7.10 with A109S, T111R, D119N, H122N, Y147D, F149Y, T166I, and D167N mutations (TadA*8e).

In some embodiments, the ABE8 is ABE8a-7, which has a heterodimeric construct containing TadA*7.10 fused to TadA*7.10 with R26C, A109S, T111R, D119, H122N, Y147D, F149Y, T166I, and D167N mutations (TadA*8a). In some embodiments, the ABE8 is ABE8b-7, which has a heterodimeric construct containing TadA*7.10 fused to TadA*7.10 with V88A, A109S, T111R, D119N, H122N, F149Y, T166I, and D167N mutations (TadA*8b). In some embodiments, the ABE8 is ABE8c-7, which has a heterodimeric construct containing TadA*7.10 fused to TadA*7.10 with R26C, A109S, T111R, D119N, H122N, F149Y, T166I, and D167N mutations (TadA*8c). In some embodiments, the ABE8 is ABE8d-7, which has a heterodimeric construct containing TadA*7.10 fused to TadA*7.10 with V88A, T11+R, D119N, and F+49Y mutations (TadA*8d). In some embodiments, the ABE8 is ABE8e-7, which has a heterodimeric construct containing TadA*7.10 fused to TadA*7.10 with A109S, T111R, D119N, H122N, YH47D, F149Y, T166I, and D167N mutations (TadA*8e).

In some embodiments, the ABE is ABE8a-m, ABE8b-m, ABE8c-m, ABE8d-m, ABE8e-m, ABE8a-d, ABE8b-d, ABE8c-d, ABE8d-d, or ABE8e-d, as shown in Table 13 below. In some embodiments, the ABE is ABE8e-m or ABE8e-d. ABE8e shows efficient adenine base editing activity and low indel formation when used with Cas homologues other than SpCas9, for example, SaCas9, SaCas9-KKH, Cas12a homologues, e.g., LbCas12a, enAs-Cas12a, SpCas9-NG and circularly permuted CP1028-SpCas9 and CP1041-SpCas9. In addition to the mutations shown for ABE8e in Table 13, off-target RNA and DNA editing were reduced by introducing a V806W substitution into the TadA domain (as described in M. Richter et al., 2020, Nature Biotechnology, doi.org/i0.1038/s41587-020-0453-z, the entire contents of which are incorporated by reference herein).

TABLE 13
Additional Adenosine Base Editor 8 Variants. In the table, “monomer”
indicates an ABE comprising a single TadA*7.10 comprising the indicated alterations
and “heterodimer” indicates an ABE comprising a TadA*7.10 comprising
the indicated alterations fused to an E. coli TadA adenosine deaminase.

| ABE8 Base | Adenosine | |
|---|---|---|
| Editor | Deaminase | Adenosine Deaminase Description |
| ABE8a-m | TadA*8a | Monomer_TadA*7.10 + R26C + A109S + T111R + D119N + |
| | | H122N + Y147D + F149Y + T166I + D167N |
| ABE8b-m | TadA*8b | Monomer_TadA*7.10 + V88A + A109S + T111R + D119N + |
| | | H122N + F149Y + T166I + D167N |
| ABE8c-m | TadA*8c | Monomer_TadA*7.10 + R26C + A109S + T111R + D119N + |
| | | H122N + F149Y + T166I + D167N |
| ABE8d-m | TadA*8d | Monomer_TadA*7.10 + V88A + T111R + D119N + F149Y |
| ABE8e-m | TadA*8e | Monomer_TadA*7.10 + A109S + T111R + D119N + H122N + |
| | | Y147D + F149Y + T166I + D167N |
| ABE8a-d | TadA*8a | Heterodimer_(WT) + (TadA*7.10 + R26C + A109S + T111R + |
| | | D119N + H122N + Y147D + F149Y + T166I + D167N) |
| ABE8b-d | TadA*8b | Heterodimer_(WT) + (TadA*7.10 + V88A + A109S + T111R + |
| | | D119N + H122N + F149Y + T166I + D167N) |
| ABE8c-d | TadA*8c | Heterodimer_(WT) + (TadA*7.10 + R26C + A109S + T111R + |
| | | D119N + H122N + F149Y + T166I + D167N) |
| ABE8d-d | TadA*8d | Heterodimer_(WT) + (TadA*7.10 + V88A + T111R + D119N + |
| | | F149Y) |
| ABE8e-d | TadA*8e | Heterodimer_(WT) + (TadA*7.10 + A109S + T111R + |
| | | D119N + H122N + Y147D + F149Y + T166I + D167N) |

In some embodiments, base editors (e.g., ABE8) are generated by cloning an adenosine deaminase variant (e.g., TadA*8) into a scaffold that includes a circular permutant Cas9 (e.g., CP5 or CP6) and a bipartite nuclear localization sequence. In some embodiments, the base editor (e.g., ABE7.9, ABE7.10, or ABE8) is an NGC PAM CP5 variant (S. pyogenes Cas9 or spVRQR Cas9). In some embodiments, the base editor (e.g., ABE7.9, ABE7.10, or ABE8) is an AGA PAM CP5 variant (S. pyogenes Cas9 or spVRQR Cas9). In some embodiments, the base editor (e.g., ABE7.9, ABE7.10, or ABE8) is an NGC PAM CP6 variant (S. pyogenes Cas9 or spVRQR Cas9). In some embodiments, the base editor (e.g. ABE7.9, ABE7.10, or ABE8) is an AGA PAM CP6 variant (S. pyogenes Cas9 or spVRQR Cas9).

In some embodiments, the ABE has a genotype as shown in Table 14 below.

TABLE 14
Genotypes of ABEs

| | 23 | 26 | 36 | 37 | 48 | 49 | 51 | 72 | 84 | 87 | 105 | 108 | 123 | 125 | 142 | 145 | 147 | 152 | 155 | 156 | 157 | 161 |
|---|---|---|---|---|---|---|---|---|---|---|---|---|---|---|---|---|---|---|---|---|---|---|
| ABE7.9 | L | R | L | N | A | | L | N | F | S | V | N | Y | G | N | C | Y | P | V | F | N | K |
| ABE7.10 | R | R | L | N | A | | L | N | F | S | V | N | Y | G | A | C | Y | P | V | F | N | K |

As shown in Table 11 below, genotypes of 40 ABE8s are described. Residue positions in the evolved E. coli TadA portion of ABE are indicated. Mutational changes in ABE8 are shown when distinct from ABE7.10 mutations. In some embodiments, the ABE has a genotype of one of the ABEs as shown in Table 15 below.

TABLE 15
Residue Identity in Evolved TadA

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| ABE7.10 | R | L | A | L | I | V | F | V | N | Y | C | Y | P | Q | V | F | N | T |
| ABE8.1-m | | | | | | | | | | | | T | | | | | | |
| ABE8.2-m | | | | | | | | | | | | R | | | | | | |
| ABE8.3-m | | | | | | | | | | | | | | S | | | | |
| ABE8.4-m | | | | | | | | | | H | | | | | | | | |
| ABE8.5-m | | | | | | S | | | | | | | | | | | | |
| ABE8.6-m | | | | | | | | | | | | | | | | | | R |
| ABE8.7-m | | | | | | | | | | | | | | R | | | | |
| ABE8.8-m | | | | | | | | | | H | | R | | R | | | | |
| ABE8.9-m | | | | | Y | | | | | | | R | | R | | | | |
| ABE8.10-m | | | | | | | | | | | | R | | R | | | | R |
| ABE8.11-m | | | | | | | | | | | | T | | R | | | | |
| ABE8.12-m | | | | | | | | | | | | T | | S | | | | |
| ABE8.13-m | | | | | Y | | | | | H | | R | | R | | | | |
| ABE8.14-m | | | | | Y | S | | | | | | | | | | | | |
| ABE8.15-m | | | | | | S | | | | | | R | | | | | | |
| ABE8.16-m | | | | | | S | | | | H | | R | | | | | | |
| ABE8.17-m | | | | | | S | | | | | | | | R | | | | |
| ABE8.18-m | | | | | | S | | | | H | | | | R | | | | |
| ABE8.19-m | | | | | | S | | | | H | | R | | R | | | | |
| ABE8.20-m | | | | | Y | S | | | | H | | R | | R | | | | |
| ABE8.21-m | | | | | | | | | | | | R | | S | | | | |
| ABE8.22-m | | | | | | S | | | | | | | | S | | | | |
| ABE8.23-m | | | | | | S | | | | H | | | | | | | | |
| ABE8.24-m | | | | | | S | | | | H | | T | | | | | | |
| ABE8.1-d | | | | | | | | | | | | T | | | | | | |
| ABE8.2-d | | | | | | | | | | | | R | | | | | | |
| ABE8.3-d | | | | | | | | | | | | | | S | | | | |
| ABE8.4-d | | | | | | | | | | H | | | | | | | | |
| ABE8.5-d | | | | | | S | | | | | | | | | | | | |
| ABE8.6-d | | | | | | | | | | | | | | | | | | R |
| ABE8.7-d | | | | | | | | | | | | | | R | | | | |
| ABE8.8-d | | | | | | | | | | H | | R | | R | | | | |
| ABE8.9-d | | | | | Y | | | | | | | R | | R | | | | |
| ABE8.10-d | | | | | | | | | | | | R | | R | | | | R |
| ABE8.11-d | | | | | | | | | | | | T | | R | | | | |
| ABE8.12-d | | | | | | | | | | | | T | | S | | | | |
| ABE8.13-d | | | | | Y | | | | | H | | R | | R | | | | |
| ABE8.14-d | | | | | Y | S | | | | | | | | | | | | |
| ABE8.15-d | | | | | | S | | | | | | R | | | | | | |
| ABE8.16-d | | | | | | S | | | | H | | R | | | | | | |
| ABE8.17-d | | | | | | S | | | | | | | | R | | | | |
| ABE8.18-d | | | | | | S | | | | H | | | | R | | | | |
| ABE8.19-d | | | | | | S | | | | H | | R | | R | | | | |
| ABE8.20-d | | | | | Y | S | | | | H | | R | | R | | | | |
| ABE8.21-d | | | | | | | | | | | | R | | S | | | | |
| ABE8.22-d | | | | | | S | | | | | | | | S | | | | |
| ABE8.23-d | | | | | | S | | | | H | | | | | | | | |
| ABE8.24-d | | | | | | S | | | | H | | T | | | | | | |

In some embodiments, the base editor is ABE8.1, which comprises or consists essentially of the following sequence or a fragment thereof having adenosine deaminase activity:

ABE8.1_Y147T_CP5_NGC PAM_monomer
(SEQ ID NO: 335)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCTFFR
MPRQVFNAQKKAQSSTDSGGSSGGSSGSETPGTSESATPESSGGSSGGSE
IGKATAKYFFYSNIMNFFKTEITLANGEIRKRPLIETNGETGEIVWDKGR
DFATVRKVLSMPQVNIVKKTEVQTGGFSKESILPKRNSDKLIARKKDWDP
KKYGGFMQPTVAYSVLVVAKVEKGKSKKLKSVKELLGITIMERSSFEKNP
IDFLEAKGYKEVKKDLIIKLPKYSLFELENGRKRMLASAKFLQKGNELAL
PSKYVNFLYLASHYEKLKGSPEDNEQKQLFVEQHKHYLDEIIEQISEFSK
RVILADANLDKVLSAYNKHRDKPIREQAENIIHLFTLTNLGAPRAFKYFD
TTIARKEYRSTKEVLDATLIHQSITGLYETRIDLSQLGGDGGSGGSGGSG
GSGGSGGSGGMDKKYSIGLAIGTNSVGWAVITDEYKVPSKKFKVLGNTDR
HSIKKNLIGALLFDSGETAEATRLKRTARRRYTRRKNRICYLQEIFSNEM
AKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRENAS
LGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQT
VKVVDELVKVMGRHKPENIVIEMARENQTTQKGQKNSRERMKRIEEGIKE
LGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMN
TKYDENDKLIREVKVITLKSKLVSDFRKDFQFYKVREINNYHHAHDAYLN
AVVGTALIKKYPKLESEFVYGDYKVYDVRKMIAKSEQEGADKRTADGSEF
ESPKKKRKV

In the above sequence, the plain text denotes an adenosine deaminase sequence, bold sequence indicates sequence derived from Cas9, the italicized sequence denotes a linker sequence, and the underlined sequence denotes a bipartite nuclear localization sequence. Other ABE8 sequences are provided in the attached sequence listing (SEQ ID NOs: 336-358).

In some embodiments, the base editor is a ninth generation ABE (ABE9). In some embodiments, the ABE9 contains a TadA*9 variant. ABE9 base editors include an adenosine deaminase variant comprising an amino acid sequence, which contains alterations relative to an ABE 7*10 reference sequence, as described herein. Exemplary ABE9 variants are listed in Table 16. Details of ABE9 base editors are described in International PCT Application No. PCT/2020/049975, which is incorporated herein by reference for its entirety.

TABLE 16
Adenosine Base Editor 9 (ABE9) Variants. In the table, “monomer”
indicates an ABE comprising a single TadA*7.10 comprising
the indicated alterations and “heterodimer” indicates
an ABE comprising a TadA*7.10 comprising the indicated alterations
fused to an E. coli TadA adenosine deaminase.

| ABE9 Description | Alterations |
|---|---|
| ABE9.1_monomer | E25F, V82S, Y123H, T133K, Y147R, Q154R |
| ABE9.2_monomer | E25F, V82S, Y123H, Y147R, Q154R |
| ABE9.3_monomer | V82S, Y123H, P124W, Y147R, Q154R |
| ABE9.4_monomer | L51W, V82S, Y123H, C146R, Y147R, Q154R |
| ABE9.5_monomer | P54C, V82S, Y123H, Y147R, Q154R |
| ABE9.6_monomer | Y73S, V82S, Y123H, Y147R, Q154R |
| ABE9.7_monomer | N38G, V82T, Y123H, Y147R, Q154R |
| ABE9.8_monomer | R23H, V82S, Y123H, Y147R, Q154R |
| ABE9.9_monomer | R21N, V82S, Y123H, Y147R, Q154R |
| ABE9.10_monomer | V82S, Y123H, Y147R, Q154R, A158K |
| ABE9.11_monomer | N72K, V82S, Y123H, D139L, Y147R, Q154R, |
| ABE9.12_monomer | E25F, V82S, Y123H, D139M, Y147R, Q154R |
| ABE9.13_monomer | M70V, V82S, M94V, Y123H, Y147R, Q154R |
| ABE9.14_monomer | Q71M, V82S, Y123H, Y147R, Q154R |
| ABE9.15_heterodimer | E25F, V82S, Y123H, T133K, Y147R, Q154R |
| ABE9.16_heterodimer | E25F, V82S, Y123H, Y147R, Q154R |
| ABE9.17_heterodimer | V82S, Y123H, P124W, Y147R, Q154R |
| ABE9.18_heterodimer | L51W, V82S, Y123H, C146R, Y147R, Q154R |
| ABE9.19_heterodimer | P54C, V82S, Y123H, Y147R, Q154R |
| ABE9.2_heterodimer | Y73S, V82S, Y123H, Y147R, Q154R |
| ABE9.21_heterodimer | N38G, V82T, Y123H, Y147R, Q154R |
| ABE9.22_heterodimer | R23H, V82S, Y123H, Y147R, Q154R |
| ABE9.23_heterodimer | R21N, V82S, Y123H, Y147R, Q154R |
| ABE9.24_heterodimer | V82S, Y123H, Y147R, Q154R, A158K |
| ABE9.25_heterodimer | N72K, V82S, Y123H, D139L, Y147R, Q154R, |
| ABE9.26_heterodimer | E25F, V82S, Y123H, D139M, Y147R, Q154R |
| ABE9.27_heterodimer | M70V, V82S, M94V, Y123H, Y147R, Q154R |
| ABE9.28_heterodimer | Q71M, V82S, Y123H, Y147R, Q154R |
| ABE9.29_monomer | E25F_I76Y_V82S_Y123H_Y147R_Q154R |
| ABE9.30_monomer | I76Y_V82T_Y123H_Y147R_Q154R |
| ABE9.31_monomer | N38G_I76Y_V82S_Y123H_Y147R_Q154R |
| ABE9.32_monomer | N38G_I76Y_V82T_Y123H_Y147R_Q154R |
| ABE9.33_monomer | R23H_I76Y_V82S_Y123H_Y147R_Q154R |
| ABE9.34_monomer | P54C_I76Y_V82S_Y123H_Y147R_Q154R |
| ABE9.35_monomer | R21N_I76Y_V82S_Y123H_Y147R_Q154R |
| ABE9.36_monomer | I76Y_V82S_Y123H_D138M_Y147R_Q154R |
| ABE9.37_monomer | Y72S_I76Y_V82S_Y123H_Y147R_Q154R |
| ABE9.38_heterodimer | E25F_I76Y_V82S_Y123H_Y147R_Q154R |
| ABE9.39_heterodimer | I76Y_V82T_Y123H_Y147R_Q154R |
| ABE9.40_heterodimer | N38G_I76Y_V82S_Y123H_Y147R_Q154R |
| ABE9.41_heterodimer | N38G_I76Y_V82T_Y123H_Y147R_Q154R |
| ABE9.42_heterodimer | R23H_I76Y_V82S_Y123H_Y147R_Q154R |
| ABE9.43_heterodimer | P54C_I76Y_V82S_Y123H_Y147R_Q154R |
| ABE9.44_heterodimer | R21N_176Y_V82S_Y123H_Y147R_Q154R |
| ABE9.45_heterodimer | I76Y_V82S_Y123H_D138M_Y147R_Q154R |
| ABE9.46_heterodimer | Y72S_I76Y_V82S_Y123H_Y147R_Q154R |
| ABE9.47_monomer | N72K_V82S, Y123H, Y147R, Q154R |
| ABE9.48_monomer | Q71M_V82S, Y123H, Y147R, Q154R |
| ABE9.49_monomer | M70V, V82S, M94V, Y123H, Y147R, Q154R |
| ABE9.50_monomer | V82S, Y123H, T133K, Y147R, Q154R |
| ABE9.51_monomer | V82S, Y123H, T133K, Y147R, Q154R, |
| | A158K |
| ABE9.52_monomer | M70V, Q71M, N72K, V82S, Y123H, Y147R, |
| | Q154R |
| ABE9.53_heterodimer | N72K_V82S, Y123H, Y147R, Q154R |
| ABE9.54_heterodimer | Q71M_V82S, Y123H, Y147R, Q154R |
| ABE9.55_heterodimer | M70V, V82S, M94V, Y123H, Y147R, Q154R |
| ABE9.56_heterodimer | V82S, Y123H, T133K, Y147R, Q154R |
| ABE9.57_heterodimer | V82S, Y123H, T133K, Y147R, Q154R, |
| | A158K |
| ABE9.58_heterodimer | M70V, Q71M, N72K, V82S, Y123H, Y147R, |
| | Q154R |

In some embodiments, the base editor includes an adenosine deaminase variant comprising an amino acid sequence, which contains alterations relative to an ABE 7*10 reference sequence, as described herein. The term “monomer” as used in Table 16.1 refers to a monomeric form of TadA*7.10 comprising the alterations described. The term “heterodimer” as used in Table 16.1 refers to the specified wild-type E. coli TadA adenosine deaminase fused to a TadA*7.10 comprising the alterations as described.

TABLE 16.1
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10 + L36H + V82G + Y147D + F149Y + |
| | | Q154S + N157K + D167N |
| ABE-827m | MSP827 | monomer_TadA*7.10 + L36H + I76Y + V82G + Y147T + |
| | | Q154S + N157K |
| ABE-828m | MSP828 | monomer_TadA*7.10 + I76Y + V82G + Y147D + F149Y + |
| | | Q154S + D167N |
| ABE-829m | MSP829 | monomer_TadA*7.10 + L36H + I76Y + V82G + Y147D + |
| | | F149Y + Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + 176Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10 + L36H + V82G + Y147D + |
| | | F149Y + Q154S + N157K + D167N) |
| ABE-827d | MSP827 | heterodimer_(WT) + (TadA*7.10 + L36H + I76Y + V82G + |
| | | Y147T + Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10 + L36H + I76Y + V82G + |
| | | Y147D + F149Y + Q154S + N157K + D167N) |

In some embodiments, the base editor comprises a domain comprising all or a portion of a uracil glycosylase inhibitor (UGI). In some embodiments, the base editor comprises a domain comprising all or a portion of a nucleic acid polymerase. In some embodiments, a base editor can comprise as a domain all or a portion of a nucleic acid polymerase (NAP). For example, a base editor can comprise all or a portion of a eukaryotic NAP. In some embodiments, a NAP or portion thereof incorporated into a base editor is a DNA polymerase. In some embodiments, a NAP or portion thereof incorporated into a base editor has translesion polymerase activity. In some embodiments, a NAP or portion thereof incorporated into a base editor is a translesion DNA polymerase. In some embodiments, a NAP or portion thereof incorporated into a base editor is a Rev7, Rev1 complex, polymerase iota, polymerase kappa, or polymerase eta. In some embodiments, a NAP or portion thereof incorporated into a base editor is a eukaryotic polymerase alpha, beta, gamma, delta, epsilon, gamma, eta, iota, kappa, lambda, mu, or nu component. In some embodiments, a NAP or portion thereof incorporated into a base editor comprises an amino acid sequence that is at least 75%, 80%, 85%, 90%, 95%, 96%, 97%, 98%, 99%, or 99.5% identical to a nucleic acid polymerase (e.g., a translesion DNA polymerase). In some embodiments, a nucleic acid polymerase or portion thereof incorporated into a base editor is a translesion DNA polymerase.

In some embodiments, a domain of the base editor can comprise multiple domains. For example, the base editor comprising a polynucleotide programmable nucleotide binding domain derived from Cas9 can comprise a REC lobe and an NUC lobe corresponding to the REC lobe and NUC lobe of a wild-type or natural Cas9. In another example, the base editor can comprise one or more of a RuvCI domain, BH domain, REC1 domain, REC2 domain, RuvCII domain, L1 domain, HNH domain, L2 domain, RuvCIII domain, WED domain, TOPO domain or CTD domain. In some embodiments, one or more domains of the base editor comprise a mutation (e.g., substitution, insertion, deletion) relative to a wild-type version of a polypeptide comprising the domain. For example, an HNH domain of a polynucleotide programmable DNA binding domain can comprise an H840A substitution. In another example, a RuvCI domain of a polynucleotide programmable DNA binding domain can comprise a D10A substitution.

Different domains (e.g., adjacent domains) of the base editor disclosed herein can be connected to each other with or without the use of one or more linker domains (e.g., an XTEN linker domain). In some embodiments, a linker domain can be a bond (e.g., covalent bond), chemical group, or a molecule linking two molecules or moieties, e.g., two domains of a fusion protein, such as, for example, a first domain (e.g., Cas9-derived domain) and a second domain (e.g., an adenosine deaminase domain or a cytidine deaminase domain). In some embodiments, a linker is a covalent bond (e.g., a carbon-carbon bond, disulfide bond, carbon-hetero atom bond, etc.). In certain embodiments, a linker is a carbon nitrogen bond of an amide linkage. In certain embodiments, a linker is a cyclic or acyclic, substituted or unsubstituted, branched or unbranched aliphatic or heteroaliphatic linker. In certain embodiments, a linker is polymeric (e.g., polyethylene, polyethylene glycol, polyamide, polyester, etc.). In certain embodiments, a linker comprises a monomer, dimer, or polymer of aminoalkanoic acid. In some embodiments, a linker comprises an aminoalkanoic acid (e.g., glycine, ethanoic acid, alanine, beta-alanine, 3-aminopropanoic acid, 4-aminobutanoic acid, 5-pentanoic acid, etc.). In some embodiments, a linker comprises a monomer, dimer, or polymer of aminohexanoic acid (Ahx). In certain embodiments, a linker is based on a carbocyclic moiety (e.g., cyclopentane, cyclohexane). In other embodiments, a linker comprises a polyethylene glycol moiety (PEG). In certain embodiments, a linker comprises an aryl or heteroaryl moiety. In certain embodiments, the linker is based on a phenyl ring. A linker can include functionalized moieties to facilitate attachment of a nucleophile (e.g., thiol, amino) from the peptide to the linker. Any electrophile can be used as part of the linker. Exemplary electrophiles include, but are not limited to, activated esters, activated amides, Michael acceptors, alkyl halides, aryl halides, acyl halides, and isothiocyanates. In some embodiments, a linker joins a gRNA binding domain of an RNA-programmable nuclease, including a Cas9 nuclease domain, and the catalytic domain of a nucleic acid editing protein. In some embodiments, a linker joins a dCas9 and a second domain (e.g., UGI, etc.).

### Linkers

In certain embodiments, linkers may be used to link any of the peptides or peptide domains of the invention. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Typically, a linker is positioned between, or flanked by, two groups, molecules, or other moieties and connected to each one via a covalent bond, thus connecting the two. In some embodiments, a linker is an amino acid or a plurality of amino acids (e.g., a peptide or protein). In some embodiments, a linker is an organic molecule, group, polymer, or chemical moiety. In some embodiments, a linker is 2-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the linker is about 3 to about 104 (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100) amino acids in length. Longer or shorter linkers are also contemplated.

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS)n (SEQ ID NO: 250), (GGGGS)n (SEQ ID NO: 251), and (G)n to more rigid linkers of the form (EAAAK)n (SEQ ID NO: 252), (SGGS)n (SEQ ID NO: 359), SGSETPGTSESATPES (SEQ ID NO: 253) (see, e.g., Guilinger J P, et al. Fusion of catalytically inactive Cas9 to FokI nuclease improves the specificity of genome modification. Nat. Biotechnol. 2014; 32(6): 577-82; the entire contents are incorporated herein by reference) and (XP)n) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 253), which can also be referred to as the XTEN linker.

In some embodiments, the domains of the base editor are fused via a linker that comprises the amino acid sequence of:

(SEQ ID NO: 361)
SGGSSGSETPGTSESATPESSGGS,
(SEQ ID NO: 362)
SGGSSGGSSGSETPGTSESATPESSGGSSGGS,
or
(SEQ ID NO: 362)
GGSGGSPGSPAGSPTSTEEGTSESATPESGPGTSTEPSEGSAPGSPA
GSPTSTEEGTSTEPSEGSAPGTSTEPSEGSAPGTSESATPESGPGSE
PATSGGSGGS.

In some embodiments, domains of the base editor are fused via a linker comprising the amino acid sequence SGSETPGTSESATPES (SEQ ID NO: 253), which may also be referred to as the XTEN linker. In some embodiments, a linker comprises the amino acid sequence SGGS. In some embodiments, the linker is 24 amino acids in length. In some embodiments, the linker comprises the amino acid sequence SGGSSGGSSGSETPGTSESATPES (SEQ ID NO: 363). In some embodiments, the linker is 40 amino acids in length. In some embodiments, the linker comprises the amino acid sequence: SGGSSGGSSGSETPGTSESATPESSGGSSGGSSGGSSGGS (SEQ ID NO: 364). In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSGGS (SEQ ID NO: 365). In some embodiments, the linker is 92 amino acids in length. In some embodiments, the linker comprises the amino acid sequence:

(SEQ ID NO: 366)
PGSPAGSPTSTEEGTSESATPESGPGTSTEPSEGSAPGSPAGSPTST
EEGTSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 367), PAPAPA (SEQ ID NO: 368), PAPAPAP (SEQ ID NO: 369), PAPAPAPA (SEQ ID NO: 370), P(AP)4 (SEQ ID NO: 371), P(AP)7 (SEQ ID NO: 372), P(AP)10 (SEQ ID NO: 373) (see, e.g., Tan J, Zhang F, Karcher D, Bock R. Engineering of high-precision base editors for site-specific single nucleotide replacement. Nat Commun. 2019 Jan. 25; 10(1):439; the entire contents are incorporated herein by reference). Such proline-rich linkers are also termed “rigid” linkers.

In another embodiment, the base editor system comprises a component (protein) that interacts non-covalently with a deaminase (DNA deaminase), e.g., an adenosine or a cytidine deaminase, and transiently attracts the adenosine or cytidine deaminase to the target nucleobase in a target polynucleotide sequence for specific editing, with minimal or reduced bystander or target-adjacent effects. Such a non-covalent system and method involving deaminase-interacting proteins serves to attract a DNA deaminase to a particular genomic target nucleobase and decouples the events of on-target and target-adjacent editing, thus enhancing the achievement of more precise single base substitution mutations. In an embodiment, the deaminase-interacting protein binds to the deaminase (e.g., adenosine deaminase or cytidine deaminase) without blocking or interfering with the active (catalytic) site of the deaminase from engaging the target nucleobase (e.g., adenosine or cytidine, respectively). Such as system, termed “MagnEdit,” involves interacting proteins tethered to a Cas9 and gRNA complex and can attract a co-expressed adenosine or cytidine deaminase (either exogenous or endogenous) to edit a specific genomic target site, and is described in McCann, J. et al., 2020, “MagnEdit—interacting factors that recruit DNA-editing enzymes to single base targets,” Life-Science-Alliance, Vol. 3, No. 4 (e201900606), (doi 10.26508/Isa.201900606), the contents of which are incorporated by reference herein in their entirety. In an embodiment, the DNA deaminase is an adenosine deaminase variant (e.g., TadA*8) as described herein.

In another embodiment, a system called “Suntag,” involves non-covalently interacting components used for recruiting protein (e.g., adenosine deaminase or cytidine deaminase) components, or multiple copies thereof, of base editors to polynucleotide target sites to achieve base editing at the site with reduced adjacent target editing, for example, as described in Tanenbaum, M. E. et al., “A protein tagging system for signal amplification in gene expression and fluorescence imaging,” Cell. 2014 Oct. 23; 159(3): 635-646. doi:10.1016/j.cell.2014.09.039; and in Huang, Y.-H. et al., 2017, “DNA epigenome editing using CRISPR-Cas SunTag-directed DNMT3A,” Genome Biol 18: 176. doi:10.1186/s13059-017-1306-z, the contents of each of which are incorporated by reference herein in their entirety. In an embodiment, the DNA deaminase is an adenosine deaminase variant (e.g., TadA*8) as described herein.

Nucleic Acid Programmable DNA Binding Proteins with Guide RNAs

Provided herein are compositions and methods for base editing in cells. Further provided herein are compositions comprising a guide polynucleic acid sequence, e.g. a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g. a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide complexes comprising any of the fusion proteins provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or40 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 7 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins provided herein, and with at least one guide RNA, wherein the guide RNA is about 15-100 nucleotides long and comprises a sequence of at least 10 contiguous nucleotides that is complementary to a target sequence. In some embodiments, the 3′ end of the target sequence is immediately adjacent to an AGC, GAG, TTT, GTG, or CAA sequence. In some embodiments, the 3′ end of the target sequence is immediately adjacent to an NGA, NGCG, NGN, NNGRRT, NNNRRT, NGCG, NGCN, NGTN, NGTN, NGTN, or 5′ (TTTV) sequence. In some embodiments, the 3′ end of the target sequence is immediately adjacent to an e.g., TTN, DTTN, GTTN, ATTN, ATTC, DTTNT, WTTN, HATY, TTTN, TTTV, TTTC, TG, RTR, or YTN PAM site.

It will be understood that the numbering of the specific positions or residues in the respective sequences depends on the particular protein and numbering scheme used. Numbering might differ, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

It will be apparent to those of skill in the art that in order to target any of the fusion proteins disclosed herein, to a target site, e.g., a site comprising a mutation to be edited, it is typically necessary to co-express the fusion protein together with a guide RNA. As explained in more detail elsewhere herein, a guide RNA typically comprises a tracrRNA framework allowing for napDNAbp (e.g., Cas9 or Cas12) binding, and a guide sequence, which confers sequence specificity to the napDNAbp:nucleic acid editing enzyme/domain fusion protein. Alternatively, the guide RNA and tracrRNA may be provided separately, as two nucleic acid molecules. In some embodiments, the guide RNA comprises a structure, wherein the guide sequence comprises a sequence that is complementary to the target sequence. The guide sequence is typically 20 nucleotides long. The sequences of suitable guide RNAs for targeting napDNAbp:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein.

Distinct portions of sgRNA are predicted to form various features that interact with Cas9 (e.g., SpyCas9) and/or the DNA target. Six conserved modules have been identified within native crRNA:tracrRNA duplexes and single guide RNAs (sgRNAs) that direct Cas9 endonuclease activity (see Briner et al., Guide RNA Functional Modules Direct Cas9 Activity and Orthogonality Mol Cell. 2014 Oct. 23; 56(2):333-339). The six modules include the spacer responsible for DNA targeting, the upper stem, bulge, lower stem formed by the CRISPR repeat:tracrRNA duplex, the nexus, and hairpins from the 3′ end of the tracrRNA. The upper and lower stems interact with Cas9 mainly through sequence-independent interactions with the phosphate backbone. In some embodiments, the upper stem is dispensable. In some embodiments, the conserved uracil nucleotide sequence at the base of the lower stem is dispensable. The bulge participates in specific side-chain interactions with the Rec domain of Cas9. The nucleobase of U44 interacts with the side chains of Tyr 325 and His 328, while G43 interacts with Tyr 329. The nexus forms the core of the sgRNA:Cas9 interactions and lies at the intersection between the sgRNA and both Cas9 and the target DNA. The nucleobases of A51 and A52 interact with the side chain of Phe 1105; U56 interacts with Arg 457 and Asn 459; the nucleobase of U59 inserts into a hydrophobic pocket defined by side chains of Arg 74, Asn 77, Pro 475, Leu 455, Phe 446, and Ile 448; C60 interacts with Leu 455, Ala 456, and Asn 459, and C61 interacts with the side chain of Arg 70, which in turn interacts with C15. In some embodiments, one or more of these mutations are made in the bulge and/or the nexus of a sgRNA for a Cas9 (e.g., spyCas9) to optimize sgRNA:Cas9 interactions.

Moreover, the tracrRNA nexus and hairpins are critical for Cas9 pairing and can be swapped to cross orthogonality barriers separating disparate Cas9 proteins, which is instrumental for further harnessing of orthogonal Cas9 proteins. In some embodiments, the nexus and hairpins are swapped to target orthogonal Cas9 proteins. In some embodiments, a sgRNA is dispensed of the upper stem, hairpin 1, and/or the sequence flexibility of the lower stem to design a guide RNA that is more compact and conformationally stable. In some embodiments, the modules are modified to optimize multiplex editing using a single Cas9 with various chimeric guides or by concurrently using orthogonal systems with different combinations of chimeric sgRNAs. Details regarding guide functional modules and methods thereof are described, for example, in Briner et al., Guide RNA Functional Modules Direct Cas9 Activity and Orthogonality Mol Cell. 2014 Oct. 23; 56(2):333-339, the contents of which is incorporated by reference herein in its entirety.

The domains of the base editor disclosed herein can be arranged in any order. Non-limiting examples of a base editor comprising a fusion protein comprising e.g., a polynucleotide-programmable nucleotide-binding domain (e.g., Cas9 or Cas12) and a deaminase domain (e.g., cytidine or adenosine deaminase) can be arranged as follows:
NH2-[nucleobase editing domain]-Linker1-[nucleobase editing domain]-COOH;
NH2-[deaminase]-Linker1-[nucleobase editing domain]-COOH;
NH2-[deaminase]-Linker1-[nucleobase editing domain]-Linker2-[UGI]-COOH;
NH2-[deaminase]-Linker1-[nucleobase editing domain]-COOH;
NH2-[adenosine deaminase]-Linker1-[nucleobase editing domain]-COOH;
NH2-[nucleobase editing domain]-[deaminase]-COOH;
NH2-[deaminase]-[nucleobase editing domain]-[inosine BER inhibitor]-COOH;
NH2-[deaminase]-[inosine BER inhibitor]-[nucleobase editing domain]-COOH;
NH2-[inosine BER inhibitor]-[deaminase]-[nucleobase editing domain]-COOH;
NH2-[nucleobase editing domain]-[deaminase]-[inosine BER inhibitor]-COOH;
NH2-[nucleobase editing domain]-[inosine BER inhibitor]-[deaminase]-COOH;
NH2-[inosine BER inhibitor]-[nucleobase editing domain]-[deaminase]-COOH;
NH2-[nucleobase editing domain]-Linker1-[deaminase]-Linker2-[nucleobase editing domain]-COOH;
NH2-[nucleobase editing domain]-Linker1-[deaminase]-[nucleobase editing domain]-COOH;
NH2-[nucleobase editing domain]-[deaminase]-Linker2-[nucleobase editing domain]-COOH;
NH2-[nucleobase editing domain]-[deaminase]-[nucleobase editing domain]-COOH;
NH2-[nucleobase editing domain]-Linker1-[deaminase]-Linker2-[nucleobase editing domain]-[inosine BER inhibitor]-COOH;
NH2-[nucleobase editing domain]-Linker1-[deaminase]-[nucleobase editing domain]-[inosine BER inhibitor]-COOH;
NH2-[nucleobase editing domain]-[deaminase]-Linker2-[nucleobase editing domain]-[inosine BER inhibitor]-COOH;
NH2-[nucleobase editing domain]-[deaminase]-[nucleobase editing domain]-[inosine BER inhibitor]-COOH;
NH2-[inosine BER inhibitor]-[nucleobase editing domain]-Linker1-[deaminase]-Linker2-[nucleobase editing domain]-COOH;
NH2-[inosine BER inhibitor]-[nucleobase editing domain]-Linker1-[deaminase]-[nucleobase editing domain]-COOH;
NH2-[inosine BER inhibitor]-[nucleobase editing domain]-[deaminase]-Linker2-[nucleobase editing domain]-COOH; or
NH2-[inosine BER inhibitor]NH2-[nucleobase editing domain]-[deaminase]-[nucleobase editing domain]-COOH.

In some embodiments, the base editing fusion proteins provided herein need to be positioned at a precise location, for example, where a target base is placed within a defined region (e.g., a “deamination window”). In some embodiments, a target can be within a 4-base region. In some embodiments, such a defined target region can be approximately 15 bases upstream of the PAM. See Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a napDNAbp domain. In some embodiments, an NLS of the base editor is localized C-terminal to a napDNAbp domain.

Non-limiting examples of protein domains which can be included in the fusion protein include a deaminase domain (e.g., adenosine deaminase or cytidine deaminase), a uracil glycosylase inhibitor (UGI) domain, epitope tags, reporter gene sequences, and/or protein domains having one or more of the activities described herein.

A domain may be detected or labeled with an epitope tag, a reporter protein, other binding domains. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). Additional protein sequences can include amino acid sequences that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions.

### Methods of Using Fusion Proteins Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins provided herein, and with at least one guide RNA described herein.

In some embodiments, a fusion protein of the invention is used for editing a target gene of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

It will be apparent to those of skill in the art that in order to target any of the fusion proteins comprising a Cas9 domain and a cytidine or adenosine deaminase, as disclosed herein, to a target site, e.g., a site comprising a mutation to be edited, it is typically necessary to co-express the fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein. Alternatively, the guide RNA and tracrRNA may be provided separately, as two nucleic acid molecules. In some embodiments, the guide RNA comprises a structure, wherein the guide sequence comprises a sequence that is complementary to the target sequence. The guide sequence is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins comprising a polynucleotide programmable nucleotide binding domain (e.g., Cas9) and a nucleobase editing domain (e.g., an adenosine deaminase domain or a cytidine deaminase domain) can be used to edit a nucleotide from A to G or C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations. In some embodiments, an intended mutation is a mutation that is generated by a specific base editor (e.g., adenosine base editor or cytidine base editor) bound to a guide polynucleotide (e.g., gRNA), specifically designed to generate the intended mutation. In some embodiments, the intended mutation is in a gene associated with a target antigen associated with a disease or disorder, e.g., an amyloid disease such as cardiomyopathy, familial amyloid polyneuropathy (FAP), familial amyloid cardiomyopathy (FAC), familial transthyretin amyloidosis (FTA), senile systemic amyloidosis (SSA), transthyretin amyloidosis, and the like. In some embodiments, the intended mutation is an adenine (A) to guanine (G) point mutation (e.g., SNP) in a gene associated with a target antigen associated with a disease or disorder, e.g., an amyloid disease such as cardiomyopathy, familial amyloid polyneuropathy (FAP), familial amyloid cardiomyopathy (FAC), familial transthyretin amyloidosis (FTA), senile systemic amyloidosis (SSA), transthyretin amyloidosis, and the like. In some embodiments, the intended mutation is an adenine (A) to guanine (G) point mutation within the coding region or non-coding region of a gene (e.g., regulatory region or element). In some embodiments, the intended mutation is a cytosine (C) to thymine (T) point mutation (e.g., SNP) in a gene associated with a target antigen associated with a disease or disorder, e.g., an amyloid disease such as cardiomyopathy, familial amyloid polyneuropathy (FAP), familial amyloid cardiomyopathy (FAC), familial transthyretin amyloidosis (FTA), senile systemic amyloidosis (SSA), transthyretin amyloidosis, and the like. In some embodiments, the intended mutation is a cytosine (C) to thymine (T) point mutation within the coding region or non-coding region of a gene (e.g., regulatory region or element). In some embodiments, the intended mutation is a point mutation that generates a STOP codon, for example, a premature STOP codon within the coding region of a gene. In some embodiments, the intended mutation is a mutation that eliminates a stop codon.

The base editors of the invention advantageously modify a specific nucleotide base encoding a protein without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g. mutate) a specific nucleotide within a nucleic acid, without generating a large number of insertions or deletions (i.e., indels) in the nucleic acid. In some embodiments, it is desirable to generate base editors that efficiently modify (e.g. mutate or methylate) a specific nucleotide within a nucleic acid, without generating a large number of insertions or deletions (i.e., indels) in the nucleic acid. In certain embodiments, any of the base editors provided herein can generate a greater proportion of intended modifications (e.g., methylations) versus indels. In certain embodiments, any of the base editors provided herein can generate a greater proportion of intended modifications (e.g., mutations) versus indels.

In some embodiments, the base editors provided herein are capable of generating a ratio of intended mutations to indels (i.e., intended point mutations:unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, a number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some aspects of the disclosure are based on the recognition that any of the base editors provided herein are capable of efficiently generating an intended mutation in a nucleic acid (e.g. a nucleic acid within a genome of a subject) without generating a considerable number of unintended mutations (e.g., spurious off-target editing or bystander editing). In some embodiments, an intended mutation is a mutation that is generated by a specific base editor bound to a gRNA, specifically designed to generate the intended mutation. In some embodiments, the intended mutation is a mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base editors provided herein are capable of generating a ratio of intended mutations to unintended mutations (e.g., intended mutations:unintended mutations) that is greater than 1:1. In some embodiments, any of the base editors provided herein are capable of generating a ratio of intended mutations to unintended mutations that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the base editors described herein may be applied to any of the fusion proteins, or methods of using the fusion proteins provided herein.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence, and may affect the gene product. In essence therefore, the gene editing modification described herein may result in a modification of the gene, structurally and/or functionally, wherein the expression of the gene product may be modified, for example, the expression of the gene is knocked out; or conversely, enhanced, or, in some circumstances, the gene function or activity may be modified. Using the methods disclosed herein, a base editing efficiency may be determined as the knockdown efficiency of the gene in which the base editing is performed, wherein the base editing is intended to knockdown the expression of the gene. A knockdown level may be validated quantitatively by determining the expression level by any detection assay, such as assay for protein expression level, for example, by flow cytometry; assay for detecting RNA expression such as quantitative RT-PCR, northern blot analysis, or any other suitable assay such as pyrosequencing; and may be validated qualitatively by nucleotide sequencing reactions.

In some embodiments, the modification, e.g., single base edit results in at least 10% reduction of the gene targeted expression. In some embodiments, the base editing efficiency may result in at least 10% reduction of the gene targeted expression. In some embodiments, the base editing efficiency may result in at least 20% reduction of the gene targeted expression. In some embodiments, the base editing efficiency may result in at least 30% reduction of the gene targeted expression. In some embodiments, the base editing efficiency may result in at least 40% reduction of the gene targeted expression. In some embodiments, the base editing efficiency may result in at least 50% reduction of the gene targeted expression. In some embodiments, the base editing efficiency may result in at least 60% reduction of the targeted gene expression. In some embodiments, the base editing efficiency may result in at least 70% reduction of the targeted gene expression. In some embodiments, the base editing efficiency may result in at least 80% reduction of the targeted gene expression. In some embodiments, the base editing efficiency may result in at least 90% reduction of the targeted gene expression. In some embodiments, the base editing efficiency may result in at least 91% reduction of the targeted gene expression. In some embodiments, the base editing efficiency may result in at least 92% reduction of the targeted gene expression. In some embodiments, the base editing efficiency may result in at least 93% reduction of the targeted gene expression. In some embodiments, the base editing efficiency may result in at least 94% reduction of the targeted gene expression. In some embodiments, the base editing efficiency may result in at least 95% reduction of the targeted gene expression. In some embodiments, the base editing efficiency may result in at least 96% reduction of the targeted gene expression. In some embodiments, the base editing efficiency may result in at least 97% reduction of the targeted gene expression. In some embodiments, the base editing efficiency may result in at least 98% reduction of the targeted gene expression. In some embodiments, the base editing efficiency may result in at least 99% reduction of the targeted gene expression. In some embodiments, the base editing efficiency may result in knockout (100% knockdown of the gene expression) of the gene that is targeted.

In some embodiments, any of the base editor systems provided herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence.

In some embodiments, targeted modifications, e.g., single base editing, are used simultaneously to target at least 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 different endogenous sequences for base editing with different guide RNAs. In some embodiments, targeted modifications, e.g. single base editing, are used to sequentially target at least 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 50, or more different endogenous gene sequences for base editing with different guide RNAs.

Some aspects of the disclosure are based on the recognition that any of the base editors provided herein are capable of efficiently generating an intended mutation, such as a point mutation, in a nucleic acid (e.g., a nucleic acid within a genome of a subject) without generating a significant number of unintended mutations, such as unintended point mutations (i.e., mutation of bystanders). In some embodiments, any of the base editors provided herein are capable of generating at least 0.01% of intended mutations (i.e., at least 0.01% base editing efficiency). In some embodiments, any of the base editors provided herein are capable of generating at least 0.01%, 1%, 2%, 3%, 4%, 5%, 10%, 15%, 20%, 25%, 30%, 40%, 45%, 50%, 60%, 70%, 80%, 90%, 95%, or 99% of intended mutations.

In some embodiments, any of the base editor systems comprising one of the ABE8 base editor variants described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence. In some embodiments, any of the base editor systems comprising one of the ABE8 base editor variants described herein result in less than 0.8% indel formation in the target polynucleotide sequence. In some embodiments, any of the base editor systems comprising one of the ABE8 base editor variants described herein result in at most 0.8% indel formation in the target polynucleotide sequence. In some embodiments, any of the base editor systems comprising one of the ABE8 base editor variants described herein result in less than 0.3% indel formation in the target polynucleotide sequence. In some embodiments, any of the base editor systems comprising one of the ABE8 base editor variants described results in lower indel formation in the target polynucleotide sequence compared to a base editor system comprising one of ABE7 base editors. In some embodiments, any of the base editor systems comprising one of the ABE8 base editor variants described herein results in lower indel formation in the target polynucleotide sequence compared to a base editor system comprising an ABE7.10.

In some embodiments, any of the base editor systems comprising one of the ABE8 base editor variants described herein has reduction in indel frequency compared to a base editor system comprising one of the ABE7 base editors. In some embodiments, any of the base editor systems comprising one of the ABE8 base editor variants described herein has at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% reduction in indel frequency compared to a base editor system comprising one of the ABE7 base editors. In some embodiments, a base editor system comprising one of the ABE8 base editor variants described herein has at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% reduction in indel frequency compared to a base editor system comprising an ABE7.10.

The invention provides adenosine deaminase variants (e.g., ABE8 variants) that have increased efficiency and specificity. In particular, the adenosine deaminase variants described herein are more likely to edit a desired base within a polynucleotide, and are less likely to edit bases that are not intended to be altered (e.g., “bystanders”).

In some embodiments, any of the base editing system comprising one of the ABE8 base editor variants described herein has reduced bystander editing or mutations. In some embodiments, an unintended editing or mutation is a bystander mutation or bystander editing, for example, base editing of a target base (e.g., A or C) in an unintended or non-target position in a target window of a target nucleotide sequence. In some embodiments, any of the base editing system comprising one of the ABE8 base editor variants described herein has reduced bystander editing or mutations compared to a base editor system comprising an ABE7 base editor, e.g., ABE7.10. In some embodiments, any of the base editing system comprising one of the ABE8 base editor variants described herein has reduced bystander editing or mutations by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% compared to a base editor system comprising an ABE7 base editor, e.g., ABE7.10. In some embodiments, any of the base editing system comprising one of the ABE8 base editor variants described herein has reduced bystander editing or mutations by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold compared to a base editor system comprising an ABE7 base editor, e.g., ABE7.10.

In some embodiments, any of the base editing system comprising one of the ABE8 base editor variants described herein has reduced spurious editing. In some embodiments, an unintended editing or mutation is a spurious mutation or spurious editing, for example, non-specific editing or guide independent editing of a target base (e.g., A or C) in an unintended or non-target region of the genome. In some embodiments, any of the base editing system comprising one of the ABE8 base editor variants described herein has reduced spurious editing compared to a base editor system comprising an ABE7 base editor, e.g., ABE7.10. In some embodiments, any of the base editing system comprising one of the ABE8 base editor variants described herein has reduced spurious editing by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% compared to a base editor system comprising an ABE7 base editor, e.g., ABE7.10. In some embodiments, any of the base editing system comprising one of the ABE8 base editor variants described herein has reduced spurious editing by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold compared to a base editor system comprising an ABE7 base editor, e.g., ABE7.10.

In some embodiments, any of the ABE8 base editor variants described herein have at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% base editing efficiency. In some embodiments, the base editing efficiency may be measured by calculating the percentage of edited nucleobases in a population of cells. In some embodiments, any of the ABE8 base editor variants described herein have base editing efficiency of at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited nucleobases in a population of cells.

In some embodiments, any of the ABE8 base editor variants described herein has higher base editing efficiency compared to the ABE7 base editors. In some embodiments, any of the ABE8 base editor variants described herein have at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 450%, or at least 500% higher base editing efficiency compared to an ABE7 base editor, e.g., ABE7.10.

In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher base editing efficiency compared to an ABE7 base editor, e.g., ABE7.10.

In some embodiments, any of the ABE8 base editor variants described herein have at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% on-target base editing efficiency. In some embodiments, any of the ABE8 base editor variants described herein have on-target base editing efficiency of at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited target nucleobases in a population of cells.

In some embodiments, any of the ABE8 base editor variants described herein has higher on-target base editing efficiency compared to the ABE7 base editors. In some embodiments, any of the ABE8 base editor variants described herein have at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 450%, or at least 500% higher on-target base editing efficiency compared to an ABE7 base editor, e.g., ABE7.10.

In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher on-target base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA. In some embodiments, an ABE8 base editor delivered via a nucleic acid based delivery system, e.g., an mRNA, has on-target editing efficiency of at least at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited nucleobases. In some embodiments, an ABE8 base editor delivered by an mRNA system has higher base editing efficiency compared to an ABE8 base editor delivered by a plasmid or vector system. In some embodiments, any of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300% higher, at least 310%, at least 320%, at least 330%, at least 340%, at least 350%, at least 360%, at least 370%, at least 380%, at least 390%, at least 400%, at least 450%, or at least 500% on-target editing efficiency when delivered by an mRNA system compared to when delivered by a plasmid or vector system. In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher on-target editing efficiency when delivered by an mRNA system compared to when delivered by a plasmid or vector system.

In some embodiments, any of the base editor systems comprising one of the ABE8 base editor variants described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in the target polynucleotide sequence.

In some embodiments, any of the ABE8 base editor variants described herein has lower guided off-target editing efficiency when delivered by an mRNA system compared to when delivered by a plasmid or vector system. In some embodiments, any of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% lower guided off-target editing efficiency when delivered by an mRNA system compared to when delivered by a plasmid or vector system. In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold lower guided off-target editing efficiency when delivered by an mRNA system compared to when delivered by a plasmid or vector system. In some embodiments, any of the ABE8 base editor variants described herein has at least about 2.2 fold decrease in guided off-target editing efficiency when delivered by an mRNA system compared to when delivered by a plasmid or vector system.

In some embodiments, any of the ABE8 base editor variants described herein has lower guide-independent off-target editing efficiency when delivered by an mRNA system compared to when delivered by a plasmid or vector system. In some embodiments, any of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% lower guide-independent off-target editing efficiency when delivered by an mRNA system compared to when delivered by a plasmid or vector system. In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 5.0 fold, at least 10.0 fold, at least 20.0 fold, at least 50.0 fold, at least 70.0 fold, at least 100.0 fold, at least 120.0 fold, at least 130.0 fold, or at least 150.0 fold lower guide-independent off-target editing efficiency when delivered by an mRNA system compared to when delivered by a plasmid or vector system. In some embodiments, ABE8 base editor variants described herein has 134.0 fold decrease in guide-independent off-target editing efficiency (e.g., spurious RNA deamination) when delivered by an mRNA system compared to when delivered by a plasmid or vector system. In some embodiments, ABE8 base editor variants described herein does not increase guide-independent mutation rates across the genome.

In some embodiments, a single gene delivery event (e.g., by transduction, transfection, electroporation or any other method) can be used to target base editing of 5 sequences within a cell's genome. In some embodiments, a single gene delivery event can be used to target base editing of 6 sequences within a cell's genome. In some embodiments, a single gene delivery event can be used to target base editing of 7 sequences within a cell's genome. In some embodiments, a single electroporation event can be used to target base editing of 8 sequences within a cell's genome. In some embodiments, a single gene delivery event can be used to target base editing of 9 sequences within a cell's genome. In some embodiments, a single gene delivery event can be used to target base editing of 10 sequences within a cell's genome. In some embodiments, a single gene delivery event can be used to target base editing of 20 sequences within a cell's genome. In some embodiments, a single gene delivery event can be used to target base editing of 30 sequences within a cell's genome. In some embodiments, a single gene delivery event can be used to target base editing of 40 sequences within a cell's genome. In some embodiments, a single gene delivery event can be used to target base editing of 50 sequences within a cell's genome.

In some embodiments, the method described herein, for example, the base editing methods has minimum to no off-target effects.

In some embodiments, the base editing method described herein results in at least 50% of a cell population that have been successfully edited (i.e., cells that have been successfully engineered). In some embodiments, the base editing method described herein results in at least 55% of a cell population that have been successfully edited. In some embodiments, the base editing method described herein results in at least 60% of a cell population that have been successfully edited. In some embodiments, the base editing method described herein results in at least 65% of a cell population that have been successfully edited. In some embodiments, the base editing method described herein results in at least 70% of a cell population that have been successfully edited. In some embodiments, the base editing method described herein results in at least 75% of a cell population that have been successfully edited. In some embodiments, the base editing method described herein results in at least 80% of a cell population that have been successfully edited. In some embodiments, the base editing method described herein results in at least 85% of a cell population that have been successfully edited. In some embodiments, the base editing method described herein results in at least 90% of a cell population that have been successfully edited. In some embodiments, the base editing method described herein results in at least 95% of a cell population that have been successfully edited. In some embodiments, the base editing method described herein results in about 91%, 92%, 93%, 94%, 95%, 96%, 97%, 98%, 99% or 100% of a cell population that have been successfully edited.

In some embodiments, the live cell recovery following a base editing intervention is greater than at least 60%, 70%, 80%, 90% of the starting cell population at the time of the base editing event. In some embodiments, the live cell recovery as described above is about 70%. In some embodiments, the live cell recovery as described above is about 75%. In some embodiments, the live cell recovery as described above is about 80%. In some embodiments, the live cell recovery as described above is about 85%. In some embodiments, the live cell recovery as described above is about 90%, or about 91%, 92%, 93%, 94% 95%, 96%, 97%, 98%, or 99%, or 100% of the cells in the population at the time of the base editing event.

In some embodiments the engineered cell population can be further expanded in vitro by about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The number of intended mutations and indels can be determined using any suitable method, for example, as described in International PCT Application Nos. PCT/2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

The number of indels formed at a target nucleotide region can depend on the amount of time a nucleic acid (e.g., a nucleic acid within the genome of a cell) is exposed to a base editor. In some embodiments, the number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing the target nucleotide sequence (e.g., a nucleic acid within the genome of a cell) to a base editor. It should be appreciated that the characteristics of the base editors as described herein can be applied to any of the fusion proteins, or methods of using the fusion proteins provided herein.

Details of base editor efficiency are described in International PCT Application Nos. PCT/2017/045381 (WO 2018/027078) and PCT/US2016/058344 (WO 2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference. In some embodiments, editing of a plurality of nucleobase pairs in one or more genes using the methods provided herein results in formation of at least one intended mutation. In some embodiments, said formation of said at least one intended mutation results in the disruption the normal function of a gene. In some embodiments, said formation of said at least one intended mutation results decreases or eliminates the expression of a protein encoded by a gene. It should be appreciated that multiplex editing can be accomplished using any method or combination of methods provided herein.

### Multiplex Editing

In some embodiments, the base editor system provided herein is capable of multiplex editing of a plurality of nucleobase pairs in one or more genes. In some embodiments, the plurality of nucleobase pairs is located in the same gene or in one or more genes, wherein at least one gene is located in a different locus. In some embodiments, the multiplex editing can comprise one or more guide polynucleotides. In some embodiments, the multiplex editing can comprise one or more base editor systems. In some embodiments, the multiplex editing can comprise one or more base editor systems with a single guide polynucleotide or a plurality of guide polynucleotides. In some embodiments, the multiplex editing can comprise one or more guide polynucleotides with a single base editor system. In some embodiments, the multiplex editing can comprise at least one guide polynucleotide that does or does not require a PAM sequence to target binding to a target polynucleotide sequence. In some embodiments, the multiplex editing can comprise a mix of at least one guide polynucleotide that does not require a PAM sequence to target binding to a target polynucleotide sequence and at least one guide polynucleotide that require a PAM sequence to target binding to a target polynucleotide sequence. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the plurality of nucleobase pairs are in one more genes. In some embodiments, the plurality of nucleobase pairs is in the same gene. In some embodiments, at least one gene in the one more genes is located in a different locus.

In some embodiments, the editing is editing of the plurality of nucleobase pairs in at least one protein coding region, in at least one protein non-coding region, or in at least one protein coding region and at least one protein non-coding region.

In some embodiments, the editing is in conjunction with one or more guide polynucleotides. In some embodiments, the base editor system can comprise one or more base editor systems. In some embodiments, the base editor system can comprise one or more base editor systems in conjunction with a single guide polynucleotide or a plurality of guide polynucleotides. In some embodiments, the editing is in conjunction with one or more guide polynucleotide with a single base editor system. In some embodiments, the editing is in conjunction with at least one guide polynucleotide that does not require a PAM sequence to target binding to a target polynucleotide sequence or with at least one guide polynucleotide that requires a PAM sequence to target binding to a target polynucleotide sequence, or with a mix of at least one guide polynucleotide that does not require a PAM sequence to target binding to a target polynucleotide sequence and at least one guide polynucleotide that does require a PAM sequence to target binding to a target polynucleotide sequence. It should be appreciated that the characteristics of the multiplex editing using any of the base editors as described herein can be applied to any of combination of the methods of using any of the base editors provided herein. It should also be appreciated that the editing can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors. In some embodiments, the base editor system capable of multiplex editing comprising one of the ABE8 base editor variants described herein has higher multiplex editing efficiency compared to the base editor system capable of multiplex editing comprising one of ABE7 base editors. In some embodiments, the base editor system capable of multiplex editing comprising one of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300% higher, at least 310%, at least 320%, at least 330%, at least 340%, at least 350%, at least 360%, at least 370%, at least 380%, at least 390%, at least 400%, at least 450%, or at least 500% higher multiplex editing efficiency compared the base editor system capable of multiplex editing comprising one of ABE7 base editors. In some embodiments, the base editor system capable of multiplex editing comprising one of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 4.0 fold, at least 4.5 fold, at least 5.0 fold, at least 5.5 fold, or at least 6.0 fold higher multiplex editing efficiency compared the base editor system capable of multiplex editing comprising one of ABE7 base editors.

### Delivery System

The suitability of nucleobase editors to target one or more nucleotides in a gene (e.g., a transthyretin (TTR) gene) is evaluated as described herein. In one embodiment, a single cell of interest is transfected, transduced, or otherwise modified with a nucleic acid molecule or molecules encoding a base editing system described herein together with a small amount of a vector encoding a reporter (e.g., GFP). These cells can be any cell line known in the art, including hepatocytes. Alternatively, primary cells (e.g., human) may be used. Cells may also be obtained from a subject or individual, such as from tissue biopsy, surgery, blood, plasma, serum, or other biological fluid. Such cells may be relevant to the eventual cell target.

Delivery may be performed using a viral vector. In one embodiment, transfection may be performed using lipid transfection (such as Lipofectamine or Fugene) or by electroporation. Following transfection, expression of a reporter (e.g., GFP) can be determined either by fluorescence microscopy or by flow cytometry to confirm consistent and high levels of transfection. These preliminary transfections can comprise different nucleobase editors to determine which combinations of editors give the greatest activity. The system can comprise one or more different vectors. In one embodiment, the base editor is codon optimized for expression of the desired cell type, preferentially a eukaryotic cell, preferably a mammalian cell or a human cell.

The activity of the nucleobase editor is assessed as described herein, i.e., by sequencing the genome of the cells to detect alterations in a target sequence. For Sanger sequencing, purified PCR amplicons are cloned into a plasmid backbone, transformed, miniprepped and sequenced with a single primer. Sequencing may also be performed using next generation sequencing (NGS) techniques. When using next generation sequencing, amplicons may be 300-500 bp with the intended cut site placed asymmetrically. Following PCR, next generation sequencing adapters and barcodes (for example Illumina multiplex adapters and indexes) may be added to the ends of the amplicon, e.g., for use in high throughput sequencing (for example on an Illumina MiSeq). The fusion proteins that induce the greatest levels of target specific alterations in initial tests can be selected for further evaluation.

In particular embodiments, the nucleobase editors are used to target polynucleotides of interest. In one embodiment, a nucleobase editor of the invention is delivered to cells (e.g., hepatocytes) in conjunction with one or more guide RNAs that are used to target one or more nucleic acid sequences of interest within the genome of a cell, thereby altering the target gene(s) (e.g., a transthyretin gene (TTR)). In some embodiments, a base editor is targeted by one or more guide RNAs to introduce one or more edits to the sequence of one or more genes of interest (e.g., a transthyretin gene (TTR)). In some embodiments, the one or more edits to the sequence of one or more genes of interest decrease or eliminate expression of the protein encoded by the gene in the host cell (e.g., a transthyretin (TTR) polypeptide). In some embodiments, expression of one or more proteins encoded by one or more genes of interest (e.g., a transthyretin (TTR) gene) is completely knocked out or eliminated in the host cell ((e.g., a hepatocyte).

In some embodiments, the host cell is a mammalian cell. In some embodiments, the host cell is a human cell.

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions.

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g. lipid and/or polymer) nanoparticles are suitable for use as delivery vehicles in certain embodiments of this disclosure. Exemplary lipids for use in nanoparticle formulations, and/or gene transfer are shown in Table 17 (below).

TABLE 17
Lipids used for gene transfer.
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N- | DOSPA | Cationic |
| dimethy1-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spermine | CCS | Cationic |
| N-t-Butyl-N0-tetradecy1-3-tetradecylaminopropionamidine | diC14-amidine | Cationic |
| Octadecenolyoxy[ethyl-2-heptadeceny1-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoley1-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Table 18 lists exemplary polymers for use in gene transfer and/or nanoparticle formulations.

TABLE 18
Polymers used for gene transfer.
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Table 19 summarizes delivery methods for a polynucleotide encoding a fusion protein described herein.

TABLE 19
Delivery methods.

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

In another aspect, the delivery of base editor system components or nucleic acids encoding such components, for example, a polynucleotide programmable nucleotide binding domain (e.g., Cas9) such as, for example, Cas9 or variants thereof, and a gRNA targeting a nucleic acid sequence of interest, may be accomplished by delivering the ribonucleoprotein (RNP) to cells. The RNP comprises a polynucleotide programmable nucleotide binding domain (e.g., Cas9), in complex with the targeting gRNA. RNPs or polynucleotides described herein may be delivered to cells using known methods, such as electroporation, nucleofection, or cationic lipid-mediated methods, for example, as reported by Zuris, J. A. et al., 2015, Nat. Biotechnology, 33(1):73-80, which is incorporated by reference in its entirety. RNPs are advantageous for use in CRISPR base editing systems, particularly for cells that are difficult to transfect, such as primary cells. In addition, RNPs can also alleviate difficulties that may occur with protein expression in cells, especially when eukaryotic promoters, e.g., CMV or EF1A, which may be used in CRISPR plasmids, are not well-expressed. Advantageously, the use of RNPs does not require the delivery of foreign DNA into cells. Moreover, because an RNP comprising a nucleic acid binding protein and gRNA complex is degraded over time, the use of RNPs has the potential to limit off-target effects. In a manner similar to that for plasmid based techniques, RNPs can be used to deliver binding protein (e.g., Cas9 variants) and to direct homology directed repair (HDR).

Nucleic acid molecules encoding a base editor system can be delivered directly to cells (e.g., hepatocytes) as naked DNA or RNA by means of transfection or electroporation, for example, or can be conjugated to molecules (e.g., N-acetylgalactosamine) promoting uptake by the target cells. Vectors encoding base editor systems and/or their components can also be used. In particular embodiments, a polynucleotide, e.g. a mRNA encoding a base editor system or a functional component thereof, may be co-electroporated with one or more guide RNAs as described herein.

Nucleic acid vectors can comprise one or more sequences encoding a domain of a fusion protein described herein. A vector can also encode a protein component of a base editor system operably linked to a nuclear localization signal, nucleolar localization signal, or mitochondrial localization signal. As one example, a vector can include a Cas9 coding sequence that includes one or more nuclear localization sequences (e.g., a nuclear localization sequence from SV40), and one or more deaminases.

The vector can also include any suitable number of regulatory/control elements, e.g., promoters, enhancers, introns, polyadenylation signals, Kozak consensus sequences, or internal ribosome entry sites (IRES). These elements are well known in the art.

Vectors according to this disclosure include recombinant viral vectors. Exemplary viral vectors are set forth herein above. Other viral vectors known in the art can also be used. In addition, viral particles can be used to deliver base editor system components in nucleic acid and/or protein form. For example, “empty” viral particles can be assembled to contain a base editor system or component as cargo. Viral vectors and viral particles can also be engineered to incorporate targeting ligands to alter target tissue specificity.

Vectors described herein may comprise regulatory elements to drive expression of a base editor system or component thereof. Such vectors include adeno-associated viruses with inverted long terminal repeats (AAV ITR). The use of AAV-ITR can be advantageous for eliminating the need for an additional promoter element, which can take up space in the vector. The additional space freed up can be used to drive the expression of additional elements, such as a guide nucleic acid or a selectable marker. ITR activity can be used to reduce potential toxicity due to over expression.

Any suitable promoter can be used to drive expression of a base editor system or component thereof and, where appropriate, the guide nucleic acid. For ubiquitous expression, promoters include CMV, CAG, CBh, PGK, SV40, Ferritin heavy or light chains. For brain or other CNS cell expression, suitable promoters include: SynapsinI for all neurons, CaMKIIalpha for excitatory neurons, GAD67 or GAD65 or VGAT for GABAergic neurons. For liver cell expression, suitable promoters include the Albumin promoter. For lung cell expression, suitable promoters include SP-B. For endothelial cells, suitable promoters include ICAM. For hematopoietic cell expression suitable promoters include IFNbeta or CD45. For osteoblast expression suitable promoters can include OG-2.

In some embodiments, a base editor system of the present disclosure is of small enough size to allow separate promoters to drive expression of the base editor and a compatible guide nucleic acid within the same nucleic acid molecule. For instance, a vector or viral vector can comprise a first promoter operably linked to a nucleic acid encoding the base editor and a second promoter operably linked to the guide nucleic acid.

The promoter used to drive expression of a guide nucleic acid can include: Pol III promoters, such as U6 or H1 Use of Pol II promoter and intronic cassettes to express gRNA Adeno Associated Virus (AAV).

In particular embodiments, a fusion protein of the invention is encoded by a polynucleotide present in a viral vector (e.g., adeno-associated virus (AAV), AAV3, AAV3b, AAV4, AAV5, AAV6, AAV7, AAV8, AAV9, AAVrh8, AAV10, and variants thereof), or a suitable capsid protein of any viral vector. Thus, in some aspects, the disclosure relates to the viral delivery of a fusion protein. Examples of viral vectors include retroviral vectors (e.g. Maloney murine leukemia virus, MML-V), adenoviral vectors (e.g. AD100), lentiviral vectors (HIV and FIV-based vectors), herpesvirus vectors (e.g. HSV-2).

In some aspects, the methods described herein for editing specific genes in a cell can be used to genetically modify the cell. In embodiments, the cell is a hepatocyte.

### Viral Vectors

A base editor described herein can therefore be delivered with viral vectors. In some embodiments, a base editor disclosed herein can be encoded on a nucleic acid that is contained in a viral vector. In some embodiments, one or more components of the base editor system can be encoded on one or more viral vectors. For example, a base editor and guide nucleic acid can be encoded on a single viral vector. In other embodiments, the base editor and guide nucleic acid are encoded on different viral vectors. In either case, the base editor and guide nucleic acid can each be operably linked to a promoter and terminator. The combination of components encoded on a viral vector can be determined by the cargo size constraints of the chosen viral vector.

The use of RNA or DNA viral based systems for the delivery of a base editor takes advantage of highly evolved processes for targeting a virus to specific cells in culture or in the host and trafficking the viral payload to the nucleus or host cell genome. Viral vectors can be administered directly to cells in culture, patients (in vivo), or they can be used to treat cells in vitro, and the modified cells can optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (See, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

Retroviral vectors, especially lentiviral vectors, can require polynucleotide sequences smaller than a given length for efficient integration into a target cell. For example, retroviral vectors of length greater than 9 kb can result in low viral titers compared with those of smaller size. In some aspects, a base editor of the present disclosure is of sufficient size so as to enable efficient packaging and delivery into a target cell via a retroviral vector. In some embodiments, a base editor is of a size so as to allow efficient packing and delivery even when expressed together with a guide nucleic acid and/or other components of a targetable nuclease system.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, Adeno-associated virus (“AAV”) vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

In applications where transient expression is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors can also be used to transduce cells with target nucleic acids, e.g., in the in vitro production of nucleic acids and peptides, and for in vivo and ex vivo gene therapy procedures (See, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

In some embodiments, AAV vectors are used to transduce a cell of interest with a polynucleotide encoding a base editor or base editor system as provided herein. AAV is a small, single-stranded DNA dependent virus belonging to the parvovirus family. The 4.7 kb wild-type (wt) AAV genome is made up of two genes that encode four replication proteins and three capsid proteins, respectively, and is flanked on either side by 145-bp inverted terminal repeats (ITRs). The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). Vp3 is the most abundant subunit in the virion and participates in receptor recognition at the cell surface defining the tropism of the virus. A phospholipase domain, which functions in viral infectivity, has been identified in the unique N terminus of Vp1.

Similar to wt AAV, recombinant AAV (rAAV) utilizes the cis-acting 145-bp ITRs to flank vector transgene cassettes, providing up to 4.5 kb for packaging of foreign DNA. Subsequent to infection, rAAV can express a fusion protein of the invention and persist without integration into the host genome by existing episomally in circular head-to-tail concatemers. Although there are numerous examples of rAAV success using this system, in vitro and in vivo, the limited packaging capacity has limited the use of AAV-mediated gene delivery when the length of the coding sequence of the gene is equal or greater in size than the wt AAV genome.

Viral vectors can be selected based on the application. For example, for in vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it doesn't integrate into the host genome. Adenoviruses are commonly used as vaccines because of the strong immunogenic response they induce. Packaging capacity of the viral vectors can limit the size of the base editor that can be packaged into the vector.

AAV has a packaging capacity of about 4.5 Kb or 4.75 Kb including two 145 base inverted terminal repeats (ITRs). This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor which is shorter in length than conventional base editors. In some examples, the base editors are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, the disclosed base editors are 4.5 kb or less in length.

An AAV can be AAV1, AAV2, AAV5 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

In some embodiments, lentiviral vectors are used to transduce a cell of interest with a polynucleotide encoding a base editor system. Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection can be done in 4 mL OptiMEM with a cationic lipid delivery agent (50 μl Lipofectamine 2000 and 100 μl Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 μm low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

To enhance expression and reduce possible toxicity, the base editor-coding sequence and/or the guide nucleic acid can be modified to include one or more modified nucleoside e.g. using pseudo-U or 5-Methyl-C.

The small packaging capacity of AAV vectors makes the delivery of a number of genes that exceed this size and/or the use of large physiological regulatory elements challenging. These challenges can be addressed, for example, by dividing the protein(s) to be delivered into two or more fragments, wherein the N-terminal fragment is fused to a split intein-N and the C-terminal fragment is fused to a split intein-C. These fragments are then packaged into two or more AAV vectors. As used herein, “intein” refers to a self-splicing protein intron (e.g., peptide) that ligates flanking N-terminal and C-terminal exteins (e.g., fragments to be joined). The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

A fragment of a fusion protein of the invention can vary in length. In some embodiments, a protein fragment ranges from 2 amino acids to about 1000 amino acids in length. In some embodiments, a protein fragment ranges from about 5 amino acids to about 500 amino acids in length. In some embodiments, a protein fragment ranges from about 20 amino acids to about 200 amino acids in length. In some embodiments, a protein fragment ranges from about 10 amino acids to about 100 amino acids in length. Suitable protein fragments of other lengths will be apparent to a person of skill in the art.

In one embodiment, dual AAV vectors are generated by splitting a large transgene expression cassette in two separate halves (5′ and 3′ ends, or head and tail), where each half of the cassette is packaged in a single AAV vector (of <5 kb). The re-assembly of the full-length transgene expression cassette is then achieved upon co-infection of the same cell by both dual AAV vectors followed by: (1) homologous recombination (HR) between 5′ and 3′ genomes (dual AAV overlapping vectors); (2) ITR-mediated tail-to-head concatemerization of 5′ and 3′ genomes (dual AAV trans-splicing vectors); or (3) a combination of these two mechanisms (dual AAV hybrid vectors). The use of dual AAV vectors in vivo results in the expression of full-length proteins. The use of the dual AAV vector platform represents an efficient and viable gene transfer strategy for transgenes of >4.7 kb in size.

### Inteins

Inteins (intervening protein) are auto-processing domains found in a variety of diverse organisms, which carry out a process known as protein splicing. Protein splicing is a multi-step biochemical reaction comprised of both the cleavage and formation of peptide bonds. While the endogenous substrates of protein splicing are proteins found in intein-containing organisms, inteins can also be used to chemically manipulate virtually any polypeptide backbone.

In protein splicing, the intein excises itself out of a precursor polypeptide by cleaving two peptide bonds, thereby ligating the flanking extein (external protein) sequences via the formation of a new peptide bond. This rearrangement occurs post-translationally (or possibly co-translationally). Intein-mediated protein splicing occurs spontaneously, requiring only the folding of the intein domain.

About 5% of inteins are split inteins, which are transcribed and translated as two separate polypeptides, the N-intein and C-intein, each fused to one extein. Upon translation, the intein fragments spontaneously and non-covalently assemble into the canonical intein structure to carry out protein splicing in trans. The mechanism of protein splicing entails a series of acyl-transfer reactions that result in the cleavage of two peptide bonds at the intein-extein junctions and the formation of a new peptide bond between the N- and C-exteins. This process is initiated by activation of the peptide bond joining the N-extein and the N-terminus of the intein. Virtually all inteins have a cysteine or serine at their N-terminus that attacks the carbonyl carbon of the C-terminal N-extein residue. This N to O/S acyl-shift is facilitated by a conserved threonine and histidine (referred to as the TXXH motif (SEQ ID NO:374)), along with a commonly found aspartate, which results in the formation of a linear (thio)ester intermediate. Next, this intermediate is subject to trans-(thio)esterification by nucleophilic attack of the first C-extein residue (+1), which is a cysteine, serine, or threonine. The resulting branched (thio)ester intermediate is resolved through a unique transformation: cyclization of the highly conserved C-terminal asparagine of the intein. This process is facilitated by the histidine (found in a highly conserved HNF motif) and the penultimate histidine and may also involve the aspartate. This succinimide formation reaction excises the intein from the reactive complex and leaves behind the exteins attached through a non-peptidic linkage. This structure rapidly rearranges into a stable peptide bond in an intein-independent fashion.

Non-limiting examples of inteins include any intein or intein-pair known in the art, which include a synthetic intein based on the dnaE intein, the Cfa-N(e.g., split intein-N) and Cfa-C (e.g., split intein-C) intein pair, has been described (e.g., in Stevens et al., J Am Chem Soc. 2016 Feb. 24; 138(7):2162-5, incorporated herein by reference), and DnaE. Non-limitine examples of pairs of inteins that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ ID NOs: 375-382.

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N-[N-terminal portion of the split Cas9]-[intein-N]-C. In some embodiments, an intein-C is fused to the N-terminus of the C-terminal portion of the split Cas9, i.e., to form a structure of N-[intein-C]-[C-terminal portion of the split Cas9]-C. The mechanism of intein-mediated protein splicing for joining the proteins the inteins are fused to (e.g., split Cas9) is known in the art, e.g., as described in Shah et al., Chem Sci. 2014; 5(1):446-461, incorporated herein by reference. Methods for designing and using inteins are known in the art and described, for example by WO2014004336, WO2017132580, US20150344549, and US20180127780, each of which is incorporated herein by reference in their entirety.

In some embodiments, a portion or fragment of a nuclease (e.g., Cas9) is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, an N-terminal fragment of a base editor (e.g., ABE, CBE) is fused to a split intein-N and a C-terminal fragment is fused to a split intein-C. These fragments are then packaged into two or more AAV vectors. In some embodiments, the N-terminus of an intein is fused to the C-terminus of a fusion protein and the C-terminus of the intein is fused to the N-terminus of an AAV capsid protein.

In one embodiment, inteins are utilized to join fragments or portions of a cytidine or adenosine base editor protein that is grafted onto an AAV capsid protein. The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminus of each fragment is fused to an intein-N and the C-terminus of each fragment is fused to an intein C at amino acid positions S303, T310, T313, S355, A456, S460, A463, T466, S469, T472, T474, C574, S577, A589, and S590, which are indicated in capital letters in the sequence below (called the “Cas9 reference sequence”).

| (SEQ ID NO: 201) | |
|---|---|
| 1 | mdkkysigld igtnsvgwav itdeykvpsk kfkvlgntdr hsikknliga llfdsgetae |
| 61 | atrlkrtarr rytrrknric ylqeifsnem akvddsffhr leesflveed kkherhpifg |
| 121 | nivdevayhe kyptiyhlrk klvdstdkad lrliylalah mikfrghfli egdlnpdnsd |
| 181 | vdklfiqlvq tynqlfeenp inasgvdaka ilsarlsksr rlenliaqlp gekknglfgn |
| 241 | lialslgltp nfksnfdlae daklqlskdt ydddldnlla qigdqyadlf laaknlsdai |
| 301 | llSdilrvnT eiTkaplsas mikrydehhq dltllkalvr qqlpekykei ffdqSkngya |
| 361 | gyidggasqe efykfikpil ekmdgteell vklnredllr kqrtfdngsi phqihlgelh |
| 421 | ailrrqedfy pflkdnreki ekiltfripy yvgplArgnS rfAwmTrkSe eTiTpwnfee |
| 481 | vvdkgasaqs fiermtnfdk nlpnekvlpk hsllyeyftv yneltkvkyv tegmrkpafl |
| 541 | sgeqkkaivd llfktnrkvt vkqlkedyfk kieCfdSvei sgvedrfnAS lgtyhdllki |
| 601 | ikdkdfldne enedilediv ltltlfedre mieerlktya hlfddkvmkq lkrrrytgwg |
| 661 | rlsrklingi rdkqsgktil dflksdgfan rnfmqlihdd sltfkediqk aqvsgqgdsl |
| 721 | hehianlags paikkgilqt vkvvdelvkv mgrhkpeniv iemarenqtt qkgqknsrer |
| 781 | mkrieegike lgsqilkehp ventqlqnek lylyylqngr dmyvdgeldi nrlsdydvdh |
| 841 | ivpqsflkdd sidnkvltrs dknrgksdnv pseevvkkmk nywrqllnak litqrkfdnl |
| 901 | tkaergglse ldkagfikrq lvetrqitkh vaqildsrmn tkydendkli revkvitlks |
| 961 | klvsdfrkdf qfykvreinn yhhahdayln avvgtalikk ypklesefvy gdykvydvrk |
| 1021 | miakseqeig katakyffys nimnffktei tlangeirkr plietngetg eivwdkgrdf |
| 1081 | atvrkvlsmp qvnivkktev qtggfskesi lpkrnsdkli arkkdwdpkk yggfdsptva |
| 1141 | ysvlvvakve kgkskklksv kellgitime rssfeknpid fleakgykev kkdliiklpk |
| 1201 | yslfelengr krmlasagel qkgnelalps kyvnflylas hyeklkgspe dneqkqlfve |
| 1261 | qhkhyldeii eqisefskrv iladanldkv lsaynkhrdk pireqaenii hlftltnlga |
| 1321 | paafkyfdtt idrkrytstk evldatlihq sitglyetri dlsqlggd |

### Pharmaceutical Compositions

In some aspects, the present invention provides a pharmaceutical composition comprising any of the genetically modified cells, base editors, fusion proteins, or the fusion protein-guide polynucleotide complexes described herein

The pharmaceutical compositions of the present invention can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g., tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In addition to a modified cell, or population thereof, and a carrier, the pharmaceutical compositions of the present invention can include at least one additional therapeutic agent useful in the treatment of disease. For example, some embodiments of the pharmaceutical composition described herein further comprises a chemotherapeutic agent. In some embodiments, the pharmaceutical composition further comprises a cytokine peptide or a nucleic acid sequence encoding a cytokine peptide. In some embodiments, the pharmaceutical compositions comprising the cell or population thereof can be administered separately from an additional therapeutic agent.

One consideration concerning the therapeutic use of genetically modified cells of the invention is the quantity of cells necessary to achieve an optimal or satisfactory effect. The quantity of cells to be administered may vary for the subject being treated. In one embodiment, between 10⁴ to 10¹⁰, between 10⁵ to 10⁹, or between 10⁶ and 10⁸ genetically modified cells of the invention are administered to a human subject. In some embodiments, at least about 1×10⁸, 2×10⁸, 3×10⁸, 4×10⁸, and 5×10⁸ genetically modified cells of the invention are administered to a human subject. Determining the precise effective dose may be based on factors for each individual subject, including their size, age, sex, weight, and condition. Dosages can be readily ascertained by those skilled in the art from this disclosure and the knowledge in the art.

The skilled artisan can readily determine the number of cells and amount of optional additives, vehicles, and/or carriers in compositions and to be administered in methods of the invention. Typically, additives (in addition to the cell(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining the lethal dose (LD) and LD50 in a suitable animal model (e.g., a rodent such as a mouse); and, the dosage of the composition(s), concentration of components therein, and the timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., a liver). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (see, e.g., Langer, 1990, Science 249: 1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228: 190; During et al., 1989, Ann. Neurol. 25:351; Howard et al., 1989, J. Neurosurg. 71: 105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6: 1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, any of the fusion proteins, gRNAs, and/or complexes described herein are provided as part of a pharmaceutical composition. In some embodiments, the pharmaceutical composition comprises any of the fusion proteins provided herein. In some embodiments, the pharmaceutical composition comprises any of the complexes provided herein. In some embodiments, the pharmaceutical composition comprises a ribonucleoprotein complex comprising an RNA-guided nuclease (e.g., Cas9) that forms a complex with a gRNA and a cationic lipid. In some embodiments pharmaceutical composition comprises a gRNA, a nucleic acid programmable DNA binding protein, a cationic lipid, and a pharmaceutically acceptable excipient. Pharmaceutical compositions can optionally comprise one or more additional therapeutically active substances.

In some embodiments, compositions provided herein are administered to a subject, for example, to a human subject, in order to effect a targeted genomic modification within the subject. In some embodiments, cells are obtained from the subject and contacted with any of the pharmaceutical compositions provided herein. In some embodiments, cells removed from a subject and contacted ex vivo with a pharmaceutical composition are re-introduced into the subject, optionally after the desired genomic modification has been effected or detected in the cells. Methods of delivering pharmaceutical compositions comprising nucleases are known, and are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts, for example, for veterinary use.

Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated in its entirety herein by reference) discloses various excipients used in formulating pharmaceutical compositions and known techniques for the preparation thereof. See also PCT application PCT/US2010/055131 (Publication number WO2011/053982 A8, filed Nov. 2, 2010), incorporated in its entirety herein by reference, for additional suitable methods, reagents, excipients and solvents for producing pharmaceutical compositions comprising a nuclease.

Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present invention provide methods of treating a subject having or having a propensity to develop amyloidosis, the method comprising administering to a subject in need an effective therapeutic amount of a pharmaceutical composition as described herein. In some embodiments, the methods of the invention comprise expressing or introducing into a cell of a subject a base editor polypeptide and one or more guide RNAs capable of targeting a nucleic acid molecule encoding a transthyretin polypeptide comprising a pathogenic mutation.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more. In any of such methods, the methods may comprise administering to the subject an effective amount of an edited cell or a base editor system or polynucleotide encoding such system. In any of such methods, the methods may comprise administering one or more doses of an effective amount of the edited cells per day. In any of such methods, the methods may comprise administering two or more doses of an effective amount of the edited cells per day. In any of such methods, the methods may comprise administering three or more doses of an effective amount of the edited cells per day. In any of such methods, the methods may comprise administering one or more doses of an effective amount of the edited cells per week. In any of such methods, the methods may comprise administering two or more doses of an effective amount of the edited cells per week. In any of such methods, the methods may comprise administering three or more doses of an effective amount of the edited cells per week. In any of such methods, the methods may comprise administering one or more doses of an effective amount of the edited cells per month. In any of such methods, the methods may comprise administering two or more doses of an effective amount of the edited cells per month. In any of such methods, the methods may comprise administering three or more doses of an effective amount of the edited cells per month.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

In some embodiments, a composition described herein (e.g., edited cell, base editor system) is administered in a dosage that is about 0.5-30 mg per kilogram body weight of the human subject. In another embodiment, the amount of the composition administered is about 0.5-20 mg per kilogram body weight of the human subject. In another embodiment, the amount of the composition administered is about 0.5-10 mg per kilogram body weight of the human subject. In another embodiment, the amount of the composition administered is about 0.04 mg, about 0.08 mg, about 0.16 mg, about 0.32 mg, about 0.64 mg, about 1.25 mg, about 1.28 mg, about 1.92 mg, about 2.5 mg, about 3.56 mg, about 3.75 mg, about 5.0 mg, about 7.12 mg, about 7.5 mg, about 10 mg, about 14.24 mg, about 15 mg, about 20 mg, or about 30 mg per kilogram body weight of the human subject. In another embodiment, the amount of the compo composition und administered is about 1.92 mg, about 3.75 mg, about 7.5 mg, about 15.0 mg, or about 30.0 mg per kilogram body weight of the human subject and the composition is administered two times a week. In another embodiment, the amount of the composition administered is about 1.28 mg, about 2.56 mg, about 5.0 mg, about 10 mg, or about 20 mg per kilogram body weight of the human subject and the composition is administered two times a week. In another embodiment, the amount of the composition administered is about 1.92 mg, about 3.75 mg, about 7.5 mg, about 15.0 mg, or about 30.0 mg per kilogram body weight of the human subject and the composition is administered once a week. In another embodiment, the amount of the composition administered is about 1.28 mg, about 2.56 mg, about 5.0 mg, about 10 mg, or about 20 mg per kilogram body weight of the human subject and the composition is administered once a week. In another embodiment, the amount of the composition administered is about 1.92 mg, about 3.75 mg, about 7.5 mg, about 15.0 mg, or about 30.0 mg per kilogram body weight of the human subject and the composition is administered once a day three, five or seven times in a seven day period. In another embodiment, the composition is administered intravenously once a day, seven times in a seven day period. In another embodiment, the amount of the composition administered is about 1.28 mg, about 2.56 mg, about 5.0 mg, about 10 mg, or about 20 mg per kilogram body weight of the human subject and the composition is administered once a day three, five or seven times in a seven day period. In another embodiment, the composition is administered intravenously once a day, seven times in a seven day period.

In some embodiments, the composition is administered over a period of 0.25 h, 0.5 h, 1 h, 2 h, 3 h, 4 h, 5 h, 6 h, 7 h, 8 h, 9 h, 10 h, 11 h, or 12 h. In another embodiment, the composition is administered over a period of 0.25-2 h. In another embodiment, the composition is gradually administered over a period of 1 h. In another embodiment, the composition is gradually administered over a period of 2 h.

The treatments of the present disclosure can result in a reduction in amyloidosis in a subject. The treatments can result in a reduction or elimination of transthyretin (TTR) in cells of the subject (e.g., hepatocytes).

### Kits

The invention provides kits for the treatment of amyloidosis in a subject. In some embodiments, the kit further includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is Cas9 or Cas12. In some embodiments, the polynucleotide encoding the base editor is a mRNA sequence. In some embodiments, the deaminase is a cytidine deaminase or an adenosine deaminase. In some embodiments, the kit comprises an edited cell and instructions regarding the use of such cell.

The kits may further comprise written instructions for using the base editor system and/or edited cell. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1. Transthyretin Gene Alterations

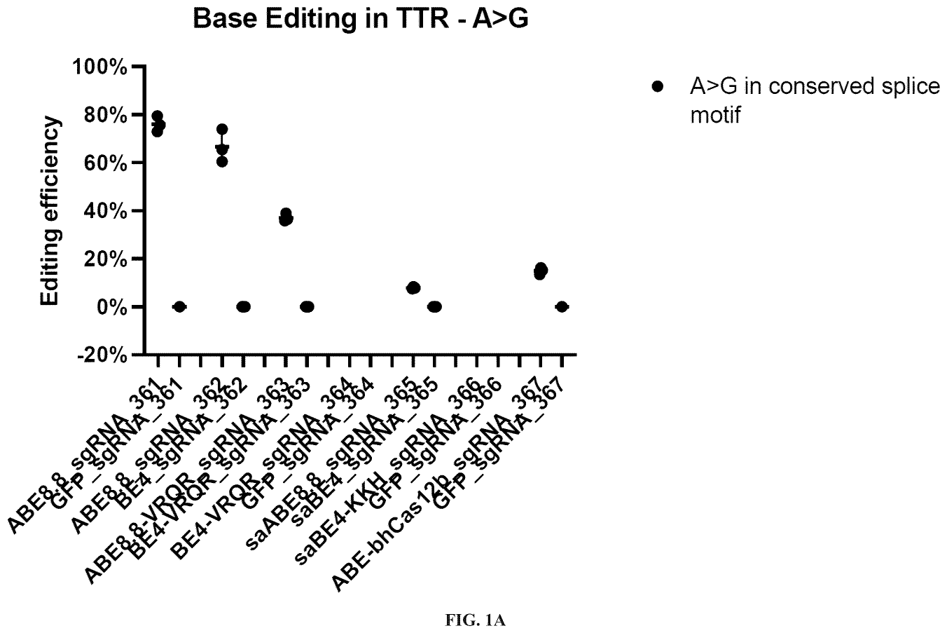

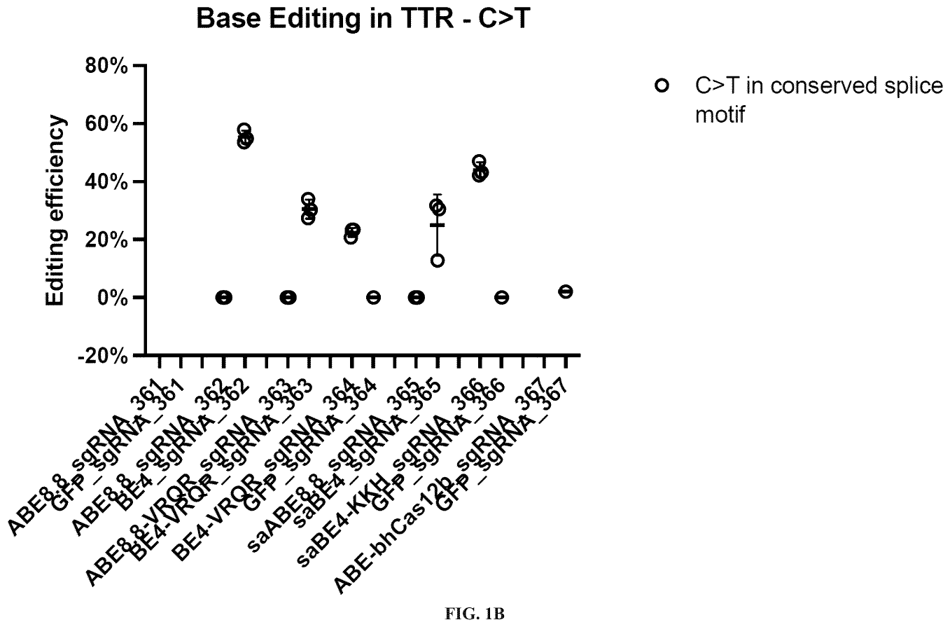

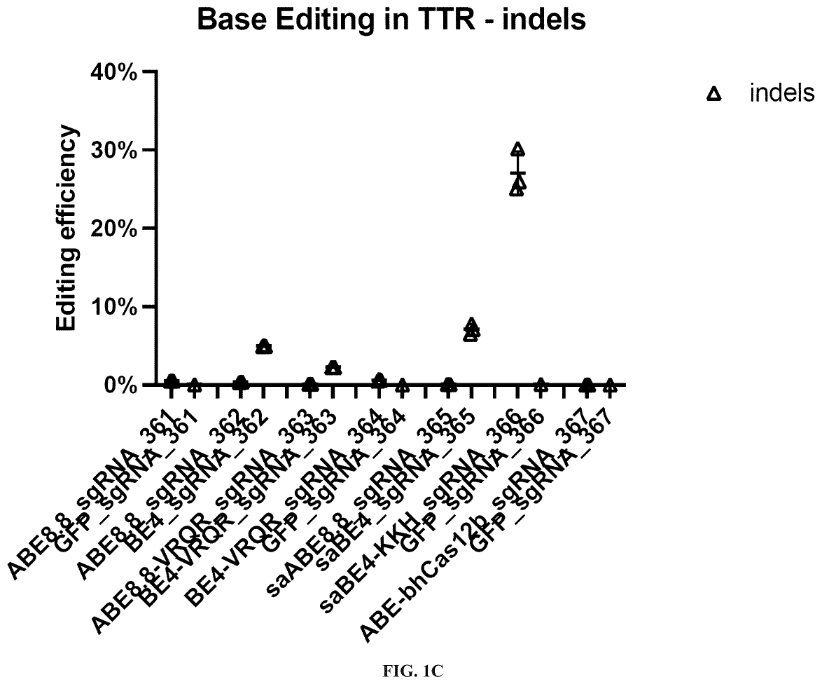

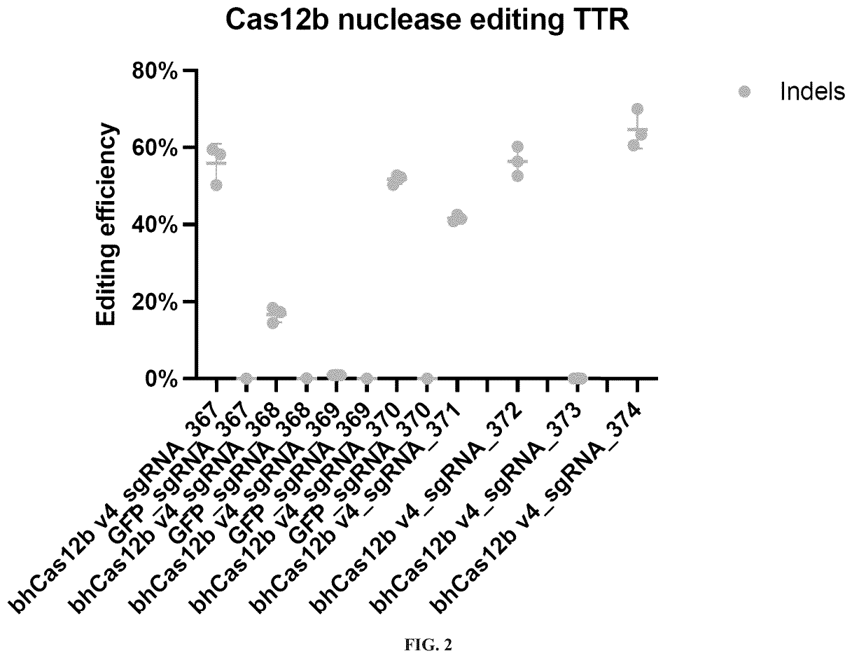

The guide RNAs listed in Table 1 were screened for use in editing the transthyretin (TTR) gene by disrupting splice sites (FIG. 1A-1C) or using a bhCas12b nuclease strategy (FIG. 2). 15 total guide RNAs were screened. The screen was performed by Lo-I in HEK293T cells using based editors and bhCas12b delivered as mRNA and the sgRNAs. The guide RNAs sgRNA_361 and sgRNA_362 worked well in splice site disruption (FIGS. 1A-1C) using ABE and/or BE4. Several of the gRNAs functioned well as bhCas12b nuclease gRNAs.

Sequences for the base editors indicated in FIGS. 1A-1C and the bhCas12b endonuclease are listed below in Table 20.

TABLE 20
Base editor and nuclease sequences.

| | | SEQ | |
|---|---|---|---|
| | | ID | |
| Name | Description | NO | Sequence |
| ABE8.8 | pMRNA-trilink- | 442 | MSEVEFSHEYWMRHALTLAKRARDEREVPVGAVLVLNNRVIGEGWNRAIGLHDPTA |
| | monoTadA- | | HAEIMALRQGGLVMQNYRLIDATLYVTFEPCVMCAGAMIHSRIGRVVFGVRNAKTG |
| | ABE8.8(TadA7.10 + | | AAGSLMDVLHHPGMNHRVEITEGILADECAALLCRFFRMPRRVFNAQKKAQSSTDSG |
| | H123H + Y147R + | | GSSGGSSGSETPGTSESATPESSGGSSGGSDKKYSIGLAIGTNSVGWAVITDEYKVPSK |
| | Q154R) 120A bbsI | | KFKVLGNTDRHSIKKNLIGALLFDSGETAEATRLKRTARRRYTRRKNRICYLQEIFSNE |
| | | | MAKVDDSFFHRLEESFLVEEDKKHERHPIFGNIVDEVAYHEKYPTIYHLRKKLVDSTD |
| | | | KADLRLIYLALAHMIKFRGHFLIEGDLNPDNSDVDKLFIQLVQTYNQLFEENPINASGV |
| | | | DAKAILSARLSKSRRLENLIAQLPGEKKNGLFGNLIALSLGLTPNFKSNFDLAEDAKLQ |
| | | | LSKDTYDDDLDNLLAQIGDQYADLFLAAKNLSDAILLSDILRVNTEITKAPLSASMIKR |
| | | | YDEHHQDLTLLKALVRQQLPEKYKEIFFDQSKNGYAGYIDGGASQEEFYKFIKPILEK |
| | | | MDGTEELLVKLNREDLLRKQRTFDNGSIPHQIHLGELHAILRRQEDFYPFLKDN [symbol] EKIE |
| | | | KILTFRIPYYVGPLARGNSRFAWMTRKSEETITPWNFEEVVDKGASAQSFIERM [symbol] FD |
| | | | KNLPNEKVLPKHSLLYEYFTVYNELTKVKYVTEGMRKPAFLSGEQKKAIVDLLFKTN |
| | | | RKVTVKQLKEDYFKKIECFDSVEISGVEDRFNASLGTYHDLLKIIKDKDFLDNE [symbol] EDI |
| | | | LEDIVLTLTLFEDREMIEERLKTYAHLFDDKVMKQLKRRRYTGWGRLSRKLIN [symbol] DK |
| | | | QSGKTILDFLKSDGFANRNFMQLIHDDSLTFKEDIQKAQVSGQGDSLHEHIANL [symbol] SP |
| | | | AIKKGILQTVKVVDELVKVMGRHKPENIVIEMARENQTTQKGQKNSRERMKR [symbol] GI |
| | | | KELGSQILKEHPVENTQLQNEKLYLYYLQNGRDMYVDQELDINRLSDYDVDHIV [symbol] QS |
| | | | FLKDDSIDNKVLTRSDKNRGKSDNVPSEEVVKKMKNYWRQLLNAKLITQRKF [symbol] LT |
| | | | KAERGGLSELDKAGFIKRQLVETRQITKHVAQILDSRMNTKYDENDKLIREVKVITLK |
| | | | SKLVSDFRKDFQFYKVREINNYHHAHDAYLNAVVGTALIKKYPKLESEFVYGDYKV |
| | | | YDVRKMIAKSEQEIGKATAKYFFYSNIMNFFKTEITLANGEIRKRPLIETNGETGEIVW |
| | | | DKGRDFATVRKVLSMPQVNIVKKTEVQTGGFSKESILPKRNSDKLIARKKDWDPKKY |
| | | | GGFDSPTVAYSVLVVAKVEKGKSKKLKSVKELLGITIMERSSFEKNPIDFLEAKGYKE |
| | | | VKKDLIIKLPKYSLFELENGRKRMLASAGELQKGNELALPSKYVNFLYLASHYEKLK |
| | | | GSPEDNEQKQLFVEQHKHYLDEIIEQISEFSKRVILADANLDKVLSAYNKHRDKPIREQ |
| | | | AENIIHLFTLTNLGAPAAFKYFDTTIDRKRYTSTKEVLDATLIHQSITGLYETRIDLSQL |
| | | | GGDEGADKRTADGSEFESPKKKRKV |
| BE4 | pMRNA-BE4 120A | 443 | MSSETGPVAVDPTLRRRIEPHEFEVFFDPRELRKETCLLYEINWGGRHSIWRHTSQNTN |
| | BbsI | | KHVEVNFIEKFTTERYFCPNTRCSITWFLSWSPCGECSRAITEFLSRYPHVTLFIYIARL |
| | | | YHHADPRNRQGLRDLISSGVTIQIMTEQESGYCWRNFVNYSPSNEAHWPRYPHLWVR |
| | | | LYVLELYCIILGLPPCLNILRRKQPQLTFFTIALQSCHYQRLPPHILWATGLKSGGSSGG |
| | | | SSGSETPGTSESATPESSGGSSGGSDKKYSIGLAIGTNSVGWAVITDEYKVPSKK [symbol] KVL |
| | | | GNTDRHSIKKNLIGALLFDSGETAEATRLKRTARRRYTRRKNRICYLQEIFSNE [symbol] KV |
| | | | DDSFFHRLEESFLVEEDKKHERHPIFGNIVDEVAYHEKYPTIYHLRKKLVDSTD [symbol] ADL |
| | | | RLIYLALAHMIKFRGHFLIEGDLNPDNSDVDKLFIQLVQTYNQLFEENPINASGVDAK |
| | | | AILSARLSKSRRLENLIAQLPGEKKNGLFGNLIALSLGLTPNFKSNFDLAEDAKL [symbol] SK |
| | | | DTYDDDLDNLLAQIGDQYADLFLAAKNLSDAILLSDILRVNTEITKAPLSASMI [symbol] YD |
| | | | EHHQDLTLLKALVRQQLPEKYKEIFFDQSKNGYAGYIDGGASQEEFYKFIKPIL [symbol] D |
| | | | GTEELLVKLNREDLLRKQRTFDNGSIPHQIHLGELHAILRRQEDFYPFLKDNRE [symbol] KIL |
| | | | TFRIPYYVGPLARGNSRFAWMTRKSEETITPWNFEEVVDKGASAQSFIERMTN [symbol] NL |
| | | | PNEKVLPKHSLLYEYFTVYNELTKVKYVTEGMRKPAFLSGEQKKAIVDLLFKTNRKV |
| | | | TVKQLKEDYFKKIECFDSVEISGVEDRFNASLGTYHDLLKIIKDKDFLDNEENEDILEDI |
| | | | VLTLTLFEDREMIEERLKTYAHLFDDKVMKQLKRRRYTGWGRLSRKLINGIRDKQSG |
| | | | KTILDFLKSDGFANRNFMQLIHDDSLTFKEDIQKAQVSGQGDSLHEHIANLAGSPAIKK |
| | | | GILQTVKVVDELVKVMGRHKPENIVIEMARENQTTQKGQKNSRERMKRIEEGIKELG |
| | | | SQILKEHPVENTQLQNEKLYLYYLQNGRDMYVDQELDINRLSDYDVDHIVPQSFLKD |
| | | | DSIDNKVLTRSDKNRGKSDNVPSEEVVKKMKNYWRQLLNAKLITQRKFDNLTKAER |
| | | | GGLSELDKAGFIKRQLVETRQITKHVAQILDSRMNTKYDENDKLIREVKVITLKSKLV |
| | | | SDFRKDFQFYKVREINNYHHAHDAYLNAVVGTALIKKYPKLESEFVYGDYKVYDVR |
| | | | KMIAKSEQEIGKATAKYFFYSNIMNFFKTEITLANGEIRKRPLIETNGETGEIVWDKGR |
| | | | DFATVRKVLSMPQVNIVKKTEVQTGGFSKESILPKRNSDKLIARKKDWDPKKYGGFD |
| | | | SPTVAYSVLVVAKVEKGKSKKLKSVKELLGITIMERSSFEKNPIDFLEAKGYKEVKKD |
| | | | LIIKLPKYSLFELENGRKRMLASAGELQKGNELALPSKYVNFLYLASHYEKLKGSPED |
| | | | NEQKQLFVEQHKHYLDEIIEQISEFSKRVILADANLDKVLSAYNKHRDKPIREQ [symbol] ENII |
| | | | HLFTLTNLGAPAAFKYFDTTIDRKRYTSTKEVLDATLIHQSITGLYETRIDLSQL [symbol] DS |
| | | | GGSGGSGGSTNLSDIIEKETGKQLVIQESILMLPEEVEEVIGNKPESDILVHTAY [symbol] TD |
| | | | ENVMLLTSDAPEYKPWALVIQDSNGENKIKMLSGGSGGSGGSTNLSDIIEKETG [symbol] LV |
| | | | IQESILMLPEEVEEVIGNKPESDILVHTAYDESTDENVMLLTSDAPEYKPWALV [symbol] SN |
| | | | GENKIKMLSGGSKRTADGSEFESPKKKRKVE |
| ABE8.8- | pMRNA-trilink- | 444 | MSEVEFSHEYWMRHALTLAKRARDEREVPVGAVLVLNNRVIGEGWNRAIGL [symbol] TA |
| VRQR | monoTadA- | | SHAEIMALRQGGLVMQNYRLIDATLYVTFEPCVMCAGAMIHSRIGRVVFGVRN [symbol] G |
| | ABE8.8(TadA7.10 + | | AAGSLMDVLHHPGMNHRVEITEGILADECAALLCRFFRMPRRVFNAQKKAQS [symbol] SG |
| | H123H + Y147R + | | GSSGGSSGSETPGTSESATPESSGGSSGGSDKKYSIGLAIGTNSVGWAVITDEYKVPSK |
| | Q154R)- | | KFKVLGNTDRHSIKKNLIGALLFDSGETAEATRLKRTARRRYTRRKNRICYLQEIFSNE |
| | VRQR 120A bbsI | | MAKVDDSFFHRLEESFLVEEDKKHERHPIFGNIVDEVAYHEKYPTIYHLRKKLVDSTD |
| | | | KADLRLIYLALAHMIKFRGHFLIEGDLNPDNSDVDKLFIQLVQTYNQLFEENPINASGV |
| | | | DAKAILSARLSKSRRLENLIAQLPGEKKNGLFGNLIALSLGLTPNFKSNFDLAEDAKLQ |
| | | | LSKDTYDDDLDNLLAQIGDQYADLFLAAKNLSDAILLSDILRVNTEITKAPLSASMIKR |
| | | | YDEHHQDLTLLKALVRQQLPEKYKEIFFDQSKNGYAGYIDGGASQEEFYKFIKPILEK |
| | | | MDGTEELLVKLNREDLLRKQRTFDNGSIPHQIHLGELHAILRRQEDFYPFLKDNREKIE |
| | | | KILTFRIPYYVGPLARGNSRFAWMTRKSEETITPWNFEEVVDKGASAQSFIERMTNFD |
| | | | KNLPNEKVLPKHSLLYEYFTVYNELTKVKYVTEGMRKPAFLSGEQKKAIVDLLFKTN |
| | | | RKVTVKQLKEDYFKKIECFDSVEISGVEDRFNASLGTYHDLLKIIKDKDFLDNEENEDI |
| | | | LEDIVLTLTLFEDREMIEERLKTYAHLFDDKVMKQLKRRRYTGWGRLSRKLINGIRDK |
| | | | QSGKTILDFLKSDGFANRNFMQLIHDDSLTFKEDIQKAQVSGQGDSLHEHIANLAGSP |
| | | | AIKKGILQTVKVVDELVKVMGRHKPENIVIEMARENQTTQKGQKNSRERMKR [symbol] EGI |
| | | | KELGSQILKEHPVENTQLQNEKLYLYYLQNGRDMYVDQELDINRLSDYDVDH [symbol] QS |
| | | | FLKDDSIDNKVLTRSDKNRGKSDNVPSEEVVKKMKNYWRQLLNAKLITQRKF [symbol] LT |
| | | | KAERGGLSELDKAGFIKRQLVETRQITKHVAQILDSRMNTKYDENDKLIREVK [symbol] LK |
| | | | SKLVSDFRKDFQFYKVREINNYHHAHDAYLNAVVGTALIKKYPKLESEFVYG [symbol] V |
| | | | YDVRKMIAKSEQEIGKATAKYFFYSNIMNFFKTEITLANGEIRKRPLIETNGETG [symbol] W |
| | | | DKGRDFATVRKVLSMPQVNIVKKTEVQTGGFSKESILPKRNSDKLIARKKDWD [symbol] KY |
| | | | GGFVSPTVAYSVLVVAKVEKGKSKKLKSVKELLGITIMERSSFEKNPIDFLEAK [symbol] KE |
| | | | VKKDLIIKLPKYSLFELENGRKRMLASARELQKGNELALPSKYVNFLYLASHY [symbol] KG |
| | | | SPEDNEQKQLFVEQHKHYLDEIIEQISEFSKRVILADANLDKVLSAYNKHRDKPIREQA |
| | | | ENIIHILFTLTNLGAPAAFKYFDTTIDRKQYRSTKEVLDATLIHQSITGLYETRIDLSQLG |
| | | | GDEGADKRTADGSEFESPKKKRKV |
| BE4- | pMRNA-BE4-VRQR | 445 | MSSETGPVAVDPTLRRRIEPHEFEVFFDPRELRKETCLLYEINWGGRHSIWRHTSQNTN |
| VRQR | 120A BsaI | | KHVEVNFIEKFTTERYFCPNTRCSITWFLSWSPCGECSRAITEFLSRYPHVTLFIYIARL |
| | | | YHHADPRNRQGLRDLISSGVTIQIMTEQESGYCWRNFVNYSPSNEAHWPRYPHLWVR |
| | | | LYVLELYCIILGLPPCLNILRRKQPQLTFFTIALQSCHYQRLPPHILWATGLKSGGSSGG |
| | | | SSGSETPGTSESATPESSGGSSGGSDKKYSIGLAIGTNSVGWAVITDEYKVPSKKFKVL |
| | | | GNTDRHSIKKNLIGALLFDSGETAEATRLKRTARRRYTRRKNRICYLQEIFSNEMAKV |
| | | | DDSFFHRLEESFLVEEDKKHERHPIFGNIVDEVAYHEKYPTIYHLRKKLVDSTDKADL |
| | | | RLIYLALAHMIKFRGHFLIEGDLNPDNSDVDKLFIQLVQTYNQLFEENPINASGVDAK |
| | | | AILSARLSKSRRLENLIAQLPGEKKNGLFGNLIALSLGLTPNFKSNFDLAEDAKLQLSK |
| | | | DTYDDDLDNLLAQIGDQYADLFLAAKNLSDAILLSDILRVNTEITKAPLSASMIKRYD |
| | | | EHHQDLTLLKALVRQQLPEKYKEIFFDQSKNGYAGYIDGGASQEEFYKFIKPIL [symbol] KMD |
| | | | GTEELLVKLNREDLLRKQRTFDNGSIPHQIHLGELHAILRRQEDFYPFLKDNRE [symbol] KIL |
| | | | TFRIPYYVGPLARGNSRFAWMTRKSEETITPWNFEEVVDKGASAQSFIERMTN [symbol] NL |
| | | | PNEKVLPKHSLLYEYFTVYNELTKVKYVTEGMRKPAFLSGEQKKAIVDLLFKT [symbol] KV |
| | | | TVKQLKEDYFKKIECFDSVEISGVEDRFNASLGTYHDLLKIIKDKDFLDNEENE [symbol] EDI |
| | | | VLTLTLFEDREMIEERLKTYAHLFDDKVMKQLKRRRYTGWGRLSRKLINGIRD [symbol] SG |
| | | | KTILDFLKSDGFANRNFMQLIHDDSLTFKEDIQKAQVSGQGDSLHEHIANLAGS [symbol] KK |
| | | | GILQTVKVVDELVKVMGRHKPENIVIEMARENQTTQKGQKNSRERMKRIEEGI [symbol] G |
| | | | SQILKEHPVENTQLQNEKLYLYYLQNGRDMYVDQELDINRLSDYDVDHIVPQS [symbol] D |
| | | | DSIDNKVLTRSDKNRGKSDNVPSEEVVKKMKNYWRQLLNAKLITQRKFDNLTKAER |
| | | | GGLSELDKAGFIKRQLVETRQITKHVAQILDSRMNTKYDENDKLIREVKVITLKSKLV |
| | | | SDFRKDFQFYKVREINNYHHAHDAYLNAVVGTALIKKYPKLESEFVYGDYKVYDVR |
| | | | KMIAKSEQEIGKATAKYFFYSNIMNFFKTEITLANGEIRKRPLIETNGETGEIVWDKGR |
| | | | DFATVRKVLSMPQVNIVKKTEVQTGGFSKESILPKRNSDKLIARKKDWDPKKYGGFV |
| | | | SPTVAYSVLVVAKVEKGKSKKLKSVKELLGITIMERSSFEKNPIDFLEAKGYKEVKKD |
| | | | LIIKLPKYSLFELENGRKRMLASARELQKGNELALPSKYVNFLYLASHYEKLKGSPED |
| | | | NEQKQLFVEQHKHYLDEIIEQISEFSKRVILADANLDKVLSAYNKHRDKPIREQAENII |
| | | | HLFTLTNLGAPAAFKYFDTTIDRKQYRSTKEVLDATLIHQSITGLYETRIDLSQLGGDS |
| | | | GGSGGSGGSTNLSDIIEKETGKQLVIQESILMLPEEVEEVIGNKPESDILVHTAYDESTD |
| | | | ENVMLLTSDAPEYKPWALVIQDSNGENKIKMLSGGSGGSGGSTNLSDIIEKETGKQLV |
| | | | IQESILMLPEEVEEVIGNKPESDILVHTAYDESTDENVMLLTSDAPEYKPWALVIQDSN |
| | | | GENKIKMLSGGSKRTADGSEFESPKKKRKVE |
| saABE8.8 | pMRNA-trilink- | 446 | MSEVEFSHEYWMRHALTLAKRARDEREVPVGAVLVLNNRVIGEGWNRAIGL [symbol] PTA |
| | monoTadA-saABE8.8 | | HAEIMALRQGGLVMQNYRLIDATLYVTFEPCVMCAGAMIHSRIGRVVFGVRN [symbol] TG |
| | (TadA7.10 + H123H + | | AAGSLMDVLHHPGMNHRVEITEGILADECAALLCRFFRMPRRVFNAQKKAQS [symbol] SG |
| | Y147R + Q154R) 120A | | GSSGGSSGSETPGTSESATPESSGGSSGGSKRNYILGLAIGITSVGYGIIDYETRD [symbol] AG |
| | bbsI | | VRLFKEANVENNEGRRSKRGARRLKRRRRHRIQRVKKLLFDYNLLTDHSELS [symbol] YE |
| | | | ARVKGLSQKLSEEEFSAALLHLAKRRGVHNVNEVEEDTGNELSTKEQISRNSK [symbol] EK |
| | | | YVAELQLERLKKDGEVRGSINRFKTSDYVKEAKQLLKVQKAYHOLDQSFIDT [symbol] L |
| | | | ETRRTYYEGPGEGSPFGWKDIKEWYEMLMGHCTYFPEELRSVKYAYNADLY [symbol] ND |
| | | | LNNLVITRDENEKLEYYEKFQIIENVFKQKKKPTLKQIAKEILVNEEDIKGYRVT [symbol] GK |
| | | | PEFTNLKVYHDIKDITARKEIIENAELLDQIAKILTIYQSSEDIQEELTNLNSELTQEEIEQ |
| | | | ISNLKGYTGTHNLSLKAINLILDELWHTNDNQIAIFNRLKLVPKKVDLSQQKEIPTTLV |
| | | | DDFILSPVVKRSFIQSIKVINAIIKKYGLPNDIIIELAREKNSKDAQKMINEMQKRNRQT |
| | | | NERIEEIIRTTGKENAKYLIEKIKLHDMQEGKCLYSLEAIPLEDLLNNPFNYEVDHIIPRS |
| | | | VSFDNSFNNKVLVKQEENSKKGNRTPFQYLSSSDSKISYETFKKHILNLAKGKGRISKT |
| | | | KKEYLLEERDINRFSVQKDFINRNLVDTRYATRGLMNLLRSYFRVNNLDVKVKSING |
| | | | GFTSFLRRKWKFKKERNKGYKHHAEDALIIANADFIFKEWKKLDKAKKVMENQMFE |
| | | | EKQAESMPEIETEQEYKEIFITPHQIKHIKDFKDYKYSHRVDKKPNRELINDTLYSTRK |
| | | | DDKGNTLIVNNLNGLYDKDNDKLKKLINKSPEKLLMYHHDPQTYQKLKLIMEQYGD |
| | | | EKNPLYKYYEETGNYLTKYSKKDNGPVIKKIKYYGNKLNAHLDITDDYPNSRNKVVK |
| | | | LSLKPYRFDVYLDNGVYKFVTVKNLDVIKKENYYEVNSKCYEEAKKLKKISNQAEFI |
| | | | ASFYNNDLIKINGELYRVIGVNNDLLNRIEVNMIDITYREYLENMNDKRPPRIIKTIASK |
| | | | TQSIKKYSTDILGNLYEVKSKKHPQIIKKGEGADKRTADGSEFESPKKKRKV |
| saBE4 | pMRNA-saBE4 120A | 447 | MSSETGPVAVDPTLRRRIEPHEFEVFFDPRELRKETCLLYEINWGGRHSIWRHT [symbol] QNTN |
| | BbsI | | KHVEVNFIEKFTTERYFCPNTRCSITWFLSWSPCGECSRAITEFLSRYPHVTLFIY [symbol] L |
| | | | YHHADPRNRQGLRDLISSGVTIQIMTEQESGYCWRNFVNYSPSNEAHWPRYPH [symbol] VR |
| | | | LYVLELYCIILGLPPCLNILRRKQPQLTFFTIALQSCHYQRLPPHILWATGLKSG [symbol] GG |
| | | | SSGSETPGTSESATPESSGGSSGGSGKRNYILGLAIGITSVGYGIIDYETRDVIDA [symbol] LF |
| | | | KEANVENNEGRRSKRGARRLKRRRRHRIQRVKKLLFDYNLLTDHSELSGINPY [symbol] RV |
| | | | KGLSQKLSEEEFSAALLHLAKRRGVHNVNEVEEDTGNELSTKEQISRNSKALE [symbol] V |
| | | | AELQLERLKKDGEVRGSINRFKTSDYVKEAKQLLKVQKAYHQLDQSFIDTYID [symbol] TR |
| | | | RTYYEGPGEGSPFGWKDIKEWYEMLMGHCTYFPEELRSVKYAYNADLYNAL [symbol] N |
| | | | NLVITRDENEKLEYYEKFQIIENVFKQKKKPTLKQIAKEILVNEEDIKGYRVTSTGKPEF |
| | | | TNLKVYHDIKDITARKEIIENAELLDQIAKILTIYQSSEDIQEELTNLNSELTQEEIEQISN |
| | | | LKGYTGTHNLSLKAINLILDELWHTNDNQIAIFNRLKLVPKKVDLSQQKEIPTTLVDDF |
| | | | ILSPVVKRSFIQSIKVINAIIKKYGLPNDIIIELAREKNSKDAQKMINEMQKRNRQTNERI |
| | | | EEIIRTTGKENAKYLIEKIKLHDMQEGKCLYSLEAIPLEDLLNNPFNYEVDHIIPRSVSF |
| | | | DNSFNNKVLVKQEENSKKGNRTPFQYLSSSDSKISYETFKKHILNLAKGKGRISKTKK |
| | | | EYLLEERDINRFSVQKDFINRNLVDTRYATRGLMNLLRSYFRVNNLDVKVKSINGGFT |
| | | | SFLRRKWKFKKERNKGYKHHAEDALIIANADFIFKEWKKLDKAKKVMENQMFEEKQ |
| | | | AESMPEIETEQEYKEIFITPHQIKHIKDFKDYKYSHRVDKKPNRELINDTLYSTRKDDK |
| | | | GNTLIVNNLNGLYDKDNDKLKKLINKSPEKLLMYHHDPQTYQKLKLIMEQYGDEKN |
| | | | PLYKYYEETGNYLTKYSKKDNGPVIKKIKYYGNKLNAHLDITDDYPNSRNKVVKLSL |
| | | | KPYRFDVYLDNGVYKFVTVKNLDVIKKENYYEVNSKCYEEAKKLKKISNQAEFIASF |
| | | | YNNDLIKINGELYRVIGVNNDLLNRIEVNMIDITYREYLENMNDKRPPRIIKTIASKTQS |
| | | | IKKYSTDILGNLYEVKSKKHPQIIKKGGSPKKKRKVSSDYKDHDGDYKDHDID [symbol] KDD |
| | | | DDKSGGSGGSGGSTNLSDIIEKETGKQLVIQESILMLPEEVEEVIGNKPESDILV [symbol] YD |
| | | | ESTDENVMLLTSDAPEYKPWALVIQDSNGENKIKMLSGGSGGSGGSTNLSDIIE [symbol] TG |
| | | | KQLVIQESILMLPEEVEEVIGNKPESDILVHTAYDESTDENVMLLTSDAPEYKP [symbol] VI |
| | | | QDSNGENKIKMLSGGSKRTADGSEFESPKKKRKVE |
| saBE4- | pMRNA-saBE4-KKH | 448 | MSSETGPVAVDPTLRRRIEPHEFEVFFDPRELRKETCLLYEINWGGRHSIWRHT [symbol] TN |
| KKH | 120A BbsI | | KHVEVNFIEKFTTERYFCPNTRCSITWFLSWSPCGECSRAITEFLSRYPHVTLFIY [symbol] L |
| | | | YHHADPRNRQGLRDLISSGVTIQIMTEQESGYCWRNFVNYSPSNEAHWPRYPH [symbol] VR |
| | | | LYVLELYCIILGLPPCLNILRRKQPQLTFFTIALQSCHYQRLPPHILWATGLKSGG [symbol] GG |
| | | | SSGSETPGTSESATPESSGGSSGGSGKRNYILGLAIGITSVGYGIIDYETRDVIDAGVRLF |
| | | | KEANVENNEGRRSKRGARRLKRRRRHRIQRVKKLLFDYNLLTDHSELSGINPYEARV |
| | | | KGLSQKLSEEEFSAALLHLAKRRGVHNVNEVEEDTGNELSTKEQISRNSKALEEKYV |
| | | | AELQLERLKKDGEVRGSINRFKTSDYVKEAKQLLKVQKAYHQLDQSFIDTYIDLLETR |
| | | | RTYYEGPGEGSPFGWKDIKEWYEMLMGHCTYFPEELRSVKYAYNADLYNALNDLN |
| | | | NLVITRDENEKLEYYEKFQIIENVFKQKKKPTLKQIAKEILVNEEDIKGYRVTSTGKPEF |
| | | | TNLKVYHDIKDITARKEIIENAELLDQIAKILTIYQSSEDIQEELTNLNSELTQEEIEQISN |
| | | | LKGYTGTHNLSLKAINLILDELWHTNDNQIAIFNRLKLVPKKVDLSQQKEIPTTLVDDF |
| | | | ILSPVVKRSFIQSIKVINAIIKKYGLPNDIIIELAREKNSKDAQKMINEMQKRNRQTNERI |
| | | | EEIIRTTGKENAKYLIEKIKLHDMQEGKCLYSLEAIPLEDLLNNPFNYEVDHIIPRSVSF |
| | | | DNSFNNKVLVKQEENSKKGNRTPFQYLSSSDSKISYETFKKHILNLAKGKGRISKTKK |
| | | | EYLLEERDINRFSVQKDFINRNLVDTRYATRGLMNLLRSYFRVNNLDVKVKSINGGFT |
| | | | SFLRRKWKFKKERNKGYKHHAEDALIIANADFIFKEWKKLDKAKKVMENQMFEEKQ |
| | | | AESMPEIETEQEYKEIFITPHQIKHIKDFKDYKYSHRVDKKPNRKLINDTLYSTR [symbol] DDK |
| | | | GNTLIVNNLNGLYDKDNDKLKKLINKSPEKLLMYHHDPQTYQKLKLIMEQYG [symbol] KN |
| | | | PLYKYYEETGNYLTKYSKKDNGPVIKKIKYYGNKLNAHLDITDDYPNSRNKV [symbol] SL |
| | | | KPYRFDVYLDNGVYKFVTVKNLDVIKKENYYEVNSKCYEEAKKLKKISNQAE [symbol] SF |
| | | | YKNDLIKINGELYRVIGVNNDLLNRIEVNMIDITYREYLENMNDKRPPHIIKTIA [symbol] QS |
| | | | IKKYSTDILGNLYEVKSKKHPQIIKKGGSPKKKRKVSSDYKDHDGDYKDHDID [symbol] D |
| | | | DDKSGGSGGSGGSTNLSDIIEKETGKQLVIQESILMLPEEVEEVIGNKPESDILVH [symbol] YD |
| | | | ESTDENVMLLTSDAPEYKPWALVIQDSNGENKIKMLSGGSGGSGGSTNLSDIIE [symbol] G |
| | | | KQLVIQESILMLPEEVEEVIGNKPESDILVHTAYDESTDENVMLLTSDAPEYKPWALVI |
| | | | QDSNGENKIKMLSGGSKRTADGSEFESPKKKRKVE |
| ABE- | pBZ517 | 449 | MSEVEFSHEYWMRHALTLAKRARDEREVPVGAVLVLNNRVIGEGWNRAIGLHDPTA |
| bhCas12b | pCV021_ABE8.13m- | | HAEIMALRQGGLVMQNYRLYDATLYVTFEPCVMCAGAMIHSRIGRVVFGVRNAKTG |
| | dBhCas12b_(D952A,_ | | AAGSLMDVLHHPGMNHRVEITEGILADECAALLCRFFRMPRRVFNAQKKAQSSTDGS |
| | S893R,_K846R,_E837G) | | SGSETPGTSESATPESSGAPKKKRKVGIHGVPAAATRSFILKIEPNEEVKKGLWKTHEV |
| | | | LNHGIAYYMNILKLIRQEAIYEHHEQDPKNPKKVSKAEIQAELWDFVLKMQKCNSFT |
| | | | HEVDKDEVFNILRELYEELVPSSVEKKGEANQLSNKFLYPLVDPNSQSGKGTASSGRK |
| | | | PRWYNIKIAGDPSWEEEKKKWEEDKKKDPLAKILGKLAEYGLIPLFIPYTDSNEPIVK |
| | | | EIKWMEKSRNQSVRRLDKDMFIQALERFLSWESWNLKVKEEYEKVEKEYKTLEERIK |
| | | | EDIQALKALEQYEKERQEQLLRDTLNTNEYRLSKRGLRGWREIIQKWLKMDENEPSE |
| | | | KYLEVFKDYQRKHPREAGDYSVYEFLSKKENHFIWRNHPEYPYLYATFCEIDKKKKD |
| | | | AKQQATFTLADPINHPLWVRFEERSGSNLNKYRILTEQLHTEKLKKKLTVQLDRLIYP |
| | | | TESGGWEEKGKVDIVLLPSRQFYNQIFLDIEEKGKHAFTYKDESIKFPLKGTLG [symbol] ARV |
| | | | QFDRDHLRRYPHKVESGNVGRIYFNMTVNIEPTESPVSKSLKIHRDDFPKVVNF [symbol] KE |
| | | | LTEWIKDSKGKKLKSGIESLEIGLRVMSIALGQRQAAAASIFEVVDQKPDIEGK [symbol] PIK |
| | | | GTELYAVHRASFNIKLPGETLVKSREVLRKAREDNLKLMNQKLNFLRNVLHF [symbol] ED |
| | | | ITEREKRVTKWISRQENSDVPLVYQDELIQIRELMYKPYKDWVAFLKQLHKRL [symbol] IG |
| | | | KEVKHWRKSLSDGRKGLYGISLKNIDEIDRTRKFLLRWSLRPTEPGEVRRLEPG [symbol] FAI |
| | | | DQLNHLNALKEDRLKKMANTIIMHALGYCYDVRKKKWQAKNPACQIILFEDL [symbol] YN |
| | | | PYKERSRFENSRLMKWSRREIPRQVALQGEIYGLQVGEVGAQFSSRFHAKTGS [symbol] RC |
| | | | RVVTKEKLQDNRFFKNLQREGRLTLDKIAVLKEGDLYPDKGGEKFISLSKDRK [symbol] T |
| | | | HADINAAQNLQKRFWTRTHGFYKVYCKAYQVDGQTVYIPESKDQKQKIIEEFGEGYF |
| | | | ILKDGVYEWVNAGKLKIKKGSSKQSSSELVDSDILKDSFDLASELKGEKLMLYRDPSG |
| | | | NVFPSDKWMAAGVFFGKLERILISKLTNQYSISTIEDDSSKQSMKRPAATKKAGQAKK |
| | | | KK |
| bhCas12b | Cas12b v4 | 450 | MAPKKKRKVGIHGVPAAATRSFILKIEPNEEVKKGLWKTHEVLNHGIAYYMNILKLIR |
| v4 | | | QEAIYEHHEQDPKNPKKVSKAEIQAELWDFVLKMQKCNSFTHEVDKDEVENILRELY |
| | | | EELVPSSVEKKGEANQLSNKFLYPLVDPNSQSGKGTASSGRKPRWYNIKIAGDPSWE |
| | | | EEKKKWEEDKKKDPLAKILGKLAEYGLIPLFIPYTDSNEPIVKEIKWMEKSRNQSVRR |
| | | | LDKDMFIQALERFLSWESWNLKVKEEYEKVEKEYKTLEERIKEDIQALKALEQYEKE |
| | | | RQEQLLRDTLNTNEYRLSKRGLRGWREIIQKWLKMDENEPSEKYLEVFKDYQRKHPR |
| | | | EAGDYSVYEFLSKKENHFIWRNHPEYPYLYATFCEIDKKKKDAKQQATFTLADPINHP |
| | | | LWVRFEERSGSNLNKYRILTEQLHTEKLKKKLTVQLDRLIYPTESGGWEEKGKVDIVL |
| | | | LPSRQFYNQIFLDIEEKGKHAFTYKDESIKFPLKGTLGGARVQFDRDHLRRYPHKVES |
| | | | GNVGRIYFNMTVNIEPTESPVSKSLKIHRDDFPKVVNFKPKELTEWIKDSKGKK [symbol] KSGI |
| | | | ESLEIGLRVMSIDLGQRQAAAASIFEVVDQKPDIEGKLFFPIKGTELYAVHRASF [symbol] LP |
| | | | GETLVKSREVLRKAREDNLKLMNQKLNFLRNVLHFQQFEDITEREKRVTKWIS [symbol] EN |
| | | | SDVPLVYQDELIQIRELMYKPYKDWVAFLKQLHKRLEVEIGKEVKHWRKSLS [symbol] K |
| | | | GLYGISLKNIDEIDRTRKFLLRWSLRPTEPGEVRRLEPGQRFAIDQLNHLNALK [symbol] LK |
| | | | KMANTIIMHALGYCYDVRKKKWQAKNPACQIILFEDLSNYNPYGERSRFENSR [symbol] K |
| | | | WSRREIPRQVALQGEIYGLQVGEVGAQFSSRFHAKTGSPGIRCRVVTKEKLQD [symbol] FK |
| | | | NLQREGRLTLDKIAVLKEGDLYPDKGGEKFISLSKDRKCVTTHADINAAQNLQ [symbol] W |
| | | | TRTHGFYKVYCKAYQVDGQTVYIPESKDQKQKIIEEFGEGYFILKDGVYEWVN [symbol] KL |
| | | | KIKKGSSKQSSSELVDSDILKDSFDLASELKGEKLMLYRDPSGNVFPSDKWMAAGVFF |
| | | | GKLERILISKLTNQYSISTIEDDSSKQSMSGGSKRTADGSEFESPKKKRKVE |

[symbol]

indicates data missing or illegible when filed

### Example 2. Confirmation of Loss of Transthyretin (TTR) Expression in Hepatocytes

The guide RNAs identified in Example 1 as working well in splice site disruption using ABE and/or BE4, or as working well with bhCas12b are used to edit transthyretin (TTR) in hepatocytes to result in loss or reduction in TTR expression. Standard methods for culturing hepatocytes are used (see, e.g., Shulman and Nahmias, “Long-term and coculture of primary rate and human hepatocytes”, Methods Mol. Biol., 945:287-302 (2013); and Castell J., Gómez-Lechón M. (2009) Liver Cell Culture Techniques. In: Dhawan A., Hughes R. (eds) Hepatocyte Transplantation. Methods in Molecular Biology, vol 481. Humana Press, Totowa, NJ. https://doi.org/10.1007/978-1-59745-201-4_4). For gene editing, the base editors and bhCas12b are delivered to the cells as mRNA in combination with sgRNA using lipid nanoparticles. Following gene editing, transthyretin (TTR) expression in the cells is confirmed as reduced or eliminated. Reduction or elimination of expression is confirmed using standard techniques in molecular biology (e.g., Real-Time Quantitative Reverse Transcription PCR).

### Example 3. Direct Correction of the Transthyretin (TTR) V122I Mutation

The mutation V122I in the mature transthyretin (TTR) polypeptide is the African American population founder mutation. The mutation is a major cause of cardiovascular mortality (i.e., cardiac amyloidosis) for the African American population. About 3.9% of African Americans have the V122I mutation. The V122I mutation can be edited using ABE. Thus, ABE is used to directly correct the V122I mutation in cells.

ABE mRNA and sgRNA are delivered to a cell (e.g., a hepatocyte or a HEK293T cell) encoding a transthyretin (TTR) polypeptide having the V122I mutation. ABE mRNA encoding the base editors indicated in Table 21 below are administered in combination with sgRNAs comprising the indicated spacer sequences. The transthyretin (TTR) gene in the cell is successfully edited to no longer encode the pathogenic V122I mutation and to encode a non-pathogenic version of transthyretin (e.g., transthyretin with a valine at position 122).

TABLE 21
Base editor and nuclease sequences.
One of skill in the art will understand that
some of the target site sequences correspond
to a reverse-complement to the above-provided
transthyretin polynucleotide sequence;
i.e., the target sequences may correspond to
either strand of a dsDNA molecule encoding
a transthyretin polynucleotide.

| Base | | Spacer | | Target | Target |
|---|---|---|---|---|---|
| Editor | sgRNA | Sequence | PAM | Sequence | Base |
| spCas9- | sgRNA_ | GGCUAUCGUC | AGG | GGCTATCGTC | 5A |
| ABE | 375 | ACCAAUCCCA | | ACCAATCCCA | |
| | | (SEQ ID | | (SEQ ID | |
| | | NO: 375) | | NO: 439) | |
| spCas9- | sgRNA_ | GCUAUCGUCA | GGA | GCTATCGTCA | 4A |
| VRQR- | 376 | CCAAUCCCAA | | CCAATCCCAA | |
| ABE | | (SEQ ID | | (SEQ ID | |
| | | NO: 376) | | NO: 440) | |
| saCas9- | sgRNA_ | GGCUAUCGUC | AGG | GGCTATCGTC | 5A |
| ABE | 377 | ACCAAUCCCA | AAT | ACCAATCCCA | |
| | | (SEQ ID | | (SEQ ID | |
| | | NO: 377) | | NO: 441) | |

In embodiments, the altered amino acid is in a splice site or start codon as illustrated in the following sequences. Alterations in splice site disrupt expression of the encoded TTR polypeptide. A description of the respective target for each of the following sequences is indicated in parentheses:
4A of the nucleotide sequence TATAGGAAAACCAGTGAGTC (SEQ ID NO: 425); (splice sites)
6A of the nucleotide sequence TACTCACCTCTGCATGCTCA (SEQ ID NO: 426); (splice sites)
5A of the nucleotide sequence ACTCACCTCTGCATGCTCAT (SEQ ID NO: 427); (splice sites)
7A of the nucleotide sequence ATACTCACCTCTGCATGCTCA (SEQ ID NO: 429); (splice sites)
6A of the nucleotide sequence TTGGCAGGATGGCTTCTCATCG (SEQ ID NO: 431); (splice sites)
9A of the sequence TTGGCAGGATGGCTTCTCATCG (SEQ ID NO: 431); (start codon)
5A of the sequence GGCTATCGTCACCAATCCCA (SEQ ID NO: 439) (correction of pathogenic mutation); or
4A of the sequence GCTATCGTCACCAATCCCAA (SEQ ID NO: 440) (correction of pathogenic mutation).
7C of the nucleotide sequence TACTCACCTCTGCATGCTCA (SEQ ID NO: 426); (splice sites)
6C of the nucleotide sequence ACTCACCTCTGCATGCTCAT (SEQ ID NO: 427); (splice sites)
7C of the nucleotide sequence TACCACCTATGAGAGAAGAC (SEQ ID NO: 428); (splice sites)
8C of the nucleotide sequence ATACTCACCTCTGCATGCTCA (SEQ ID NO: 429); or (splice sites)
11C of the nucleotide sequence ACTGGTTTTCCTATAAGGTGT (SEQ ID NO: 430). (splice sites)

### Example 4. Transthyretin (TTR) Guide Screening and Functional Knockdown Assessment in Primary Hepatocytes

Experiments were undertaken to determine the efficacy of the base editor systems developed in the above Examples in editing human or primate primary hepatocytes. As described above, fifteen guide RNAs were designed to knockdown transthyretin (TTR) protein expression in HEK293T cells. These guides used either a base editing strategy for splice site disruption or a nuclease-based bhCas12b strategy. A base editing strategy was initially prioritized. Base editing guides were used with either an ABE (adenosine base editor) or CBE (cytidine base editor) for splice site disruption, and a subset of guides was suitable for use with both an ABE and a CBE. Six guide editor combinations exhibited good editing efficiency in HEK293T cells (FIG. 1): ABE8.8_sgRNA_361; ABE8.8_sgRNA_362; BE4_sgRNA_362; ABE8.8-VRQR_sgRNA_363; BE4-VRQR_sgRNA_363; and BE4-KKH_sgRNA_366. Experiments were undertaken to evaluate these four guides (sgRNAs 361, 362, 363, 366; sequences are listed in Table 1) in primary hepatocytes (both human and Macaca fascicularis) to assess editing efficiency in primary cells and the capacity for functional knockdown of TTR protein expression.

### Screening Hek293T-Validated TTR Knockdown Guides in PXIB-Cell Primary Human Hepatocytes

Editor mRNA_sgRNA combinations (i.e. base editor systems) were transfected in triplicate in human hepatocytes extracted from humanized mouse livers (PXB-cells, PhoenixBio) following a 3-day cell incubation. In addition to the 6 guide-editor pairs of interest (ABE8.8_sgRNA_361; ABE8.8_sgRNA_362; BE4_sgRNA_362; ABE8.8-VRQR_sgRNA_363; BE4-VRQR_sgRNA_363; and BE4-KKH_sgRNA_366), two positive control guide-editor pairs were also transfected. These positive controls included ABE8.8_sgRNA_088, which conained the spacer sequence CAGGAUCCGCACAGACUCCA (SEQ ID NO: 581) and is known to be effective at editing sites outside of the TTR gene, and Cas9_gRNA991 (Gillmore, J. D. et al. “CRISPR-Cas9 In Vivo Gene Editing for Transthyretin Amyloidosis,” New Engl J Med 385, 493-502 (2021)), which contained the spacer sequence AAAGGCUGCUGAUGACACCU (SEQ ID NO: 565) corresponding to the target sequence AAAGGCTGCTGATGACACCT (SEQ ID NO: 580). The guide gRNA991 is known to be effective for use in inducing functional TTR knockdown in hepatocytes. An untreated condition was also included as a negative control. To assess functional TTR knockdown, cell supernatants were collected and stored at −80° C. Collections were performed prior to transfection (3-day incubation), as well as 4-, 7-, 10-, and 13-days post-transfection. An additional media change was performed 1 day post-transfection, but the supernatant was discarded. Genomic DNA was harvested from cells 13 days post-transfection and editing efficiency was assessed by Next Generation Sequencing (NGS). A human TTR ELISA assay was used to assess TTR protein concentration in cell supernatants pre-transfection, as well as 7-days and 13-days post-transfection.

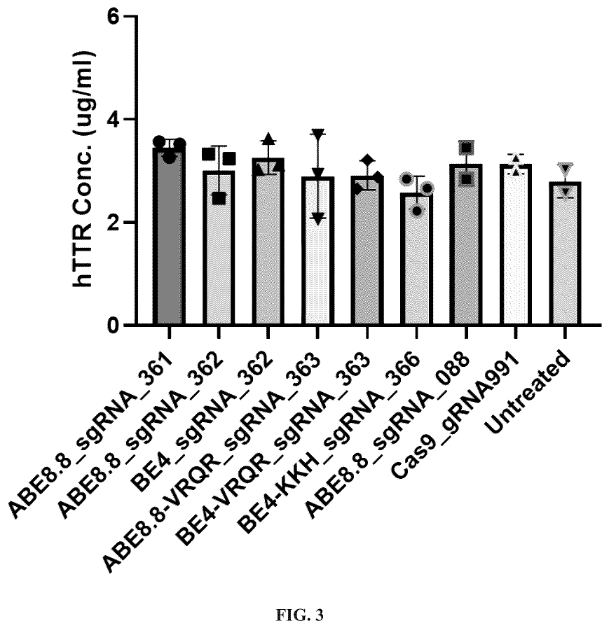

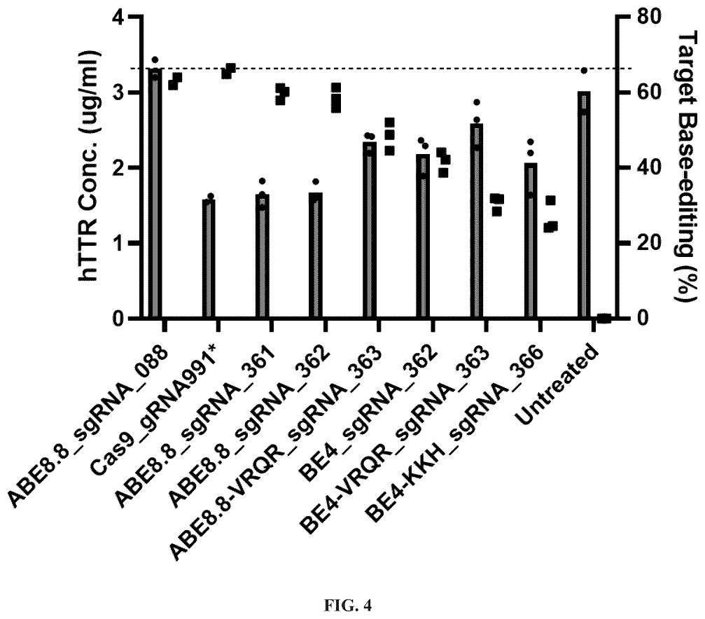

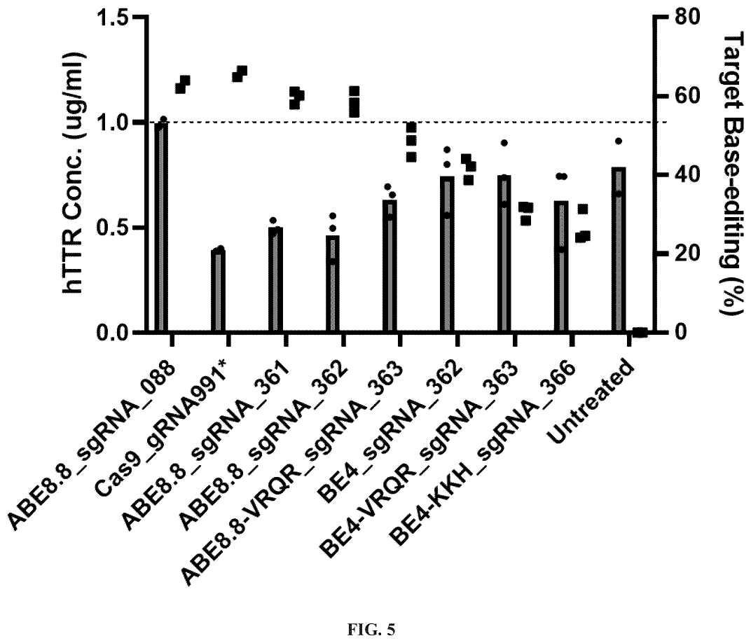

Pre-transfection, no significant difference in TTR concentration was observed between samples (FIG. 3). By 7-days post-transfection, a roughly 50% reduction in TTR levels was observed for ABE8.8_sgRNA_361 and ABE8.8_sgRNA_362 as compared to the control ABE8.8_sgRNA_088, which did not edit within the TTR gene (FIG. 4). This reduction was comparable to the positive control Cas9_gRNA991 (FIG. 4). Similar trends were observed 13-days post-transfection (FIG. 5). Editing efficiencies for ABE8.8_sgRNA_361 and ABE8.8_sgRNA_362 were both high, at approximately 60% (FIGS. 2 and 5). This was comparable to the controls ABE8.8_sgRNA_088 and Cas9_gRNA991 (FIGS. 4 and 5). TTR protein knockdown was positively correlated with editing rates across samples (FIGS. 4 and 5).

Assessing Editingperformance and Functional Knockdown Generation for ABE8.8 sgRNAA361 and ABE8.8 sgRNA362 in Primary Cyno Hepatocytes

ABE8.8_sgRNA_361 and ABE8.8_sgRNA_362, both of which exhibited high target base-editing and functional TTR protein knockdown in PXB-cells, were transfected in triplicate in primary cyno (Macaca fascicularis) hepatocyte co-cultures. ABE8.8_sgRNA_088 was transfected as a positive control, and an untreated condition was included as a negative control, both in triplicate. To assess functional TTR knockdown, cell supernatants were collected and stored at −80° C. Collections were performed prior to transfection (3-day incubation), as well as 4-, 7-, 10-, and 13-days post-transfection. An additional media change was performed 1 day post-transfection, but the supernatant was discarded. Genomic DNA was harvested from cells 13 days post-transfection and editing efficiency was assessed by Next Generation Sequencing (NGS). A modified TTR ELISA assay was used to assess cyno TTR protein concentration in cell supernatants pre-transfection, as well as 7-days and 13-days post-transfection.

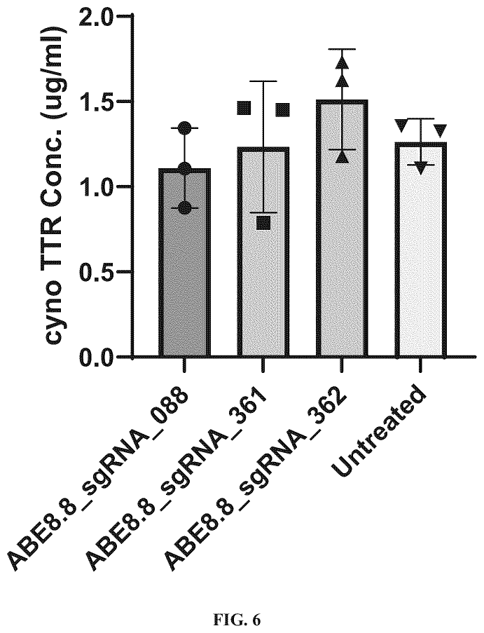

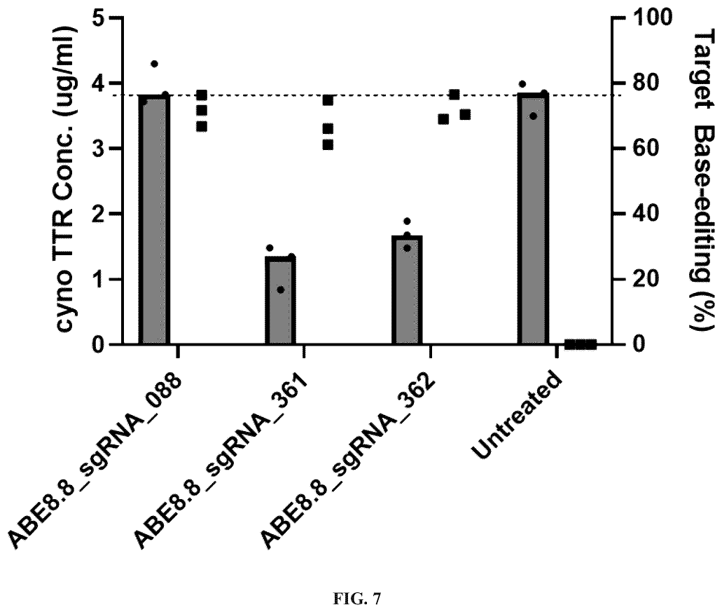

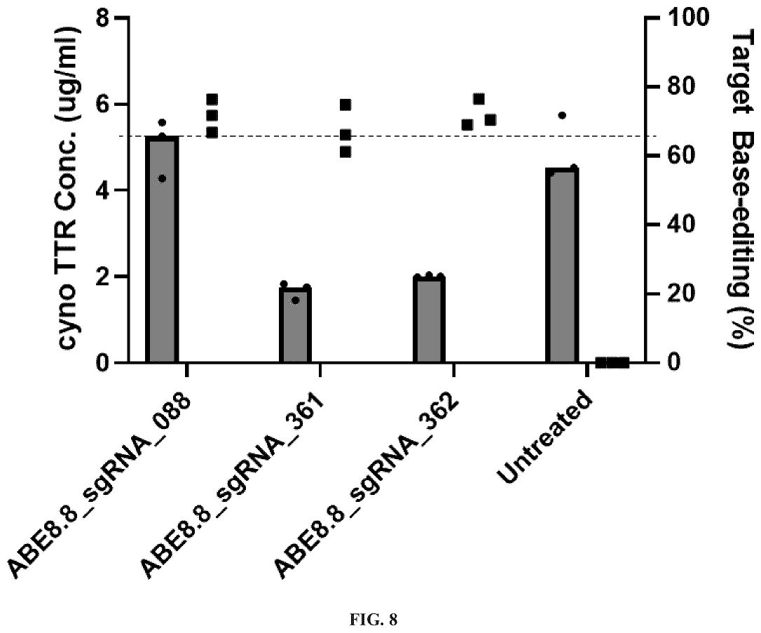

Pre-transfection, no significant difference in cyno TTR concentration was observed between samples (FIG. 6). By 7-days post-transfection, roughly₆₀-7₀% reductions in cyno TTR levels were observed for ABE8.8_sgRNA_361 and ABE8.8_sgRNA_362 as compared to ABE8.8_sgRNA_088, which did not edit within the TTR gene (FIG. 7). Similar trends were observed 13-days post-transfection (FIG. 8). Editing efficiencies for ABE8.8_sgRNA_361 and ABE8.8_sgRNA_362 were both high, at approximately 70% (FIGS. 7 and 8). This was comparable to the ABE8.8_sgRNA_088 positive control (FIGS. 7 and 8).

The following materials and methods were employed in this Example.

### PXIB-Cell Maintenance

One 24-well plate of PXB-cell hepatocytes was ordered from PhoenixBio. After receipt of cells, media was changed twice with pre-warmed dHCGM media (PhoenixBio)+10% Fetal Bovine Serum (Thermo Fisher, A3160401). Cells were then incubated according to the manufacturer's instructions, changing the media every 3 days. An extra media change was performed the day following transfection, after which a 3-day media change schedule was resumed. For all media changes other than the two initial changes and the day following transfection (pre-transfection and 4, 7, 10, and 13 days post-transfection), media was collected, distributed across multiple 96-well plates, and stored at −80° C.

### Primary Cyno Hepatocyte (PCH) Co-Culture Generation and Maintenance

A frozen vial of primary cyno hepatocytes (IVAL, A75245, Lot #10286011) was thawed and mixed with 50 mL pre-warmed CHRM medium (Invitrogen, CM7000). Tube was centrifuged at 100×g for 10 minutes at room temperature. CHRM media was discarded and cell pellet was resuspended in 4 mL INVITROGRO CP Medium (Bio IVT, Z990003)+2.2% Torpedo Antibiotic Mix (Bio IVT, Z99000). Cells were counted using a Neubauer Improved hemocytometer (SKC, Inc., DHCN015) and 350,000 cells/well were plated in a 24-well BioCoat Rat Collagen I plate (Corning, 354408). There was a sufficient number of cells for 18 wells. Co-cultures were generated 5 hours after plating through the addition of 20,000 3T3-J2 cells (Stem Cell Technologies, 100-0353) in fresh CP+Torpedo media to each well. Following a media change the next day, cells were incubated according to the manufacturer's instructions, changing CP+Torpedo media every 3 days. An extra media change was performed the day following transfection, after which a 3-day media change schedule was resumed.

### Cell Transection

PXB-cells were transfected 3 days following their receipt. Prior to transfection, a media change was performed for all wells. Spent media was aliquoted across multiple 96 well plates and stored at −80° C. For each condition, 200 ng sgRNA (Agilent and Synthego) and 600 ng editor mRNA (produced at Beam) were diluted to 25 μl with OPTIMEM (Thermo Fisher, 31985062) in a 96-well plate. Separately, the transfection reagent lipofectamine MessengerMAX Reagent (Thermo Fisher, LMRNA015) at 1.5× the total volume of RNA was diluted in the reduced-serum medium OPTIMEM to 25 μl for each condition, mixed thoroughly, and incubated at room temperature for 10 minutes. MessengerMAX solutions were then combined with the corresponding sgRNA+editor solution and thoroughly mixed. Following a 5-minute incubation at room temperature, the lipid encapsulated mRNA+sgRNA mixes were added dropwise onto the PXB-cells. Media was changed and spent media was discarded <16 hours following transfection.

PCH samples were transfected 4 days following the addition of 3T3-J2 feeder cells. Prior to transfection, a media change was performed for all wells. The same transfection protocol as that used for PXB-cells was used for PCH.

### Next Generation DNA Sequencing (NGS)

Following media collection, genomic DNA was isolated from each PXB-cell well 13-days post-transfection according to the following protocol. 200 μl of QuickExtract DNA Extraction Solution (Lucigen, QE09050) was added to each well. Cells were incubated for 5 minutes at 37° C., after which the cells were manually dislodged from the bottom of each well by pipetting. The cells were incubated again for 5 minutes at 37° C., after which the buffer-cell mixture was thoroughly mixed, and 150 μl was transferred to a 96-well plate. The 96-well plate was incubated at 65° C. for 15 mins and then at 98° C. for 10 mins.

PCR was performed using Phusion U Green Multiplex PCR Master Mix (Fisher Scientific, F564L) and region-specific primers. A second round of PCR was then performed on the first round PCR products to add barcoded Illumina adaptor sequences to each sample. Second round PCR products were purified using SPRIselect beads (Thermo Fisher Scientific, B23317) at a 1:1 bead to PCR ratio. The combined library concentration was quantified using a Qubit 1× dsDNA HS Assay Kit (Thermo Fisher Scientific, Q33231), and the library was sequenced using a Miseq Reagent Micro Kit v2 (300-cycles) (Illumina, MS-103-1002). Reads were aligned to appropriate reference sequences and editing efficiency was assessed at the appropriate sites.

Genomic DNA isolation, NGS, and analysis were performed as above for PCH. The library was sequenced using a Miseq Reagent Nano Kit v2 (300-cycles) (Illumina, MS-103-1001).

### TTR Protein Quantification

A human prealbumin (TTR) ELISA kit (Abcam, ab231920) was used to measure TTR protein levels in PXB-cell supernatants at various timepoints pre- and post-transfection. PXB-cell supernatants were thawed at room temperature and centrifuged at 2000×g for 10 minutes at 4° C. Supernatants were then diluted 1:1000 in provided Sample Diluent NS buffer prior to loading on the ELISA plate. The ELISA assay was then performed according to manufacturer's instructions. Samples were allowed to develop for 18 minutes in Development solution prior to addition of Stop solution. Absorbance was read at 450 nm using an Infinite M Plex plate reader (Tecan).

For the detection of cyno (Macacafascicularis) TTR protein in primary cyno hepatocyte co-culture supernatants, known concentrations of purified cyno TTR protein (Abcam, ab239566) were used to assess cross reactivity of the human TTR ELISA kit (Abcam, ab231920). Through this approach, it was determined that the kit was approximately 4% cross-reactive with cyno TTR protein. Purified cyno TTR protein was then used to generate a new set of standards (20 ng-0.3125 ng for standards 1-7) capable of accurately measuring cyno TTR protein levels. The assay was otherwise performed identically to manufacturer's instructions. Supernatants were diluted 1:1000 and were developed for 17 minutes in Development solution prior to addition of Stop solution.

### Example 5. Transthyretin (TTR) Promoter Screening for Gene Expression Knockdown

Experiments were undertaken to develop base editor systems suitable for knocking out expression of the TTR gene in humans through introducing alterations to the promoter region of the gene.

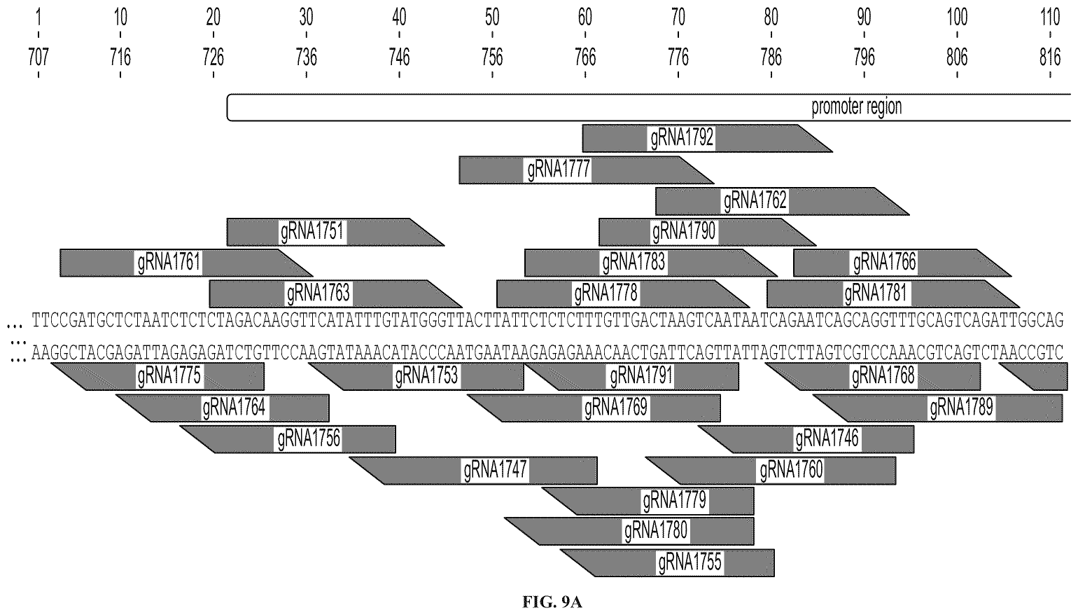

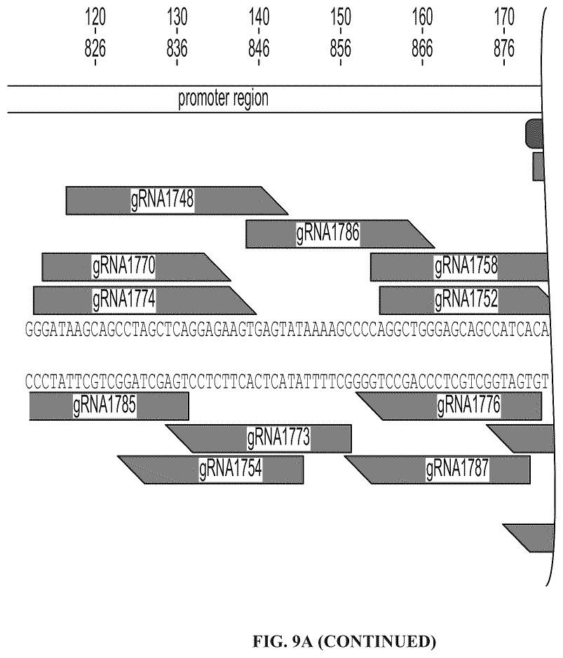

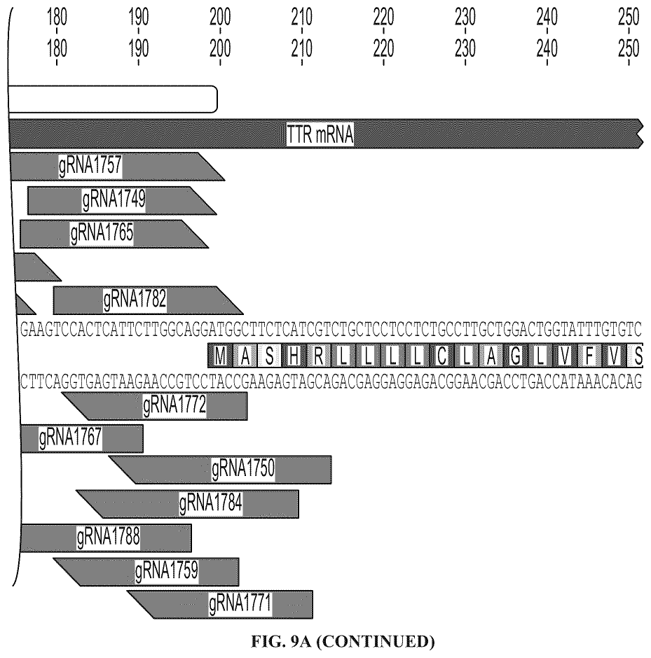

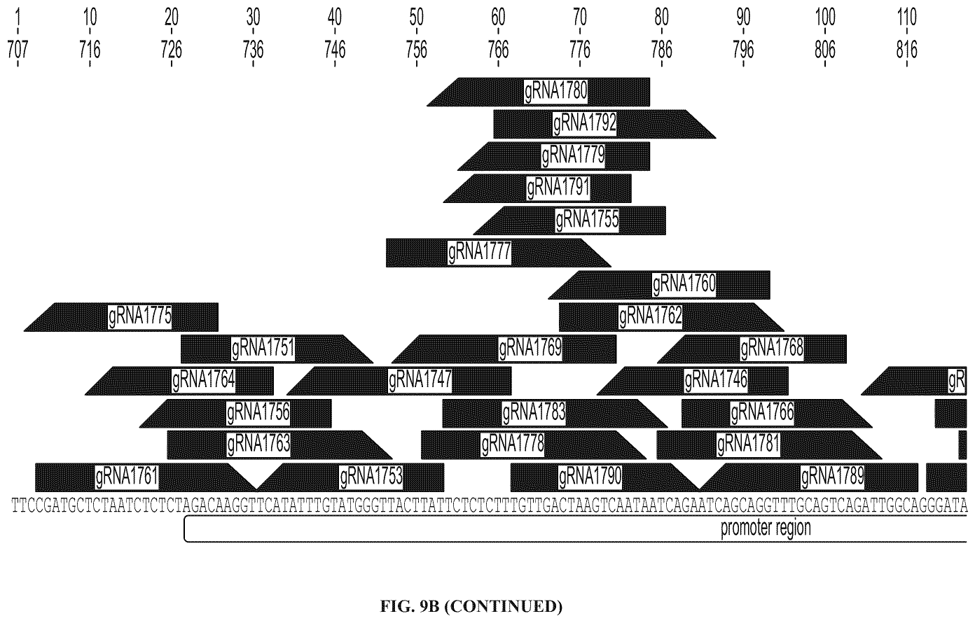

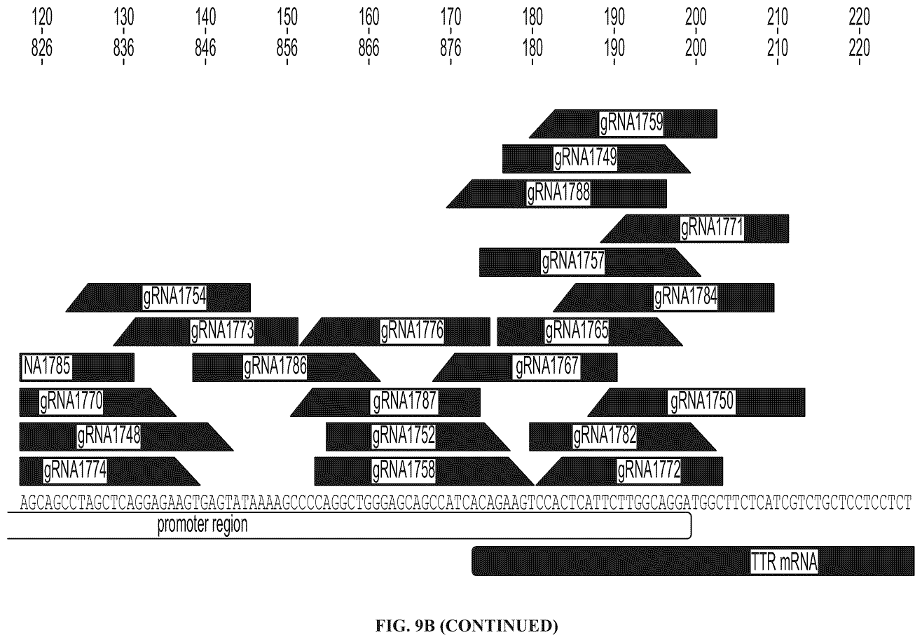

Sequence homology between the murine (see, Costa, R. H. & Grayson, D. R. Site-directed mutagenesis of hepatocyte nuclear factor (HNF) binding sites in the mouse transthyretin (TTR) promoter reveal synergistic interactions with its enhancer region. Nucleic Acids Res 19, 4139-4145 (1991), the disclosure of which is incorporated herein by reference in its entirety for all purposes; GGCAAGGTTCATATTTGTGTAGGTTACTTATTCTCCTTTTGTTGACTAAGTCAATAATCAGAAT CAGCAGG (SEQ ID NO: 582)) and human TTR promoter regions was used to define the human promoter region to guide the design of guide RNA sequences for use in knocking out TTR in humans (FIGS. 9A and 9B).

gRNAs corresponding to four CRISPR-Cas enzymes with 3′ PAMs NGG, NGA, NNGRRT and NNNRRT were designed to tile the reported promoter region (FIGS. 9A and 9B). A base editing strategy was designed to generate mutations within the promoter region that would knock down TTR mRNA expression. 3′ NGG PAM gRNAs were designed to be paired with an S. pyogenes CRISPR-Cas9-containing base editor. 3′ NGA PAM gRNAs were designed to be paired with a mutated S. pyogenes CRISPR-Cas9-containing base editor. 3′ NNGRRT PAM gRNAs were designed to be paired with an S. aureus CRISPR-Cas9-containing base editor. 3′ NNNRRT PAM gRNAs were designed to be paired with a mutated S. aureus CRISPR-Cas9-containing base editor.

An in silico off-target analysis of these gRNAs was run and any gRNAs with a 0, 1, 2 or 3 nucleotide mismatch to a tumor suppressor gene were excluded from the screen due to potential off-target effects. The gRNA list was filtered further to remove any gRNAs with 0 or 1 mismatch to any location in the human genome and 0, 1 or 2 mismatches to any exon in the human genome. This filtered list contained 47 unique gRNAs that covering the target promoter region (FIG. 9). These 47 gRNAs could be paired with either an Adenine Base Editor (ABE) or Cytosine Base Editor (CBE) to make 94 unique guide-base editor type combinations.

DNA Editing Efficiency for gRNAs with Base Editors

A cellular screen for gRNA potency was undertaken. This screen used mRNA encoding for the base editor of interest and a chemically synthesized, chemically end-protected gRNA. The screening was performed in HepG2 human cells. Three replicates were transfected into cells on the same day. DNA was harvested for next generation sequencing three days post-transfection.

Positive controls for genome editing were the following: a gRNA-mRNA pair that was known to have good editing efficiencies and did not target DNA predicted to have any impact on TTR mRNA expression (sgRNA_088 paired with NGG-SpCas9-ABE8.8), three gRNA-base editor pairs targeting splice sites within the TTR gene (gRNAs sg_361, sg_362, gRNA1597 and gRNA1604 [reference prior filings on these gRNAs]), and one Cas9 nuclease combined with a gRNA known to be suitable for inducing TTR knockdown in human (Cas9 nuclease+gRNA991) (Gillmore, J. D. et al. CRISPR-Cas9 In Vivo Gene Editing for Transthyretin Amyloidosis. New Engl.J Med 385, 493-502 (2021)).

Negative controls for genome editing were the following: no treatment, and a catalytically dead Cas9 nuclease plus gRNA991 (dead Cas9 nuclease+gRNA991).

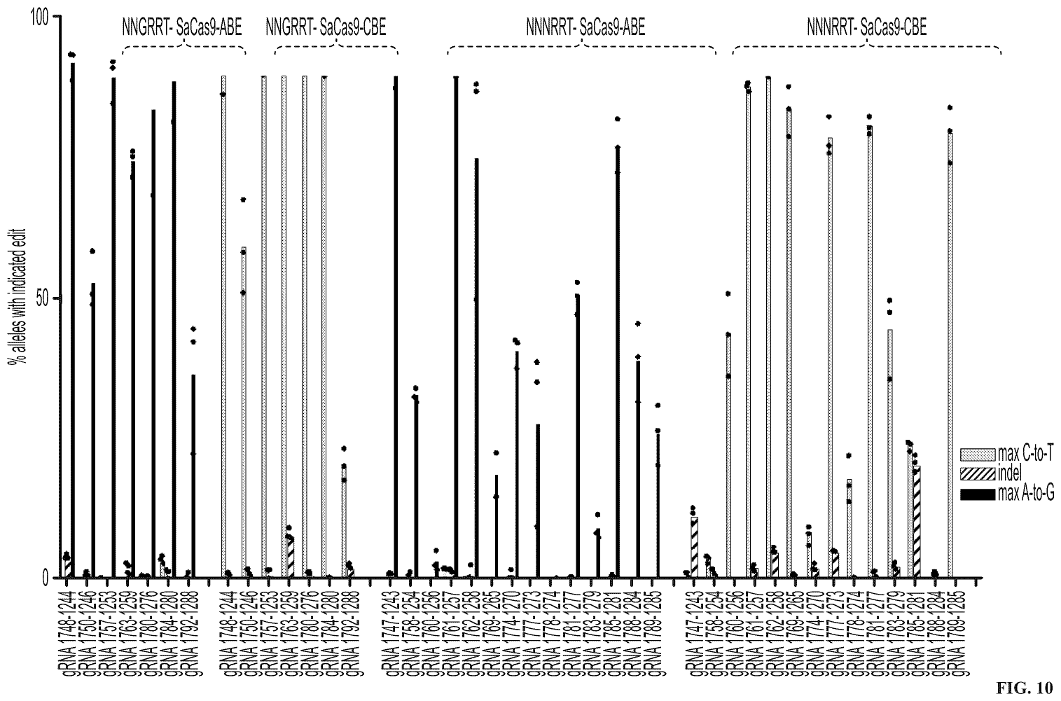

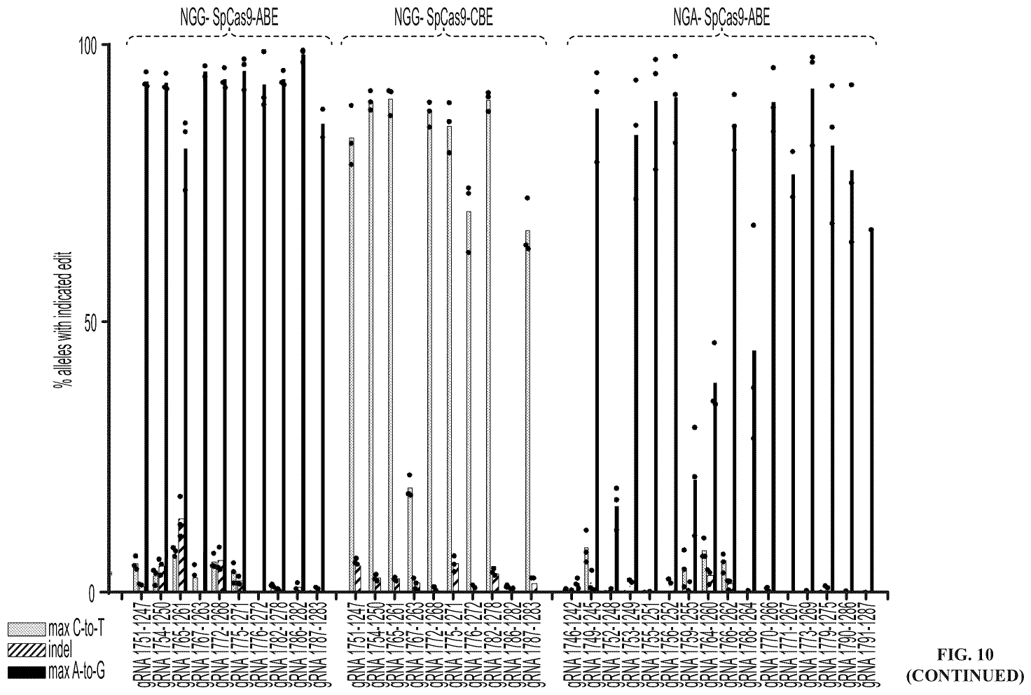

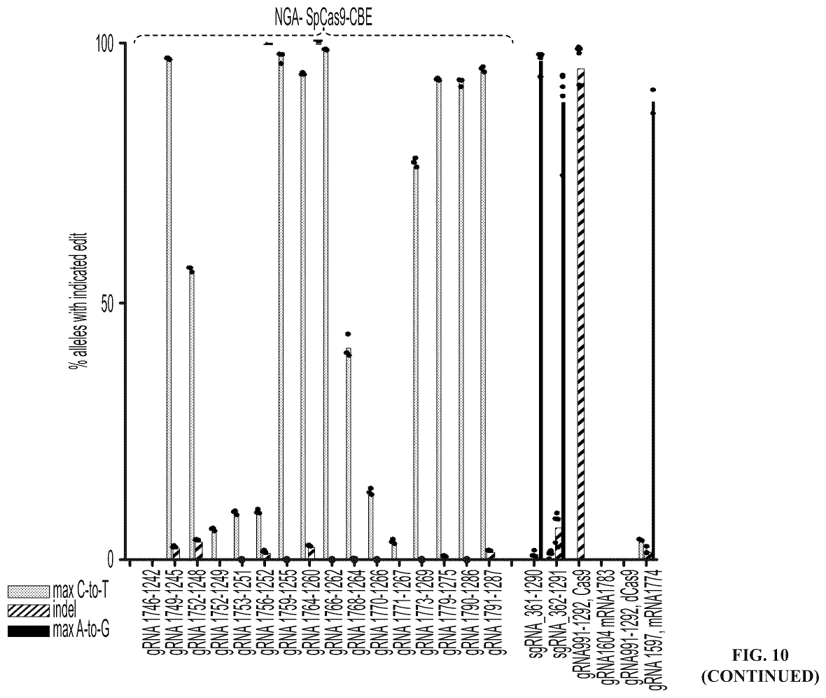

Each gRNA for the promoter screen was paired with either a CBE (here using the ppAPOBEC1 deaminase described in Yu, Y. et al. Cytosine base editors with minimized unguided DNA and RNA off-target events and high on-target activity. Nat Commun 11, 2052 (2020)) or an ABE (here using ABE8.20, described in Gaudelli, N. M. et al. Directed evolution of adenine base editors with increased activity and therapeutic application. Nat Biotechnol 38, 892-900 (2020)). Next-generation sequencing (NGS) data indicated that, when paired with CBEs, 22/46 promoter tiling gRNAs yielded mean editing frequencies >80% and 9/46 gRNAs yielded editing frequencies <10%. (FIG. 10). When paired with ABEs, 24/47 promoter tiling gRNAs yielded >80% mean editing frequency, and 4/47 gRNAs yielded mean editing frequencies <10%.

TTR Knockdown Efficiency Resulting from Promoter Editing

TTR Knockdown efficiency was measured using RT-qPCR for all promoter screening gRNAs and the control gRNAs. One of the gRNAs that served as a positive control for DNA editing also served as a negative control for TTR knockdown: the gRNA-mRNA pair that typically yielded high editing efficiencies and did not target DNA known to have any impact on TTR mRNA expression (sgRNA_088 paired with NGG-SpCas9-ABE8.8). The other negative controls included no treatment controls, which were used in each plate run for RT-qPCR, and a catalytically dead Cas9 combined with gRNA_991.

Positive controls for TTR knockdown were the following: three previously identified gRNA-base editor pairs targeting splice sites within the TTR gene (gRNAs sg_361, sg_362, gRNA1597) and one Cas9 nuclease combined with a gRNA known to induce TTR knockdown in humans (Cas9 nuclease+gRNA991).

An internal control (ACTB) with an orthogonal fluorescent probe to the test probe (TTR) was used to enable RT-qPCR samples to be accurately compared between wells. Fold-change differences in TTR mRNA abundance between the no treatment controls and each test treatment well was measured using the mean of the ΔCt(TTR−ACTB)_{control} for the no treatment wells present in each plate. The approach used to find relative TTR expression level was 2\hat{ }(−1*(ΔCt(TTR−ACTB)ₛₐₘₚₗₑ−ΔCt(TTR−ACTB)_{control}) (Livak, K. J. & Schmittgen, T. D. Analysis of Relative Gene Expression Data Using Real-Time Quantitative PCR and the 2-ΔΔC T Method. Methods 25, 402-408 (2001)). Untreated cells had a different TTR:ACTB ratio from transfected cells, which led to an artificially reduced relative TTR expression (0.30-0.42) in cells transfected with the negative control catalytically dead Cas9 editor or gRNA that did not affect TTR expression. Nonetheless, this approach was suitable as a relative approach to compare TTR knockdown efficacy between different transfection conditions.

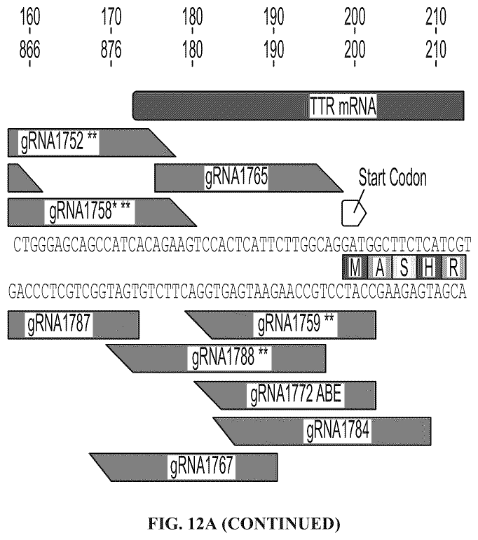

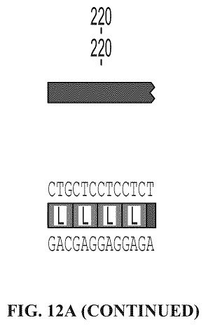

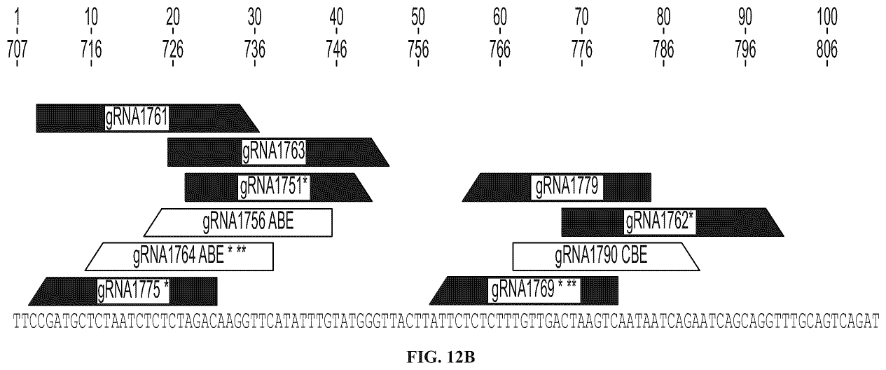

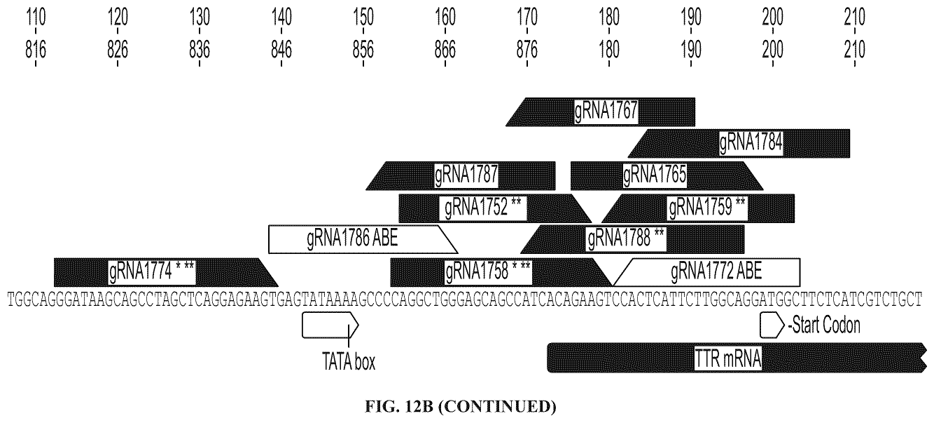

In total, 21/94 base editor-gRNA combinations (which are notated throughout this disclosure as “Base_Editor_Name_gRNA_name”) tested showed comparable or greater TTR knockdown than the positive control gRNA_991 (FIGS. 12A and 12B). Of five potent promoter tiling gRNAs, one, when combined with an ABE, edited the sequence proposed to be the TATA box for TTR (gRNA1786), and one, when combined with an ABE, disrupted the ATG start codon (gRNA1772). The other three bind elsewhere in the promoter region.

The following materials and methods were employed in this Example.

### Cell Transection

HepG2 cells were plated into a 48-well poly-D-lysine (PDL)-coated plate (Corning, 354509) at a density of 25,000 cells/well in 200 μL of supplemented media 24-hours prior to transfection. On the day of transfection, 600 ng of mRNA encoding for the desired editor (produced at Beam) and 200 ng chemically end-protected gRNA (IDT) was aliquoted out and into 96-well plates. Lipofectamine MessengerMax (Thermo Fisher, LMRNA015) was diluted in Optimem (Thermo Fisher, 31985062), vortexed thoroughly and incubated at room temperature for at least 5 minutes before being added onto the pre-aliquoted mRNA and gRNA mix at a final concentration of 1.5 μL MessengerMax lipid per well. The lipid encapsulated mRNA and gRNA mix was incubated at room temperature for 10-20 mins before being added onto cell plates.

### Cell Culture

HepG2 cells (ATCC, HB8065) were cultured according to the manufacturer's protocols and split at least every four days. Cells were cultured in EMEM (Gibco, 670086), supplemented with 10% Fetal Bovine Serum (Thermo Fisher, A3160401).

Next Generation DNA sequencing (NGS)

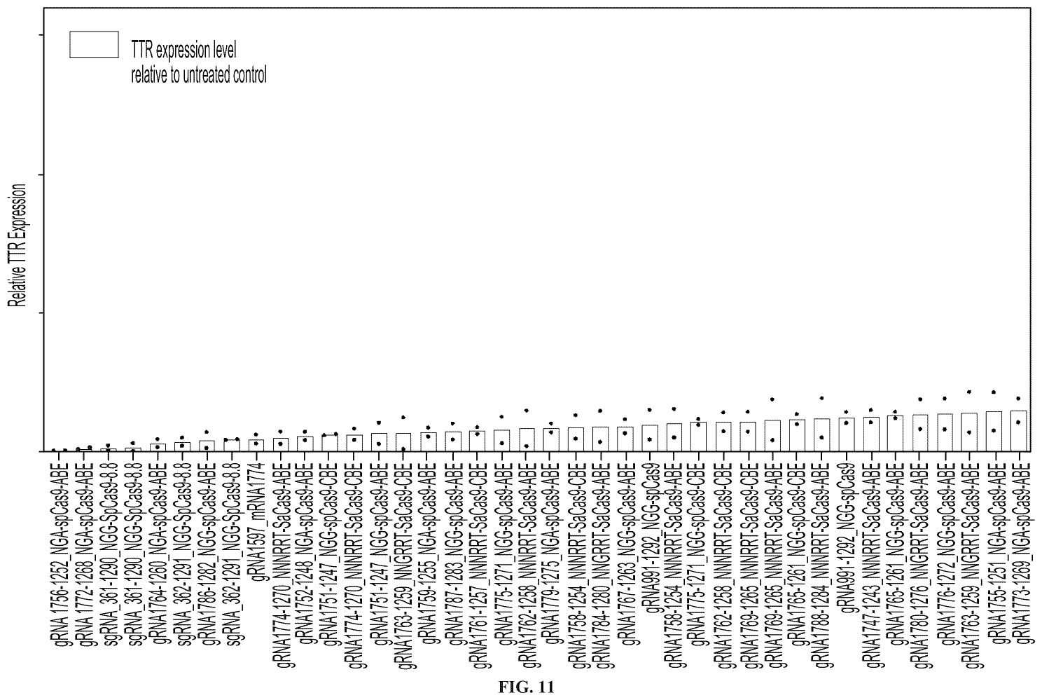

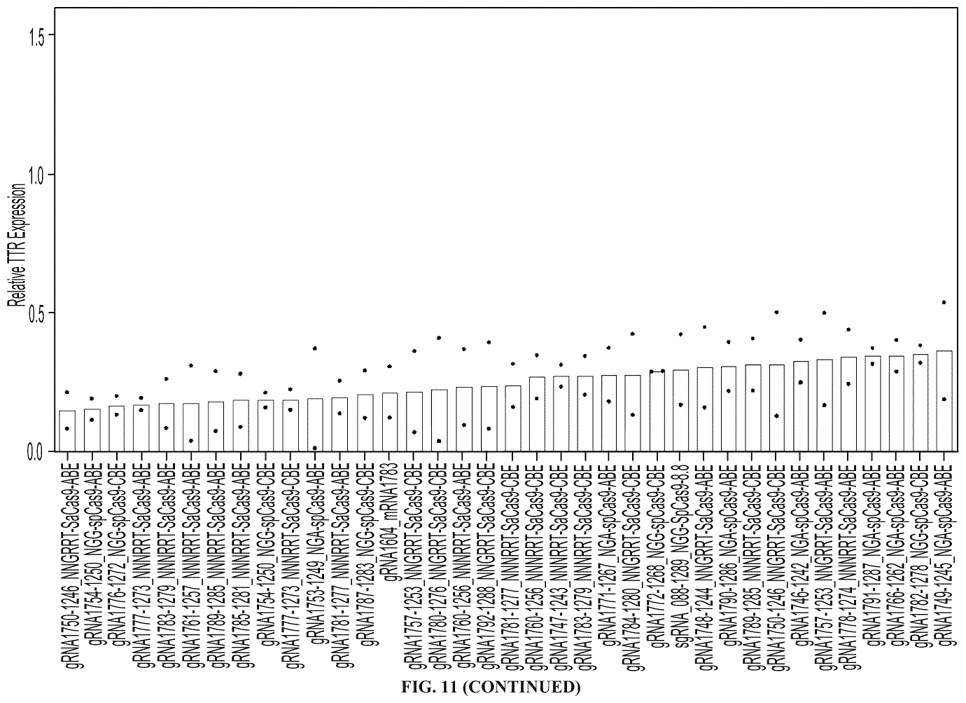

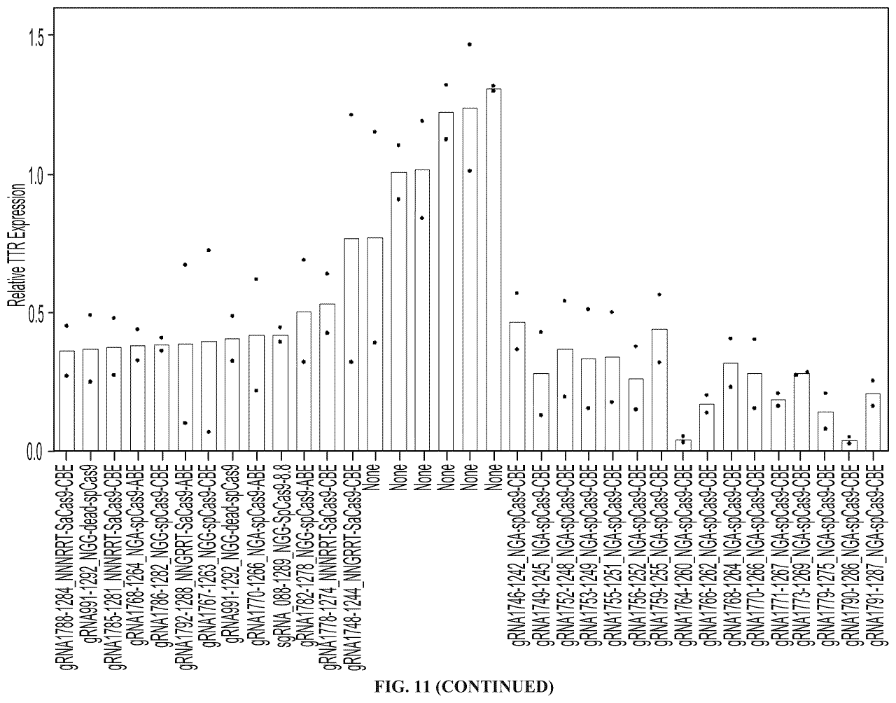

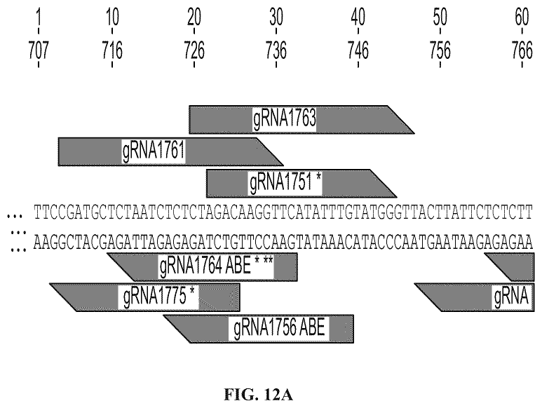

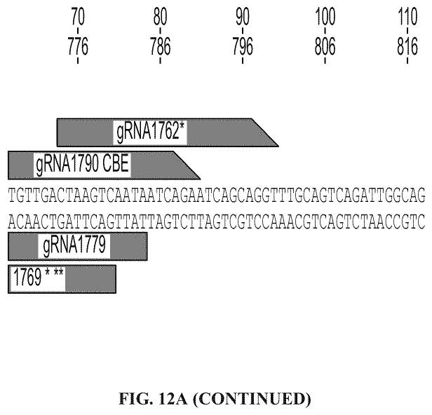

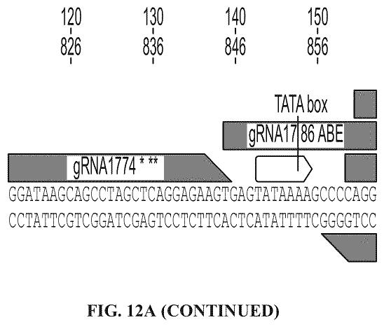

DNA was harvested from transfected cells 3 days post-transfection. Media was removed from cells and 100 μL of thawed Quick Extract lysis buffer (Lucigen, QEP70750) was added to each well. The buffer-cell mixture was incubated at 65° C. for 8 mins and then at 98° C. for 15 mins. PCR was performed to amplify the gRNA target region each sample. A second round of PCR was performed to add barcoding adapters onto the product from PCR1. The resulting product was purified and sequenced using a 300-kit on a Miseq (Illumina). DNA sequence alignment with a reference sequence and editing quantification was performed on the resulting sequences. Maximum editing (plotted in FIGS. 10 and 11) corresponded to the highest value for either an A-to-G edit or a C-to-T edit for any base within a gRNA protospacer and PAM region.

### RT-qPCR

Cells were frozen down 5 days post-transfection. Media was removed from each well and the resulting plates were sealed and stored at −80° C. RNA was harvested subsequently using the RNeasy PLUS kit (Qiagen) in 96 well plate format according to the manufacturer's instructions (74192). After RNA was isolated, Taqpath 1-step RT-qPCR Master Mix CG (Thermo Fisher, A15299) with two probes: ACTB with VIC (4448489) and TTR with FAM (4331182), all Thermo Fisher. The probes were used according to the manufacturer's instructions with 0.5 μL of RNA input in a 20 μL reaction to assess relative expression level of TTR. Quantstudio 7 (Thermo Fisher) was used to run the RT-qPCR assay. Three technical replicates were run per plate. Auto thresholds for Ct values were used for each individual value. Any replicates indicating no amplification or inconclusive amplification were excluded from the analysis, resulting in a few samples having only two technical replicates. To calculate relative expression of TTR, the \hat{ }(−1*(ΔCt(TTR−ACTB)ₛₐₘₚₗₑ−ΔCt(TTR−ACTB)_{control}) approach (Livak, K. J. & Schmittgen, T. D. Analysis of Relative Gene Expression Data Using Real-Time Quantitative PCR and the 2-ΔΔC T Method. Methods 25, 402-408 (2001)) was used.

### Example 6. Transthyretin (TRR) Guide Screening and Functional Knockdown Assessment in Hek293T Cells

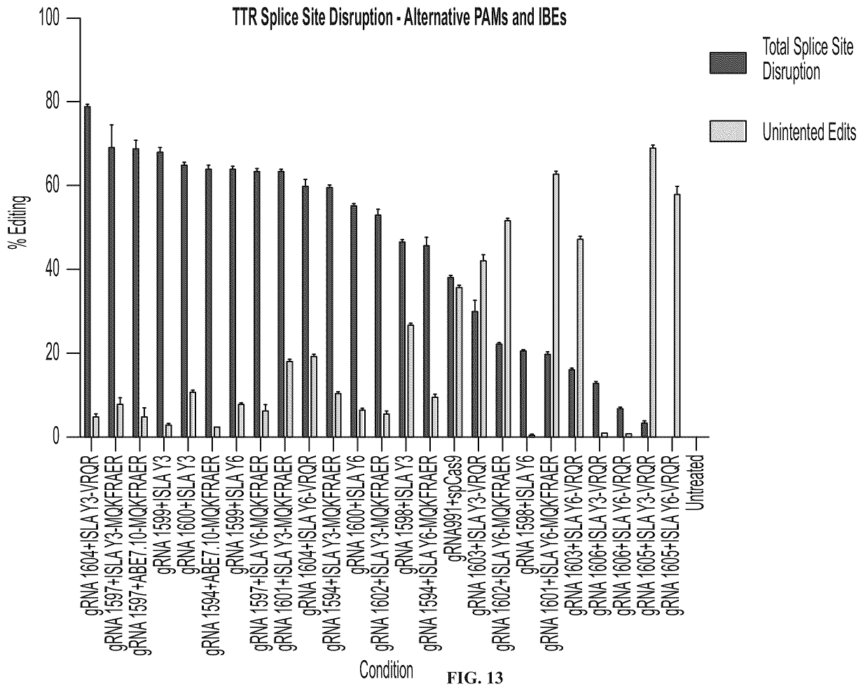

Fourteen guide RNAs were designed using a base-editing strategy for splice-site disruption using ABE7.10 alternative PAM editors or IBE variants, for a total of 26 new experimental combinations. Nine (9) tested combinations demonstrated good editing efficiencies in Hek293T cells (FIG. 13).

Editor mRNA and sgRNAs were transfected in triplicate into Hek293T cells. Spacer sequences for the sgRNAs are provided in Table 2B. All sgRNAs were ordered from IDT with 80-mer spCas9 scaffolds. In addition to the 26 experimental combinations, gRNA991 known to induce TTR knockdown in humans (Gillmore, J. D. et al. CRISPR-Cas9 In Vivo Gene Editing for Transthyretin Amyloidosis. New Engl J Med 385, 493-502 (2021)) combined with spCas9, and no treatment were used as positive and negative controls, respectively. Genomic DNA was harvested 72 hours after transfection and sequenced using Next Generation Sequencing. Total editing resulting in splice site disruption was detected in a range from ˜79%-0.4% depending on the condition, with some combinations yielding total editing resulting in splice disruption in a range between 79% to 63.5%. Most editor variants exhibited detectable editing at target loci. The following combinations displayed relatively high levels of editing: ISLAY3-VRQR_gRNA1604; ISLAY3-MQKFRAER_gRNA1597; ABE7.10-MQKFRAER_gRNA1597; ISLAY3_gRNA1599; ISLAY3_gRNA1600; ABE7.10-MQKFRAER_gRNA1594; ISLAY6_gRNA1599; ISLAY6-MQKFRAER_gRNA1597; ISLAY3-MQKFRAER_gRNA1601. For a description of the internal base editors (ISLAY) see Tables 4 and 7. The internal base editors (i.e., ISLAY3 and ISLAY6, each contained a TadA*7.10 deaminase domain. The internal base editors are described in PCT/US20/16285, the disclosure of which is incorporated herein by reference in its entirety for all purposes. In particular, the combination of gRNA1604 and ISLAY3-VRQR exhibited editing efficiencies at ˜79%. The combination of gRNA1597 with both ISLAY3-MQKFRAER and ABE7.10 exhibited good editing efficiencies as well.

The following materials and methods were employed in Example.

### Hek293T Cell Culture and Maintenance

A frozen vial of Hek293T cells at passage count 3 was thawed and mixed with 15 mL of pre-warmed DMEM high glucose pyruvate medium (Thermofisher, 11995065) with 10% Fetal Bovine Serum (Thermofisher, A3160401) and Pen/Strep (Thermofisher, 10378016), and plated on a T75 tissue-culture treated flask (Corning, 430641U) at 37° C. in a 5% CO2 incubator (Thermofisher 51033547). The media was aspirated and replaced the next morning, and every other day thereafter. Upon reaching 70-80% confluency after 3 days, the cells were split at 1:20 via aspiration of media followed by incubation with 2 mL TrypLE (Thermofisher 12605036) for 3 minutes, gentle agitation and pipette mixing, and transfer of 100 μL into 15 mL pre-warmed media again. This process was repeated after another 5 days, during which time cell counts were obtained by averaging two results obtained from a NucleoCounter NC-200 after diluting the 2 mL of TrypLE cell suspension obtained from the flask in 10 mL of media. The cells were then seeded into Poly-D-Lysine 48-well plates (Corning, 354509) at 25 k cells/well in 200 μL of media.

### Cell Transection

Hek293T cells were transfected the day after seeding. The media was changed prior to transfection. Each well received 200 ng gRNA (Synthego custom order) (sequences for the guide RNA's are provided in Tables 1 and 2B; gRNA991 contained the spacer sequence AAAGGCUGCUGAUGACACCU (SEQ ID NO: 565) and 600 ng mRNA with 1.5 μL Lipofectamine MessengerMax (Thermofisher, LMRNA150). Guide RNAs were reconstituted from lyophilized form in water at lmg/mL, and mRNA was received at 2 mg/mL. gRNA/mRNA and reagent were separately added to 26 μL OptiMEM (Thermofisher, 31985062) per well as half mixes and incubated for 10 minutes, after which the RNA and reagent half mixes were combined and incubated for another 5 min. 54 μL of the combined mastermix was added dropwise to each target culture well. The plates were then briefly and gently nutated and placed at 37° C. and 5% CO₂ in the incubator. Media was changed the following day.

### Next Generation DNA Sequencing (NGS)

72 hours after transfection, media was aspirated and genomic DNA was isolated with lysis buffer solution of 10 mM Tris-HCl pH8.0, 0.05% SDS, 50 ug/mL proteinase K (Thermofisher, E00491). 200 μL of lysis buffer was added per well, and the plates were incubated at 37° C. for 45 minutes, after which the samples were vigorously mixed and 100 μL of the volume was transferred to a 96-well PCR plate. The plate was incubated at 95° C. for 15 minutes and 1 μL was transferred into a PCR mixture. PCR was performed using Q5 Hotstart 2× Mastermix (M0494L) and target site-specific amplicon primers. 25 μL of mastermix, SuM each of forward and reverse primer, and to 50 μL of water were used per well. A second round of barcoding PCR was performed with half the volume. PCR products were pooled by amplicon sequence and 166 μL was added to 33 μL Purple 6× Dye (B7024S) and gel extracted in 1% agarose, then purified twice using Zymo Gel Extraction (D4007) and PCR Cleanup (D4013) kits, eluting in 150 μL 10 mM Tris pH7.5. The library concentration was quantified via NanoDrop (Thermofisher, ND-ONE-W), and standardized to 4 nM. Sequencing was performed using a MiSeq Reagent Kit v2 (500 cycles) (Illumina, MS-102-2003), with read alignment to reference sequences and editing efficiency was computationally analyzed.

The following methods were employed in the above examples.

### General HEK293T Mammalian Culture Conditions

Cells were cultured at 37° C. with 5% CO₂. HEK293T cells [CLBT×013, American Type Cell Culture Collection (ATCC)] were cultured in Dulbecco's modified Eagles medium plus Glutamax (10566-016, Thermo Fisher Scientific) with 10% (v/v) fetal bovine serum (A31606-02, Thermo Fisher Scientific). Cells were tested negative for mycoplasma after receipt from supplier.

### Lipotransfection

HEK293T cells were seeded onto 48-well well Poly-D-Lysine treated BioCoat plates (Corning) at a density of 35,000 cells/well and transfected 18-24 hours after plating. Cells were counted using a NucleoCounter NC-200 (Chemometec). A solution was prepared containing Opti-MEM reduced serum media (ThermoFisher Scientific), the base editor, nuclease, or control mRNA, and sgRNA. The solution was combined with Lipofectamine MessengerMAX (ThermoFisher) in Opti-MEM reduced serum media and left to rest at room temperature for 15 min. The resulting mixture was then transferred to the pre-seeded Hek293T cells and left to incubate for about 120 h.

### DNA Extraction and Analysis of Editing

Cells were harvested and DNA was extracted. For DNA analysis, cells were washed once in 1×PBS, and then lysed in 100 μl QuickExtract™ Buffer (Lucigen) according to the manufacturer's instructions.

Genomic DNA was sequences using Illumina Miseq sequencers following PCR to amplify edited regions.

mRNA Production

All base editor and bhCas12b mRNA was generated using the following synthesis protocol. Base editors or bhCas12b were cloned into a plasmid encoding a dT7 promoter followed by a 5′UTR, Kozak sequence, ORF, and 3′UTR. The dT7 promoter carries an inactivating point mutation within the T7 promoter that prevents transcription from circular plasmid. This plasmid templated a PCR reaction (Q5 Hot Start 2× Master Mix), in which the forward primer corrected the SNP within the T7 promoter and the reverse primer appended a polyA tail to the 3′ UTR. The resulting PCR product was purified on a Zymo Research 25 μg DCC column and used as mRNA template in the subsequent in vitro transcription. The NEB HiScribe High-Yield Kit was used according to the instruction manual, but with full substitution of N1-methyl-pseudouridine for uridine and co-transcriptional capping with CleanCap AG (Trilink). Reaction cleanup was performed by lithium chloride precipitation. Primers used for amplification can be found in Table 22.

TABLE 22
Primers used for ABE8 T7
in vitro transcription reactions

| Name | Sequence |
|---|---|
| fwd_IVT | TCGAGCTCGGTACCTAATACGACTCAC |
| | (SEQ ID NO: 451) |
| rev_IVT | TTTTTTTTTTTTTTTTTTTTTTTTTTT |
| | TTTTTTTTTTTTTTTTTTTTTTTTTTT |
| | TTTTTTTTTTTTTTTTTTTTTTTTTTT |
| | TTTTTTTTTTTTTTTTTTTTTTTTTTT |
| | TTTTTTTTTTTTCTTCCTACTCAGGCT |
| | TTATTCAAAGACCA |
| | (SEQ ID NO: 452) |

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed:

1. A method for editing a transthyretin (TTR) polynucleotide sequence, the method comprising: contacting the polynucleotide sequence with a guide RNA and a base editor comprising a polynucleotide programmable DNA binding polypeptide and an adenosine or cytidine deaminase, wherein said guide RNA targets said base editor to effect an alteration of a nucleobase of the TTR polynucleotide sequence.

2. The method of claim 1, wherein the editing introduces an alteration that corrects a mutation in a TTR polynucleotide and/or wherein the editing introduces an alteration that reduces or eliminates expression of a TTR polypeptide.

3. The method of claim 1, wherein the alteration is in a splice acceptor, splice donor, intronic sequence, exonic sequence, enhancer, or promoter.

4. A method for editing a transthyretin (TTR) polynucleotide sequence, the method comprising: contacting the polynucleotide sequence with a guide RNA and a fusion protein comprising a polynucleotide programmable DNA binding domain and an adenosine deaminase domain, wherein the adenosine deaminase domain comprises an arginine (R) or a threonine (T) at amino acid position 147 of the following amino acid sequence, and the adenosine deaminase domain has at least about 85% sequence identity to the following amino acid sequence: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 4; TadA*7.10), or wherein the cytidine deaminase domain comprises an amino acid sequence with at least about 85% sequence identity to the amino acid sequence: MSSETGPVAVDPTLRRRIEPHEFEVFFDPRELRKETCLLYEINWGGRHSIWRHTSQNTNKHVEV NFIEKFTTERYFCPNTRCSITWFLSWSPCGECSRAITEFLSRYPHVTLFIYIARLYHHADPRNR QGLRDLISSGVTIQIMTEQESGYCWRNFVNYSPSNEAHWPRYPHLWVRLYVLELYCIILGLPPC LNILRRKQPQLTFFTIALQSCHYQRLPPHILWATGLK (SEQ ID NO: 15; BE4 cytidine deaminase domain), wherein said guide RNA targets said fusion protein to effect an alteration of a nucleobase of the TTR polynucleotide sequence.

5. The method of claim 4, wherein the alteration is in a region of the TTR promoter corresponding to nucleotide positions +1 to −225 of the TTR promoter, wherein position +1 corresponds to A of the start codon (ATG) of the TTR polynucleotide sequence;
wherein the alteration is in a region of the TTR promoter corresponding to nucleotide positions +1 to −198 of the TTR promoter, wherein position +1 corresponds to A of the start codon (ATG) of the TTR polynucleotide sequence; wherein the alteration is in a region of the TTR promoter corresponding to nucleotide positions +1 to −177 of the TTR promoter, wherein position +1 corresponds to A of the start codon (ATG) of the TTR polynucleotide sequence; or
wherein the alteration is in a region of the TTR promoter corresponding to nucleotide positions −106 to −176 of the TTR promoter, wherein position +1 corresponds to A of the start codon (ATG) of the TTR polynucleotide sequence.

6. The method of claim 1, wherein the TTR polynucleotide sequence encodes a mature TTR polypeptide comprising a pathogenic alteration selected from the group consisting of T60A, V30M, V30A, V30G, V30L, V122I, and V122A.

7. The method of claim 1, wherein the altered nucleobase is
4A of the nucleotide sequence TATAGGAAAACCAGTGAGTC (SEQ ID NO: 425; TSBT×2602/gRNA1598 target site sequence corresponding to sgRNA_361);
6A of the nucleotide sequence TACTCACCTCTGCATGCTCA (SEQ ID NO: 426; TSBT×2603/gRNA1599 target site sequence corresponding to sgRNA_362);
5A of the nucleotide sequence ACTCACCTCTGCATGCTCAT (SEQ ID NO: 427; TSBT×2604/gRNA1606 target site sequence corresponding to sgRNA_363);
7A of the nucleotide sequence ATACTCACCTCTGCATGCTCA (SEQ ID NO: 429; TSBT×2606 target site sequence corresponding to sgRNA_365);
6A of the nucleotide sequence TTGGCAGGATGGCTTCTCATCG (SEQ ID NO: 431; TSBT×2608/gRNA-#19 target site corresponding to sgRNA_367);
9A of the sequence TTGGCAGGATGGCTTCTCATCG (SEQ ID NO: 431; TSBT×2608/gRNA-#19 target site corresponding to sgRNA_367);
5A of the sequence GGCTATCGTCACCAATCCCA (SEQ ID NO: 439; corresponding to sgRNA_375); or
4A of the sequence GCTATCGTCACCAATCCCAA (SEQ ID NO: 440; corresponding to sgRNA_376);
7C of the nucleotide sequence TACTCACCTCTGCATGCTCA (SEQ ID NO: 426; TSBT×2603/gRNA1599 target site corresponding to sgRNA_362);
6C of the nucleotide sequence ACTCACCTCTGCATGCTCAT (SEQ ID NO: 427; TSBT×2604/gRNA1606 target site corresponding to sgRNA_363);
7C of the nucleotide sequence TACCACCTATGAGAGAAGAC (SEQ ID NO: 428; TSBT×2605 target site corresponding to sgRNA_364);
8C of the nucleotide sequence ATACTCACCTCTGCATGCTCA (SEQ ID NO: 429; TSBT×2606 target site corresponding to sgRNA 365); or
11C of the nucleotide sequence ACTGGTTTTCCTATAAGGTGT (SEQ ID NO: 430; TSBT×2607 target site corresponding to sgRNA_366).

8. The method of claim 4, wherein the TadA deaminase is TadA*7.10, TadA*8.1, TadA*8.2, TadA*8.8, TadA*8.9, TadA*8.10, TadA*8.11, TadA*8.12, TadA*8.13, TadA*8.15, TadA*8.16, TadA*8.19, TadA*8.20, TadA*8.21, or TadA*8.24.

9. The method of claim 1, wherein the guide RNA(s) comprises a nucleotide sequence selected from one or more of those sequences listed in Table 1, Table 2A, or Table 2B; or any of the aforementioned sequences wherein 1, 2, 3, 4, or 5 nucleotides is deleted from the 5′ and/or 3′ terminus of the nucleotide sequence.

10. The method of claim 1, wherein the guide RNA(s) comprises a nucleotide sequence, selected from the group consisting of:
(SEQ ID NO: 408; sgRNA_361/gRNA1598)
5′-UAUAGGAAAACCAGUGAGUC-3′;
(SEQ ID NO: 409; sgRNA_362/gRNA1599)
5′-UACUCACCUCUGCAUGCUCA-3′;
(SEQ ID NO: 410; sgRNA_363/gRNA1606)
5′-ACUCACCUCUGCAUGCUCAU-3′;
(SEQ ID NO: 412; sgRNA_365)
5′-AUACUCACCUCUGCAUGCUCA-3′;
(SEQ ID NO: 414; sgRNA_367/gRNA-#19)
5′-UUGGCAGGAUGGCUUCUCAUCG-3′;
(SEQ ID NO: 422; sgRNA_375)
5′-GGCUAUCGUCACCAAUCCCA-3′;
(SEQ ID NO: 423; sgRNA_376)
5′-GCUAUCGUCACCAAUCCCAA-3′;
(SEQ ID NO: 561; gRNA1604)
5′-ACACCUUAUAGGAAAACCAG-3′;
(SEQ ID NO: 554; gRNA1597)
5′-CUCUCAUAGGUGGUAUUCAC-3′;
(SEQ ID NO: 557; gRNA1600)
5′-GCAACUUACCCAGAGGCAAA-3′;
(SEQ ID NO: 551; gRNA1594)
5′-CAACUUACCCAGAGGCAAAU-3′;
(SEQ ID NO: 558; gRNA1601)
5′-UCUGUAUACUCACCUCUGCA-3′;
(SEQ ID NO: 462; gRNA1756)
5′-CAAAUAUGAACCUUGUCUAG-3′;
(SEQ ID NO: 470; gRNA1764)
5′-GAACCUUGUCUAGAGAGAUU-3′;
(SEQ ID NO: 492; gRNA1786)
5′-UGAGUAUAAAAGCCCCAGGC-3′;
and
(SEQ ID NO: 478; gRNA1772)
5′-GCCAUCCUGCCAAGAAUGAG-3′;
(SEQ ID NO: 409; sgRNA_362/gRNA1599)
5′-UACUCACCUCUGCAUGCUCA-3′,
(SEQ ID NO: 410; sgRNA_363/gRNA1606)
5′-ACUCACCUCUGCAUGCUCAU-3′,
(SEQ ID NO: 411; sgRNA_364)
5′-UACCACCUAUGAGAGAAGAC-3′,
(SEQ ID NO: 412; sgRNA_365)
5′-AUACUCACCUCUGCAUGCUCA-3′,
(SEQ ID NO: 413; sgRNA_366)
5′-ACUGGUUUUCCUAUAAGGUGU-3′,
(SEQ ID NO: 551; gRNA1594)
5′-CAACUUACCCAGAGGCAAAU-3′,
and
(SEQ ID NO: 496; gRNA1790)
5′-UGUUGACUAAGUCAAUAAUC-3′;
or any of the aforementioned sequences wherein 1, 2, 3, 4, or 5 nucleotides is deleted from the 5′ and/or 3′ terminus of the nucleotide sequence.

11. A method for editing a transthyretin (TTR) polynucleotide sequence, the method comprising: contacting the polynucleotide sequence with a guide RNA and a Cas12b endonuclease, wherein said guide RNA targets said endonuclease to effect a double-stranded break of the TTR polynucleotide sequence.

12. The method of claim 11, wherein the guide RNA comprises a nucleotide sequence, selected from the group consisting of:
(SEQ ID NO: 415; sgRNA_368)
5′-UCCUAUAAGGUGUGAAAGUCUG-3′,
(SEQ ID NO: 416; sgRNA_369)
5′-UGAGCCCAUGCAGCUCUCCAGA-3′,
(SEQ ID NO: 417; sgRNA_370)
5′-CUCCUCAGUUGUGAGCCCAUGC-3′,
(SEQ ID NO: 418; sgRNA_371)
5′-GUAGAAGGGAUAUACAAAGUGG-3′,
(SEQ ID NO: 419; sgRNA_372)
5′-CCACUUUGUAUAUCCCUUCUAC-3′,
(SEQ ID NO: 420; sgRNA_373)
5′-GGUGUCUAUUUCCACUUUGUAU-3′,
and
(SEQ ID NO: 421; sgRNA_374)
5′-CAUGAGCAUGCAGAGGUGAGUA-3′;
or any of the aforementioned sequences wherein 1, 2, 3, 4, or 5 nucleotides is deleted from the 5′ and/or 3′ terminus of the nucleotide sequence.

13. A method for treating amyloidosis in a subject, the method comprising administering to the subject a guide RNA and a polynucleotide encoding a base editor comprising a polynucleotide programmable DNA binding polypeptide and an adenosine or cytidine deaminase, wherein said guide RNA targets said base editor to effect an alteration of a nucleobase of the TTR polynucleotide sequence, wherein the adenosine deaminase domain comprises an arginine (R) or a threonine (T) at amino acid position 147 of the following amino acid sequence, and the adenosine deaminase domain has at least about 85% sequence identity to the following amino acid sequence
(SEQ ID NO: 4; TadA*7.10)
MSEVEFSHEYWMRHALTLAKRARDEREVPVGAVLVLNNRVIGEGWNR
AIGLHDPTAHAEIMALRQGGLVMQNYRLIDATLYVTFEPCVMCAGAM
IHSRIGRVVFGVRNAKTGAAGSLMDVLHYPGMNHRVEITEGILADEC
AALLCYFFRMPRQVFNAQKKAQSSTD,
wherein said guide RNA targets said fusion protein to effect an alteration of a nucleobase of the TTR polynucleotide sequence or
wherein the cytidine deaminase domain comprises an amino acid sequence with at least about 85% sequence identity to the amino acid sequence: MSSETGPVAVDPTLRRRIEPHEFEVFFDPRELRKETCLLYEINWGGRHSIWRHTSQNTNKHVEV NFIEKFTTERYFCPNTRCSITWFLSWSPCGECSRAITEFLSRYPHVTLFIYIARLYHHADPRNR QGLRDLISSGVTIQIMTEQESGYCWRNFVNYSPSNEAHWPRYPHLWVRLYVLELYCIILGLPPC LNILRRKQPQLTFFTIALQSCHYQRLPPHILWATGLK (SEQ ID NO: 15), wherein said guide RNA targets said fusion protein to effect an alteration of a nucleobase of the TTR polynucleotide sequence.

14. The method of claim 13, wherein the altered nucleobase is
4A of the nucleotide sequence TATAGGAAAACCAGTGAGTC (SEQ ID NO: 425; TSBT×2602/gRNA1598 target site sequence corresponding to sgRNA_361);
6A of the nucleotide sequence TACTCACCTCTGCATGCTCA (SEQ ID NO: 426; TSBT×2603/gRNA1599 target site sequence corresponding to sgRNA_362);
5A of the nucleotide sequence ACTCACCTCTGCATGCTCAT (SEQ ID NO: 427; TSBT×2604/gRNA1606 target site sequence corresponding to sgRNA_363);
7A of the nucleotide sequence ATACTCACCTCTGCATGCTCA (SEQ ID NO: 429; TSBT×2606 target site sequence corresponding to sgRNA_365);
6A of the nucleotide sequence TTGGCAGGATGGCTTCTCATCG (SEQ ID NO: 431; TSBT×2608/gRNA-#19 target site corresponding to sgRNA_367);
9A of the sequence TTGGCAGGATGGCTTCTCATCG (SEQ ID NO: 431; TSBT×2608/gRNA-#19 target site corresponding to sgRNA_367);
5A of the sequence GGCTATCGTCACCAATCCCA (SEQ ID NO: 439; corresponding to sgRNA_375); or
4A of the sequence GCTATCGTCACCAATCCCAA (SEQ ID NO: 440; corresponding to sgRNA_376);
7C of the nucleotide sequence TACTCACCTCTGCATGCTCA (SEQ ID NO: 426; TSBT×2603/gRNA1599 target site corresponding to sgRNA_362);
6C of the nucleotide sequence ACTCACCTCTGCATGCTCAT (SEQ ID NO: 427; TSBT×2604/gRNA1606 target site corresponding to sgRNA_363);
7C of the nucleotide sequence TACCACCTATGAGAGAAGAC (SEQ ID NO: 428; TSBT×2605 target site corresponding to sgRNA_364);
8C of the nucleotide sequence ATACTCACCTCTGCATGCTCA (SEQ ID NO: 429; TSBT×2606 target site corresponding to sgRNA 365); or
11C of the nucleotide sequence ACTGGTTTTCCTATAAGGTGT (SEQ ID NO: 430; TSBT×2607 target site corresponding to sgRNA_366).

15. The method of claim 13, wherein the adenosine deaminase is a TadA deaminase selected from the group consisting of TadA7*10, TadA*8.1, TadA*8.2, TadA*8.8, TadA*8.9, TadA*8.10, TadA*8.11, TadA*8.12, TadA*8.13, TadA*8.15, TadA*8.16, TadA*8.19, TadA*8.20, TadA*8.21, or TadA*8.24.

16. The method of claim 13, wherein the guide RNA(s) comprises a nucleotide sequence selected from one or more of those sequences listed in Table 1, Table 2A, or Table 2B; or any of the aforementioned sequences wherein 1, 2, 3, 4, or 5 nucleotides is deleted from the 5′ and/or 3′ terminus of the nucleotide sequence.

17. The method of claim 13, wherein the guide RNA(s) comprises a nucleotide sequence, selected from the group consisting of:
(SEQ ID NO: 408; sgRNA_361/gRNA1598)
5′-UAUAGGAAAACCAGUGAGUC-3′;
(SEQ ID NO: 409; sgRNA_362/gRNA1599)
5′-UACUCACCUCUGCAUGCUCA-3′;
(SEQ ID NO: 410; sgRNA_363/gRNA1606)
5′-ACUCACCUCUGCAUGCUCAU-3′;
(SEQ ID NO: 412; sgRNA_365)
5′-AUACUCACCUCUGCAUGCUCA-3′;
(SEQ ID NO: 414; sgRNA_367/gRNA-#19)
5′-UUGGCAGGAUGGCUUCUCAUCG-3′;
(SEQ ID NO: 422; sgRNA_375)
5′-GGCUAUCGUCACCAAUCCCA-3′;
(SEQ ID NO: 423; sgRNA_376)
5′-GCUAUCGUCACCAAUCCCAA-3′;
(SEQ ID NO: 561; gRNA1604)
5′-ACACCUUAUAGGAAAACCAG-3′;
(SEQ ID NO: 554; gRNA1597)
5′-CUCUCAUAGGUGGUAUUCAC-3′;
(SEQ ID NO: 557; gRNA1600)
5′-GCAACUUACCCAGAGGCAAA-3′;
(SEQ ID NO: 551; gRNA1594)
5′-CAACUUACCCAGAGGCAAAU-3′;
(SEQ ID NO: 558; gRNA1601)
5′-UCUGUAUACUCACCUCUGCA-3′;
(SEQ ID NO: 462; gRNA1756)
5′-CAAAUAUGAACCUUGUCUAG-3′;
(SEQ ID NO: 470; gRNA1764)
5′-GAACCUUGUCUAGAGAGAUU-3′;
(SEQ ID NO: 492; gRNA1786)
5′-UGAGUAUAAAAGCCCCAGGC-3′;
and
(SEQ ID NO: 478; gRNA1772)
5′-GCCAUCCUGCCAAGAAUGAG-3′;
(SEQ ID NO: 409; sgRNA_362/gRNA1599)
5′-UACUCACCUCUGCAUGCUCA-3′,
(SEQ ID NO: 410; sgRNA_363/gRNA1606)
5′-ACUCACCUCUGCAUGCUCAU-3′,
(SEQ ID NO: 411; sgRNA_364)
5′-UACCACCUAUGAGAGAAGAC-3′,
(SEQ ID NO: 412; sgRNA_365)
5′-AUACUCACCUCUGCAUGCUCA-3′,
(SEQ ID NO: 413; sgRNA_366)
5′-ACUGGUUUUCCUAUAAGGUGU-3′,
(SEQ ID NO: 551; gRNA1594)
5′-CAACUUACCCAGAGGCAAAU-3′,
and
(SEQ ID NO: 496; gRNA1790)
5′-UGUUGACUAAGUCAAUAAUC-3′;
or any of the aforementioned sequences wherein 1, 2, 3, 4, or 5 nucleotides is deleted from the 5′ and/or 3′ terminus of the nucleotide sequence.

18. A method for editing a transthyretin (TTR) polynucleotide sequence in a subject, the method comprising administering to a subject a guide RNA and a Cas12b endonuclease, wherein said guide RNA targets said endonuclease to effect a double-stranded break of the TTR polynucleotide sequence, wherein the TTR polynucleotide sequence encodes a mature TTR polynucleotide comprising a pathogenic alteration selected from the group consisting of T60A, V30M, V30A, V30G, V30L, V122I, and V122A, wherein the guide RNA(s) comprises a nucleotide sequence selected from one or more of those sequences listed in Table 1, Table 2A, or Table 2B; or any of the aforementioned sequences wherein 1, 2, 3, 4, or 5 nucleotides is deleted from the 5′ and/or 3′ terminus of the nucleotide sequence.

19. The method of claim 18, wherein the guide RNA comprises a nucleotide sequence, selected from the group consisting of:
(SEQ ID NO: 415; sgRNA_368)
5′-UCCUAUAAGGUGUGAAAGUCUG-3′,
(SEQ ID NO: 416; sgRNA_369)
5′-UGAGCCCAUGCAGCUCUCCAGA-3′,
(SEQ ID NO: 417; sgRNA_370)
5′-CUCCUCAGUUGUGAGCCCAUGC-3′,
(SEQ ID NO: 418; sgRNA_371)
5′-GUAGAAGGGAUAUACAAAGUGG-3′,
(SEQ ID NO: 419; sgRNA_372)
5′-CCACUUUGUAUAUCCCUUCUAC-3′,
(SEQ ID NO: 420; sgRNA_373)
5′-GGUGUCUAUUUCCACUUUGUAU-3′,
and
(SEQ ID NO: 421; sgRNA_374)
5′-CAUGAGCAUGCAGAGGUGAGUA-3′;
or any of the aforementioned sequences wherein 1, 2, 3, 4, or 5 nucleotides is deleted from the 5′ and/or 3′ terminus of the nucleotide sequence.

20. A composition comprising one or more polynucleotides encoding a fusion protein and a guide RNA, wherein the guide RNA comprises a nucleic acid sequence that is complementary to a transthyretin (TTR) polynucleotide, and wherein the fusion protein comprises a polynucleotide programmable DNA binding domain and an adenosine or cytidine deaminase domain.

21. The composition of claim 20, wherein the adenosine deaminase domain comprises an arginine (R) or a threonine (T) at amino acid position 147 of the following amino acid sequence, and the adenosine deaminase domain has at least about 85% sequence identity to the following amino acid sequence:
(SEQ ID NO: 4; TadA*7.10)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCY
FFRMPRQVENAQKKAQSSTD,
wherein said guide RNA targets said fusion protein to effect an alteration of a nucleobase of a TTR polynucleotide sequence.

22. The composition of claim 20, wherein the fusion protein:
(i) comprises an amino acid sequence that is at least 80%, 85%, 90%, 95%, 96%, 97%, 98%, 99%, or 100% identical to:
ABE8.8
(SEQ ID NO: 442)
MSEVEFSHEYWMRHALTLAKRARDEREVPVGAVLVLNNRVIGEGWNRAIGLHDPTAHAEIMALR
QGGLVMQNYRLIDATLYVTFEPCVMCAGAMIHSRIGRVVFGVRNAKTGAAGSLMDVLHHPGMNH
RVEITEGILADECAALLCRFFRMPRRVENAQKKAQSSTDSGGSSGGSSGSETPGTSESATPESS
GGSSGGSDKKYSIGLAIGTNSVGWAVITDEYKVPSKKFKVLGNTDRHSIKKNLIGALLEDSGET
AEATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKERGHFLIEGDLNPDNSDVDKLFI
QLVQTYNQLFEENPINASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAILLSDILRVNTEITK
APLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPELKDNREKIEK
ILTFRIPYYVGPLARGNSRFAWMTRKSEETITPWNFEEVVDKGASAQSFIERMTNEDKNLPNEK
VLPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDELDNEENEDILEDIVLTLTLFEDREMIEE
RLKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMNTKYDENDKLIREVK
VITLKSKLVSDFRKDFQFYKVREINNYHHAHDAYLNAVVGTALIKKYPKLESEFVYGDYKVYDV
RKMIAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAAFKYFDTTIDRKRYTS
TKEVLDATLIHQSITGLYETRIDLSQLGGDEGADKRTADGSEFESPKKKRKV;
(ii) comprises an amino acid sequence that is at least 80%, 85%, 90%, 95%, 96%, 97%, 98%, 99%, or 100% identical to:
BE4
(SEQ ID NO: 443)
MSSETGPVAVDPTLRRRIEPHEFEVFFDPRELRKETCLLYEINWGGRHSIWRHTSQNTNKHVEV
NFIEKFTTERYFCPNTRCSITWELSWSPCGECSRAITEFLSRYPHVTLFIYIARLYHHADPRNR
QGLRDLISSGVTIQIMTEQESGYCWRNFVNYSPSNEAHWPRYPHLWVRLYVLELYCIILGLPPC
LNILRRKQPQLTFFTIALQSCHYQRLPPHILWATGLKSGGSSGGSSGSETPGTSESATPESSGG
SSGGSDKKYSIGLAIGTNSVGWAVITDEYKVPSKKFKVLGNTDRHSIKKNLIGALLEDSGETAE
ATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKERGHFLIEGDLNPDNSDVDKLFIQL
VQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKAP
LSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNEDKNLPNEKVL
PKHSLLYEYFTVYNELTKVKYVTEGMRKPAFLSGEQKKAIVDLLFKTNRKVTVKQLKEDYFKKI
ECFDSVEISGVEDRENASLGTYHDLLKIIKDKDELDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVR
KVLSMPQVNIVKKTEVQTGGESKESILPKRNSDKLIARKKDWDPKKYGGFDSPTVAYSVLVVAK
VEKGKSKKLKSVKELLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQHKHYLDEIIEQISEF
SKRVILADANLDKVLSAYNKHRDKPIREQAENIIHLFTLTNLGAPAAFKYEDTTIDRKRYTSTK
EVLDATLIHQSITGLYETRIDLSQLGGDSGGSGGSGGSTNLSDIIEKETGKQLVIQESILMLPE
EVEEVIGNKPESDILVHTAYDESTDENVMLLTSDAPEYKPWALVIQDSNGENKIKMLSGGSGGS
GGSTNLSDIIEKETGKQLVIQESILMLPEEVEEVIGNKPESDILVHTAYDESTDENVMLLTSDA
PEYKPWALVIQDSNGENKIKMLSGGSKRTADGSEFESPKKKRKVE;
(iii) comprises an amino acid sequence that is at least 80%, 85%, 90%, 95%, 96%, 97%, 98%, 99%, or 100% identical to:
ABE8.8-VRQR
(SEQ ID NO: 444)
MSEVEFSHEYWMRHALTLAKRARDEREVPVGAVLVLNNRVIGEGWNRAIGLHDPTAHAEIMALR
QGGLVMQNYRLIDATLYVTFEPCVMCAGAMIHSRIGRVVFGVRNAKTGAAGSLMDVLHHPGMNH
RVEITEGILADECAALLCRFFRMPRRVENAQKKAQSSTDSGGSSGGSSGSETPGTSESATPESS
GGSSGGSDKKYSIGLAIGTNSVGWAVITDEYKVPSKKFKVLGNTDRHSIKKNLIGALLEDSGET
AEATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAILLSDILRVNTEITK
APLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMNTKYDENDKLIREVK
VITLKSKLVSDFRKDFQFYKVREINNYHHAHDAYLNAVVGTALIKKYPKLESEFVYGDYKVYDV
RKMIAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFVSPTVAYSVLVV
AKVEKGKSKKLKSVKELLGITIMERSSFEKNPIDFLEAKGYKEVKKDLIIKLPKYSLFELENGR
KRMLASARELQKGNELALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAAFKYEDTTIDRKQYRS
TKEVLDATLIHQSITGLYETRIDLSQLGGDEGADKRTADGSEFESPKKKRKV;
(iv) comprises an amino acid sequence that is at least 80%, 85%, 90%, 95%, 96%, 97%, 98%, 99%, or 100% identical to:
BE4-VRQR
(SEQ ID NO: 445)
MSSETGPVAVDPTLRRRIEPHEFEVFFDPRELRKETCLLYEINWGGRHSIWRHTSQNTNKHVEV
NFIEKFTTERYFCPNTRCSITWFLSWSPCGECSRAITEFLSRYPHVTLFIYIARLYHHADPRNR
QGLRDLISSGVTIQIMTEQESGYCWRNFVNYSPSNEAHWPRYPHLWVRLYVLELYCIILGLPPC
LNILRRKQPQLTFFTIALQSCHYQRLPPHILWATGLKSGGSSGGSSGSETPGTSESATPESSGG
SSGGSDKKYSIGLAIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKAP
LSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNEDKNLPNEKVL
PKHSLLYEYFTVYNELTKVKYVTEGMRKPAFLSGEQKKAIVDLLFKTNRKVTVKQLKEDYFKKI
ECFDSVEISGVEDRENASLGTYHDLLKIIKDKDELDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGFVSPTVAYSVLVVAK
VEKGKSKKLKSVKELLGITIMERSSFEKNPIDFLEAKGYKEVKKDLIIKLPKYSLFELENGRKR
MLASARELQKGNELALPSKYVNFLYLASHYEKLKGSPEDNEQKQLFVEQHKHYLDEIIEQISEF
SKRVILADANLDKVLSAYNKHRDKPIREQAENIIHLFTLTNLGAPAAFKYEDTTIDRKQYRSTK
EVLDATLIHQSITGLYETRIDLSQLGGDSGGSGGSGGSTNLSDIIEKETGKQLVIQESILMLPE
EVEEVIGNKPESDILVHTAYDESTDENVMLLTSDAPEYKPWALVIQDSNGENKIKMLSGGSGGS
GGSTNLSDIIEKETGKQLVIQESILMLPEEVEEVIGNKPESDILVHTAYDESTDENVMLLTSDA
PEYKPWALVIQDSNGENKIKMLSGGSKRTADGSEFESPKKKRKVE;
(v) comprises an amino acid sequence that is at least 80%, 85%, 90%, 95%, 96%, 97%, 98%, 99%, or 100% identical to:
saABE8.8
(SEQ ID NO: 446)
MSEVEFSHEYWMRHALTLAKRARDEREVPVGAVLVLNNRVIGEGWNRAIGLHDPTAHAEIMALR
QGGLVMQNYRLIDATLYVTFEPCVMCAGAMIHSRIGRVVFGVRNAKTGAAGSLMDVLHHPGMNH
RVEITEGILADECAALLCRFERMPRRVENAQKKAQSSTDSGGSSGGSSGSETPGTSESATPESS
GGSSGGSKRNYILGLAIGITSVGYGIIDYETRDVIDAGVRLFKEANVENNEGRRSKRGARRLKR
RRRHRIQRVKKLLFDYNLLTDHSELSGINPYEARVKGLSQKLSEEEFSAALLHLAKRRGVHNVN
EVEEDTGNELSTKEQISRNSKALEEKYVAELQLERLKKDGEVRGSINRFKTSDYVKEAKQLLKV
QKAYHQLDQSFIDTYIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIAKEILVNEEDIKGYR
VTSTGKPEFTNLKVYHDIKDITARKEIIENAELLDQIAKILTIYQSSEDIQEELTNLNSELTQE
EIEQISNLKGYTGTHNLSLKAINLILDELWHTNDNQIAIFNRLKLVPKKVDLSQQKEIPTTLVD
DFILSPVVKRSFIQSIKVINAIIKKYGLPNDIIIELAREKNSKDAQKMINEMQKRNRQTNERIE
EIIRTTGKENAKYLIEKIKLHDMQEGKCLYSLEAIPLEDLLNNPFNYEVDHIIPRSVSEDNSEN
NKVLVKQEENSKKGNRTPFQYLSSSDSKISYETFKKHILNLAKGKGRISKTKKEYLLEERDINR
FSVQKDFINRNLVDTRYATRGLMNLLRSYFRVNNLDVKVKSINGGFTSFLRRKWKFKKERNKGY
KHHAEDALIIANADFIFKEWKKLDKAKKVMENQMFEEKQAESMPEIETEQEYKEIFITPHQIKH
IKDFKDYKYSHRVDKKPNRELINDTLYSTRKDDKGNTLIVNNLNGLYDKDNDKLKKLINKSPEK
LLMYHHDPQTYQKLKLIMEQYGDEKNPLYKYYEETGNYLTKYSKKDNGPVIKKIKYYGNKLNAH
LDITDDYPNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPRIIK
TIASKTQSIKKYSTDILGNLYEVKSKKHPQIIKKGEGADKRTADGSEFESPKKKRKV;
(vi) comprises an amino acid sequence that is at least 80%, 85%, 90%, 95%, 96%, 97%, 98%, 99%, or 100% identical to:
saBE4
(SEQ ID NO: 447)
MSSETGPVAVDPTLRRRIEPHEFEVFFDPRELRKETCLLYEINWGGRHSIWRHTSQNTNKHVEV
NFIEKFTTERYFCPNTRCSITWFLSWSPCGECSRAITEFLSRYPHVTLFIYIARLYHHADPRNR
QGLRDLISSGVTIQIMTEQESGYCWRNFVNYSPSNEAHWPRYPHLWVRLYVLELYCIILGLPPC
LNILRRKQPQLTFFTIALQSCHYQRLPPHILWATGLKSGGSSGGSSGSETPGTSESATPESSGG
SSGGSGKRNYILGLAIGITSVGYGIIDYETRDVIDAGVRLFKEANVENNEGRRSKRGARRLKRR
RRHRIQRVKKLLFDYNLLTDHSELSGINPYEARVKGLSQKLSEEEFSAALLHLAKRRGVHNVNE
VEEDTGNELSTKEQISRNSKALEEKYVAELQLERLKKDGEVRGSINRFKTSDYVKEAKQLLKVQ
KAYHQLDQSFIDTYIDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAKEILVNEEDIKGYRV
TSTGKPEFTNLKVYHDIKDITARKEIIENAELLDQIAKILTIYQSSEDIQEELTNLNSELTQEE
IEQISNLKGYTGTHNLSLKAINLILDELWHTNDNQIAIFNRLKLVPKKVDLSQQKEIPTTLVDD
FILSPVVKRSFIQSIKVINAIIKKYGLPNDIIIELAREKNSKDAQKMINEMQKRNRQTNERIEE
IIRTTGKENAKYLIEKIKLHDMQEGKCLYSLEAIPLEDLLNNPFNYEVDHIIPRSVSEDNSENN
KVLVKQEENSKKGNRTPFQYLSSSDSKISYETFKKHILNLAKGKGRISKTKKEYLLEERDINRF
SVQKDFINRNLVDTRYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRELINDTLYSTRKDDKGNTLIVNNLNGLYDKDNDKLKKLINKSPEKL
LMYHHDPQTYQKLKLIMEQYGDEKNPLYKYYEETGNYLTKYSKKDNGPVIKKIKYYGNKLNAHL
DITDDYPNSRNKVVKLSLKPYRFDVYLDNGVYKFVTVKNLDVIKKENYYEVNSKCYEEAKKLKK
ISNQAEFIASFYNNDLIKINGELYRVIGVNNDLLNRIEVNMIDITYREYLENMNDKRPPRIIKT
IASKTQSIKKYSTDILGNLYEVKSKKHPQIIKKGGSPKKKRKVSSDYKDHDGDYKDHDIDYKDD
DDKSGGSGGSGGSTNLSDIIEKETGKQLVIQESILMLPEEVEEVIGNKPESDILVHTAYDESTD
ENVMLLTSDAPEYKPWALVIQDSNGENKIKMLSGGSGGSGGSTNLSDIIEKETGKQLVIQESIL
MLPEEVEEVIGNKPESDILVHTAYDESTDENVMLLTSDAPEYKPWALVIQDSNGENKIKMLSGG
SKRTADGSEFESPKKKRKVE;
(vii) comprises an amino acid sequence that is at least 80%, 85%, 90%, 95%, 96%, 97%, 98%, 99%, or 100% identical to:
saBE4-KKH
(SEQ ID NO: 448)
MSSETGPVAVDPTLRRRIEPHEFEVFFDPRELRKETCLLYEINWGGRHSIWRHTSQNTNKHVEV
NFIEKFTTERYFCPNTRCSITWFLSWSPCGECSRAITEFLSRYPHVTLFIYIARLYHHADPRNR
QGLRDLISSGVTIQIMTEQESGYCWRNFVNYSPSNEAHWPRYPHLWVRLYVLELYCIILGLPPC
LNILRRKQPQLTFFTIALQSCHYQRLPPHILWATGLKSGGSSGGSSGSETPGTSESATPESSGG
SSGGSGKRNYILGLAIGITSVGYGIIDYETRDVIDAGVRLFKEANVENNEGRRSKRGARRLKRR
RRHRIQRVKKLLFDYNLLTDHSELSGINPYEARVKGLSQKLSEEEFSAALLHLAKRRGVHNVNE
VEEDTGNELSTKEQISRNSKALEEKYVAELQLERLKKDGEVRGSINRFKTSDYVKEAKQLLKVQ
KAYHQLDQSFIDTYIDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAKEILVNEEDIKGYRV
TSTGKPEFTNLKVYHDIKDITARKEIIENAELLDQIAKILTIYQSSEDIQEELTNLNSELTQEE
IEQISNLKGYTGTHNLSLKAINLILDELWHTNDNQIAIFNRLKLVPKKVDLSQQKEIPTTLVDD
FILSPVVKRSFIQSIKVINAIIKKYGLPNDIIIELAREKNSKDAQKMINEMQKRNRQTNERIEE
IIRTTGKENAKYLIEKIKLHDMQEGKCLYSLEAIPLEDLLNNPENYEVDHIIPRSVSEDNSENN
KVLVKQEENSKKGNRTPFQYLSSSDSKISYETFKKHILNLAKGKGRISKTKKEYLLEERDINRF
SVQKDFINRNLVDTRYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRKLINDTLYSTRKDDKGNTLIVNNLNGLYDKDNDKLKKLINKSPEKL
LMYHHDPQTYQKLKLIMEQYGDEKNPLYKYYEETGNYLTKYSKKDNGPVIKKIKYYGNKLNAHL
DITDDYPNSRNKVVKLSLKPYRFDVYLDNGVYKFVTVKNLDVIKKENYYEVNSKCYEEAKKLKK
ISNQAEFIASFYKNDLIKINGELYRVIGVNNDLLNRIEVNMIDITYREYLENMNDKRPPHIIKT
IASKTQSIKKYSTDILGNLYEVKSKKHPQIIKKGGSPKKKRKVSSDYKDHDGDYKDHDIDYKDD
DDKSGGSGGSGGSTNLSDIIEKETGKQLVIQESILMLPEEVEEVIGNKPESDILVHTAYDESTD
ENVMLLTSDAPEYKPWALVIQDSNGENKIKMLSGGSGGSGGSTNLSDIIEKETGKQLVIQESIL
MLPEEVEEVIGNKPESDILVHTAYDESTDENVMLLTSDAPEYKPWALVIQDSNGENKIKMLSGG
SKRTADGSEFESPKKKRKVE;
or
(viii) comprises an amino acid sequence that is at least 80%, 85%, 90%, 95%, 96%, 97%, 98%, 99%, or 100% identical to:
ABE-bhCAS12b
(SEQ ID NO: 449)
MSEVEFSHEYWMRHALTLAKRARDEREVPVGAVLVLNNRVIGEGWNRAIGLHDPTAHAEIMALR
QGGLVMQNYRLYDATLYVTFEPCVMCAGAMIHSRIGRVVFGVRNAKTGAAGSLMDVLHHPGMNH
RVEITEGILADECAALLCRFFRMPRRVENAQKKAQSSTDGSSGSETPGTSESATPESSGAPKKK
RKVGIHGVPAAATRSFILKIEPNEEVKKGLWKTHEVLNHGIAYYMNILKLIRQEAIYEHHEQDP
KNPKKVSKAEIQAELWDFVLKMQKCNSFTHEVDKDEVENILRELYEELVPSSVEKKGEANQLSN
KFLYPLVDPNSQSGKGTASSGRKPRWYNLKIAGDPSWEEEKKKWEEDKKKDPLAKILGKLAEYG
LIPLFIPYTDSNEPIVKEIKWMEKSRNQSVRRLDKDMFIQALERFLSWESWNLKVKEEYEKVEK
EYKTLEERIKEDIQALKALEQYEKERQEQLLRDTLNTNEYRLSKRGLRGWREIIQKWLKMDENE
PSEKYLEVEKDYQRKHPREAGDYSVYEFLSKKENHFIWRNHPEYPYLYATFCEIDKKKKDAKQQ
ATFTLADPINHPLWVRFEERSGSNLNKYRILTEQLHTEKLKKKLTVQLDRLIYPTESGGWEEKG
KVDIVLLPSRQFYNQIFLDIEEKGKHAFTYKDESIKFPLKGTLGGARVQFDRDHLRRYPHKVES
GNVGRIYENMTVNIEPTESPVSKSLKIHRDDFPKVVNFKPKELTEWIKDSKGKKLKSGIESLEI
GLRVMSIALGQRQAAAASIFEVVDQKPDIEGKLFFPIKGTELYAVHRASFNIKLPGETLVKSRE
VLRKAREDNLKLMNQKLNFLRNVLHFQQFEDITEREKRVTKWISRQENSDVPLVYQDELIQIRE
LMYKPYKDWVAFLKQLHKRLEVEIGKEVKHWRKSLSDGRKGLYGISLKNIDEIDRTRKELLRWS
LRPTEPGEVRRLEPGQRFAIDQLNHLNALKEDRLKKMANTIIMHALGYCYDVRKKKWQAKNPAC
QIILFEDLSNYNPYKERSRFENSRLMKWSRREIPRQVALQGEIYGLQVGEVGAQFSSRFHAKTG
SPGIRCRVVTKEKLQDNRFFKNLQREGRLTLDKIAVLKEGDLYPDKGGEKFISLSKDRKCVTTH
ADINAAQNLQKRFWTRTHGFYKVYCKAYQVDGQTVYIPESKDQKQKIIEEFGEGYFILKDGVYE
WVNAGKLKIKKGSSKQSSSELVDSDILKDSFDLASELKGEKLMLYRDPSGNVEPSDKWMAAGVE
FGKLERILISKLTNQYSISTIEDDSSKQSMKRPAATKKAGQAKKKK.

23. The composition of claim 20, wherein the guide RNA(s) comprises a nucleotide sequence selected from one or more of those sequences listed in Table 1, Table 2A, or Table 2B; or any of the aforementioned sequences wherein 1, 2, 3, 4, or 5 nucleotides is deleted from the 5′ and/or 3′ terminus of the nucleotide sequence.

24. The composition of claim 20, wherein the guide RNA(s) comprises a nucleotide sequence selected from the group consisting of:
(SEQ ID NO: 408; sgRNA_361/gRNA1598)
5′-UAUAGGAAAACCAGUGAGUC-3′;
(SEQ ID NO: 409; sgRNA_362/gRNA1599)
5′-UACUCACCUCUGCAUGCUCA-3′;
(SEQ ID NO: 410; sgRNA_363/gRNA1606)
5′-ACUCACCUCUGCAUGCUCAU-3′;
(SEQ ID NO: 412; sgRNA_365)
5′-AUACUCACCUCUGCAUGCUCA-3′;
(SEQ ID NO: 414; sgRNA_367/gRNA-#19)
5′-UUGGCAGGAUGGCUUCUCAUCG-3′;
(SEQ ID NO: 422; sgRNA_375)
5′-GGCUAUCGUCACCAAUCCCA-3′;
(SEQ ID NO: 423; sgRNA_376)
5′-GCUAUCGUCACCAAUCCCAA-3′;
(SEQ ID NO: 561; gRNA1604)
5′-ACACCUUAUAGGAAAACCAG-3′;
(SEQ ID NO: 554; gRNA1597)
5′-CUCUCAUAGGUGGUAUUCAC-3′;
(SEQ ID NO: 557; gRNA1600)
5′-GCAACUUACCCAGAGGCAAA-3′;
(SEQ ID NO: 551; gRNA1594)
5′-CAACUUACCCAGAGGCAAAU-3′;
(SEQ ID NO: 558; gRNA1601)
5′-UCUGUAUACUCACCUCUGCA-3′;
(SEQ ID NO: 462; gRNA1756)
5′-CAAAUAUGAACCUUGUCUAG-3′;
(SEQ ID NO: 470; gRNA1764)
5′-GAACCUUGUCUAGAGAGAUU-3′;
(SEQ ID NO: 492; gRNA1786)
5′-UGAGUAUAAAAGCCCCAGGC-3′;
and
(SEQ ID NO: 478; gRNA1772)
5′-GCCAUCCUGCCAAGAAUGAG-3′;
(SEQ ID NO: 409; sgRNA_362/gRNA1599)
5′-UACUCACCUCUGCAUGCUCA-3′,
(SEQ ID NO: 410; sgRNA_363/gRNA1606)
5′-ACUCACCUCUGCAUGCUCAU-3′,
(SEQ ID NO: 411; sgRNA_364)
5′-UACCACCUAUGAGAGAAGAC-3′,
(SEQ ID NO: 412; sgRNA_365)
5′-AUACUCACCUCUGCAUGCUCA-3′,
(SEQ ID NO: 413; sgRNA_366)
5′-ACUGGUUUUCCUAUAAGGUGU-3′,
(SEQ ID NO: 551; gRNA1594)
5′-CAACUUACCCAGAGGCAAAU-3′,
and
(SEQ ID NO: 496; gRNA1790)
5′-UGUUGACUAAGUCAAUAAUC-3′;
or any of the aforementioned sequences wherein 1, 2, 3, 4, or 5 nucleotides is deleted from the 5′ and/or 3′ terminus of the nucleotide sequence.

25. A composition comprising one or more polynucleotides encoding an endonuclease and a guide RNA, wherein the guide RNA comprises a nucleic acid sequence that is complementary to a transthyretin (TTR) polynucleotide, and wherein the endonuclease comprises the amino acid sequence:
bhCas12b
(SEQ ID NO: 450)
v4MAPKKKRKVGIHGVPAAATRSFILKIEPNEEVKKGLWKTHEVLNHGIAYYMNILKLIRQEAI
YEHHEQDPKNPKKVSKAEIQAELWDFVLKMQKCNSFTHEVDKDEVENILRELYEELVPSSVEKK
GEANQLSNKFLYPLVDPNSQSGKGTASSGRKPRWYNLKIAGDPSWEEEKKKWEEDKKKDPLAKI
LGKLAEYGLIPLFIPYTDSNEPIVKEIKWMEKSRNQSVRRLDKDMFIQALERFLSWESWNLKVK
EEYEKVEKEYKTLEERIKEDIQALKALEQYEKERQEQLLRDTLNTNEYRLSKRGLRGWREIIQK
WLKMDENEPSEKYLEVFKDYQRKHPREAGDYSVYEFLSKKENHFIWRNHPEYPYLYATFCEIDK
KKKDAKQQATFTLADPINHPLWVRFEERSGSNLNKYRILTEQLHTEKLKKKLTVQLDRLIYPTE
SGGWEEKGKVDIVLLPSRQFYNQIFLDIEEKGKHAFTYKDESIKFPLKGTLGGARVQFDRDHLR
RYPHKVESGNVGRIYFNMTVNIEPTESPVSKSLKIHRDDFPKVVNFKPKELTEWIKDSKGKKLK
SGIESLEIGLRVMSIDLGQRQAAAASIFEVVDQKPDIEGKLFFPIKGTELYAVHRASENIKLPG
ETLVKSREVLRKAREDNLKLMNQKLNFLRNVLHFQQFEDITEREKRVTKWISRQENSDVPLVYQ
DELIQIRELMYKPYKDWVAFLKQLHKRLEVEIGKEVKHWRKSLSDGRKGLYGISLKNIDEIDRT
RKFLLRWSLRPTEPGEVRRLEPGQRFAIDQLNHLNALKEDRLKKMANTIIMHALGYCYDVRKKK
WQAKNPACQIILFEDLSNYNPYGERSRFENSRLMKWSRREIPRQVALQGEIYGLQVGEVGAQFS
SRFHAKTGSPGIRCRVVTKEKLQDNRFFKNLQREGRLTLDKIAVLKEGDLYPDKGGEKFISLSK
DRKCVTTHADINAAQNLQKRFWTRTHGFYKVYCKAYQVDGQTVYIPESKDQKQKIIEEFGEGYF
ILKDGVYEWVNAGKLKIKKGSSKQSSSELVDSDILKDSFDLASELKGEKLMLYRDPSGNVEPSD
KWMAAGVFFGKLERILISKLTNQYSISTIEDDSSKQSMSGGSKRTADGSEFESPKKKRKVE,
wherein said guide RNA targets said endonuclease to effect a double-stranded break of the TTR polynucleotide sequence.

26. The composition of claim 25, wherein the guide RNA comprises a nucleotide sequence, selected from the group consisting of:
(SEQ ID NO: 415; sgRNA_368)
5′-UCCUAUAAGGUGUGAAAGUCUG-3′,
(SEQ ID NO: 416; sgRNA_369)
5′-UGAGCCCAUGCAGCUCUCCAGA-3′,
(SEQ ID NO: 417; sgRNA_370)
5′-CUCCUCAGUUGUGAGCCCAUGC-3′,
(SEQ ID NO: 418; sgRNA_371)
5′-GUAGAAGGGAUAUACAAAGUGG-3′,
(SEQ ID NO: 419; sgRNA_372)
5′-CCACUUUGUAUAUCCCUUCUAC-3′,
(SEQ ID NO: 420; sgRNA_373)
5′-GGUGUCUAUUUCCACUUUGUAU-3′,
and
(SEQ ID NO: 421; sgRNA_374)
5′-CAUGAGCAUGCAGAGGUGAGUA-3′;
or any of the aforementioned sequences wherein nucleobases 1, 2, 3, 4, or 5 nucleotides is deleted from the 5′ and/or 3′ terminus of the nucleotide sequence.

27. A pharmaceutical composition for the treatment of transthyretin (TTR) amyloidosis, the pharmaceutical composition comprising: an endonuclease, or a nucleic acid encoding the endonuclease, and a guide RNA (gRNA) comprising a nucleic acid sequence complementary to an transthyretin (TTR) polynucleotide in a pharmaceutically acceptable excipient, wherein the endonuclease comprises the amino acid sequence:
bhCas12b
(SEQ ID NO: 450)
v4MAPKKKRKVGIHGVPAAATRSFILKIEPNEEVKKGLWKTHEVLNHGIAYYMNILKLIRQEAI
YEHHEQDPKNPKKVSKAEIQAELWDFVLKMQKCNSFTHEVDKDEVENILRELYEELVPSSVEKK
GEANQLSNKFLYPLVDPNSQSGKGTASSGRKPRWYNLKIAGDPSWEEEKKKWEEDKKKDPLAKI
LGKLAEYGLIPLFIPYTDSNEPIVKEIKWMEKSRNQSVRRLDKDMFIQALERFLSWESWNLKVK
EEYEKVEKEYKTLEERIKEDIQALKALEQYEKERQEQLLRDTLNTNEYRLSKRGLRGWREIIQK
WLKMDENEPSEKYLEVFKDYQRKHPREAGDYSVYEFLSKKENHFIWRNHPEYPYLYATFCEIDK
KKKDAKQQATFTLADPINHPLWVRFEERSGSNLNKYRILTEQLHTEKLKKKLTVQLDRLIYPTE
SGGWEEKGKVDIVLLPSRQFYNQIFLDIEEKGKHAFTYKDESIKFPLKGTLGGARVQFDRDHLR
RYPHKVESGNVGRIYFNMTVNIEPTESPVSKSLKIHRDDFPKVVNFKPKELTEWIKDSKGKKLK
SGIESLEIGLRVMSIDLGQRQAAAASIFEVVDQKPDIEGKLFFPIKGTELYAVHRASENIKLPG
ETLVKSREVLRKAREDNLKLMNQKLNFLRNVLHFQQFEDITEREKRVTKWISRQENSDVPLVYQ
DELIQIRELMYKPYKDWVAFLKQLHKRLEVEIGKEVKHWRKSLSDGRKGLYGISLKNIDEIDRT
RKFLLRWSLRPTEPGEVRRLEPGQRFAIDQLNHLNALKEDRLKKMANTIIMHALGYCYDVRKKK
WQAKNPACQIILFEDLSNYNPYGERSRFENSRLMKWSRREIPRQVALQGEIYGLQVGEVGAQFS
SRFHAKTGSPGIRCRVVTKEKLQDNRFFKNLQREGRLTLDKIAVLKEGDLYPDKGGEKFISLSK
DRKCVTTHADINAAQNLQKRFWTRTHGFYKVYCKAYQVDGQTVYIPESKDQKQKIIEEFGEGYF
ILKDGVYEWVNAGKLKIKKGSSKQSSSELVDSDILKDSFDLASELKGEKLMLYRDPSGNVEPSD
KWMAAGVFFGKLERILISKLTNQYSISTIEDDSSKQSMSGGSKRTADGSEFESPKKKRKVE,
wherein said guide RNA targets said endonuclease to effect a double-stranded break of the TTR polynucleotide sequence wherein the guide RNA(s) comprises a nucleotide sequence selected from one or more of those sequences listed in Table 1, Table 2A, or Table 2B; or any of the aforementioned sequences wherein 1, 2, 3, 4, or 5 nucleotides is deleted from the 5′ and/or 3′ terminus of the nucleotide sequence.

28. A method for treating amyloidosis in a subject, the method comprising systemically administering to the subject a guide RNA and a fusion protein comprising a polynucleotide programmable DNA binding domain and a deaminase domain, wherein said guide RNA targets said base editor to effect an alteration of a nucleobase of the TTR polynucleotide sequence present in a liver cell of the subject.
